(19) United States
(12) Patent Application Publication
(10) Pub. No.: US 2021/0000119 A1
(43) Pub. Date: Jan. 7, 2021

(54) SYNTHESIS AND ANTI-CANCER ACTIVITY OF COMMUNESIN ALKALOIDS

(71) Applicant: Massachusetts Institute of Technology, Cambridge, MA (US)

(72) Inventors: Mohammad Movassaghi, Lincoln, MA (US); Matthew M. Pompeo, Cambridge, MA (US); Jaime Cheah, Medford, MA (US)

(21) Appl. No.: 16/863,015

(22) Filed: Apr. 30, 2020

Related U.S. Application Data
(60) Provisional application No. 62/869,382, filed on Jul. 1, 2019.

(51) Int. Cl. A01N 43/90; C07D 471/22; C07D 519/00

(52) U.S. Cl. CPC A01N 43/90; C07D 519/00; C07D 471/22

(57) ABSTRACT

Described herein are compounds of Formula (I):

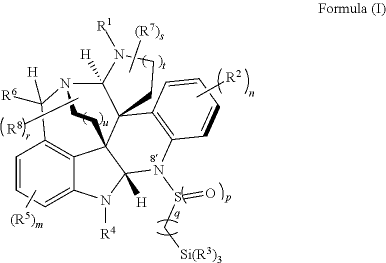

and salts, tautomers, and stereoisomers thereof. Methods of making the compounds, salts, tautomers, and stereoisomers are also described. Further described herein are composition, kits, methods, and uses involving the compounds, salts, tautomers, and stereoisomers. The methods include methods of treating and preventing diseases (e.g., cancers) in subjects (e.g., humans).

### RELATED APPLICATIONS

The present application claims priority under 35 U.S.C. § 119(e) to U.S. provisional application, U.S. Ser. No. 62/869,382, filed Jul. 1, 2019, which is incorporated herein by reference.

### GOVERNMENT SUPPORT

This invention was made with Government support under Grant No. R01 GM089732 awarded by the National Institutes of Health (NIH). The Government has certain rights in the invention.

### BACKGROUND

The communesin alkaloids are a family of nine structurally complex natural products isolated from various marine and terrestrial Penicillium fungi (FIG. 1). Some members have been shown to possess insecticidal and antiproliferative activities, as well as significant cytotoxicity against lymphocytic leukemia. The core structures of these alkaloids feature seven contiguous rings, two sensitive aminal linkages, and up to six stereogenic centers, of which two are vicinal and quaternary (C3a/C3a′). This formidable structural complexity coupled with an array of important biological properties initiated a burst of research activity directed towards their total chemical synthesis, culminating in solutions for the preparation of racemic¹ and enantioenriched² samples of communesins.

Communesins A (2) and B (4), first isolated in 1993 bp Numata were found to exhibit moderate to potent cytotoxicity against cultured mouse P-388 lymphocytic leukemia cells (ED₅₀=3.5 μg/mL and 0.45 μg/mL, respectively) (Numata, A.; Takahashi, C.; Ito, Y.; Takada, T.; Kawai, K.; Usami, Y.; Imachi, M.; Ito, T.; Hasegawa, T. Tetrahedron Lett. 1993, 34, 2355-2358). In 2004, Jadulco and co-workers isolated communesins C (5) and D (6) and, together with 4, were shown to possess moderate anti-proliferative activity against an array of human leukemia cell lines. Furthermore, compounds 4, 5 and 6 exhibited toxicity against the brine shrimp Artemia salina with LD₅₀ values of 0.30, 1.96, and 0.57 μg/mL, respectively. (Jadulco, R.; Edrada, R. A.; Ebel, R.; Berg, A.; Schaumann, K.; Wray, V.; Steube, K.; Proksch, P. J. Nat. Prod. 2004, 67, 78-81).

Later in 2004, Hayashi and co-workers isolated communesins E (3) and F (1) and studied the insecticidal properties of these new derivatives together with 2, 4, and 6 (Hayashi, H.; Matsumoto, H.; Akiyama, K. Biosci. Biotechnol. Biochem. 2004, 68, 753-756). Communesin B (4) was found to be the most active against third instar larvae of silkworms with an LD₅₀ value of 5 μg/g of diet by oral administration. Communesins A (2), D (6), E (3), and F (1) were found to exhibit lower insecticidal activities.

Recently, in 2015, Fan and co-workers isolated communesin I (9) and studied the cardiovascular effects of this new alkaloid, together with co-isolates 2 and 4 (Fan, Y.-Q.; Li, P.-H.; Chao, Y.-X.; Chen, H.; Du, N.; He, Q.-X.; Liu, K.-C. Mar. Drugs. 2015, 13, 6489-6504). All three compounds showed a mitigative effect on bradycardia caused by astemidazole at different concentrations. In addition, communesins I (9) and A (2) exhibited moderate vasculogenetic activity. Finally, compounds 9 and 2 were found to moderately promote the function of cardiovascular vessels.

To date, the total synthesis of (±)-communesin F (1) has been completed by Qin, Weinreb, and Funk, in addition to a formal synthesis by Stoltz. Ma's total synthesis of (−)-communesin F (1) was the first enantioselective solution for this archetypical alkaloid. However, these total syntheses were complex, low yielding, and did not readily lend themselves to the synthesis of analogs or derivatives, which would be necessary to support a rational drug development program.

A concise enantioselective total syntheses of several representative communesins, ready for adaption toward a wide range of analogs, and (−)-communsein F was recently reported.^{2b} The highly convergent route established methods that allowed for unprecedented efficiency in constructing the complex heptacyclic ring system from two densely functionalized building blocks. In addition, the use of flexible stereochemical control elements enabled access to any selected enantiomer or diastereomer without dramatic alterations to the strategy, and was easily generalized and applied to the synthesis of a wide variety of analogs. This novel chemical synthesis allowed, for the first time, the opportunity to fully explore the promising biological properties of this class of compounds.

Despite this remarkable progress, efficient access to the more complex epoxide-containing analogues continues to remain a challenge. Indeed, since Zuo and Ma's pioneering 2011 total synthesis of (−)-2 and (−)-4,³ no further reports describing the synthesis of sensitive epoxy-communesins 2.10 have been disclosed. Therefore, in order to fully evaluate the efficacy of these structurally unprecedented alkaloids in the treatment of human disease, a unified and convergent synthesis is needed to provide all members of the communesin family and related complex derivatives.

### SUMMARY OF THE INVENTION

In one aspect, the present disclosure provides derivatized communesin alkaloids, including epoxy-communesins, and the synthesis thereof. These compounds may be biologically active and used to treat and prevent diseases. In some aspects, the epoxy-communesins may be advantageous over known and/or natural communesins for treating or preventing diseases. In another aspect, the present disclosure provides compositions, kits, methods of preparation, and methods of use including methods of treating and preventing diseases.

In one aspect, the present disclosure provides compounds of Formula (I):

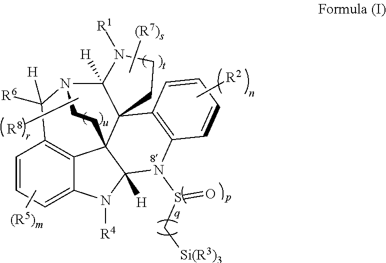

or a salt, tautomer or stereoisomer thereof, wherein R¹, R², R³, R₄, R⁵, R⁶, R⁷, R⁸, m, n, p, q, r, s, t, and u are as described herein.

In another aspect, the present disclosure also provides compounds of Formula (V):

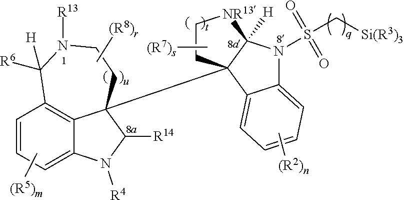

or a salt, tautomer or stereoisomer thereof wherein R¹³, R^{13′}, R¹⁴, R², R³, R⁴, R⁵, R⁶, R⁷, R⁸, m, n, q, r, s, t, and u are as described herein.

Also provided herein are compounds of Formula (III′):

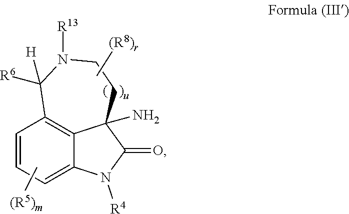

or a salt, tautomer or stereoisomer thereof wherein R¹³, R¹⁵, R¹⁶, R⁴, R⁵, R⁶, R⁸, K X, m, r, and u are as described herein.

Other aspects of the disclosure provide compounds of Formula (XIV):

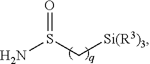

or a salt, tautomer, or stereoisomer thereof, wherein R³ and q are as defined herein.

The present disclosure also provides methods of making a compound of Formula (I), methods of making a compound of Formula (I) from a compound of Formula (V), methods of making a compound of Formula (V), methods of making a compound of Formula (V) comprising a compound of Formula (III′), methods of making a compound of Formula (III′), and other methods of synthesis.

Also provided herein are methods of making a compound of Formula (I′):

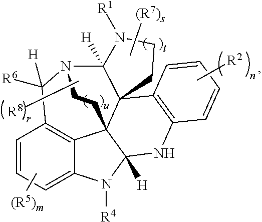

or a salt, tautomer, or stereoisomer thereof, wherein R¹, R², R³, R⁵, R⁶, R⁷, R⁸, m, n, p, q, r, s, t, and u are as described herein.

In one embodiment, the present disclosure relates to a pharmaceutical composition comprising a compound as described herein and a pharmaceutically acceptable excipient.

In another embodiment, the present disclosure provides a method of treating a disease comprising administering an effective amount of the pharmaceutical composition to a subject. In some embodiments, the disease is cancer. In other embodiments, the disease is a bacterial infection. In other embodiments, the disease is a fungal infection. In another embodiment, the disease is a viral infection. In still other embodiments, the disease is abnormal cardiovascular function. In yet another embodiment, the pharmaceutical compositions are used to treat insect infestations.

The details of certain embodiments of the disclosure are set forth in the Detailed Description of Certain Embodiments, as described below. Other features, objects, and advantages of the disclosure will be apparent from the Definitions, Figures, Examples, and Claims. It should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

### BRIEF DESCRIPTION OF THE FIGURES

The skilled artisan will understand that the drawings primarily are for illustrative purposes and are not intended to limit the scope of the subject matter described herein.

FIG. 1 shows the chemical structures of the communesin alkaloids.

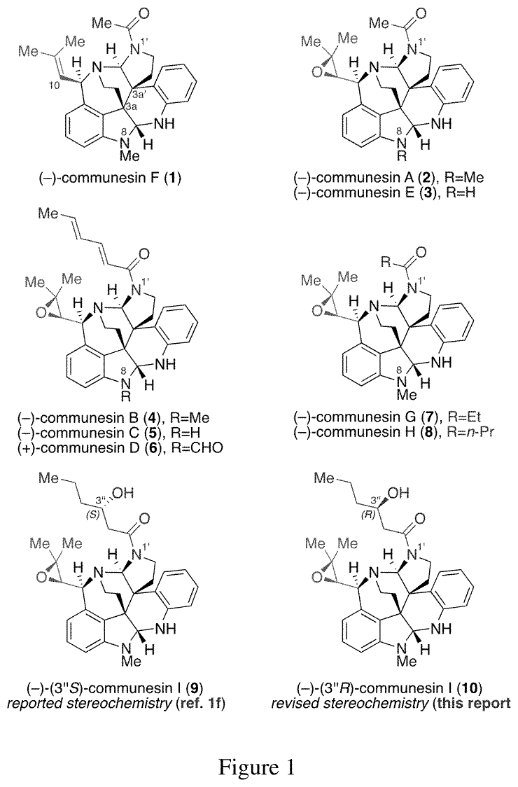

FIG. 2 shows the synthesis of all known epoxy-communesin alkaloids and the stereochemical revision of (−)-communesin I (10). Reagents and Conditions: (a) t-BuOLi, EtOH 60° C.; PPTS, Ac₂O, 23° C., 82%. (b) t-BuOLi, EtOH, 60° C.; PPTS, sorbic anhydride, 23° C., 82%. (c) t-BuOLi, EtOH, 60° C.; PPTS, propionic anhydride, 23° C., 86% (d) t-BuOLi, EtOH, 60° C.; PPTS, butyric anhydride, 23° C., 84%. (e) t-BuOLi, EtOH, 60° C.; PPTS, (+)-48, 23° C., 84%. (f) t-BuOLi, EtOH, 60° C.; PPTS, (+)-49, 23° C., 48%. (g) pyridinium dichromate (PDC), K₂CO₃, 1,2-dichloroethane, 60° C. (h) TASF, DMF, 23° C. (i) (i) KOH, H₂O-DMSO; (ii) TASF, DMF, 45° C. (j) (i) KOH, H₂O-DMSO; (ii) TASF, DMF, 23° C. In the ORTEP representations of sulfonamide (−)-42 and (−)-44, the thermal ellipsoids are drawn at 30% probability.

### DETAILED DESCRIPTION OF CERTAIN ASPECTS OF THE INVENTION

In the following description, certain specific details are set forth in order to provide a thorough understanding of various embodiments. However, one skilled in the art will understand that the subject matter described herein can be practiced without these details. In other instances, well-known structures have not been shown or described in detail to avoid unnecessarily obscuring descriptions of the embodiments.

### Definitions

For convenience, certain terms employed herein, in the specification, examples and appended claims are collected herein.

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 93^{rd} Ed. Additionally, general principles of organic chemistry are described in “Organic Chemistry”, 2^{nd} Ed, Thomas N. Sorrell, University Science Books, Sausalito: 2005, and “March's Advanced Organic Chemistry”, 6^{th} Ed., a Smith, M. B. and March, J., John Wiley & Sons, New York: 2007, the entire contents of which are hereby incorporated by reference.

For convenience, certain terms employed herein, in the specification, examples and appended claims are collected herein.

Unless otherwise required by context, singular terms shall include pluralities, and plural terms shall include the singular.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2% or 1% of a given value or range of values.

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can include one or more asymmetric centers or stereogenic centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGrawHill, N.Y., 1962); and Wilen, S. H. Tables of Resolving Agents and Optical Resolutions p. 268 (E. L. Eliel, Ed., Univ. of Notre Dame Press, Notre Dame, Ind. 1972). The disclosure additionally encompasses compounds as individual isomers substantially free of other isomers, and alternatively, as mixtures of various isomers.

When a range of values is listed, it is intended to encompass each value and sub range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C2), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in CH). The moieties ═CH₂ and ═CH are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include CF₃, CF₂CF₃, CF₂CF₂CF₃, CCl₃, CFCl₂, CF₂Cl, and the like.

“Alkenyl” refers to a radical of a straightchain or branched hydrocarbon group having from 2 to 20 carbon atoms, one or more (e.g., two, three, or four, as valency permits) carboncarbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carboncarbon double bonds can be internal (such as in 2-butenyl) or terminal (such as in 1-butenyl). Examples of C₂₋₄ alkenyl groups include ethenyl (C2), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

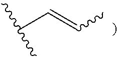

may be in the (E)- or (Z)-configuration.

“Alkynyl” refers to a radical of a straightchain or branched hydrocarbon group having from 2 to 20 carbon atoms, one or more (e.g., two, three, or four, as valency permits) carboncarbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂-4 alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carboncarbon triple bonds can be internal (such as in 2-butynyl) or terminal (such as in 1-butynyl). Examples of C₂₋₄ alkynyl groups include ethynyl (C2), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C1-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

The term “amine” or “amino” refers to the group —NH— or —NH₂.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6.14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. The term “aryl” used alone or as part of a larger moiety as in “aralkyl,” “aralkoxy,” or “aryloxyalkyl,” refers to monocyclic, bicyclic or polycyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more nonaromatic rings, such as indanyl, phthalimidyl, naphthimidyl, phenanthridinyl, or tetrahydronaphthyl, and the like.

“Aralkyl” refers to a radical of the formula —R_{b}-R_{c}, where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. In certain embodiments, carbocyclic rings can comprise from 3 to 20 carbon atoms in the ring. The term “carbocyclyl” or “carbocyclic” or “cycloalkyl” refers to a radical of a nonaromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”), 3 to 7 ring carbon atoms (“C₃₋₇ carbocyclyl”), 3 to 6 ring carbon atoms (“C₃₋₆ carbocyclyl”), 4 to 6 ring carbon atoms (“C₄₋₆ carbocyclyl”), 5 to 6 ring carbon atoms (“C₅₋₆ carbocyclyl”), or 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carboncarbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents.

In certain embodiments, “cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

The term “halo” or “halogen” refers to fluorine (fluoro, —F), chlorine (chloro, —Cl), bromine (bromo, —Br), or iodine (iodo, —I).

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkenyl group can be optionally substituted.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl” or “C₁₋₂₀ heteroalkyl”). In certain embodiments, a heteroalkyl group refers to a saturated group having from 1 to 12 carbon atoms and 1 or more heteroatoms within the parent chain (“heteroC₁₋₁₂ alkyl”). In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and for 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroCi alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl” or “C₂₋₂₀ heteroalkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and for 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl” or “C₂₋₂₀ heteralkynyl”). In certain embodiments, a heteroalkynyl group refers to a group having from 2 to 12 carbon atoms, at least one triple bond, and 1 or more heteroatoms within the parent chain (“heteroC₂₋₁₂ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and for 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. The term “heterocyclyl” or “heterocyclic” refers to a radical of a 3 to 14-membered nonaromatic ring system having ring carbon atoms and 1 to 4 ring heteroatoms, wherein each heteroatom is independently selected from nitrogen, oxygen, phosphorus, and sulfur (“3.14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carboncarbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

In some embodiments, a heterocyclyl group is a 5.10 membered nonaromatic ring system having ring carbon atoms and 1.4 ring heteroatoms, wherein each heteroatom is independently selected from nitrogen, oxygen, phosphorus, and sulfur (“5.10 membered heterocyclyl”). In some embodiments, a heterocyclyl group is a 5.8 membered nonaromatic ring system having ring carbon atoms and 1.4 ring heteroatoms, wherein each heteroatom is independently selected from nitrogen, oxygen, phosphorus, and sulfur (“5.8 membered heterocyclyl”). In some embodiments, a heterocyclyl group is a 5.6 membered nonaromatic ring system having ring carbon atoms and 1.4 ring heteroatoms, wherein each heteroatom is independently selected from nitrogen, oxygen, phosphorus, and sulfur (“5.6 membered heterocyclyl”). In some embodiments, the 5.6 membered heterocyclyl has 1.3 ring heteroatoms selected from nitrogen, oxygen, phosphorus, and sulfur. In some embodiments, the 5-6 membered heterocyclyl has 1.2 ring heteroatoms selected from nitrogen, oxygen, phosphorus, and sulfur. In some embodiments, the 5.6 membered heterocyclyl has 1 ring heteroatom selected from nitrogen, oxygen, phosphorus, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl, and thiepanyl. Exemplary 8 membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” refers to a radical of the formula —R_{b}-R_{c} where R_{b} is an alkylene, alkenylene, or alkynylene chain as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

The term “heteroaryl” refers to a radical of a 5.14 membered monocyclic or polycyclic (e.g., bicyclic, tricyclic) 4n+2 aromatic ring system (e.g., having 6, 10, or 14 π electrons shared in a cyclic array) having ring carbon atoms and 1.5 ring heteroatoms provided in the aromatic ring system, wherein each heteroatom is independently selected from nitrogen, oxygen, and sulfur (“5.14 membered heteroaryl”). The terms “heteroaryl” and “heteroar-,” used alone or as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group be monovalent or may have more than one point of attachment to another moiety (e.g., it may be divalent, trivalent, etc), although the valency may be specified directly in the name of the group. For example, “triazoldiyl” and “triazolylene” refer to a divalent triazolyl moiety. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. In certain embodiments, “heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this disclosure, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Unless stated otherwise specifically in this disclosure, a heteroaryl group can be optionally substituted.

In some embodiments, a heteroaryl group is a 5.10 membered aromatic ring system having ring carbon atoms and 1.4 ring heteroatoms provided in the aromatic ring system, wherein each heteroatom is independently selected from nitrogen, oxygen, and sulfur (“5-10 membered heteroaryl”). In some embodiments, a heteroaryl group is a 5.8 membered aromatic ring system having ring carbon atoms and 1.4 ring heteroatoms provided in the aromatic ring system, wherein each heteroatom is independently selected from nitrogen, oxygen, and sulfur (“5.8 membered heteroaryl”). In some embodiments, a heteroaryl group is a 5.6 membered aromatic ring system having ring carbon atoms and 1.4 ring heteroatoms provided in the aromatic ring system, wherein each heteroatom is independently selected from nitrogen, oxygen, and sulfur (“5.6 membered heteroaryl”). In some embodiments, the 5.6 membered heteroaryl has 1.3 ring heteroatoms selected from nitrogen, oxygen, and sulfur. In some embodiments, the 5.6 membered heteroaryl has 1.2 ring heteroatoms selected from nitrogen, oxygen, and sulfur. In some embodiments, the 5.6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7 membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}-R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

The term “hydroxyl” or “hydroxy” refers to the group —OH.

The term “thiol” or “thio” refers to the group —SH.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkyl aryl amines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. Optionally substituted refers to a group which may be substituted or unsubstituted (e.g., “substituted” or “unsubstituted” alkyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Affixing the suffix “ene” to a group indicates the group is a polyvalent (e.g., bivalent, trivalent, tetravalent, or pentavalent) moiety. In certain embodiments, affixing the suffix “ene” to a group indicates the group is a bivalent moiety.

Exemplary carbon atom substituents include, but are not limited to, halogen, —CN, —NO₂, —N₃, —SO₂H, —SO₃H, —OH, —OR^{aa}, ON(R^{bb})₂, N(R^{bb})₂, N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}OC(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}—, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂—OP(═O)(R^{aa})₂—OP(═O)(OR^{cc})₂, —P(═O)(N(R^{bb})₂)₂, —OP(═O)(N(R^{bb})₂)₂, —NR^{bb}P(═O)(R^{aa})₂—NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5.14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion;

or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3.14 membered heterocyclyl, C₆₋₁₄ aryl, and 5.14 membered heteroaryl, or two R^{aa} groups are joined to form a 3.14 membered heterocyclyl or 5.14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂—CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —S₂R^{cc}, —S₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3.14 membered heterocyclyl, C₆₋₁₄ aryl, and 5.14 membered heteroaryl, or two R^{bb} groups are joined to form a 3.14 membered heterocyclyl or 5.14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3.14 membered heterocyclyl, C₆₋₁₄ aryl, and 5.14 membered heteroaryl, or two R^{cc} groups are joined to form a 3.14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ee}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ee}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3.10 membered heterocyclyl, C₆₋₁₀ aryl, 5.10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3.10 membered heterocyclyl, and 3.10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3.10 membered heterocyclyl, C₆₋₁₀ aryl and 5.10 membered heteroaryl, or two R^{ff} groups are joined to form a 3.10 membered heterocyclyl or 5.10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3.10 membered heterocyclyl, 5.10 membered heteroaryl; or two geminal R^{gg} substituents can be joined to form ═O or ═S; wherein X⁻ is a counterion.

In certain embodiments, the carbon atom substituents are independently halogen, substituted or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted or unsubstituted C₁₋₆ alkyl, —OR^{aa}, —SR^{aa}, —N(R^{bb})₂, —CN, —SCN, or —NO₂.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)(OR_{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5.14 membered heteroaryl, or two R^{cc} groups attached to an N atom are joined to form a 3.14 membered heterocyclyl or 5.14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R)₂, —C(═O)R^{aa}, —C(═O)N(R^{cc})₂, —CO₂R^{aa}, —SO₂R^{aa}, —C(═NR)R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3.14 membered heterocyclyl, C₆₋₁₄ aryl, and 5.14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3d edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a, 4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, the symbol

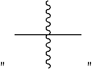

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

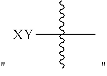

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

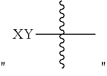

infers that when R³ is “XY”, the point of attachment bond is the same bond as the bond by which R³ is depicted as being bonded to CH₃.

“Fused” refers to any ring structure described herein which is fused to an existing ring structure in the compounds described herein. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

Total synthesis refers to the complete chemical synthesis of a complex molecule, typically a natural product or a structurally similar analog or derivative thereof, starting from commercially available precursor compounds. It is often desirable to perform total syntheses in a “convergent” manner, where efficiency and overall chemical yield are improved by synthesizing several complex individual components in stage one, followed by combination of the components in a subsequent stage to yield a more advanced compound or final product. While convergent synthetic methods are desirable, for complex molecular frameworks such as communesins generally, or enantioenriched communesins specifically specifically, there can be many different possible convergent approaches. The success of any particular approach is highly unpredictable.

The compounds described herein, or their salts can contain one or more stereogenic centers or asymmetric centers and can thus give rise to enantiomers, diastereomers, and other stereoisomeric forms that can be defined, in terms of absolute stereochemistry, as (R)- or (S)- or, as (D)- or (L)- for amino acids. The subject matter described herein is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include asymmetric synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The subject matter described herein contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The subject matter described herein includes tautomers of any said compounds.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. In certain embodiments, a pharmaceutically acceptable salt is a pharmaceutically acceptable acid addition salt. In certain embodiments, a pharmaceutically acceptable salt is a pharmaceutically acceptable base addition salt.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Crystallization is a method commonly used to isolate a reaction product, for example one of the compounds disclosed herein, in purified form. Often, crystallization produces a solvate of the compound described herein. As used herein, the term “solvate” refers to an aggregate that comprises one or more molecules of a compound described herein with one or more molecules of solvent, typically in co-crystallized form. The solvent can be water, in which case the solvate can be a hydrate. Alternatively, the solvent can be an organic solvent. Thus, the compounds described herein can exist as a hydrate, including a monohydrate, dihydrate, hemihydrate, sesquihydrate, trihydrate, tetrahydrate and the like, as well as the corresponding solvated forms. The compound described herein can be true solvates, while in other cases, the compound described herein can merely retain adventitious water or be a mixture of water plus some adventitious solvent.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 and/or ChemDraw Professional 16.0.0.82 software naming program (CambridgeSoft), or the like. For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

The subject matter described herein is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the subject matter described herein includes compounds produced by a process comprising administering a compound described herein to a mammal for a period of time sufficient to yield a metabolic product thereof. Such products are typically identified by administering a radiolabeled compound described herein in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust (e.g., with a half-life under ambient conditions of about: 1 day, 3 days, 7 days, 1 month, 3 months, or 1 year) to survive isolation to a useful degree of purity from a reaction mixture, and formulation into an efficacious therapeutic agent.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. As used herein, a “subject” can be a human, non-human primate, mammal, rat, mouse, cow, horse, pig, sheep, goat, dog, cat, insect and the like. The subject can be suspected of having or at risk for having a cancer, such as a blood cancer, or another disease or condition. Diagnostic methods for various cancers, and the clinical delineation of cancer, are known to those of ordinary skill in the art. The subject can also be suspected of having an infection or abnormal cardiovascular function.

“Mammal” includes humans and both domestic animals such as laboratory animals and household pets (e.g., cats, dogs, swine, cattle, sheep, goats, horses, rabbits), and non-domestic animals such as wildlife and the like.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence. “Treating” or “treatment” as used herein covers the treatment of the disease or condition of interest in a mammal, preferably a human, having the disease or condition of interest, and includes (but is not limited to):
1. preventing the disease or condition from occurring in a mammal, in particular, when such mammal is predisposed to the condition but has not yet been diagnosed as having it;
2. inhibiting the disease or condition, i.e., arresting its development;
3. relieving the disease or condition, i.e., causing regression of the disease or condition (ranging from reducing the severity of the disease or condition to curing the disease of condition); or
4. relieving the symptoms resulting from the disease or condition, i.e., relieving pain without addressing the underlying disease or condition. As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

The terms “composition” and “formulation” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or pharmaceutical composition described herein in a single dose. In certain embodiments, an effective amount is the combined amounts of a compound or pharmaceutical composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

A “pharmaceutical composition” refers to a formulation of a compound described herein and a medium generally accepted in the art for the delivery of the biologically active compound to mammals, e.g., humans. Such a medium includes all pharmaceutically acceptable carriers, diluents or excipients therefor.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50.80 includes the ranges with endpoints such as 55-80, 50-75, etc.).

The disclosure is not intended to be limited in any manner by the above exemplary listing of definitions. Additional terms may be defined in other sections of this disclosure.

### Compounds

In one embodiment, the present disclosure relates to compounds of Formula (I):

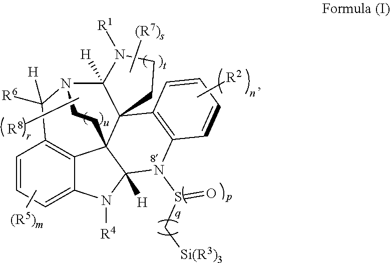

or a salt, tautomer, or stereoisomer thereof, wherein:
R¹ and R⁴ are each independently selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², substituted or unsubstituted aryl, substituted or unsubstituted heteroaryl, substituted or unsubstituted carbocyclyl, and substituted or unsubstituted heterocyclyl;
each instance of R² and R⁵ is independently selected from F, Cl, Br, I, —OH, —OR⁹, —OC(═O)R⁹, —S(═O)_{b}R¹², —NR⁹R¹⁰, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl;
each instance of R³ is independently selected from substituted or unsubstituted C₁-C₁₂ alkyl, substituted or unsubstituted aryl, substituted or unsubstituted heteroaryl, substituted or unsubstituted carbocyclyl, or substituted or unsubstituted heterocyclyl; R⁶ is H, —OH, —OR⁹, —OC(═O)R⁹, —S(═O)_{b}R¹², —NR⁹R¹⁰, substituted or unsubstituted C₁-C₁₂ alkyl, substituted or unsubstituted C₁-C₁₂ heteroalkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, or substituted or unsubstituted heterocyclyl;
each instance of R⁷ and R₈ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl, or wherein two R⁷ or two R⁸ groups taken together with the carbon atoms to which they are attached form a substituted or unsubstituted aryl, substituted or unsubstituted heteroaryl, substituted or unsubstituted carbocyclic, or substituted or unsubstituted heterocyclic ring;
each instance of R⁹ and R¹⁰ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R⁹ and R¹⁰ taken together with the carbon atoms to which they are attached form a substituted or unsubstituted heteroaryl or substituted or unsubstituted heterocyclic ring;
each instance of R¹² is independently substituted or unsubstituted C₁-C₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)_{c}SiMe₃, or —(CH₂)_{c}R⁹;
m and t are each independently an integer from 0 to 3, inclusive;
n, r, and s are each independently an integer from 0 to 4, inclusive;
each instance of c is independently an integer from 0 to 6, inclusive;
each instance of b is independently 0, 1, or 2;
u is 0, 1, or 2;
p is an integer selected from 1 or 2; and
q is an integer from 1 to 6, inclusive.

Further provided are compounds of Formula (V):

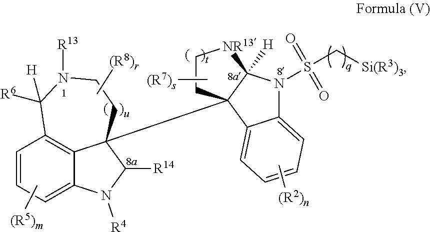

or a salt, tautomer, or stereoisomer thereof, wherein
R⁴ is selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², substituted or unsubstituted aryl, substituted or unsubstituted heteroaryl, substituted or unsubstituted carbocyclyl, and substituted or unsubstituted heterocyclyl;
each instance of R² and R⁵ are independently selected from F, Cl, Br, I, —OH, —OR⁹, —OC(═O)R⁹, —S(═O)_{b}R¹², —NR⁹R¹⁰, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted and heterocyclyl;
each instance of R³ is independently selected from substituted or unsubstituted C₁-C₁₂ alkyl, substituted or unsubstituted aryl, substituted or unsubstituted heteroaryl, substituted or unsubstituted carbocyclyl, and substituted or unsubstituted heterocyclyl;

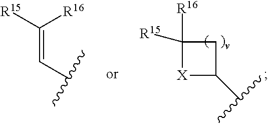

X is O, NR⁹, or —S(═O)_{b}R¹²;
each instance of R⁷ and R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl, or wherein two R⁷ or two R⁸ groups taken together with the carbon atoms to which they are attached form a substituted or unsubstituted aryl, substituted or unsubstituted heteroaryl, substituted or unsubstituted carbocyclic, or substituted or unsubstituted heterocyclic ring;
each instance of R⁹ and R¹⁰ are independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R⁹ and R¹⁰ taken together with the carbon atoms to which they are attached form a substituted or unsubstituted heteroaryl or substituted or unsubstituted heterocyclic ring;
each instance of R¹² is independently substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)_{c}SiMe₃, or —(CH₂)_{c}R⁹;
R¹³ and R^{13′} are each independently

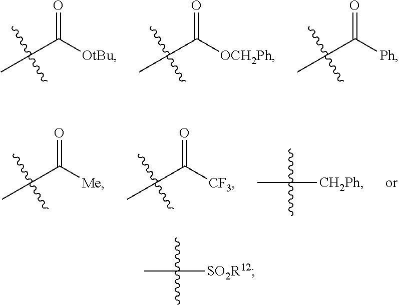

R¹⁴ is —CN, —OH, —OR⁹, —NR⁹R¹⁰, S(═O)_{b}R¹², or P(═O)(OR⁹)₂
each instance of R¹⁵ and R¹⁶ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R¹⁵ and R¹⁶ taken together with the carbon atoms to which they are attached form a substituted or unsubstituted carbocyclic ring, or substituted or unsubstituted heterocyclic ring;
m and t are each independently an integer from 0 to 3, inclusive;
n, r, and s are each independently an integer from 0 to 4, inclusive;
v is an integer from 0 to 4, inclusive;
each instance of c is independently an integer from 0 to 6, inclusive;
each instance of b is independently 0, 1, or 2;
u is 0, 1, or 2; and
q is an integer from 1 to 6, inclusive.

The disclosure further provides compounds of Formula (III′):

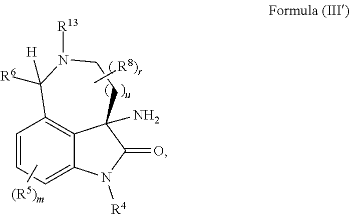

or a salt, tautomer, or stereoisomer thereof, wherein
R⁴ is selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰,
—S(═O)_{b}R¹², substituted or unsubstituted aryl, substituted or unsubstituted heteroaryl, substituted or unsubstituted carbocyclyl, and substituted or unsubstituted heterocyclyl; each instance of R⁵ is independently selected from F, Cl, Br, I, —OH, —OR⁹, —OC(═O)R⁹,
—S(═O)_{b}R¹², —NR⁹R¹⁰, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl;

R⁶ is or

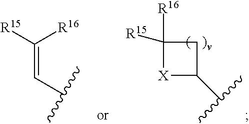

X is O, NR⁹, or —S(═O)_{b}R¹²;
each instance of R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl;
each instance of R⁹ and R¹⁰ is independently selected from H, C₁-C₁₂ alkyl, C₂-C₁₂ alkenyl, C₂-C₁₂ alkynyl, aryl, heteroaryl, carbocyclyl, and heterocyclyl, or wherein R⁹ and R¹⁰ taken together with the carbon atoms to which they are attached form a substituted or unsubstituted heteroaryl or substituted or unsubstituted heterocyclic ring;
each instance of R¹² is independently substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)ₑSiMe₃, or —(CH₂)ₑR⁹;
R¹³ is

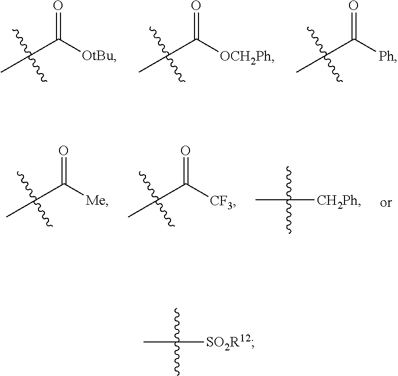

each instance of R¹⁵ and R¹⁶ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R¹⁵ and R¹⁶ taken together with the carbon atoms to which they are attached form a substituted or unsubstituted carbocyclic, or substituted or unsubstituted heterocyclic ring;
m is an integer from 0 to 3, inclusive;
v is an integer from 0 to 4, inclusive;
r is an integer from 0 to 4, inclusive;
each instance of c is independently an integer from 0 to 6, inclusive;
each instance of b is independently 0, 1, or 2; and
u is 0, 1, or 2.

In certain aspects, the present disclosure provides compounds of Formula (XIV):

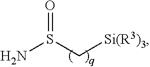

or a salt, tautomer, or stereoisomer thereof, wherein
each instance of R³ is independently substituted or unsubstituted C₁-C₁₂ alkyl, substituted or unsubstituted aryl, substituted or unsubstituted heteroaryl, substituted or unsubstituted carbocyclyl, and substituted or unsubstituted heterocyclyl; and
q is an integer from 1 to 6, inclusive.

Compounds of Formula (VI), (VII), (VII′), (VIII), (III), (III′), (IX), (X), (XI), (XII), (XIII), and (XIV) are also disclosed herein.

In the compounds and formulae disclosed herein, wherein more than one instance of a particular variable (e.g., R¹, R², R³, R⁴, R⁵, R⁶, R⁷, R⁸, R⁹, R¹⁰, R¹², R¹³, R^{13′}, R¹⁴, R¹⁵, R¹⁶, b, c m, n, p, q, r, s, t, u, v, x, and Y) is present, each instance of the variable is independent from one another (i.e., each instance of the variable is independently selected from the definition of the variable as described herein). In certain embodiments, at least two instances of a variable are different from each other. In certain embodiments, all instances of a variable are different from each other. In certain embodiments, all instances of a variable are the same.

In certain embodiments, R¹ is H. In some embodiments, R¹ is unsubstituted C₁-C₁₂ alkyl (e.g., methyl, ethyl, propyl). In some embodiments, R¹ is methyl. In some embodiments, R¹ is substituted C₁-C₁₂ alkyl (e.g., C₁-C₁₂ alkyl substituted with —OH, C₁-C₁₂ alkyl substituted with —CN, C₁-C₁₂ alkyl substituted with one or more halo, —CF₃, —CHF₂, —CH₂F). In certain embodiments, R¹ is unsubstituted C₂-C₁₂ alkenyl. In certain embodiments, R¹ is substituted C₂-C₁₂alkenyl (e.g., C₂-C₁₂ alkenyl substituted with —OH, C₂-C₁₂ alkenyl substituted with —CN). In certain embodiments, R¹ is —C(═O)R⁹. In some embodiments, R¹ is —C(═O)NR⁹R¹⁰. In some embodiments, R¹ is —S(═O)_{b}R¹².

In certain embodiments, R¹ is —C(═O)R⁹, and R⁹ is hydrogen. In certain embodiments, R¹ is —C(═O)R⁹, and R⁹ is unsubstituted C₁-C₁₂ alkyl. In certain embodiments, R¹ is —C(═O)R⁹, and R⁹ is substituted C₁-C₁₂ alkyl (e.g., wherein the substituent is —OH, —CN, or -halo). In certain embodiments, R¹ is —C(═O)R⁹, and R⁹ is methyl. In certain embodiments, R¹ is —C(═O)R⁹, and R⁹ is substituted methyl. In certain embodiments, R¹ is —C(═O)R⁹, and R⁹ is ethyl. In certain embodiments, R¹ is —C(═O)R⁹, and R⁹ is propyl. In certain embodiments, R¹ is —C(═O)R⁹, and R⁹ is unsubstituted C₂-C₁₂ alkenyl. In certain embodiments, R¹ is —C(═O)R⁹, and R⁹ is substituted C₂-C₁₂ alkenyl (e.g., wherein the substituent is —OH, —CN, or -halo). In certain embodiments, R¹ is —C(═O)R⁹, and R⁹ is a C₁-C₆ alkenyl. In certain embodiments, R¹ is —C(═O)R⁹, and R⁹ is

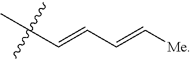 [SMILES: */C=C/C=C/C]

In certain embodiments, R is —C(═O)R⁹, and R⁹ is

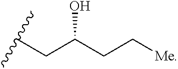 [SMILES: *C[C@H](O)CCC]

In certain embodiments, R¹ is —C(═O)R⁹, and R⁹ is

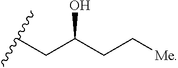 [SMILES: *C[C@@H](O)CCC]

In some embodiments, R¹ is —C(═O)NR⁹R¹⁰, R⁹ is hydrogen, and R¹⁰ is hydrogen. In some embodiments, R¹ is —C(═O)NR⁹R¹⁰, R⁹ is hydrogen, and R¹⁰ is substituted or unsubstituted C₁-C₁₂ alkyl. In some embodiments, R¹ is —C(═O)NR⁹R¹⁰, R⁹ is substituted or unsubstituted C₁-C₁₂ alkyl, and R¹⁰ is C₁-C₁₂ alkyl. In some embodiments, R¹ is —C(═O)NR⁹R¹⁰, R⁹ is hydrogen, and R¹⁰ is methyl. In some embodiments, R¹ is —C(═O)NR⁹R¹⁰, R⁹ is methyl, and R¹⁰ is methyl.

In certain embodiments, R¹ is —S(═O)_{b}R¹², and R¹² is unsubstituted C₁-C₁₂ alkyl. In certain embodiments, R¹ is —S(═O)_{b}R¹², and R¹² is substituted C₁-C₁₂ alkyl (e.g., wherein the substituent is —OH, —CN, or -halo). In certain embodiments, R¹ is —S(═O)_{b}R¹², and R¹² is methyl. In certain embodiments, R¹ is —S(═O)_{b}R¹², and R¹² is substituted methyl. In certain embodiments, R¹ is —S(═O)_{b}R¹², and R¹² is ethyl. In certain embodiments, R¹ is —S(═O)_{b}R¹², and R¹² is propyl. In certain embodiments, R¹ is —S(═O)_{b}R¹², and R¹² is a C₁-C₆ alkenyl. In certain embodiments, R is —S(═O)_{b}R¹², and R¹² is unsubstituted C₂-C₁₂ alkenyl. In certain embodiments, R¹ is —S(═O)_{b}R¹², and R¹² is substituted C₂-C₁₂ alkenyl (e.g., wherein the substituent is —OH, —CN, or -halo). In certain embodiments, R is —S(═O)_{b}R¹², and R¹² is

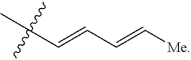 [SMILES: */C=C/C=C/C]

In certain embodiments, R¹ is —S(═O)_{b}R¹², and R¹² is

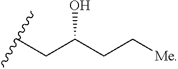 [SMILES: *C[C@H](O)CCC]

In certain embodiments, R¹ is —S(═O)_{b}R¹², and R¹² is

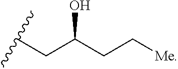 [SMILES: *C[C@@H](O)CCC]

In certain embodiments, each instance of R² is the same. In some embodiments, each instance of R² is different. In some embodiments, some instances of R² are the same and some instances of R² are different.

In certain embodiments, R² is F. In some embodiments, R² is Cl. In certain embodiments, R² is Br. In some embodiments, R² is —OH. In some embodiments, R² is —OR⁹. In some embodiments, R² is —OCH₃. In some embodiments, R² is unsubstituted C₁-C₁₂ alkyl (e.g., methyl, ethyl, propyl). In some embodiments, R² is methyl. In some embodiments, R² is substituted C₁-C₁₂ alkyl (e.g., C₁-C₁₂ alkyl substituted with —OH, C₁-C₁₂ alkyl substituted with —CN, C₁-C₁₂ alkyl substituted with one or more halo, —CF₃, —CHF₂, —CH₂F). In certain embodiments, R² is unsubstituted C₂-C₁₂ alkenyl. In certain embodiments, R² is substituted C₂-C₁₂alkenyl (e.g., C₂-C₁₂ alkenyl substituted with —OH, C₂-C₁₂ alkenyl substituted with —CN). In certain embodiments, R² is —C(═O)R⁹. In some embodiments, R² is —C(═O)NR⁹R¹⁰. In some embodiments, R² is —S(═O)_{b}R¹².

In certain embodiments, R² is —C(═O)R⁹, and R⁹ is hydrogen. In certain embodiments, R² is —C(═O)R⁹, and R⁹ is unsubstituted C₁-C₁₂ alkyl. In certain embodiments, R² is —C(═O)R⁹, and R⁹ is substituted C₁-C₁₂ alkyl (e.g., wherein the substituent is —OH, —CN, or -halo). In certain embodiments, R² is —C(═O)R⁹, and R⁹ is methyl. In certain embodiments, R² is —C(═O)R⁹, and R⁹ is substituted methyl. In certain embodiments, R² is —C(═O)R⁹, and R⁹ is ethyl. In certain embodiments, R² is —C(═O)R⁹, and R⁹ is propyl. In certain embodiments, R² is —C(═O)R⁹, and R⁹ is unsubstituted C₂-C₁₂ alkenyl. In certain embodiments, R² is —C(═O)R⁹, and R⁹ is substituted C₂-C₁₂ alkenyl (e.g., wherein the substituent is —OH, —CN, or -halo). In certain embodiments, R² is —C(═O)R⁹, and R⁹ is a C₁-C₆ alkenyl.

In some embodiments, R² is —NR⁹R¹⁰, R⁹ is hydrogen, and R¹⁰ is hydrogen. In some embodiments, R² is —NR⁹R¹⁰, R⁹ is hydrogen, and R¹⁰ is substituted or unsubstituted C₁-C₁₂ alkyl. In some embodiments, R² is —NR⁹R¹⁰, R⁹ is substituted or unsubstituted C₁-C₁₂ alkyl, and R¹⁰ is C₁-C₁₂ alkyl. In some embodiments, R² is —NR⁹R¹⁰, R⁹ is hydrogen, and R¹⁰ is methyl. In some embodiments, R² is —NR⁹R¹⁰, R⁹ is methyl, and R¹⁰ is methyl.

In certain embodiments, R² is —S(═O)_{b}R¹², and R¹² is unsubstituted C₁-C₁₂ alkyl. In certain embodiments, R² is —S(═O)_{b}R¹², and R¹² is substituted C₁-C₁₂ alkyl (e.g., wherein the substituent is —OH, —CN, or -halo). In certain embodiments, R² is —S(═O)_{b}R¹², and R¹² is methyl. In certain embodiments, R² is —S(═O)_{b}R¹², and R¹² is substituted methyl. In certain embodiments, R² is —S(═O)_{b}R¹², and R¹² is ethyl. In certain embodiments, R² is —S(═O)_{b}R¹², and R¹² is propyl. In certain embodiments, R² is —S(═O)_{b}R¹², and R¹² is a C₁-C₆ alkenyl.

In certain embodiments, each instance of R³ is the same. In some embodiments, each instance of R³ is different. In some embodiments, some instances of R³ are the same and some instances of R³ are different.

In some embodiments, each instance of R is substituted or unsubstituted C₁-C₁₂ alkyl. In some embodiments, each instance of R³ is substituted or unsubstituted C₁-C₆ alkyl. In some embodiments, each instance of R³ is unsubstituted methyl. In some embodiments, each instance of R³ is substituted methyl. In some embodiments, each instance of R³ is unsubstituted ethyl. In some embodiments, each instance of R³ is substituted ethyl. In some embodiments, each instance of R³ is substituted or unsubstituted aryl. In some embodiments, each instance of R³ is unsubstituted phenyl. In some embodiments, each instance of R³ is substituted phenyl (e.g., substituted with —OH, —OCH₃, —CN, halo, —CF₃, —CHF₂, —CH₂F, or —NO₂).

In certain embodiments, two instances of R³ are methyl and one instance of R³ is isopropyl. In certain embodiments, two instances of R³ are methyl and one instance of R³ is tert-butyl. In certain embodiments, two instances of R³ are C₁-C₁₂ alkyl and one instance of R³ is aryl. In certain embodiments, two instances of R³ are C₁-C₆ alkyl and one instance of R³ is aryl. In certain embodiments, one instances of R³ is C₁-C₁₂ alkyl and two instances of R³ are aryl. In certain embodiments, one instance of R³ is C₁-C₆ alkyl and two instances of R³ are aryl. In certain embodiments, one instance of R³ is tert-butyl and two instances of R³ are phenyl.

In some embodiments, R³ is methyl, q is 2, and p is 1. In some embodiments, R³ is methyl, q is 2, and p is 2. In some embodiments, R³ is ethyl, q is 2, and p is 1. In some embodiments, R³ is ethyl, q is 2, and p is 2. In some embodiments, R³ is phenyl, q is 2, and p is 1. In some embodiments, R³ is phenyl, q is 2, and p is 2.

In certain embodiments, R⁴ is H. In some embodiments, R⁴ is unsubstituted C₁-C₁₂ alkyl (e.g., methyl, ethyl, propyl). In some embodiments, R⁴ is methyl. In some embodiments, R⁴ is substituted C₁-C₁₂ alkyl (e.g., C₁-C₁₂ alkyl substituted with —OH, C₁-C₁₂ alkyl substituted with —CN, C₁-C₁₂ alkyl substituted with one or more halo (e.g., —CF₃, —CHF₂, —CH₂F)). In certain embodiments, R⁴ is unsubstituted C₂-C₁₂ alkenyl. In certain embodiments, R⁴ is substituted C₂-C₁₂alkenyl (e.g., C₂-C₁₂ alkenyl substituted with —OH, C₂-C₁₂ alkenyl substituted with —CN). In certain embodiments, R⁴ is —C(═O)R⁹. In some embodiments, R⁴ is —C(═O)NR⁹R¹⁰. In some embodiments, R⁴ is —S(═O)_{b}R¹².

In certain embodiments, R⁴ is —C(═O)R⁹, and R⁹ is hydrogen. In certain embodiments, R⁴ is —C(═O)R⁹, and R⁹ is unsubstituted C₁-C₁₂ alkyl. In certain embodiments, R⁴ is —C(═O)R⁹, and R⁹ is substituted C₁-C₁₂ alkyl (e.g., wherein the substituent is —OH, —CN, or -halo). In certain embodiments, R⁴ is —C(═O)R⁹, and R⁹ is methyl. In certain embodiments, R⁴ is —C(═O)R⁹, and R⁹ is substituted methyl. In certain embodiments, R⁴ is —C(═O)R⁹, and R⁹ is ethyl. In certain embodiments, R⁴ is —C(═O)R⁹, and R⁹ is propyl. In certain embodiments, R⁴ is —C(═O)R⁹, and R⁹ is unsubstituted C₂-C₁₂ alkenyl. In certain embodiments, R⁴ is —C(═O)R⁹, and R⁹ is substituted C₂-C₁₂ alkenyl (e.g., wherein the substituent is —OH, —CN, or -halo). In certain embodiments, R⁴ is —C(═O)R⁹, and R⁹ is a C₁-C₆ alkenyl. In certain embodiments, R⁴ is —C(═O)R⁹, and R⁹ is

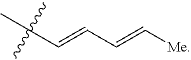 [SMILES: */C=C/C=C/C]

In certain embodiments R⁴ is —C(═O)R⁹, and R⁹ is

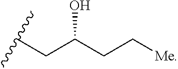 [SMILES: *C[C@H](O)CCC]

In certain embodiments, R⁴ is —C(═O)R⁹, and R⁹ is

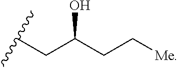 [SMILES: *C[C@@H](O)CCC]

In some embodiments, R⁴ is —C(═O)NR⁹R¹⁰, R⁹ is hydrogen, and R¹⁰ is hydrogen. In some embodiments, R⁴ is —C(═O)NR⁹R¹⁰, R⁹ is hydrogen, and R¹⁰ is substituted or unsubstituted C₁-C₁₂ alkyl. In some embodiments, R⁴ is —C(═O)NR⁹R¹⁰, R⁹ is substituted or unsubstituted C₁-C₁₂ alkyl, and R¹⁰ is C₁-C₁₂ alkyl. In some embodiments, R⁴ is —C(═O)NR⁹R¹⁰, R⁹ is hydrogen, and R¹⁰ is methyl. In some embodiments, R⁴ is —C(═O)NR⁹R¹⁰, R⁹ is methyl, and R¹⁰ is methyl.

In certain embodiments, R⁴ is —S(═O)_{b}R¹², and R¹² is unsubstituted C₁-C₁₂ alkyl. In certain embodiments, R⁴ is —S(═O)_{b}R¹², and R¹² is substituted C₁-C₁₂ alkyl (e.g., wherein the substituent is —OH, —CN, or -halo). In certain embodiments, R⁴ is —S(═O)_{b}R¹², and R¹² is methyl. In certain embodiments, R⁴ is —S(═O)_{b}R¹², and R¹² is substituted methyl. In certain embodiments, R⁴ is —S(═O)_{b}R¹², and R¹² is ethyl. In certain embodiments, R⁴ is —S(═O)_{b}R¹², and R¹² is propyl. In certain embodiments, R⁴ is —S(═O)_{b}R¹², and R¹² is a C₁-C₆ alkenyl. In certain embodiments, R⁴ is —S(═O)_{b}R¹², and R¹² is unsubstituted C₂-C₁₂ alkenyl. In certain embodiments, R⁴ is —S(═O)_{b}R¹², and R¹² is substituted C₂-C₁₂ alkenyl (e.g., wherein the substituent is —OH, —CN, or -halo). In certain embodiments, R⁴ is —S(═O)R¹², and R¹² is

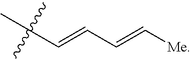 [SMILES: */C=C/C=C/C]

In certain embodiments, R⁴ is —S(═O)_{b}R¹², and R¹² is

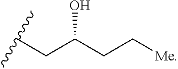 [SMILES: *C[C@H](O)CCC]

In certain embodiments, R⁴ is —S(═O)_{b}R¹², and R¹² is

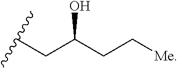 [SMILES: *C[C@@H](O)CCC]

In certain embodiments, R⁴ is substituted or unsubstituted aryl (e.g., substituted or unsubstituted phenyl). In certain embodiments, R⁴ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R⁴ is substituted or unsubstituted carbocyclyl (e.g., substituted or unsubstituted cyclopropyl, substituted or unsubstituted cyclohexyl).

In certain embodiments, each instance of R⁵ is the same. In some embodiments, each instance of R⁵ is different. In some embodiments, some instances of R⁵ are the same and some instances of R⁵ are different.

In certain embodiments, R⁵ is F. In some embodiments, R⁵ is Cl. In certain embodiments, R⁵ is Br. In some embodiments, R⁵ is —OH. In some embodiments, R⁵ is —OR⁹. In some embodiments, R⁵ is —OCH₃. In some embodiments, R⁵ is unsubstituted C₁-C₁₂ alkyl (e.g., methyl, ethyl, propyl). In some embodiments, R⁵ is methyl. In some embodiments, R⁵ is substituted C₁-C₁₂ alkyl (e.g., C₁-C₁₂ alkyl substituted with —OH, C₁-C₁₂ alkyl substituted with —CN, C₁-C₁₂ alkyl substituted with one or more halo, —CF₃, —CHF₂, —CH₂F). In certain embodiments, R⁵ is unsubstituted C₂-C₁₂ alkenyl. In certain embodiments, R⁵ is substituted C₂-C₁₂ alkenyl (e.g., C₂-C₁₂ alkenyl substituted with —OH, C₂-C₁₂ alkenyl substituted with —CN). In certain embodiments, R⁵ is —C(═O)R⁹. In some embodiments, R⁵ is —C(═O)NR⁹R¹⁰. In some embodiments, R⁵ is —S(═O)_{b}R¹².

In certain embodiments, R⁵ is —C(═O)R⁹, and R⁹ is hydrogen. In certain embodiments, R⁵ is —C(═O)R⁹, and R⁹ is unsubstituted C₁-C₁₂ alkyl. In certain embodiments, R⁵ is —C(═O)R⁹, and R⁹ is substituted C₁-C₁₂ alkyl (e.g., wherein the substituent is —OH, —CN, or -halo). In certain embodiments, R⁵ is —C(═O)R⁹, and R⁹ is methyl. In certain embodiments, R⁵ is —C(═O)R⁹, and R⁹ is substituted methyl. In certain embodiments, R⁵ is —C(═O)R⁹, and R⁹ is ethyl. In certain embodiments, R⁵ is —C(═O)R⁹, and R⁹ is propyl. In certain embodiments, R⁵ is —C(═O)R⁹, and R⁹ is unsubstituted C₂-C₁₂ alkenyl. In certain embodiments, R⁵ is —C(═O)R⁹, and R⁹ is substituted C₂-C₁₂ alkenyl (e.g., wherein the substituent is —OH, —CN, or -halo). In certain embodiments, R⁵ is —C(═O)R⁹, and R⁹ is a C₁-C₆ alkenyl.

In some embodiments, R⁵ is —NR⁹R¹⁰, R⁹ is hydrogen, and R¹⁰ is hydrogen. In some embodiments, R⁵ is —NR⁹R¹⁰, R⁹ is hydrogen, and R¹⁰ is substituted or unsubstituted C₁-C₁₂ alkyl. In some embodiments, R⁵ is —NR⁹R¹⁰, R⁹ is substituted or unsubstituted C₁-C₁₂ alkyl, and R¹⁰ is C₁-C₁₂ alkyl. In some embodiments, R⁵ is —NR⁹R¹⁰, R⁹ is hydrogen, and R¹⁰ is methyl. In some embodiments, R⁵ is —NR⁹R¹⁰, R⁹ is methyl, and R¹⁰ is methyl.

In certain embodiments, R⁵ is —S(═O)_{b}R¹², and R¹² is unsubstituted C₁-C₁₂ alkyl. In certain embodiments, R⁵ is —S(═O)_{b}R¹², and R¹² is substituted C₁-C₁₂ alkyl (e.g., wherein the substituent is —OH, —CN, or -halo). In certain embodiments, R⁵ is —S(═O)_{b}R¹², and R¹² is methyl. In certain embodiments, R⁵ is —S(═O)_{b}R¹², and R¹² is substituted methyl. In certain embodiments, R⁵ is —S(═O)_{b}R¹², and R¹² is ethyl. In certain embodiments, R⁵ is —S(═O)_{b}R¹², and R¹² is propyl. In certain embodiments, R⁵ is —S(═O)_{b}R¹², and R¹² is a C₁-C₆ alkenyl.

In certain embodiments, R⁶ is hydrogen. In some embodiments, R⁶ is —OH. In certain embodiments, R⁶ is —OR⁹ (e.g., —OCH₃). In certain embodiments, R⁶ is —NR⁹R¹⁰ (e.g., —NH₂). In some embodiments, R⁶ is substituted or unsubstituted C₁-C₁₂ alkyl (e.g., methyl, —CF₃). In certain embodiments, R⁶ is substituted or unsubstituted C₁-C₁₂ heteroalkyl. In some embodiments, R⁶ is substituted or unsubstituted C₂-C₁₂ alkenyl. In some embodiments, R⁶ is substituted or unsubstituted C₂-C₆ alkenyl. In some embodiments, R⁶ is substituted C₂-C₆ alkenyl. In some embodiments, R⁶ is substituted C₂-C₆ alkenyl. In some embodiments, R⁶ is substituted or unsubstituted C₂-C₁₂ alkynyl. In some embodiments, R⁶ is substituted or unsubstituted C₂-C₆ alkynyl. In certain embodiments, R⁶ is substituted or unsubstituted aryl (e.g., substituted or unsubstituted phenyl). In certain embodiments, R⁶ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R⁶ is substituted or unsubstituted carbocyclyl (e.g., substituted or unsubstituted cyclopropyl, substituted or unsubstituted cyclohexyl). In certain embodiments, R⁶ is substituted or unsubstituted heterocyclyl (e.g., substituted or unsubstituted aziridinyl, substituted or unsubstituted thiiranyl, substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl). In certain embodiments, R⁶ is substituted or unsubstituted oxiranyl. In certain embodiments, R⁶ is substituted oxiranyl.

In some embodiments, R⁶ is

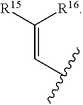

In some embodiments, R⁶ is

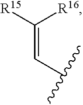

and R¹⁵ and R¹⁶ are different. In some embodiments, R⁶ is

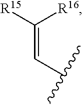

and R¹⁵ and R¹⁶ are the same. In some embodiments, R⁶ is

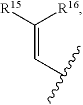

and both R¹⁵ and R¹⁶ are hydrogen. In some embodiments, R⁶

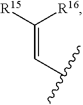

R¹⁵ is methyl, and R¹⁶ is hydrogen. In some embodiments, R⁶ is

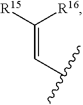

R¹⁵ is hydrogen, and R¹⁶ is methyl. In certain embodiments, R⁶ is

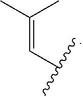 [SMILES: *C=C(C)C]

In certain embodiments, X is O. In some embodiments, X is NR⁹. In some embodiments, X is NH. In some embodiments, X is NMe. In some embodiments, X is S. In some embodiments, X is S(═O)₂.

In certain embodiments, R⁶ is

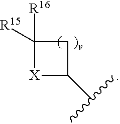

In certain embodiments, R⁶ is

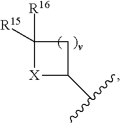

and v is 0. In certain embodiments, R⁶ is

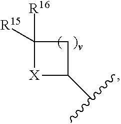

In certain embodiments, R⁶ is

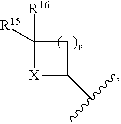

and v is 1. In certain embodiments, R⁶ is

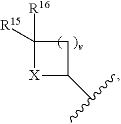

and v is 2. In certain embodiments, R⁶ is

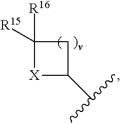

and v is 3. In certain embodiments, R⁶ is

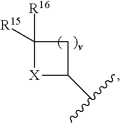

and X is O. In certain embodiments, R⁶ is

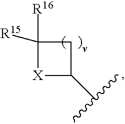

X is O, and v is 0. In certain embodiments, R⁶ is

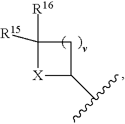

X is O, and v is 2. In certain embodiments, R⁶ is

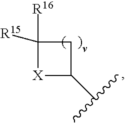

X is O, and v is 3. In certain embodiments, R⁶ is

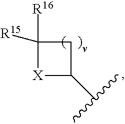

and X is NR⁹. In certain embodiments, R⁶ is

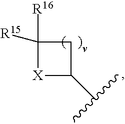

and X is NH. In certain embodiments, R⁶ is

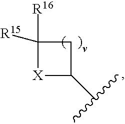

X is NH, and v is 0. In certain embodiments, R⁶ is

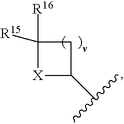

X is NH, and v is 2. In certain embodiments, R⁶ is

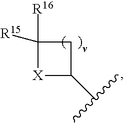

X is NH, and v is 3. In certain embodiments, R⁶ is

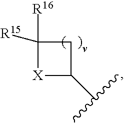

and X is —S(═O)₂. In certain embodiments, R⁶ is

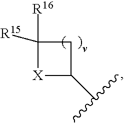

and X is —S. In some embodiments, R⁶ is

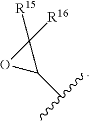

In some embodiments, R⁶ is

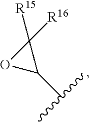

and R¹⁵ and R¹⁶ are different. In some embodiments, R⁶ is

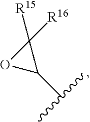

and R¹⁵ and R¹⁶ are the same. In some embodiments, R⁶ is

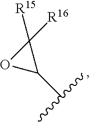

and both R¹⁵ and R¹⁶ are hydrogen. In some embodiments, R⁶ is

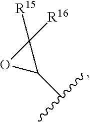

R¹⁵ is methyl, and R¹⁶ is hydrogen. In some embodiments, R⁶ is

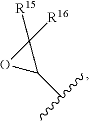

R¹⁵ is hydrogen, and R¹⁶ is methyl. In some embodiments, R⁶ is

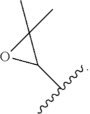 [SMILES: *C1OC1(C)C]

In some embodiments, R⁶ is

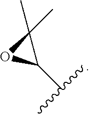

In some embodiments, R⁶ is

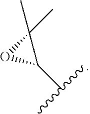

In certain embodiments, each instance of R⁷ is the same. In some embodiments, each instance of R⁷ is different. In some embodiments, some instances of R⁷ are the same and some instances of R⁷ are different.

In certain embodiments, each instance of R⁷ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl.

In certain embodiments, R⁷ is halogen. In certain embodiments, R⁷ is F. In some embodiments, R⁷ is Cl. In certain embodiments, R⁷ is Br. In some embodiments, R⁷ is —OH. In some embodiments, R⁷ is —OR⁹. In some embodiments, R⁷ is —OCH₃. In some embodiments, R⁷ is unsubstituted C₁-C₁₂ alkyl (e.g., methyl, ethyl, propyl). In some embodiments, R⁷ is methyl. In some embodiments, R⁷ is substituted C₁-C₁₂ alkyl (e.g., C₁-C₁₂ alkyl substituted with —OH, C₁-C₁₂ alkyl substituted with —CN, C₁-C₁₂ alkyl substituted with one or more halo, —CF₃, —CHF₂, —CH₂F). In certain embodiments, R⁷ is unsubstituted C₂-C₁₂ alkenyl. In certain embodiments, R⁷ is substituted C₂-C₁₂ alkenyl (e.g., C₂-C₁₂ alkenyl substituted with —OH, C₂-C₁₂ alkenyl substituted with —CN). In certain embodiments, R⁷ is —C(═O)R⁹. In some embodiments, R⁷ is —C(═O)NR⁹R¹⁰. In some embodiments, R⁷ is —S(═O)_{b}R¹². In certain embodiments, R⁷ is substituted or unsubstituted aryl (e.g., substituted or unsubstituted phenyl). In certain embodiments, R⁷ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R⁷ is substituted or unsubstituted carbocyclyl (e.g., substituted or unsubstituted cyclopropyl, substituted or unsubstituted cyclohexyl). In certain embodiments, R⁷ is substituted or unsubstituted heterocyclyl (e.g., substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl).

In certain embodiments, R⁷ is —C(═O)R⁹, and R⁹ is hydrogen. In certain embodiments, R⁷ is —C(═O)R⁹, and R⁹ is unsubstituted C₁-C₁₂ alkyl. In certain embodiments, R⁷ is —C(═O)R⁹, and R⁹ is substituted C₁-C₁₂ alkyl (e.g., wherein the substituent is —OH, —CN, or -halo). In certain embodiments, R⁷ is —C(═O)R⁹, and R⁹ is methyl. In certain embodiments, R⁷ is —C(═O)R⁹, and R⁹ is substituted methyl. In certain embodiments, R⁷ is —C(═O)R⁹, and R⁹ is unsubstituted C₂-C₁₂ alkenyl. In certain embodiments, R⁷ is —C(═O)R⁹, and R⁹ is substituted C₂-C₁₂ alkenyl (e.g., wherein the substituent is —OH, —CN, or -halo). In certain embodiments, R⁷ is —C(═O)R⁹, and R⁹ is a C₁-C₆ alkenyl.

In some embodiments, R⁷ is —C(═O)NR⁹R¹⁰, R⁹ is hydrogen, and R¹⁰ is hydrogen. In some embodiments, R⁷ is —C(═O)NR⁹R¹⁰, R⁹ is hydrogen, and R¹⁰ is methyl. In some embodiments, R⁷ is —C(═O)NR⁹R¹⁰, R⁹ is methyl, and R¹⁰ is methyl.

In certain embodiments, R⁷ is —S(═O)_{b}R¹², and R¹² is methyl.

In certain embodiments, each instance of R⁸ is the same. In some embodiments, each instance of R⁸ is different. In some embodiments, some instances of R⁸ are the same and some instances of R⁸ are different.

In certain embodiments, each instance of R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl.

In certain embodiments, R⁸ is halogen. In certain embodiments, R⁸ is F. In some embodiments, R⁸ is Cl. In certain embodiments, R⁸ is Br. In some embodiments, R⁸ is —OH. In some embodiments, R⁸ is —OR⁹. In some embodiments, R⁸ is —OCH₃. In some embodiments, R⁸ is unsubstituted C₁-C₁₂ alkyl (e.g., methyl, ethyl, propyl). In some embodiments, R⁸ is methyl. In some embodiments, R⁸ is substituted C₁-C₁₂ alkyl (e.g., C₁-C₁₂ alkyl substituted with —OH, C₁-C₁₂ alkyl substituted with —CN, C₁-C₁₂ alkyl substituted with one or more halo, —CF₃, —CHF₂, —CH₂F). In certain embodiments, R⁸ is unsubstituted C₂-C₁₂ alkenyl. In certain embodiments, R⁸ is substituted C₂-C₁₂ alkenyl (e.g., C₂-C₁₂ alkenyl substituted with —OH, C₂-C₁₂ alkenyl substituted with —CN). In certain embodiments, R⁸ is —C(═O)R⁹. In some embodiments, R is —C(═O)NR⁹R¹⁰. In some embodiments, R⁸ is —S(═O)_{b}R¹². In certain embodiments, R⁸ is substituted or unsubstituted aryl (e.g., substituted or unsubstituted phenyl). In certain embodiments, R⁸ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R⁸ is substituted or unsubstituted carbocyclyl (e.g., substituted or unsubstituted cyclopropyl, substituted or unsubstituted cyclohexyl). In certain embodiments, R⁸ is substituted or unsubstituted heterocyclyl (e.g., substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl).

In certain embodiments, R⁸ is —C(═O)R⁹, and R⁹ is hydrogen. In certain embodiments, R⁸ is —C(═O)R⁹, and R⁹ is unsubstituted C₁-C₁₂ alkyl. In certain embodiments, R⁹ is —C(═O)R⁹, and R⁹ is substituted C₁-C₁₂ alkyl (e.g., wherein the substituent is —OH, —CN, or -halo). In certain embodiments, R⁹ is —C(═O)R⁹, and R⁹ is methyl. In certain embodiments, R⁸ is —C(═O)R⁹, and R⁹ is substituted methyl. In certain embodiments, R⁸ is —C(═O)R⁹, and R⁹ is unsubstituted C₂-C₁₂ alkenyl. In certain embodiments, R⁸ is —C(═O)R⁹, and R⁹ is substituted C₂-C₁₂ alkenyl (e.g., wherein the substituent is —OH, —CN, or -halo). In certain embodiments, R⁸ is —C(═O)R⁹, and R⁹ is a C₁-C₆ alkenyl.

In some embodiments, R⁸ is —C(═O)NR⁹R¹⁰, R⁹ is hydrogen, and R¹⁰ is hydrogen. In some embodiments, R⁸ is —C(═O)NR⁹R¹⁰, R⁹ is hydrogen, and R¹⁰ is methyl. In some embodiments, R⁸ is —C(═O)NR⁹R¹⁰, R⁹ is methyl, and R¹⁰ is methyl.

In certain embodiments, R⁸ is —S(═O)_{b}R¹², and R¹² is methyl.

In certain embodiments, R⁹ is hydrogen. In certain embodiments, R⁹ is unsubstituted C₁-C₁₂ alkyl. In certain embodiments, R⁹ is substituted C₁-C₁₂ alkyl (e.g., wherein the substituent is —OH, —CN, or -halo). In certain embodiments, R⁹ is

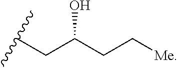 [SMILES: *C[C@H](O)CCC]

In certain embodiments, R⁹ is

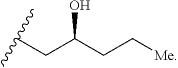 [SMILES: *C[C@@H](O)CCC]

In certain embodiments, R⁹ is methyl. In certain embodiments, R⁹ is substituted methyl. In certain embodiments, R⁹ is ethyl. In certain embodiments, R⁹ is propyl. In certain embodiments, R⁹ is unsubstituted C₂-C₁₂ alkenyl. In certain embodiments, R⁹ is substituted C₂-C₁₂ alkenyl (e.g., wherein the substituent is —OH, —CN, or -halo). In certain embodiments, R⁹ is a C₂-C₆ alkenyl. In certain embodiments, R⁹ is

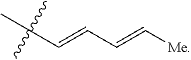 [SMILES: */C=C/C=C/C]

In certain embodiments, R⁹ is substituted or unsubstituted aryl (e.g., substituted or unsubstituted phenyl). In certain embodiments, R⁹ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R⁹ is substituted or unsubstituted carbocyclyl (e.g., substituted or unsubstituted cyclopropyl, substituted or unsubstituted cyclohexyl). In certain embodiments, R⁹ is substituted or unsubstituted heterocyclyl (e.g., substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl).

In some embodiments, R¹⁰ is hydrogen. In some embodiments, R¹⁰ is substituted or unsubstituted C₁-C₁₂ alkyl. In some embodiments, R¹⁰ is unsubstituted C₁-C₁₂ alkyl. In some embodiments, R¹⁰ is substituted C₁-C₁₂ alkyl. In some embodiments, R¹⁰ is methyl. In some embodiments, R¹⁰ is ethyl. In some embodiments, R¹⁰ is propyl.

In certain embodiments, R⁹ and R¹⁰ are taken together with the carbon atoms to which they are attached to form a substituted or unsubstituted heteroaryl (e.g., substituted or unsubstituted pyrrolyl). In certain embodiments, R⁹ and R¹⁰ are taken together with the carbon atoms to which they are attached to form substituted or unsubstituted heterocyclic ring. In certain embodiments, R⁹ and R¹⁰ are taken together with the carbon atoms to which they are attached to form a substituted or unsubstituted 4-membered ring (e.g., azetidinyl), substituted or unsubstituted 5-membered ring (e.g., pyrrolidinyl, indolinyl), or substituted or unsubstituted 6-membered ring (e.g., piperadinyl, piperazinyl, morpholinyl).

In certain embodiments, R¹² is unsubstituted C₁-C₁₂ alkyl. In certain embodiments, R¹² is substituted C₁-C₁₂ alkyl (e.g., wherein the substituent is —OH, —CN, or -halo). In certain embodiments, R¹² is methyl. In certain embodiments, R¹² is substituted methyl. In certain embodiments, R¹² is ethyl. In certain embodiments, R¹² is propyl. In certain embodiments, R¹² is a C₁-C₆ alkenyl. In certain embodiments, R¹² is unsubstituted C₂-C₁₂ alkenyl. In certain embodiments, R¹² is substituted C₂-C₁₂ alkenyl (e.g., wherein the substituent is —OH, —CN, or -halo). In certain embodiments, R¹² is

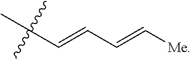 [SMILES: */C=C/C=C/C]

In certain embodiments, R¹² is

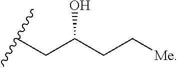 [SMILES: *C[C@H](O)CCC]

In certain embodiments, R¹² is

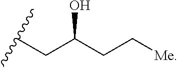 [SMILES: *C[C@@H](O)CCC]

In certain embodiments, R¹² is substituted or unsubstituted aryl (e.g., substituted or unsubstituted phenyl). In certain embodiments, R¹² is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R¹² is substituted or unsubstituted carbocyclyl (e.g., substituted or unsubstituted cyclopropyl, substituted or unsubstituted cyclohexyl). In certain embodiments, R¹² is substituted or unsubstituted heterocyclyl (e.g., substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl). In some embodiments, R¹² is —(CH₂)_{c}SiMe₃. In some embodiments, R¹² is —(CH₂)_{c}R⁹.

In some embodiments, R¹³ is a nitrogen protecting group. In some embodiments, R¹³ is

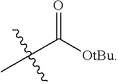 [SMILES: *C(=O)OC(C)(C)C]

In certain embodiments, R¹³ is

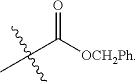

In some embodiments, R¹³ is

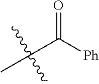 [SMILES: *C(=O)c1ccccc1]

In certain embodiments, R¹³ is

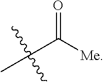 [SMILES: *C(C)=O]

In some embodiments, R¹³ is

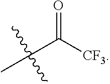 [SMILES: *C(=O)C(F)(F)F]

In certain embodiments, R¹³ is

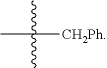

In some embodiments, R¹³ is

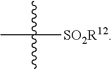

In some embodiments, R¹³ is -SES.

In some embodiments, R^{13′} is a nitrogen protecting group. In some embodiments, R^{13′} is

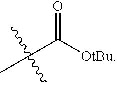 [SMILES: *C(=O)OC(C)(C)C]

In certain embodiments, R¹³ is

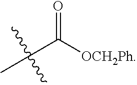

In some embodiments, R^{13′} is

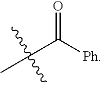 [SMILES: *C(=O)c1ccccc1]

In certain embodiments, R^{13′} is

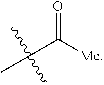 [SMILES: *C(C)=O]

In some embodiments, R^{13′} is

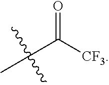 [SMILES: *C(=O)C(F)(F)F]

In certain embodiments, R^{13′} is

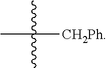

In some embodiments, R^{13′}is

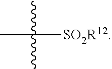

In some embodiments, R^{13′} is -SES.

In certain embodiments, both R¹³ and R^{13′} are

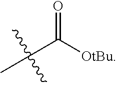 [SMILES: *C(=O)OC(C)(C)C]

In some embodiments, R¹⁴ is —CN. In certain embodiments, R¹⁴ is —OH. In some embodiments, R¹⁴ is —OR⁹ (e.g., —OCH₃). In certain embodiments, R¹⁴ is —NR⁹R¹⁰. In some embodiments, R¹⁴ is —NH₂. In certain embodiments, R¹⁴ is —N(CH₃)₂. In certain embodiments, R¹⁴ is S(═O)_{b}R¹². In certain embodiments, R¹⁴ is -SES. In certain embodiments, R¹⁴ is P(═O)(OR⁹)₂.

In some embodiments, R¹⁵ is or is the same as R⁹. In certain embodiments, R¹⁵ is hydrogen. In certain embodiments, R¹⁵ is unsubstituted C₁-C₁₂ alkyl. In certain embodiments, R¹⁵ is substituted C₁-C₁₂ alkyl (e.g., wherein the substituent is —OH, —CN, or -halo). In certain embodiments, R¹⁵ is methyl. In certain embodiments, R¹⁵ is substituted methyl. In certain embodiments, R¹⁵ is ethyl. In certain embodiments, R¹⁵ is propyl. In certain embodiments, R¹⁵ is unsubstituted C₂-C₁₂ alkenyl. In certain embodiments, R¹⁵ is substituted C₂-C₁₂ alkenyl (e.g., wherein the substituent is —OH, —CN, or -halo). In certain embodiments, R¹⁵ is substituted or unsubstituted aryl (e.g., substituted or unsubstituted phenyl). In certain embodiments, R¹⁵ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R¹⁵ is substituted or unsubstituted carbocyclyl (e.g., substituted or unsubstituted cyclopropyl, substituted or unsubstituted cyclohexyl). In certain embodiments, R¹⁵ is substituted or unsubstituted heterocyclyl (e.g., substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl).

In some embodiments, R¹⁶ is or is the same as R¹⁰. In certain embodiments, R¹⁶ is hydrogen. In certain embodiments, R¹⁶ is unsubstituted C₁-C₁₂ alkyl. In certain embodiments, R¹⁶ is substituted C₁-C₁₂ alkyl (e.g., wherein the substituent is —OH, —CN, or -halo). In certain embodiments, R¹⁶ is methyl. In certain embodiments, R¹⁶ is substituted methyl. In certain embodiments, R¹⁶ is ethyl. In certain embodiments, R¹⁶ is propyl. In certain embodiments, R¹⁶ is unsubstituted C₂-C₁₂ alkenyl. In certain embodiments, R¹⁶ is substituted C₂-C₁₂ alkenyl (e.g., wherein the substituent is —OH, —CN, or -halo). In certain embodiments, R¹⁶ is substituted or unsubstituted aryl (e.g., substituted or unsubstituted phenyl). In certain embodiments, R¹⁶ is substituted or unsubstituted heteroaryl (e.g., substituted or unsubstituted pyrrolyl, substituted or unsubstituted imidazolyl, substituted or unsubstituted triazolyl, substituted or unsubstituted pyridinyl, substituted or unsubstituted indolyl, substituted or unsubstituted purinyl, substituted or unsubstituted indolyl). In certain embodiments, R¹⁶ is substituted or unsubstituted carbocyclyl (e.g., substituted or unsubstituted cyclopropyl, substituted or unsubstituted cyclohexyl). In certain embodiments, R¹⁶ is substituted or unsubstituted heterocyclyl (e.g., substituted or unsubstituted piperidinyl, substituted or unsubstituted piperazinyl, substituted or unsubstituted morpholinyl, substituted or unsubstituted indolinyl, substituted or unsubstituted tetrahydroquinolinyl, substituted or unsubstituted quinoxalinyl).

In certain embodiments, R¹⁵ and R¹⁶ are taken together with the carbon atoms to which they are attached to form substituted or unsubstituted heterocyclic ring. In certain embodiments, R¹⁵ and R¹⁶ are taken together with the carbon atoms to which they are attached to form a substituted or unsubstituted 4-membered ring (e.g., cyclobutyl), substituted or unsubstituted 5-membered ring (e.g., cyclopently), or substituted or unsubstituted 6-membered ring (e.g., cyclohexyl). In certain embodiments, R¹⁵ and R¹⁶ are taken together with the carbon atoms to which they are attached to form substituted or unsubstituted heterocyclic ring. In certain embodiments, R¹⁵ and R¹⁶ are taken together with the carbon atoms to which they are attached to form a substituted or unsubstituted 4-membered ring (e.g., azetidinyl), substituted or unsubstituted 5-membered ring (e.g., pyrrolidinyl, indolinyl), or substituted or unsubstituted 6-membered ring (e.g., piperadinyl, piperazinyl, morpholinyl).

In certain embodiments, R¹⁵ is hydrogen, and R¹⁶ is hydrogen. In certain embodiments, R¹⁵ is methyl, and R¹⁶ is hydrogen. In certain embodiments, R¹⁵ is methyl, and R¹⁶ is methyl. In some embodiments, R¹⁵ is hydrogen, and R¹⁶ is substituted or unsubstituted C₁-C₁₂ alkyl. In some embodiments, R¹⁵ is hydrogen, and R¹⁶ is unsubstituted C₁-C₁₂ alkyl. In some embodiments, R¹⁵ is hydrogen, and R¹⁶ is substituted C₁-C₁₂ alkyl. In some embodiments, R¹⁵ is methyl, and R¹⁶ is substituted or unsubstituted C₁-C₁₂ alkyl. In some embodiments, R¹⁵ is methyl, and R¹⁶ is unsubstituted C₁-C₁₂ alkyl. In some embodiments, R¹⁵ is methyl, and R¹⁶ is substituted C₁-C₁₂ alkyl. In some embodiments, R¹⁵ is substituted or unsubstituted C₁-C₁₂ alkyl, and R¹⁶ is substituted or unsubstituted C₁-C₁₂ alkyl.

In some embodiments, m is 0. In certain embodiments, m is 1. In some embodiments, m is 2. In certain embodiments, m is 3.

In some embodiments, t is 0. In certain embodiments, t is 1. In some embodiments, t is 2. In certain embodiments, t is 3.

In some embodiments, n is 0. In certain embodiments, n is 1. In some embodiments, n is 2. In certain embodiments, n is 3. In some embodiments, n is 4.

In some embodiments, r is 0. In certain embodiments, r is 1. In some embodiments, r is 2. In certain embodiments, r is 3. In some embodiments, r is 4.

In some embodiments, s is 0. In certain embodiments, s is 1. In some embodiments, s is 2. In certain embodiments, s is 3. In some embodiments, s is 4.

In some embodiments, c is 0. In certain embodiments, c is 1. In some embodiments, c is 2. In certain embodiments, c is 3. In some embodiments, c is 4. In some embodiments, c is 5. In certain embodiments, c is 6.

In some embodiments, b is 0. In certain embodiments, b is 1. In some embodiments, b is 2.

In some embodiments, u is 0. In certain embodiments, u is 1. In some embodiments, u is 2.

In some embodiments, p is 1. In certain embodiments, p is 2.

In certain embodiments, q is 1. In some embodiments, q is 2. In certain embodiments, q is 3. In some embodiments, q is 4. In some embodiments, q is 5. In certain embodiments, q is 6.

In some embodiments, v is 0. In certain embodiments, v is 1. In some embodiments, v is 2. In certain embodiments, v is 3. In some embodiments, v is 4.

In some embodiments, Y is fluoro. In some embodiments, Y is chloro. In some embodiments, Y is bromo. In some embodiments, Y is iodo.

In certain embodiments, each instance of R³ is methyl, q is 2, p is 2, R⁴ is methyl, R¹ is —C(═O)R⁹, R⁹ is methyl, and R⁶ is

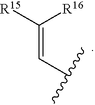

In certain embodiments, each instance of R³ is methyl, q is 2, p is 2, R⁴ is methyl, R¹ is —C(═O)R⁹, R⁹ is ethyl, and R⁶ is

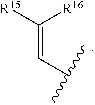

In certain embodiments, each instance of R³ is methyl, q is 2, p is 2, R⁴ is methyl, R¹ is —C(═O)R⁹, R⁹ is propyl, and R⁶ is

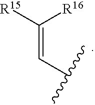

In certain embodiments, each instance of R³ is methyl, q is 2, p is 2, R⁴ is methyl, R¹ is —C(═O)R⁹, R⁹ is

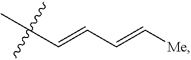 [SMILES: */C=C/C=C/C]

### and R⁶ is

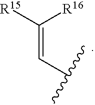

In certain embodiments, each instance of R³ is methyl, q is 2, p is 2, R⁴ is methyl, R¹ is —C(═O)R⁹, R⁹ is

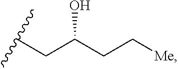 [SMILES: *C[C@H](O)CCC]

### and R⁶ is

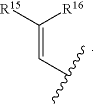

In certain embodiments, each instance of R³ is methyl, q is 2, p is 2, R⁴ is methyl, R¹ is —C(═O)R⁹, R⁹ is

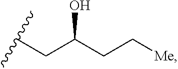 [SMILES: *C[C@@H](O)CCC]

### and R⁶ is

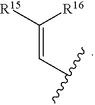

In certain embodiments, each instance of R³ is methyl, q is 2, p is 2, R⁴ is methyl, R¹ is —C(═O)R⁹, R⁹ is methyl, and R⁶ is

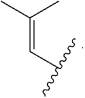 [SMILES: *C=C(C)C]

In certain embodiments, each instance of R³ is methyl, q is 2, p is 2, R⁴ is methyl, R¹ is —C(═O)R⁹, R⁹ is ethyl, and R⁶ is

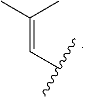 [SMILES: *C=C(C)C]

In certain embodiments, each instance of R³ is methyl, q is 2, p is 2, R⁴ is methyl, R¹ is —C(═O)R⁹, R⁹ is propyl, and R⁶ is

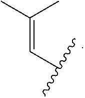 [SMILES: *C=C(C)C]

In certain embodiments, each instance of R³ is methyl, q is 2, p is 2, R⁴ is methyl, R¹ is —C(═O)R⁹, R⁹ is

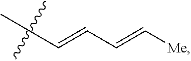 [SMILES: */C=C/C=C/C]

### and R⁶ is

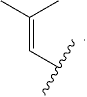 [SMILES: *C=C(C)C]

In certain embodiments, each instance of R³ is methyl, q is 2, p is 2, R⁴ is methyl, R¹ is —C(═O)R⁹, R⁹ is

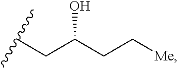 [SMILES: *C[C@H](O)CCC]

### and R⁶ is

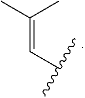 [SMILES: *C=C(C)C]

In certain embodiments, each instance of R³ is methyl, q is 2, p is 2, R⁴ is methyl, R¹ is —C(═O)R⁹, R⁹ is

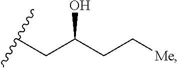 [SMILES: *C[C@@H](O)CCC]

### and R⁶ is

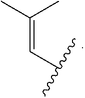 [SMILES: *C=C(C)C]

In certain embodiments, each instance of R³ is methyl, q is 2, p is 2, R⁴ is methyl, R¹ is —C(═O)R⁹, R⁹ is methyl, and R⁶ is

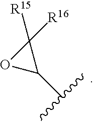

In certain embodiments, each instance of R³ is methyl, q is 2, p is 2, R⁴ is methyl, R¹ is —C(═O)R⁹, R⁹ is ethyl, and R⁶ is

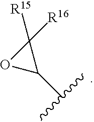

In certain embodiments, each instance of R³ is methyl, q is 2, p is 2, R⁴ is methyl, R¹ is —C(═O)R⁹, R⁹ is propyl, and R⁶ is

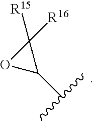

In certain embodiments, each instance of R³ is methyl, q is 2, p is 2, R⁴ is methyl, R is —C(═O)R⁹, R⁹ is

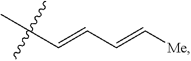 [SMILES: */C=C/C=C/C]

### and R⁶ is

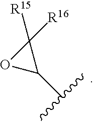

In certain embodiments, each instance of R³ is methyl, q is 2, p is 2, R⁴ is methyl, R¹ is —C(═O)R⁹, R⁹ is

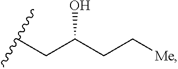 [SMILES: *C[C@H](O)CCC]

### and R⁶ is

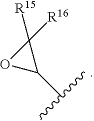

In certain embodiments, each instance of R³ is methyl, q is 2, p is 2, R⁴ is methyl, R¹ is —C(═O)R⁹, R⁹ is

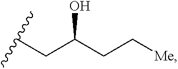 [SMILES: *C[C@@H](O)CCC]

### and R⁶ is

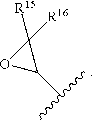

In certain embodiments, each instance of R³ is methyl, q is 2, p is 2, R⁴ is methyl, R¹ is —C(═O)R⁹, R⁹ is methyl, and R⁶ is

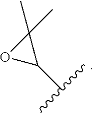 [SMILES: *C1OC1(C)C]

In certain embodiments, each instance of R³ is methyl, q is 2, p is 2, R⁴ is methyl, R¹ is —C(═O)R⁹, R⁹ is ethyl, and R⁶ is

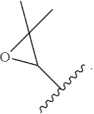 [SMILES: *C1OC1(C)C]

In certain embodiments, each instance of R³ is methyl, q is 2, p is 2, R⁴ is methyl, R¹ is —C(═O)R⁹, R⁹ is propyl, and R⁶ is

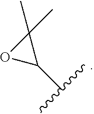 [SMILES: *C1OC1(C)C]

In certain embodiments, each instance of R³ is methyl, q is 2, p is 2, R⁴ is methyl, R is —C(═O)R⁹, R⁹ is

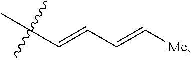 [SMILES: */C=C/C=C/C]

### and R⁶ is

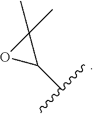 [SMILES: *C1OC1(C)C]

In certain embodiments, each instance of R³ is methyl, q is 2, p is 2, R⁴ is methyl, R¹ is —C(═O)R⁹, R⁹ is

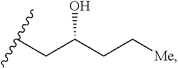 [SMILES: *C[C@H](O)CCC]

### and R⁶ is

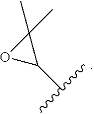 [SMILES: *C1OC1(C)C]

In certain embodiments, each instance of R³ is methyl, q is 2, p is 2, R⁴ is methyl, R¹ is —C(═O)R⁹, R⁹ is

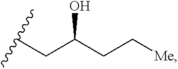 [SMILES: *C[C@@H](O)CCC]

### and R⁶ is

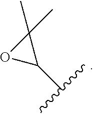 [SMILES: *C1OC1(C)C]

In certain embodiments, each instance of R³ is methyl, q is 2, p is 2, R⁴ is methyl, R¹ is —C(═O)R⁹, R⁹ is methyl, and R⁶ is

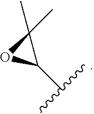

In certain embodiments, each instance of R³ is methyl, q is 2, p is 2, R⁴ is methyl, R is —C(═O)R⁹, R⁹ is ethyl, and R⁶ is

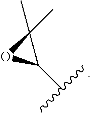

In certain embodiments, each instance of R³ is methyl, q is 2, p is 2, R⁴ is methyl, R is —C(═O)R⁹, R⁹ is propyl, and R⁶ is

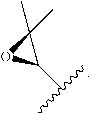

In certain embodiments, each instance of R³ is methyl, q is 2, p is 2, R⁴ is methyl, R¹ is —C(═O)R⁹, R⁹ is

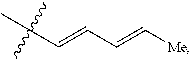 [SMILES: */C=C/C=C/C]

### and R⁶ is

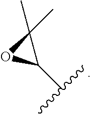

In certain embodiments, each instance of R³ is methyl, q is 2, p is 2, R⁴ is methyl, R¹ is —C(═O)R⁹, R⁹ is

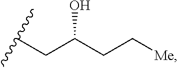 [SMILES: *C[C@H](O)CCC]

### and R⁶ is

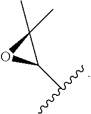

In certain embodiments, each instance of R³ is methyl, q is 2, p is 2, R⁴ is methyl, R¹ is —C(═O)R⁹, R⁹ is

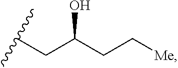 [SMILES: *C[C@@H](O)CCC]

### and R⁶ is

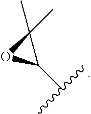

In certain aspects, a compound of Formula (I) is of the formula:

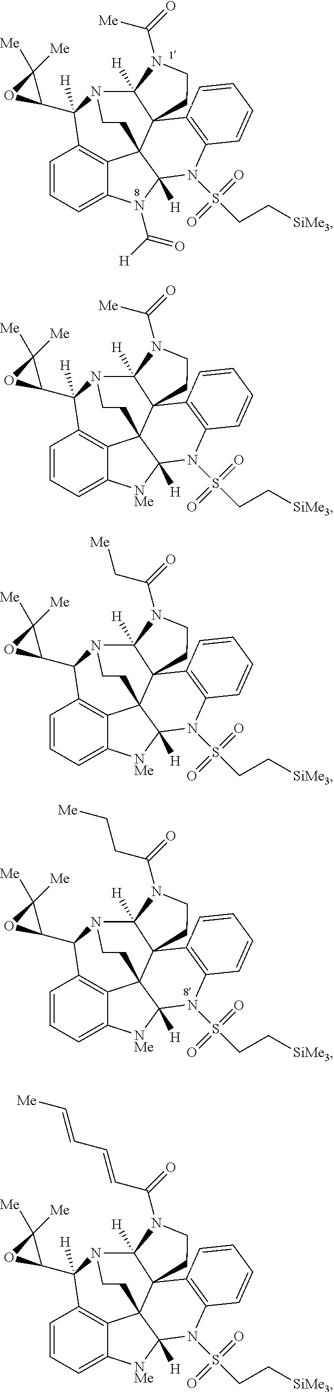

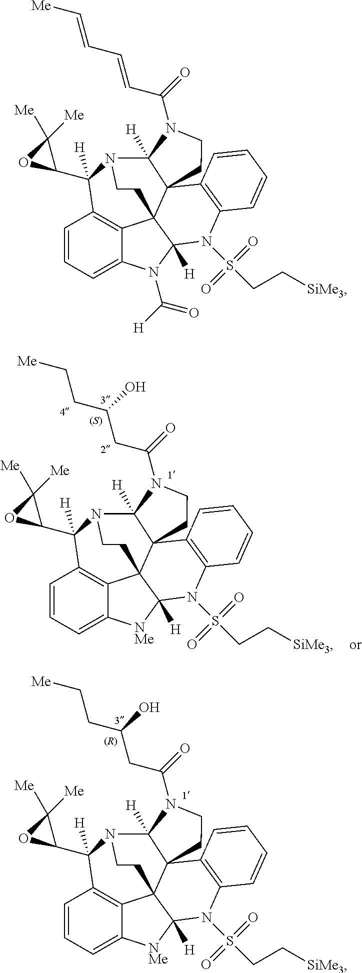

or a salt, tautomer, or stereoisomer thereof.

In certain aspects, the disclosure provides a compound having the formula:

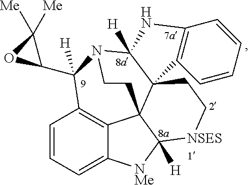

or a salt, tautomer, or stereoisomer thereof.

In certain embodiments of a compound of Formula (V), R¹⁴ is —CN. In certain embodiments of a compound of Formula (V), R¹⁴ is —CN, and R⁶ is

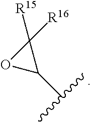

In certain embodiments of a compound of Formula (V), R¹⁴ is —CN, and R⁶ is

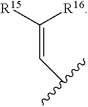

In certain embodiments of a compound of Formula (V), R¹³ is hydrogen, R^{13′} is hydrogen, and R⁴ is hydrogen. In certain embodiments of a compound of Formula (V), R¹³ is

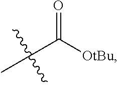 [SMILES: *C(=O)OC(C)(C)C]

### R^{13′} is

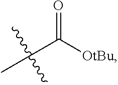 [SMILES: *C(=O)OC(C)(C)C]

and R⁴ is hydrogen. In certain embodiments of a compound of Formula (V), R¹³ is hydrogen, R^{13′} is hydrogen, and R⁴ is methyl. In certain embodiments of a compound of Formula (V), R¹³ is

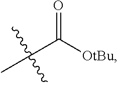 [SMILES: *C(=O)OC(C)(C)C]

### R^{13′} is

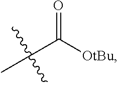 [SMILES: *C(=O)OC(C)(C)C]

and R⁴ is methyl. In certain embodiments of a compound of Formula (V), R¹³ is

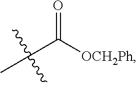

### R^{13′} is

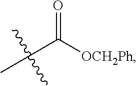

and R⁴ is hydrogen. In certain embodiments of a compound of Formula (V), R¹³ is

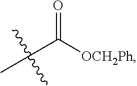

### R^{13′} is

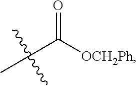

and R⁴ is methyl.

In certain embodiments, a compound of Formula (V) is of the formula:

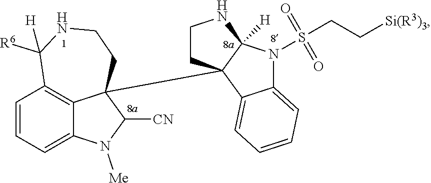

or a salt, tautomer, or stereoisomer thereof. In certain embodiments, a compound of Formula (V) is of the formula:

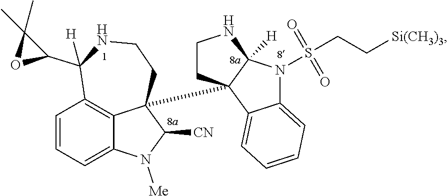

or a salt, tautomer, or stereoisomer thereof. In certain embodiments, a compound of Formula (V) is of the formula:

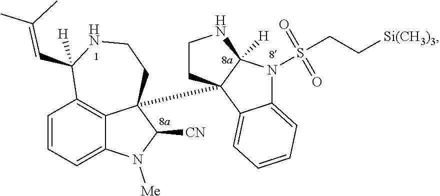

or a salt, tautomer, or stereoisomer thereof.

In certain embodiments of a compound of Formula (III′), R⁶ is

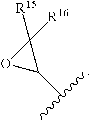

In certain embodiments of a compound of Formula (III′), R⁶ is

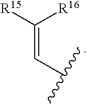

In certain embodiments of a compound of Formula (III′), R¹³ is hydrogen, and R⁴ is hydrogen. In certain embodiments of a compound of Formula (III′), R¹³ is

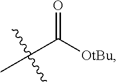 [SMILES: *C(=O)OC(C)(C)C]

and R⁴ is hydrogen. In certain embodiments of a compound of Formula (III′), R¹³ is hydrogen, and R⁴ is methyl. In certain embodiments of a compound of Formula (III′), R¹³ is

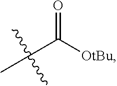 [SMILES: *C(=O)OC(C)(C)C]

and R⁴ is methyl. In certain embodiments of a compound of Formula (III′), R¹³ is

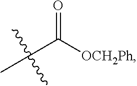

and R⁴ is hydrogen. In certain embodiments of a compound of Formula (III′), R¹³ is

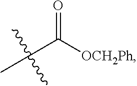

and R⁴ is methyl.

In certain embodiments, a compound of Formula (III′) is of the formula:

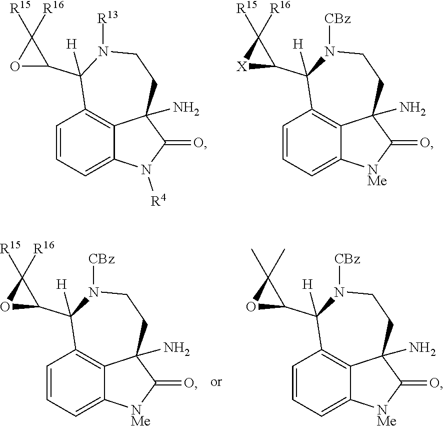

or a salt, tautomer, or stereoisomer thereof. In certain embodiments, a compound of Formula (III′) is of the formula:

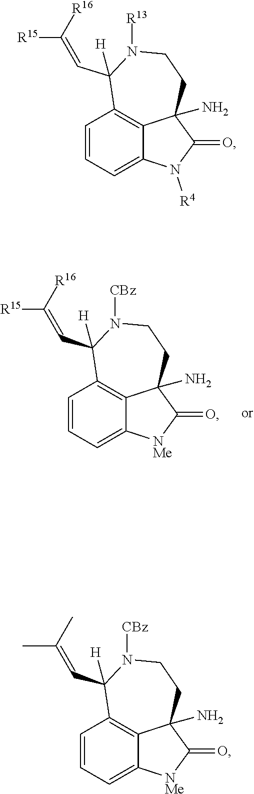

or a salt, tautomer, or stereoisomer thereof.

In certain embodiments of a compound of Formula (XIV), q is 1, and R³ is substituted or unsubstituted C₁-C₁₂ alkyl. In certain embodiments of a compound of Formula (XIV), q is 2, and R³ is substituted or unsubstituted C₁-C₁₂ alkyl. In certain embodiments of a compound of Formula (XIV), q is 3, and R³ is substituted or unsubstituted C₁-C₁₂ alkyl. In certain embodiments of a compound of Formula (XIV), q is 4, and R³ is substituted or unsubstituted C₁-C₁₂ alkyl. In certain embodiments of a compound of Formula (XIV), q is 5, and R³ is substituted or unsubstituted C₁-C₁₂ alkyl. In certain embodiments of a compound of Formula (XIV), q is 6, and R³ is substituted or unsubstituted C₁-C₁₂ alkyl.

In certain embodiments of a compound of Formula (XIV), q is 1, and R is substituted or unsubstituted aryl. In certain embodiments of a compound of Formula (XIV), q is 2, and R³ is substituted or unsubstituted aryl. In certain embodiments of a compound of Formula (XIV), q is 3, and R³ is substituted or unsubstituted aryl. In certain embodiments of a compound of Formula (XIV), q is 4, and R³ is substituted or unsubstituted aryl. In certain embodiments of a compound of Formula (XIV), q is 5, and R³ is substituted or unsubstituted aryl. In certain embodiments of a compound of Formula (XIV), q is 6, and R³ is substituted or unsubstituted aryl.

In certain embodiments of a compound of Formula (XIV), q is 1, and R³ is substituted or unsubstituted heteroaryl. In certain embodiments of a compound of Formula (XIV), q is 2, and R³ is substituted or unsubstituted heteroaryl. In certain embodiments of a compound of Formula (XIV), q is 3, and R³ is substituted or unsubstituted heteroaryl. In certain embodiments of a compound of Formula (XIV), q is 4, and R³ is substituted or unsubstituted heteroaryl. In certain embodiments of a compound of Formula (XIV), q is 5, and R³ is substituted or unsubstituted heteroaryl. In certain embodiments of a compound of Formula (XIV), q is 6, and R³ is substituted or unsubstituted heteroaryl.

In certain embodiments, a compound of Formula (XIV) is of the formula:

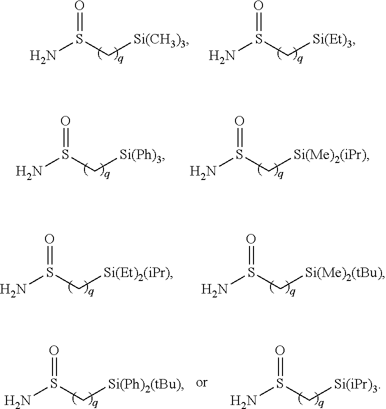

In certain embodiments, a compound of Formula (XIV) is of the formula:

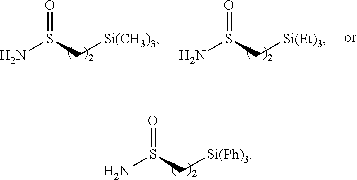

In certain embodiments, a compound of Formula (XIV) is of the formula:

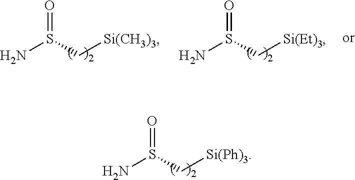

In certain embodiments, a compound of Formula (XIV) is of the formula:

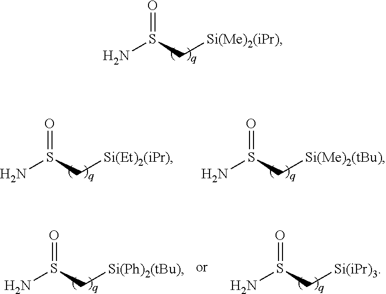

In certain embodiments, a compound of Formula (XIV) is of the formula:

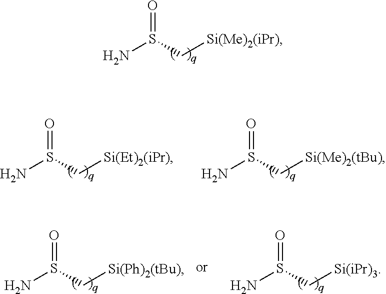

In certain embodiments, the moiety

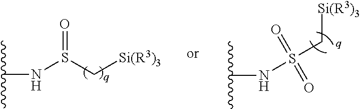

in a compound of any of Formulae (I), (V) to (XII), and (VII′) is derived from a compound of Formula (XIV):

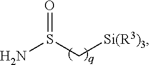

or a salt, tautomer, or stereoisomer thereof.

### Methods of Preparation

The present disclosure is in various embodiments directed to a unified and convergent approach to the synthesis of communesin alkaloids involving the stereocontrolled oxidative union of two dissimilar tryptamine derivatives followed by reorganization of a C3a-C3a′ linked heterodimer. This method involves the directed and stereocontrolled union of two dissimilar fragments followed by selective reorganization of a C3a-C3a′ linked heterodimer to a single constitutional isomer consistent with the communesin skeleton (Scheme A).

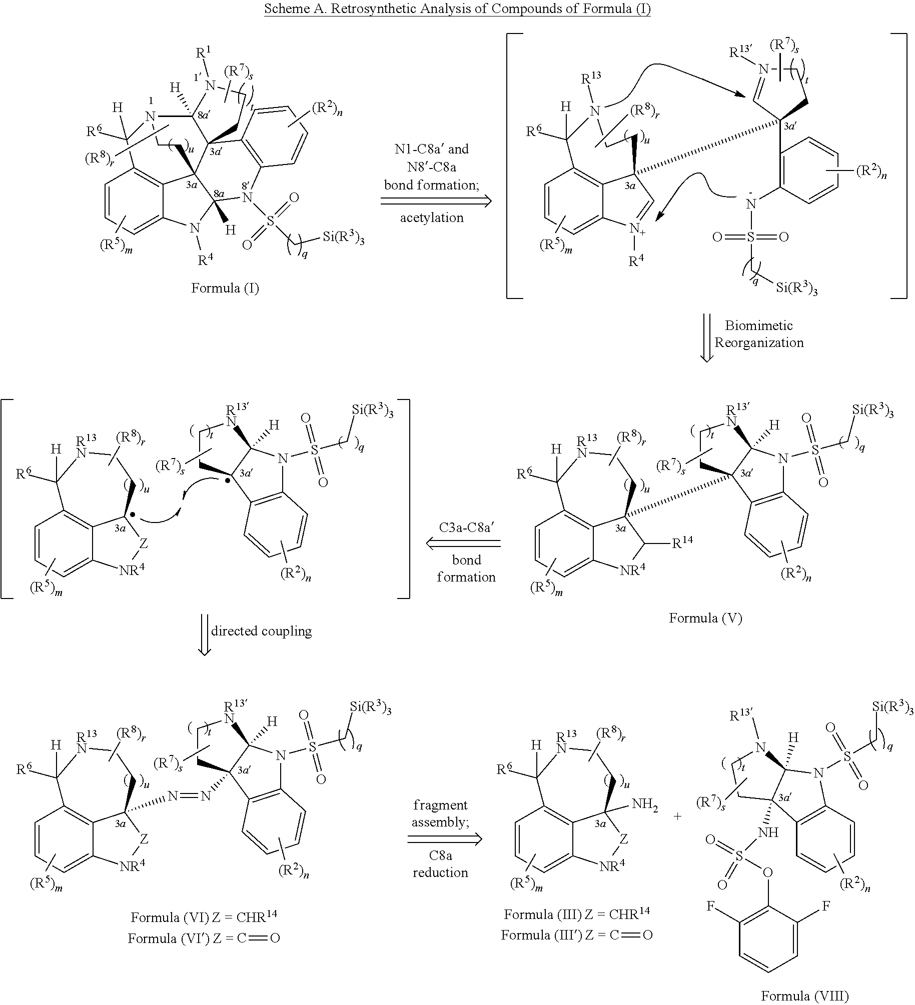

In certain aspects, the present disclosure provides a method of making a compound of Formula (XIV):

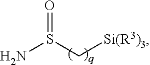

or a salt, tautomer, or stereoisomer thereof, wherein R³ and q are as defined herein. In some aspects, the method comprising one or more process or reaction is referred to as “step a”. In some embodiments, the method is a step in the synthesis of communesin alkaloids and derivatives thereof. In certain aspects, the method comprised reacting

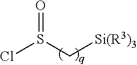

with a chiral controller in the presence of a base, wherein the chiral controller comprises a hydroxy moiety, and then reacting with a H₂N⁻ source. In some embodiments,

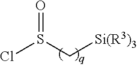

is reacted with (−)-diacetone-D-glucose in the presence of an amine base, and then reacted with lithium bis(trimethylsilyl)amide to yield a compound of Formula (XIV), wherein the compound is of the formula:

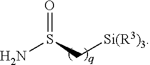

In certain embodiments,

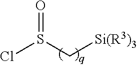

is reacted with (+)-diacetone-D-glucose in the presence of an amine base, and then reacted with lithium bis(trimethylsilyl)amide to yield a compound of Formula (XIV), wherein the compound is of the formula:

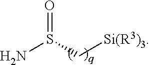

In some embodiments,

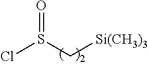

is reacted with (−)-diacetone-D-glucose in the presence of an amine base, and then reacted with lithium bis(trimethylsilyl)amide to yield a compound of Formula (XIV), wherein the compound is of the formula:

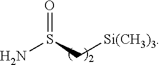

The present disclosure further provides methods of synthesizing a compound of Formula (XII):

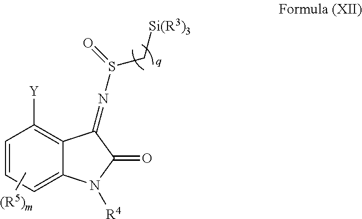

or a salt, tautomer, or stereoisomer thereof, wherein Y, R⁵, m, R⁴, q, and R³ are as defined herein. In some aspects, the method comprising one or more process or reaction is referred to as “step b”. In some embodiments, the method is a step in the synthesis of communesin alkaloids and derivatives thereof. In certain embodiments, the method comprises reacting a compound of Formula (XIII):

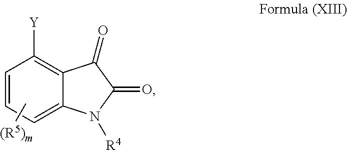

or a salt, tautomer, or stereoisomer thereof, with a compound of Formula (XIV):

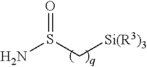

or a salt, tautomer, or stereoisomer thereof. In certain embodiments, the method further comprises the presence of a Ti or Zr alkoxide. In some embodiments, the method comprises reacting a compound of Formula (XIII) with a compound of Formula (XIV) in the presence of titanium(IV) ethoxide to generate a compound of Formula (XII). In certain embodiments, the reaction is carried out at or about room temperature.

Further provided by the present disclosure are methods of synthesizing a compound of Formula (XI):

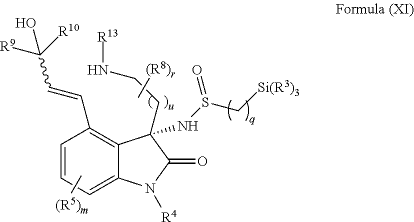

or a salt, tautomer, or stereoisomer thereof, wherein R⁹, R¹⁰, R⁵, m, R⁴, q, R³, u, R⁸, r, and R¹³ are as defined herein. In some aspects, the method comprising one or more process or reaction is referred to as “step c”. In some embodiments, the method is a step in the synthesis of communesin alkaloids and derivatives thereof. In certain embodiments, the method comprises a compound of Formula (XII):

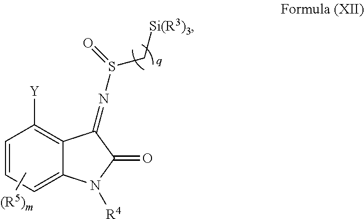

or a salt, tautomer, or stereoisomer thereof, wherein Y is a halogen (e.g., bromo), comprising the steps of: (1) allylation; (2) ozonolysis; (3) ozonide reduction; (4) Mitsunobu displacement; (5) desulfonylation; and (6) Mizoroki-Heck reaction. In certain embodiments, a compound of Formula (XII) undergoes an allylation reaction. In certain embodiments, a compound of Formula (XII) is reacted with allylmagnesium bromide. In some embodiments, a compound of Formula (XII) is reacted with allylmagnesium bromide in the presence of MgBr₂, wherein the reaction occurs at low temperatures (e.g., about −85 to −70° C.). In certain embodiments, after the allylation reaction, the product undergoes an ozonolysis reaction. In some embodiments, the product of the allylation reaction is reacted with O₃. In some embodiments, the product of the allylation reaction is reacted with O₃ in the presence of an alcohol, wherein the reaction occurs at low temperatures (e.g., about −85 to −70° C.). In some embodiments, after the ozonolysis reaction, ozonide reduction occurs. In some embodiments, the ozonide reduction is in situ. In certain embodiments, the ozonide reduction comprises a reducing agent. In certain aspects, the reducing agent is NaBH₄. In some embodiments the ozonide reduction is carried out at low temperatures (e.g., about −85 to −70° C.). In some embodiments, the product of the ozonide reduction is subjected to a Mitsunobu displacement reaction. In certain embodiments, the Mitsunobu displacement reaction comprises isopropyl azodicarboxylate, and triphenylphosphine. In some embodiments, the Mitsunobu displacement reaction further comprises N-carbobenoxy-2-nitrobenzenesulfonamide. In certain embodiments, the Mitsunobu displacement is carried out at elevated temperatures (e.g., about 30-70° C. (e.g., 50° C.)). In certain embodiments, after the Mitsunobu reaction, the product undergoes desulfonylation. In some embodiments, the desulfonylation occurs in situ. In certain embodiments, the desulfonylation comprises PhSH and a base. In some embodiments, the desulfonylation is carried out at elevated temperatures (e.g., about 30-70° C. (e.g., 50° C.)). In certain embodiments, the product of the Mitsunobu displacement followed by desulfonylation undergoes a Mizoroki-Heck reaction. In some embodiments, the Mizoroki-Heck reaction comprises 1,1-dimethylallyl alcohol, a palladium catalyst, and a base. In some embodiments, the base is Ag₂CO₃. In certain aspects, the palladium catalyst is generated in situ starting from a palladium source. In some embodiments, the palladium source is Pd(OAc)₂. In certain embodiments, the Mizoroki-Heck reaction is carried out at elevated temperature (e.g., about 30-110° C. (e.g., 90° C.)).

In certain aspects, the present disclosure provides a method of synthesizing a compound of Formula (X):

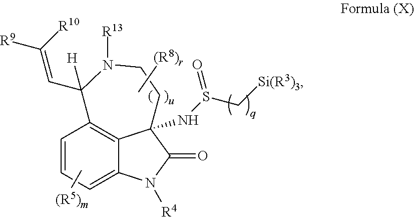

or a salt, tautomer, or stereoisomer thereof, wherein R⁹, R¹⁰, R⁵, m, R⁴, q, R³, u, R⁸, r, and R¹³ are as defined herein. In some aspects, the method comprising one or more process or reaction is referred to as “step d”. In some embodiments, the method is a step in the synthesis of communesin alkaloids and derivatives thereof. In certain embodiments, the method of synthesizing a compound of Formula (X) comprises allylic amination of a compound of Formula (XI):

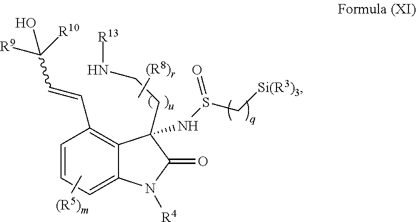

or a salt, tautomer, or stereoisomer thereof. In certain embodiments, the allylic amination comprises a Lewis acid. In some embodiments, the allylic amination comprises calcium (II) trifluoromethansulfonate as the Lewis acid. In some embodiments, the allylic amination comprises magnesium(II) trifluoromethanesulfonate or magnesium(II) perchlorate as the Lewis acid. In some embodiments, the allylic amination is carried out at elevated temperatures (e.g., about 30-110° C. (e.g., 80° C.)). In some embodiments, the allylic amination comprises calcium (II) trifluoromethanesulfonate at 80° C. in acetonitrile. In some embodiments, the allylic amination comprises PdCl₂MeCN₂ at elevated temperature.

In certain aspects, the present disclosure provides methods of synthesizing a compound of Formula (IX):

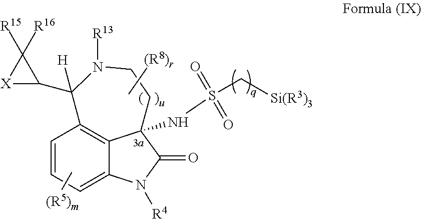

or a salt, tautomer, or stereoisomer thereof, wherein X, R¹⁵, R¹⁶, R⁵, m, R⁴, q, R³, u, R⁸, r, and R¹³ are as defined herein. In some aspects, the method comprising one or more process or reaction is referred to as “step e”. In some embodiments, the method is a step in the synthesis of communesin alkaloids and derivatives thereof. In certain embodiments, the method of synthesizing a compound of Formula (IX) comprises epoxidation of a compound of Formula (X):

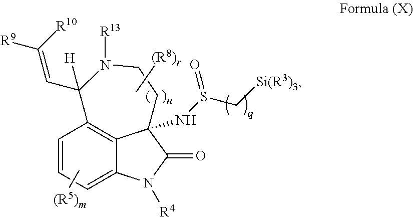

or a salt, tautomer, or stereoisomer thereof. In certain embodiments, the epoxidation comprises reacting a compound of Formula (X) with methyl(trifluoromethyl)dioxirane (TFDO). In some embodiments, TFDO is generated in situ. In certain embodiments, the epoxidation comprises reacting a compound of Formula (X) with a peroxy acid. In certain embodiments, the epoxidation comprises reacting a compound of Formula (X) with meta-Chloroperoxybenzoic acid.

The present disclosure also provides methods of synthesizing a compound of Formula (III′):

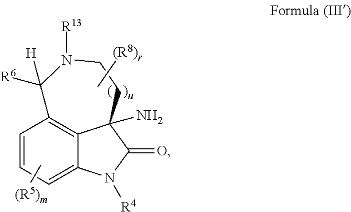

or a salt, tautomer, or stereoisomer thereof, wherein X, R¹⁵, R¹⁶, R⁵, R⁶, m, R⁴, u, R⁸, r, and R¹³ are as defined herein. In some aspects, the method comprising one or more process or reaction is referred to as “step f”. In some embodiments, the method is a step in the synthesis of communesin alkaloids and derivatives thereof. In certain aspects, a compound of Formula (III′) is synthesized by a method comprising desulfonylation of a compound of Formula (IX):

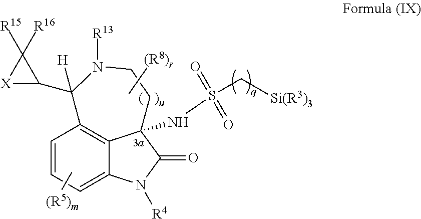

or a salt, tautomer, or stereoisomer thereof. In certain embodiments, the desulfonylation occurs at the sulfonamide at C3a in a compound of Formula (IX). In certain embodiments, the desulfonylation comprises a fluoride source. In certain embodiments, the desulfonylation comprises cesium fluoride (CsF). In certain embodiments, the desulfonylation comprises tris(dimethylamino)sulfonium difluorotrimethylsilicate (TASF). In certain embodiments, the desulfonylation further comprises wet DMF at elevated temperatures (e.g., about 70-110° C. (e.g., 100° C.)).

Also provided herein are methods of synthesizing a compound of Formula (VII′):

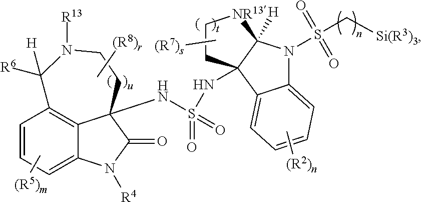

or a salt, tautomer, or stereoisomer thereof, wherein R⁶, R⁵, m, R⁴, u, R⁸, r, R¹³, R², n, q, R³, t, R^{13′}, t, R⁷, and s are as defined herein. In some aspects, the method comprising one or more process or reaction is referred to as “step g”. In some embodiments, the method is a step in the synthesis of communesin alkaloids and derivatives thereof. In some aspects, the present disclosure provides methods of synthesizing a compound of Formula (VII′) comprising reacting a compound of Formula (III′):

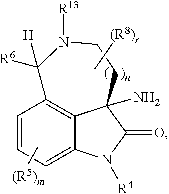

or a salt, tautomer, or stereoisomer thereof, and a compound of Formula (VIII):

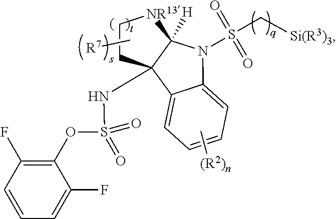

or a salt, tautomer, or stereoisomer thereof. In some embodiments, the reaction between a compound of Formula (III′) and a compound of Formula (VIII) is a nucleophilic substitution reaction. In some embodiments, the reaction between a compound of Formula (III′) and a compound of Formula (VIII) comprises a base. In some embodiments, the reaction between a compound of Formula (III′) and a compound of Formula (VIII) comprises 4-(dimethylamino)pyridine (DMAP). In some embodiments, the reaction between a compound of Formula (III′) and a compound of Formula (VIII) comprises DMAP wherein the reaction is performed at about room temperature.

In certain aspects, the present disclosure provides methods of synthesizing a compound of Formula (VII):

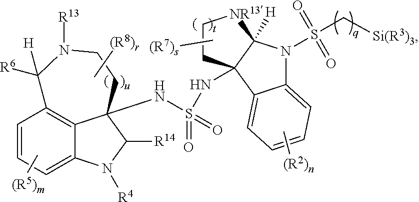

or a salt, tautomer, or stereoisomer thereof, wherein R⁶, R⁵, m, R⁴, u, R⁸, r, R¹³, R², n, q, R³, t, R^{13′}, t, R⁷, s, and R¹⁴ are as defined herein. In some aspects, the method comprising one or more process or reaction is referred to as “step h”. In some embodiments, the method is a step in the synthesis of communesin alkaloids and derivatives thereof. In certain embodiments, the method of synthesizing a compound of Formula (VII) comprises partial reduction of a compound of Formula (VII′):

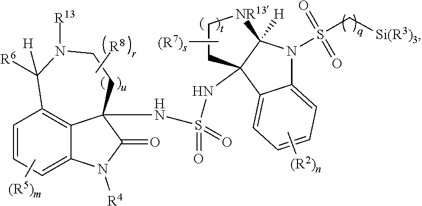

or a salt, tautomer, or stereoisomer thereof. In certain embodiments, the partial reduction comprises a reducing agent. In some embodiments, the reducing agent is a borohydride reducing agent. In certain embodiments, a compound of Formula (VII′) is reduced with LiBH₄. In some embodiments, after partial reduction, the method further comprises reaction with a cyanide source. In some embodiments, the cyanide source is trimethylsilyl cyanide. In certain embodiments, a compound of Formula (VII′) is reduced with LiBH₄ followed by reaction with a cyanide source (e.g., trimethylsilyl cyanide) in wet hexafluoroisopropanol to produce a compound of Formula (VII).

Also provided herein are methods of synthesizing a compound of Formula (VII):

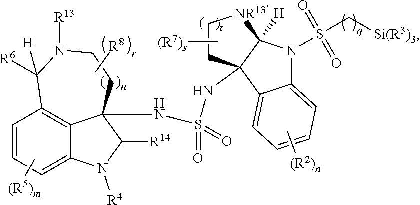

or a salt, tautomer, or stereoisomer thereof, wherein R⁶, R⁵, m, R⁴, u, R⁸, r, R¹³, R², n, q, R³, t, R^{13′}, t, R⁷, s, and R¹⁴ are as defined herein. In some aspects, the method comprising one or more process or reaction is referred to as “step g”. In some embodiments, the method is a step in the synthesis of communesin alkaloids and derivatives thereof. In some aspects, the present disclosure provides methods of synthesizing a compound of Formula (VII) comprising reacting a compound of Formula (III):

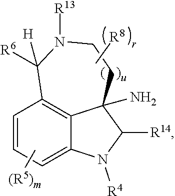

or a salt, tautomer, or stereoisomer thereof, and a compound of Formula (VIII):

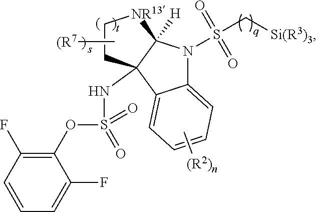

or a salt, tautomer, or stereoisomer thereof. In some embodiments, the reaction between a compound of Formula (III) and a compound of Formula (VIII) comprises a base. In some embodiments, the reaction between a compound of Formula (III) and a compound of Formula (VIII) is a nucleophilic substitution reaction. comprises 4-(dimethylamino)pyridine (DMAP). In some embodiments, the reaction between a compound of Formula (III′) and a compound of Formula (VIII) comprises DMAP wherein the reaction is performed at about room temperature.

Further provided herein are methods of synthesizing a compound of Formula (VI):

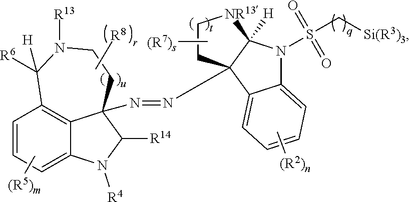

or a salt, tautomer, or stereoisomer thereof, wherein R⁶, R⁵, m, R⁴, R¹⁴, u, R⁸, r, R¹³, R², n, q, R³, t, R^{13′}, t, R⁷, and s are as defined herein. In some aspects, the method comprising one or more process or reaction is referred to as “step i”. In some embodiments, the method is a step in the synthesis of communesin alkaloids and derivatives thereof. In some aspects, the present disclosure provides methods of synthesizing a compound of Formula (VI) comprising extrusion of dinitrogen from a compound of extrusion of sulfur dioxide from a compound of Formula (VII):

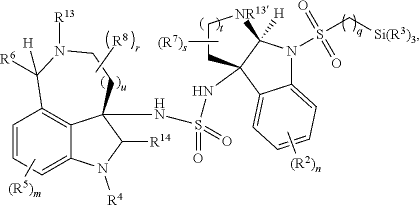

or a salt, tautomer, or stereoisomer thereof. In certain embodiments, the extrusion of sulfur dioxide from a compound of Formula (VII) comprises reacting a phosphazene base with a compound of Formula (VII). In certain embodiments, the method of synthesizing a compound of Formula (VI), comprises reacting a compound of Formula (VII) with a phosphazene base. In certain embodiments, the method of synthesizing a compound of Formula (VI), comprises reacting a compound of Formula (VII) with 2-tert-butylimino-2-diethylamino-1,3-dimethylperhydro-1,3,2,-diazaphosphorine (BEMP). In certain embodiments, the method of synthesizing a compound of Formula (VI), comprises reacting a compound of Formula (VII) with N-chloro-N-methylbenzamide and BEMP. In certain embodiments, the method of synthesizing a compound of Formula (VI), comprises reacting a compound of Formula (VII) with N-chloro-N-methylbenzamide and BEMP in an alcohol. In certain embodiments, the method of synthesizing a compound of Formula (VI), comprises reacting a compound of Formula (VII) with N-chloro-N-methylbenzamide and BEMP in methanol.

In some aspects, the present disclosure provides methods of synthesizing a compound of Formula (V):

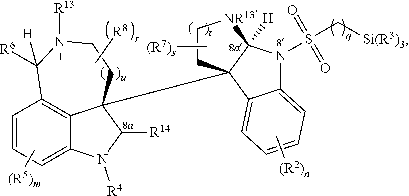

or a salt, tautomer, or stereoisomer thereof, wherein R⁶, R⁵, m, R⁴, R¹⁴, u, R⁸, r, R¹³, R², n, q, R³, t, R^{13′}, t, R⁷, and s are as defined herein. In some aspects, the method comprising one or more process or reaction is referred to as “step j”. In some embodiments, the method is a step in the synthesis of communesin alkaloids and derivatives thereof. In some aspects, the present disclosure provides methods of synthesizing a compound of Formula (V) comprising extrusion of dinitrogen from a compound of Formula (VI):

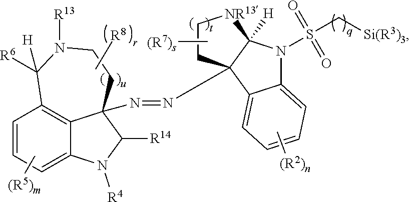

or a salt, tautomer, or stereoisomer thereof. In certain embodiments, N₂ is extruded from a compound of Formula (VI) to produce a compound of Formula (V). In certain embodiments, a compound of Formula (VI) is photoexcited to extrude N₂ to give a compound of Formula (V). In certain embodiments, a compound of Formula (VI) is photoexcited to extrude N₂ followed by radical recombination to give a compound of Formula (V). In some embodiments, the photoexcitation comprises 380 nm light. In some embodiments, the photoexcitation comprises 350 nm light. In some embodiments, the photoexcitation comprises 300 nm light.

In certain embodiments the present disclosure provides methods of deprotecting a compound of Formula (V), wherein the compound is of the formula:

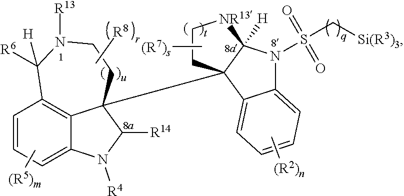

or a salt, tautomer, or stereoisomer thereof, wherein R¹³ and R^{13′} are nitrogen protecting groups

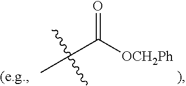

to generate a compound of Formula (V), wherein R¹³ and R^{13′} are both hydrogen. In certain embodiments, the deprotection comprises hydrogen gas and a palladium catalyst. In certain embodiments, the palladium catalyst is Pd(OH)₂/C.

Also provided herein are methods of making a compound of Formula (I):

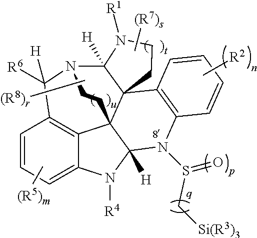

or a salt, tautomer, or stereoisomer thereof, wherein R⁶, R⁵, m, R⁴, u, R⁸, r, R¹, R², n, q, R³, t, t, R⁷, and s are as defined herein. In some aspects, the method comprising one or more process or reaction is referred to as “step k”. In some embodiments, the method is a step in the synthesis of communesin alkaloids and derivatives thereof. In some aspects, the present disclosure provides methods of synthesizing a compound of Formula (I) comprising forming a bond between the nitrogen atom at the position NI and the carbon atom at the position C8a′, and a bond between the nitrogen atom at the position N8′ and the carbon atom at the position C8a in a compound of Formula (V):

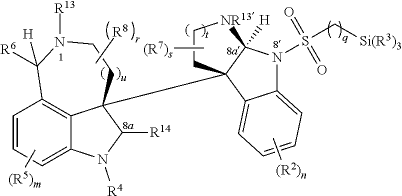

or a salt, tautomer, or stereoisomer thereof. In certain embodiments, the method is referred to a rearrangement reaction. In some embodiments, the method comprises reacting a compound of Formula (V) with an alkoxide, followed by acetylation. In certain embodiments, the reaction further comprises neutralization of excess alkoxide before acetylation. In certain embodiments, pyridinium p-toluenesulfonate (PPTS) is used to neutralize the excess alkoxide. In some embodiments, the method comprises reacting a compound of Formula (V) to ethanolic lithium tert-butoxide, followed by in situ neutralization of excess alkoxide with pyridinium p-toluenesulfonate (PPTS), which is followed by acetylation. In some embodiments, the method comprises reacting a compound of Formula (V) to methanolic lithium tert-butoxide, followed by in situ neutralization of excess alkoxide with pyridinium p-toluenesulfonate (PPTS), which is followed by acetylation. In some embodiments, the method comprises reacting a compound of Formula (V) to deuteromethanolic lithium tert-butoxide, followed by in situ neutralization of excess alkoxide with pyridinium p-toluenesulfonate (PPTS), which is followed by acetylation. In certain embodiments, the acetylation reaction comprises an anhydride or an aldol adduct. In certain embodiments, the anhydride is selected from acetic anhydride, sorbic anhydride, propionic anhydride, and butyric anhydride. In certain embodiments, the aldol adduct is selected from an (S),(R)-aldol adduct

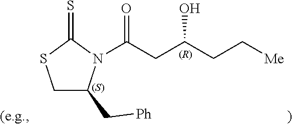

and an (S),(S)-aldol adduct

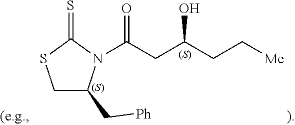

In certain embodiments, the reaction with an alkoxide is carried out at elevated temperature (e.g., 30-80° C. (e.g., 60° C.)). In certain embodiments, the neutralization and acetylation reactions are carried out at about room temperature.

The present disclosure also provides methods of making a compound of Formula (I′):

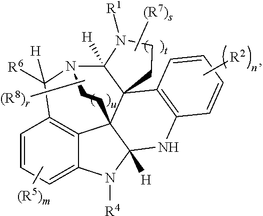

or a salt, tautomer, or stereoisomer thereof, wherein R⁶, R⁵, m, R⁴, u, R⁸, r, R¹, R², n, t, R⁷, and s are as defined herein. In some aspects, the method comprising one or more process or reaction is referred to as “step 1”. In some embodiments, the method is a step in the synthesis of communesin alkaloids and derivatives thereof. In some aspects, the present disclosure provides methods of synthesizing a compound of Formula (I′) comprising desulfonylation of position N8′ in a compound of Formula (I):

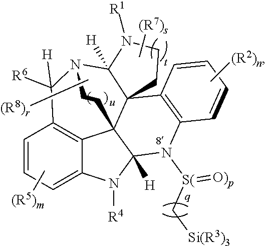

or a salt, tautomer, or stereoisomer thereof. In certain embodiments, a compound of Formula (I) is desulfonylated to produce a natural product. In certain embodiments, a compound of Formula (I) is desulfonylated to produce a compound of Formula (I′) which is communesin A, communesin B, communesin C, communesin D, communesin E, communesin F, communesin G, communesin H, or communesin I. In certain embodiments, a compound of Formula (I) is desulfonylated to produce a compound of Formula (I′) which is (−)-communesin A, (−)-communesin B, (−)-communesin C, (+)-communesin D, (−)-communesin E, (−)-communesin F, (−)-communesin G, (−)-communesin H, or (−)-communesin I. In certain embodiments, the desulfonylation of a compound of Formula (I) comprises a fluoride source. In some embodiments, the desulfonylation comprises tris(dimethylamino)sulfonium difluorotrimethylsilicate. In certain embodiments, the desulfonylation is carried out at about room temperature. In some embodiments, the desulfonylation is carried out at elevated temperature (e.g., 30-90° C. (e.g., 60° C., 45° C.)).

In certain aspects, disclosed herein are methods synthesizing communesin alkaloids and derivatives thereof (e.g., compounds of Formula (I) or (I′)), comprising a single method comprising one or more process or reaction (i.e., “step”) or any number of individual steps disclosed herein. In certain aspects, the disclosure provides methods of synthesizing communesin alkaloids and derivatives thereof, comprising steps a, b, c, d, e, f, g′, h, i, j, k, and l to produce a compound of Formula (I′). In certain aspects, the disclosure provides methods of synthesizing communesin alkaloids and derivatives thereof, comprising steps a, b, c, d, e, f, g′, h, i, j, and k to produce a compound of Formula (I). In certain aspects, the disclosure provides methods of synthesizing communesin alkaloids and derivatives thereof, comprising steps g′, h, i, j, k, and l to produce a compound of Formula (I′). In certain aspects, the disclosure provides methods of synthesizing communesin alkaloids and derivatives thereof, comprising steps g′, h, i, j, and k to produce a compound of Formula (I). In certain aspects, the disclosure provides methods of synthesizing communesin alkaloids and derivatives thereof, comprising steps g, i, j, k, and l to produce a compound of Formula (I′). In certain aspects, the disclosure provides methods of synthesizing communesin alkaloids and derivatives thereof, comprising steps g, i, j, and k to produce a compound of Formula (I). In certain aspects, the disclosure provides methods of synthesizing communesin alkaloids and derivatives thereof, comprising steps k and l to produce a compound of Formula (I′). In certain aspects, the disclosure provides methods of synthesizing communesin alkaloids and derivatives thereof, comprising step k to produce a compound of Formula (I).

In certain embodiments, a compound described herein is a compound of any one of the formulae described herein, or a salt, tautomer, or stereoisomer thereof. In certain embodiments, a compound described herein is a compound of any one of the formulae described herein, or a pharmaceutically acceptable salt, tautomer, or stereoisomer thereof. In certain embodiments, a compound described herein is a compound of any one of the formulae described herein, or a salt thereof. In certain embodiments, a compound described herein is a compound of any one of the formulae described herein, or a pharmaceutically acceptable salt thereof.

### Compositions and Kits

The present disclosure provides compositions (e.g., pharmaceutical compositions) comprising a compound as described herein, and optionally an excipient (e.g., pharmaceutically acceptable excipient). In certain embodiments, the composition is a pharmaceutical composition. In certain embodiments, the excipient is a pharmaceutically acceptable excipient. In certain embodiments, the composition comprises a compound described herein, or a pharmaceutically acceptable salt, tautomer, or stereoisomer thereof, and a pharmaceutically acceptable excipient.

In certain embodiments, the pharmaceutical compositions are useful for treating a disease in a subject in need thereof. In certain embodiments, the pharmaceutical compositions are useful for preventing a disease in a subject. In certain embodiments, the compositions are useful for treating an insect infestation.

In certain embodiments, the compound described herein is provided in an effective amount in the pharmaceutical composition. In certain embodiments, the effective amount is a therapeutically effective amount. In certain embodiments, the effective amount is a prophylactically effective amount.

In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a cancer in a subject in need thereof. In certain embodiments, the effective amount is an amount effective for preventing cancer in a subject in need thereof. In some embodiments, the cancer is cervical cancer, lung cancer, breast cancer, colorectal cancer, or prostate cancer. In some embodiments, the cancer is a cancer of the blood (e.g., lymphocytic leukemia.

In certain embodiments, the effective amount is an amount effective for treating an infectious disease in a subject in need thereof. In certain embodiments, the effective amount is an amount effective for preventing an infectious disease in a subject in need thereof. In certain embodiments, the effective amount is an amount effective for treating a bacterial infection (e.g., Escherichia coli, Klebsiella pneumoniae, Pseudomonas aeruginosa, Acinetobacter baumanii, Neisseria gonorrhoeae, or Bacillus subtilis) in a subject in need thereof. In certain embodiments, the effective amount is an amount effective for preventing a bacterial infection (e.g., Escherichia coli, Klebsiella pneumoniae, Pseudomonas aeruginosa, Acinetobacter baumanii, Neisseria gonorrhoeae, or Bacillus subtilis) in a subject in need thereof. In certain embodiments, the effective amount is an amount effective for treating a fungal infection (e.g., Candida albicans, Trichophyton mentagrophytes, or Amorphotheca resinae) in a subject in need thereof. In certain embodiments, the effective amount is an amount effective for preventing a fungal infection (e.g., Candida albicans, Trichophyton mentagrophytes, or Amorphotheca resinae) in a subject in need thereof. In certain embodiments, the effective amount is an amount effective for treating a viral infection (e.g., Herpes simplex type 1) in a subject in need thereof. In certain embodiments, the effective amount is an amount effective for preventing a viral infection (e.g., Herpes simplex type 1) in a subject in need thereof.

In certain embodiments, the effective amount is an amount effective for treating abnormal cardiovascular function in a subject in need thereof. In certain embodiments, the effective amount is an amount effective for preventing abnormal cardiovascular function in a subject in need thereof. In certain embodiments, the effective amount is an amount effective for treating bradycardia in a subject in need thereof. In certain embodiments, the effective amount is an amount effective for preventing bradycardia in a subject in need thereof.

In certain embodiments, the effective amount is an amount effective for reducing the risk of developing a disease (e.g., proliferative disease, autoimmune disease, hematological disease, neurological disease, painful condition, psychiatric disorder, or metabolic disorder) in a subject in need thereof. In certain embodiments, the effective amount is an amount effective for preventing a disease in a subject in need thereof.

In certain embodiments, the effective amount is an amount effective for treating an insect infestation. In certain embodiments, the insect infestation is caused by silkworms. In certain embodiments, the insect infestation is caused by silkworms in the third instar larval stage.

In certain embodiments, the effective amount is an amount effective for delivering a pharmaceutical agent to a biological sample or cell. In certain embodiments, the cell is in vitro. In certain embodiments, the cell is in vivo. In certain embodiments, the cell is a malignant cell. In some embodiments, the cell is a premalignant cell.

Pharmaceutical compositions described herein can be prepared by any method known in the art of pharmacology. In general, such preparatory methods include bringing the compound described herein (which may include a therapeutic agent (the “active ingredient”)) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the polymer in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound, or pharmaceutical compositions described herein is suitable for topical administration to the eye of a subject. In some embodiments, provided pharmaceutical formulations of provided compounds are typically prepared for parenteral administration, i.e. bolus, intravenous, intratumor injection with a pharmaceutically acceptable parenteral vehicle and in a unit dosage injectable form. In some embodiments, the compounds having the desired degree of purity is optionally mixed with pharmaceutically acceptable diluents, carriers, excipients or stabilizers (Remington's Pharmaceutical Sciences (1980) 16th edition, Osol, A. Ed.), in the form of a lyophilized formulation or an aqueous solution.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

A provided pharmaceutical composition may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 pg and 1 g, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A compound or composition as described herein, can be administered in combination with one or more additional pharmaceutical agents (e.g., therapeutically and/or prophylactically active agents). The compound or composition can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in diagnosing a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or compositions can be administered concurrently with, prior to, or subsequent to one or more additional pharmaceutical agents, which are different from the compound or composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation.

In certain embodiments, the compounds described herein or pharmaceutical compositions can be administered in combination with an anti-cancer therapy including surgery, radiation therapy, transplantation (e.g., stem cell transplantation, bone marrow transplantation), immunotherapy, and chemotherapy. In certain embodiments, the compounds described herein or pharmaceutical compositions can be administered in combination with an additional therapy. In some embodiments, the compounds described herein or pharmaceutical compositions can be administered in combination with radiation therapy.

Also encompassed by the disclosure are kits (e.g., pharmaceutical packs). The kits provided may comprise a composition or compound described herein and instructions for use. The kits may further comprise a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In another aspect, provided are kits including a first container comprising a compound or composition described herein. In certain embodiments, the kits are useful for delivering an agent (e.g., to a subject, cell, biological sample,). In certain embodiments, the kits are useful for treating a disease (e.g., cancer, bacterial infection, viral infection, fungal infection, cardiovascular abnormalities) in a subject in need thereof. In certain embodiments, the kits are useful for preventing a disease (e.g., cancer, bacterial infection, viral infection, fungal infection, cardiovascular abnormalities) in a subject in need thereof. In certain embodiments, the kits are useful for reducing the risk of developing a disease (e.g., cancer, bacterial infection, viral infection, fungal infection, cardiovascular abnormalities) in a subject in need thereof. In certain embodiments, the kits are useful for inhibiting the activity (e.g., aberrant activity, such as increased activity) of a protein in a subject or cell, tissue, or biological sample. In certain embodiments, the kits are useful for inducing apoptosis of a cell, a cell in a subject, or a cell in a tissue or biological sample. In certain embodiments, the kits are useful for inhibiting proliferation of a cell, a cell in a subject, or a cell in a tissue or biological sample. In certain embodiments, the kits are useful for treating an insect infestation.

In certain embodiments, a kit described herein further includes instructions for using the kit. A kit described herein may also include information as required by a regulatory agency such as the U.S. Food and Drug Administration (FDA). In some embodiments, a kit comprises a compound or composition as described herein and instructions for using the compound or composition. In certain embodiments, the information included in the kits is prescribing information. In certain embodiments, the kits and instructions provide for delivering an agent. In certain embodiments, the kits and instructions provide for treating a disease (e.g., cancer, bacterial infection, viral infection, fungal infection, cardiovascular abnormalities) in a subject in need thereof. In certain embodiments, the kits and instructions provide for preventing a disease(e.g., cancer, bacterial infection, viral infection, fungal infection, cardiovascular abnormalities) in a subject in need thereof. In certain embodiments, the kits and instructions provide for reducing the risk of developing a disease (e.g., cancer, bacterial infection, viral infection, fungal infection, cardiovascular abnormalities) in a subject in need thereof. In certain embodiments, the kits and instructions provide for inhibiting the activity (e.g., aberrant activity, such as increased activity) of a protein in a subject, cell, tissue, or biological sample. In certain embodiments, the kits are useful for inducing apoptosis of a cell, a cell in a subject, or a cell in a tissue or biological sample. In certain embodiments, the kits are useful for inhibiting proliferation of a cell, a cell in a subject, or a cell in a tissue or biological sample. A kit described herein may include one or more additional pharmaceutical agents described herein as a separate composition.

In certain embodiments, the kits are useful for treating an insect infestation. In certain embodiments, the kits are useful for treating an insect infestation caused by silkworms. In certain embodiments, the kits are useful for treating an insect infestation caused by silkworms in the third instar larval stage. In certain embodiments, a kit described herein further includes instructions for using the kit. A kit described herein may also include information as required by a regulatory agency. In some embodiments, a kit comprises a compound or composition as described herein and instructions for using the compound or composition. In certain embodiments, the kits and instructions provide for treating an insect infestation. In certain embodiments, the kits and instructions provide for treating an insect infestation caused by silkworms. In certain embodiments, the kits and instructions provide for treating an insect infestation caused by silkworms in the third instar larval stage.

In some embodiments, the present disclosure provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Methods and Uses

The compounds and compositions of the present disclosure may be used to treat or prevent various diseases or conditions. In certain embodiments, the disease is a proliferative disease. In certain embodiments, the present disclosure provides methods for treating or preventing cancer including breast cancer, lung cancer, prostate cancer, colorectal cancer, cervical cancer, or cancer of the blood (e.g., lymphocytic leukemia) in a subject in need thereof. In certain embodiments, the present disclosure provides methods for treating or preventing a bacterial infection (e.g., Escherichia coli, Klebsiella pneumoniae, Pseudomonas aeruginosa, Acinetobacter baumanii, Neisseria gonorrhoeae, or Bacillus subtilis) in a subject in need thereof. In certain embodiments, the present disclosure provides methods for treating or preventing a fungal infection (e.g., Candida albicans, Trichophyton mentagrophytes, or Amorphotheca resinae) in a subject in need thereof. In certain embodiments, the present disclosure provides methods for treating or preventing a viral infection (e.g., Herpes simplex type 1) in a subject in need thereof. In certain embodiments, the present disclosure provides methods for treating or preventing abnormal cardiovascular function in a subject in need thereof. In certain embodiments, the present disclosure provides methods for treating or preventing bradycardia in a subject in need thereof.

In certain embodiments, the present disclosure provides methods for treating an insect infestation. In certain embodiments, the insect infestation is caused by silkworms. In certain embodiments, the insect infestation is caused by silkworms in the third instar larval stage.

In some embodiments, the present disclosure provides a method for killing or inhibiting proliferation of cells comprising treating the cells with an amount of a provided compound, or a pharmaceutically acceptable salt, tautomer, or stereoisomer thereof, being effective to kill or inhibit proliferation of the cells. In some embodiments, the cells are tumor cells or cancer cells. In some embodiments, the present disclosure provides a method of treating a disease, comprising administering to a subject in need an effective amount of a provided compound or a pharmaceutically acceptable salt, tautomer, or stereoisomer thereof. In some embodiments, the present disclosure provides a method of treating a disease, comprising administering to a subject suffering therefrom or susceptible thereto an effective amount of a provided compound or pharmaceutically salt, tautomer, or stereoisomer thereof. In some embodiments, a disease is a cancer, a bacterial infection, a fungal infection, a viral infection, or abnormal cardiovascular function. In some embodiments, a disease is cancer. In some embodiments, a disease is an infectious disease. In some embodiments, a disease is abnormal cardiovascular function (e.g., bradycardia). In some embodiments, a provided is a compound of Formula (I). In some embodiments, a provided is a compound of Formula (I′).

A provided compound of the disclosure may be combined in a pharmaceutical combination formulation, or dosing regimen as combination therapy, with a second compound having therapeutic properties. A second compound of the pharmaceutical combination formulation or dosing regimen preferably has complementary activities to a provided compound of the combination such that they do not adversely affect each other.

In some embodiments, a second compound is a chemotherapeutic agent, cytotoxic agent, cytokine, growth inhibitory agent, anti-hormonal, a drug for an autoimmune disease, a drug for an infectious disease, and/or cardioprotectant. Such molecules are suitably present in combination in amounts that are effective for the purpose intended.

A combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Suitable dosages for co-administered agents are those presently used and may be lowered due to the combined action (synergy) of the newly identified agent and other chemotherapeutic agents or treatments.

A provided combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

In some embodiments, provided compounds and/or compositions are useful for treating or preventing a proliferative disease. In certain embodiments, provided compounds and/or compositions are useful for inhibiting the multiplication of a tumor cell or cancer cell, causing apoptosis in a tumor or cancer cell, or for treating cancer in a subject. In some embodiments, provided compounds and compositions can be used in a variety of settings for the treatment of cancers.

In some embodiments, the proliferative disease is a benign neoplasm. All types of benign neoplasms disclosed herein or known in the art are contemplated as being within the scope of the disclosure. In some embodiments, the proliferative disease is associated with angiogenesis. All types of angiogenesis disclosed herein or known in the art are contemplated as being within the scope of the disclosure. In certain embodiments, the proliferative disease is an inflammatory disease. All types of inflammatory diseases disclosed herein or known in the art are contemplated as being within the scope of the disclosure. In certain embodiments, the inflammatory disease is rheumatoid arthritis. In some embodiments, the proliferative disease is an autoinflammatory disease. All types of autoinflammatory diseases disclosed herein or known in the art are contemplated as being within the scope of the disclosure. In some embodiments, the proliferative disease is an autoimmune disease. All types of autoimmune diseases disclosed herein or known in the art are contemplated as being within the scope of the disclosure. In some embodiments, the proliferative disease is cancer. In some embodiments, the present disclosure provides a method of treating a proliferative disease in a subject suffering therefrom, comprising administering to the subject a therapeutically effective amount of a provided compound. In some embodiments, a provided compound has the structure of Formula (I). In some embodiments, a provided compound has the structure of Formula (I′).

In some embodiments, the compounds described herein, or a pharmaceutical composition thereof are useful for treating a cancer. In some embodiments, the compounds described herein, or a pharmaceutical composition thereof are useful for preventing a cancer. In certain embodiments, the present disclosure provides methods for treating breast cancer. In certain embodiments, the present disclosure provides methods for preventing breast cancer. In certain embodiments, the present disclosure provides methods for treating lung cancer. In certain embodiments, the present disclosure provides methods for preventing lung cancer. In certain embodiments, the present disclosure provides methods for treating prostate cancer. In certain embodiments, the present disclosure provides methods for preventing prostate cancer. In certain embodiments, the present disclosure provides methods for treating colorectal cancer. In certain embodiments, the present disclosure provides methods for preventing colorectal cancer. In certain embodiments, the present disclosure provides methods for treating cervical cancer. In certain embodiments, the present disclosure provides methods for preventing cervical cancer. In certain embodiments, the present disclosure provides methods for treating breast cancer. In certain embodiments, the present disclosure provides methods for preventing breast cancer. In certain embodiments, the present disclosure provides methods for treating a cancer of the blood. In certain embodiments, the present disclosure provides methods for preventing a cancer of the blood. In certain embodiments, the present disclosure provides methods for treating lymphocytic leukemia. In certain embodiments, the present disclosure provides methods for preventing lymphocytic leukemia. In certain embodiments, exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM), a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendocrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

Other particular types of cancers that can be treated with provided compounds and/or compositions include, but are not limited to, those listed below: Solid tumors, including but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma ultiforme, astrocytoma, medulloblastoma, craniopharyngioma, ependymoma, pinealoma, emangioblastoma, acoustic neuroma, oligodendroglioma, meningioma, skin cancer, melanoma, neuroblastoma, retinoblastoma. Blood-borne cancers, including but not limited to: acute lymphoblastic leukemia “ALL”, acute lymphoblastic B-cell leukemia, acute lymphoblastic T-cell leukemia, acute myeloblastic leukemia AML″, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, lymphoblastic, myelogenous, lymphocytic and myelocytic leukemias. Lymphomas: Hodgkin's disease, non-Hodgkin's Lymphoma, Multiple myeloma, Waldenstrbm's macroglobulinemia, Heavy chain disease, and Polycythemia vera.

In some embodiments, a cancer being treated is carcinoma, lymphoma, blastoma, sarcoma, leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Cancers, including, but not limited to, a tumor, metastasis, or other disease or disorder characterized by uncontrolled cell growth, can be treated or prevented by administration of a provided compound or composition. In some embodiments, a provided compound or composition is administered with another cancer treatment. In some embodiments, the other cancer treatment (e.g., an anti-cancer agent) is an agent including, but not limited to, abiraterone acetate, ABVD, ABVE, ABVE-PC, AC, AC-T, ADE, ado-trastuzumab emtansine, afatinib dimaleate, aldesleukin, alemtuzumab, anastrozole, arsenic trioxide, asparaginase Erwinia chrysanthemi, axitinib, azacitidine, BEACOPP, belinostat, bendamustine hydrochloride, BEP, bevacizumab, bicalutamide, bleomycin, blinatumomab, bortezomib, bosutinib, brentuximab vedotin, busulfan, cabazitaxel, cabozantinib-s-malate, CAF, capecitabine, CAPOX, carboplatin, carboplatin-taxol, carfilzomibcarmustine, carmustine implant, ceritinib, cetuximab, chlorambucil, chlorambucil-prednisone, CHOP, cisplatin, clofarabine, CMF, COPP, COPP-ABV, crizotinib, CVP, cyclophosphamide, cytarabine, dabrafenib, dacarbazine, dactinomycin, dasatinib, daunorubicin hydrochloride, decitabine, degarelix, denileukin diftitox, denosumab, Dinutuximab, docetaxel, doxorubicin hydrochloride, doxorubicin hydrochloride liposome, enzalutamide, epirubicin hydrochloride, EPOCH, erlotinib hydrochloride, etoposide, etoposide phosphate, everolimus, exemestane, FEC, fludarabine phosphate, fluorouracil, FOLFIRI, FOLFIRI-BEVACIZUMAB, FOLFIRI-CETUXIMAB, FOLFIRINOX, FOLFOX, FU-LV, fulvestrant, gefitinib, gemcitabine hydrochloride, gemcitabine-cisplatin, gemcitabine-oxaliplatin, goserelin acetate, Hyper-CVAD, ibritumomab tiuxetan, ibrutinib, ICE, idelalisib, ifosfamide, imatinib mesylate, imiquimod, ipilimumab, irinotecan hydrochloride, ixabepilone, lanreotide acetate, lapatinib ditosylate, lenalidomide, lenvatinib, letrozole, leucovorin calcium, leuprolide acetate, liposomal cytarabine, lomustine, mechlorethamine hydrochloride, megestrol acetate, mercaptopurine, methotrexate, mitomycin c, mitoxantrone hydrochloride, MOPP, nelarabine, nilotinib, nivolumab, obinutuzumab, OEPA, ofatumumab, OFF, olaparib, omacetaxine mepesuccinate, OPPA, oxaliplatin, paclitaxel, paclitaxel albumin-stabilized nanoparticle formulation, PAD, palbociclib, pamidronate disodium, panitumumab, panobinostat, pazopanib hydrochloride, pegaspargase, peginterferon alfa-2b, peginterferon alfa-2b, pembrolizumab, pemetrexed disodium, pertuzumab, plerixafor, pomalidomide, ponatinib hydrochloride, pralatrexate, prednisone, procarbazine hydrochloride, radium 223 dichloride, raloxifene hydrochloride, ramucirumab, R-CHOP, recombinant HPV bivalent vaccine, recombinant human papillomavirus, nonavalent vaccine, recombinant human papillomavirus, quadrivalent vaccine, recombinant interferon alfa-2b, regorafenib, rituximab, romidepsin, ruxolitinib phosphate, siltuximab, sipuleucel-t, sorafenib tosylate, STANFORD V, sunitinib malate, TAC, tamoxifen citrate, temozolomide, temsirolimus, thalidomide, thiotepa, topotecan hydrochloride, toremifene, tositumomab and iodine I 131, tositumomab, TPF, trametinib, trastuzumab, VAMP, vandetanib, VEIP, vemurafenib, vinblastine sulfate, vincristine sulfate, vincristine sulfate liposome, vinorelbine tartrate, vismodegib, vorinostat, XELIRI, XELOX, ziv-aflibercept, and zoledronic acid. Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon a, interferon 7), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), EIP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g., 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g., mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTINm, AZD2171), dasatinib (SPRYCEL®, BMS-354825), erlotinib (TARCEVA®), gefitinib (RESSA®), imatinib (Gleevec®, CGP57148B, STI-571), lapatinib (TYKERB®, TYVERB), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In some embodiments, methods for treating or preventing cancer are provided, comprising administering to a subject in need thereof an effective amount of a provided compound or composition. In some embodiments, a provided compound is administered prior to, concurrently with, or subsequent to, a chemotherapeutic agent. In some embodiments, a chemotherapeutic agent is that with which treatment of the cancer has not been found to be refractory. In some embodiments, a chemotherapeutic agent is that with which the treatment of cancer has been found to be refractory. In some embodiments, a provided compound is administered to a patient that has also undergone surgery as treatment for the cancer.

In some embodiments, an additional method of treatment is radiation therapy. In some embodiments, a provided compound or composition is administered prior to, concurrently with or subsequent to radiation.

In some embodiments, a provided compound or composition is administered concurrently with a chemotherapeutic agent or with radiation therapy. In some embodiments, a chemotherapeutic agent or radiation therapy is administered prior or subsequent to administration of a provided compound or composition. In some embodiments, a chemotherapeutic agent or radiation therapy is administered concurrently with administration of a provided compound or composition. In some embodiments, a provided compound or composition is administered at least one hour, five hours, 12 hours, a day, a week, a month, or several months (e.g., up to three months), prior or subsequent to administration of a provided compound or composition.

A chemotherapeutic agent can be administered over a series of sessions. Any one or a combination of the chemotherapeutic agents can be administered.

Exemplary chemotherapy drugs are widely known in the art, including but not limited to tubulin-binding drugs, kinase inhibitors, alkylating agents, DNA topoisomerase inhibitors, anti-folates, pyrimidine analogs, purine analogs, DNA antimetabolites, hormonal therapies, retinoids/deltoids, photodynamic therapies, cytokines, angiogenesis inhibitors, histone modifying enzyme inhibitors, and antimitotic agents. Examples are extensively described in the art, including but not limited to those in PCT Application Publication No. WO2010/025272. In some embodiments, a “tubulin-binding drug” refers to a ligand of tubulin or to a compound capable of binding a or β-tubulin monomers or oligomers thereof, αβ-tubulin heterodimers or oligomers thereof, or polymerized microtubules. Exemplary tubulin-binding drugs include, but are not limited to: (a) Combretastatins or other stilbene analogs (e.g., described in Pettit et al, Can. J. Chem., 1982; Pettit et al, J. Org. Chern., 1985; Pettit et al, J. Nat. Prod., 1987; Lin et al, Biochemistry, 1989; Singh et al, J. Org. Chem., 1989; Cushman et al, J. Med. Chern., 1991; Getahun et al, J. Med. Chem., 1992; Andres et al, Bioorg. Med. Chern. Lett., 1993; Mannila, Liebigs. Ann. Chern., 1993; Shirai et al, Bioorg. Med. Chem. Lett., 1994; Medarde et al., Bioorg. Med. Che. Lett., 1995; Pettit et al, J. Med. Chem., 1995; Wood et al, Br. J. Cancer., 1995; Bedford et al, Bioorg. Med. Chem. Lett., 1996; Dorr et al, Invest. New Drugs, 1996; Jonnalagadda et al., Bioorg. Med. Chern. Lett., 1996; Shirai et al, Heterocycles, 1997; Aleksandrzak K, Anticancer Drugs, 1998; Chen et al, Biochem. Pharmacal., 1998; Ducki et al, Bioorg. Med. Chem. Lett., 1998; Hatanaka et al, Bioorg. Med. Chem. Lett., 1998; Medarde, Eur. J. Med. Chem., 1998; Medina et al, Bioorg. Med. Chem. Lett., 1998; Ohsumi et al, Bioorg. Med. Chem. Lett., 1998; Ohsumi et al., J. Med. Chem., 1998; Pettit G R et al., J. Med. Chem., 1998; Shirai et al, Bioorg. Med. Chern. Lett., 1998; Banwell et al, Aust. J. Chem., 1999; Medarde et al, Bioorg. Med. Chem. Lett., 1999; Shan et al, PNAS, 1999; Combeau et al, Mol. Pharmacal, 2000; Pettit et al, J. Med Chern, 2000; Pettit et al, Anticancer Drug Design, 2000; Pinney et al, Bioorg. Med. Chem. Lett., 2000; Flynn et al., Bioorg. Med. Chem. Lett., 2001; Gwaltney et al, Bioorg. Med. Chem. Lett., 2001; Lawrence et al, 2001; Nguyen-Hai et al, Bioorg. Med. Chern. Lett., 2001; Xia et al, J. Med. Chern., 2001; Tahir et al., Cancer Res., 2001; Wu-Wong et al., Cancer Res., 2001; Janik et al, Biooorg. Med. Chern. Lett., 2002; Kim et al., Bioorg Med Chem Lett., 2002; Li et al, Biooorg. Med. Chern. Lett., 2002; Nam et al, Bioorg. Med. Chern. Lett., 2002; Wang et al, J. Med. Chern. 2002; Hsieh et al, Biooorg. Med. Chem. Lett., 2003; Hadimani et al., Bioorg. Med. Che. Lett., 2003; Mu et al, J. Med. Chern, 2003; Nam, Curr. Med. Chern., 2003; Pettit et al, J. Med. Chem., 2003; WO 02/50007, WO 02/22626, WO 02/14329, WO 01/81355, WO 01/12579, WO 01/09103, WO 01/81288, WO 01/84929, WO 00/48591, WO 00/48590, WO 00/73264, WO 00/06556, WO 00/35865, WO 00/48590, WO 99/51246, WO 99/34788, WO 99/35150, WO 99/48495, WO 92/16486, U.S. Pat. Nos. 6,433,012, 6,201,001, 6,150,407,6,169,104, 5,731,353, 5,674,906, 5,569,786, 5,561,122, 5,430,062, 5,409,953, 5,525,632, 4,996,237 and 4,940,726 and U.S. patent application Ser. No. 10/281,528); (b) 2,3-substituted Benzo[b]thiophenes (e.g., described in Pinney et al, Bioorg. Med. Chern. Lett., 1999; Chen et al, J. Org. Chem., 2000; U.S. Pat. Nos. 5,886,025; 6,162,930, and 6,350,777; WO 98/39323); (c) 2,3-disubstituted Benzo[b]furans (e.g., described in WO 98/39323, WO 02/060872); (d) Disubstituted Indoles (e.g., described in Gastpar R, J. Med. Chem., 1998; Bacher et al, Cancer Res., 2001; Flynn et al, Bioorg. Med. Chern. Lett, 2001; WO 99/51224, WO 01/19794, WO 01/92224, WO 01/22954; WO 02/060872, WO 02/12228, WO 02/22576, and U.S. Pat. No. 6,232,327); (e) 2-Aroylindoles (e.g., described in Mahboobi et al, J. Med. Chern., 2001; Gastpar et al., J. Med. Chem., 1998; WO 01/82909); (f) 2,3-disubstituted Dihydronaphthalenes (e.g., described in WO 01/68654, WO 02/060872); (g) Benzamidazoles (e.g., described in WO 00/41669); (h) Chalcones (e.g., described in Lawrence et al, Anti-Cancer Drug Des, 2000; WO 02/47604); (i) Colchicine, Allocolchicine, Thiocolcichine, Halichondrin B, and Colchicine derivatives (e.g., described in WO 99/02166, WO 00/40529, WO 02/04434, WO 02/08213, U.S. Pat. Nos. 5,423,753, 6,423,753) in particular the N-acetyl colchinol prodrug, ZD-6126; (j) Curacin A and its derivatives (e.g., described in Gerwick et al, J. Org. Chem., 1994, Blokhin et al, Mol. Phamacol., 1995; Verdier-Pinard, Arch. Biochem. Biophys., 1999; WO 02/06267); (k) Dolastatins such as Dolastatin-10, Dolastatin-15, and their analogs (e.g., described in Pettit et al, J. Am. Chern. Soc., 1987; Bai et al, Mol. Pharmacal, 1995; Pettit et al, Anti-Cancer Drug Des., 1998; Poncet, Curr. Pharm. Design, 1999; WO 99/35164; WO 01/40268; U.S. Pat. No. 5,985,837); (l) Epothilones such as Epothilones A, B, C, D, and Desoxyepothilones A and B, Fludelone (e.g., described in Chou et al. Cancer Res. 65:9445-9454, 2005, the entirety of which is hereby incorporated by reference), 9,10-dehydro-desoxyepothilone B (dehydelone), iso-oxazole-dehydelone (17-isooxazole-dehydelone), fludelone, iso-oxazolefludelone (17-isooxazole-fludelone), (Danishefsky, et al., PNAS, v. 105, 35:13157-62, 2008; WO 99/02514, U.S. Pat. No. 6,262,094, Nicolau et al., Nature, 1997, Pub. No. US2005/0 143429); (m) Inadones (e.g., described in Leoni et al., J. Natl. Cancer Inst., 2000; U.S. Pat. No. 6,162,810); (n) Lavendustin A and its derivatives (Mu F et al, J. Med. Chern., 2003, the entirety of which is hereby incorporated by reference); (o) 2-Methoxyestradiol and its derivatives (e.g., described in Fotsis et al, Nature, 1994; Schumacher et al, Clin. Cancer Res., 1999; Cushman et al, J. Med. Chem., 1997; Verdier-Pinard et al, Mol. Pharmacal, 2000; Wang et al, J. Med. Chem., 2000; WO 95/04535, WO 01/30803, WO 00/26229, WO 02/42319 and U.S. Pat. Nos. 6,528,676, 6,271,220, 5,892,069, 5,661,143, and 5,504,074); (p) Monotetrahydrofurans (e.g., “COBRAs”; Uckun, Bioorg. Med. Chem. Lett., 2000; U.S. Pat. No. 6,329,420); (q) Phenylhistin and its derivatives (e.g., described in Kanoh et al, J. Antibiot., 1999; Kano et al, Bioorg. Med. Chem., 1999 and U.S. Pat. No. 6,358,957); (r) Podophyllotoxins such as Epidophyllotoxin (e.g., described in Hammonds et al, J. Med. Microbial, 1996; Coretese et al, J. Biol. Chem., 1977); (s) Rhizoxins (e.g., described in Nakada et al, Tetrahedron Lett., 1993; Boger et al, J. Org. Chern., 1992; Rao, et al, Tetrahedron Lett., 1992; Kobayashi et al, Pure Appl. Chern., 1992; Kobayashi et al, Indian J. Chem., 1993; Rao et al, Tetrahedron Lett., 1993); (t) 2-strylquinazolin-4(3H)-ones (e.g., “SQOs”, Jiang et al, J. Med. Chem., 1990, the entirety of which is hereby incorporated by reference); (u) Spongistatin and Synthetic spiroketal pyrans (e.g., “SPIKETs”; Pettit et al, J. Org. Chem., 1993; Uckun et al, Bioorgn. Med. Chem. Lett., 2000; U.S. Pat. No. 6,335,364, WO00/00514); (v) Taxanes such as Paclitaxel (TAXOL®), Docetaxel (TAXOTERE®), and Paclitaxel derivatives (e.g., described in U.S. Pat. No. 5,646,176, WIPO Publication No. WO 94/14787, Kingston, J. Nat. Prod., 1990; Schiff et al, Nature, 1979; Swindell et al, J. Cell Biol., 1981); (x) Vinca Alkaloids such as Vinblastine, Vincristine, Vindesine, Vinflunine, Vinorelbine (NAVELBINE) (e.g., described in Owellen et al, Cancer Res., 1976; Lavielle et al, J. Med. Chem., 1991; Holwell et al, Br. J. Cancer., 2001); and (y) Welwistatin (e.g., described in Zhang et al, Molecular Pharmacology, 1996, the entirety of which is hereby incorporated by reference).

Exemplary specific examples of tubulin-binding drugs include, but are not limited to, allocolchicine, amphethinile, chelidonine, colchicide, colchicine, combrestatin A1, combretastin A4, combretastain A4 phosphate, combrestatin 3, combrestatin 4, cryptophycin, curacin A, deo-dolastatin 10, desoxyepothilone A, desoxyepothilone B, dihydroxypentamethoxyflananone, docetaxel, dolastatin 10, dolastatin 15, epidophyllotoxin, epothilone A, epothilone B, epothilone C, epothilone D, etoposide, 9,10-dehydro-desoxyepothilone B (dehydelone), iso-oxazole-dehydelone (17-isooxazole-dehydelone), fludelone, iso-oxazolefludelone (17-isooxazole-fludelone), griseofulvin, halichondrin B, isocolchicine, lavendustin A, methyl-3,5-diiodo-4-(4′-methoxyphenoxy)benzoate, N-acetylcolchinol, N-acetylcolchinol-0-phosphate, N-[2-[(4-hydroxyphenyl)amino]-3-pyridyl]-4-methoxybenzenesulfonamide, nocodazole, paclitaxel, phenstatin, phenylhistin, piceid, podophyllotoxin, resveratrol, rhizoxin, sanguinarine, spongistatin 1, steganacin, TAXOL, teniposide, thiocolchicine, vincristine, vinblastine, welwistatin, (Z)-2-methoxy-5-[2-(3,4,5- trimethoxyphenyl)vinyl] phenylamine, (Z)-3,5,4′-trimethoxystilbene (R3), 2-aryl-1,8-naphthyridin-4(1H)-one, 2-(41-methoxyphenyl)-3-(3 1,4 1,5 1-rimethoxybenzoyl)-6- methoxybenzo[b]thiophene, 2-methoxy estradiol, 2-strylquinazolin-4(3H)-one, 5,6- dihydroindolo(2, 1-a)isoquinoline, and 1 0-deacetylbaccatin III.

In some other embodiments, exemplary chemotherapy drugs include but are not limited to nitrogen mustards, nitrosoureas, alkylsulphonates, triazenes, platinum complexes, epipodophyllins, mitomycins, DIFR inhibitors, IMP dehydrogenase inhibitors, ribonucleotide reductase inhibitors, uracil analogs, cytosine analogs, purine analogs, receptor antagonists (for example, anti-estrogen, LHRH agonists, anti-androgens), vitamin derivative or analogs, isoprenylation inhibitors, dopaminergic neurotoxins, cell cycle inhibitors, actinomycins, bleomycins, anthracyclines, MDR inhibitors, Ca²⁺ ATPase inhibitors, and anti-metastatis agents. In some embodiments, exemplary specific examples of tubulin-binding drugs include, but are not limited to, Cyclophosphamide, Ifosfamide, Trofosfamide, Chlorambucil, Carmustine, Lomustine, Busulfan, Treosulfan, Dacarbazine, Procarbazine, Temozolomide, Cisplatin, Carboplatin, Aroplatin, Oxaliplatin, Topotecan, Irinotecan, 9-aminocamptothecin, Camptothecin, Crisnatol, Mitomycin C, Methotrexate, Trimetrexate, Mycophenolic acid, Tiazofurin, Ribavirin, 5-Ethynyl-1-beta-D-ribofuranosylimidazole-4- carboxamide (EICAR), Hydroxyurea, Deferoxamine, 5-Fluorouracil, Fluoxuridine, Doxifluridine, Ralitrexed, Cytarabine, Cytosine arabinoside, Fludarabine, Gemcitabine, Capecitabine, Mercaptopurine, Thioguanine, O-6-benzylguanine, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, inosine glycodialdehyde, macebecin II, Pyrazoloimidazole, Tamoxifen, Raloxifene, Megestrol, Goserelin, Leuprolide acetate, Flutamide, Bicalutamide, Cis-retinoic acid, All-trans retinoic acid (ATRA-IV), EB 1089, CB 1093, KH 1060, Vertoporfin, Phthalocyanine, Photosensitizer Pc4, Demethoxy-hypocrellin A, ABT-627, Bay 12-9566, Benefin, BMS-275291, cartilage-derived inhibitor, CAI, CEP-7055, Col 3, Halofuginone, Heparin hexasaccharide fragment, IM-862, Marimastat, Metalloproteinase inhibitors, 2-Methoxyestra diol, MMI 270, Neovastat, NM-3, Panzem, PI-88, Placental ribonuclease inhibitor, Plasminogen activator inhibitor, Prinomastat, Retinoids, Solimastat, Squalamine, SS 3304, SU 5416, SU 6668, SU 11248, Tetrahydrocortisol-S, Tetrathiomolybdate, Thalidomide, TNP-470, ZD 6126, ZD 6474, farnesyl transferase inhibitors, Bisphosphonates, trityl cysteine, 1-methyl-4-phenylpyridinium ion, Staurosporine, Actinomycin D, Dactinomycin, Bleomycin A2, Bleomycin B2, Peplomycin, Daunorubicin, Doxorubicin, Idarubicin, Epirubicin, Pirarubicin, Zorubicin, Mitoxantrone, Verapamil, Ardeemin, Ningalin, Thapsigargin, Metastatin, GLiY-SD-ME-1, Sorafenib, Imatinib, Gefinitib, Lapatinib, Dasatinib, Nilotinib, Temsirolimus, Erlotinib, Pomalidomide, Regorafenib, Paclitaxel Protein-Bound Particles For Injectable Suspension, Everolimus, Bosutinib, Cabozantinib, Cabozantinib, Ponatinib, Axitinib, Carfilzomib, Ingenol Mebutate, Regorafenib, Fentanyl, Omacetaxine Mepesuccinate, Cephalotaxine, Pazopanib, Enzalutamide, Fentanyl Citrate, Sunitinib, Vandetanib, Crizotinib, Vemurafenib, Abiraterone Acetate, Eribulin Mesylate, Cabazitaxel, Ondansetron, Pralatrexate, Romidepsin, Plerixafor, Granisetron, Bendamustine Hydrochloride, Raloxifene Hydrochloride, Topotecan, Ixabepilone, Nilotinib, Temsirolimus, Lapatinib, Nelarabine, Sorafenib, Clofarabine, Cinacalcet, Erlotinib, Palonosetron, Tositumomab, Aprepitant, Gefitinib, Abarelix, Conjugated Estrogens, Alfuzosin, Bortezomib, Leucovorin, Fulvestrant, Ibritumomab Tiuxetan, Zoledronic Acid, Triptorelin Pamoate, Arsenic Trioxide, Aromasin, Busulfan, Amifostine, Temozolomide, Odansetron, Dolasetron, Irinotecan, Gemcitabine, Porfimer Sodium, Valrubicin, Capecitabine, Zofran, Bromfenac, Letrozole, Leuprolide, Samarium (¹⁵³sm) Lexidronam, Pamidronate, Anastrozole, Levoleucovorin, Flutamide And Goserelin.

In some embodiments, a provided compound or composition is administered prior to, concurrently with or subsequent to a polypeptide or protein. In some embodiments, a polypeptide or protein is a recombinant polypeptide or protein. Exemplary polypeptides or proteins include but are not limited to cytokines, interferon alfa-2b, interleukin 2, filgrastim, rasburicase, secretin, asparaginase Erwinia chrysanthemi, and ziv-aflibercept. In some embodiments, a polypeptide or protein comprises an antibody or a fragment of an antibody. In some embodiments, a polypeptide or protein is an antibody or a fragment of an antibody. Examples include but are not limited to rituximab, trastuzumab, tositumomab, alemtuzumab, bevacizumab, cetuximab, panitumumab, ofatumumab, denosumab, ipilimumab, pertuzumab. In some embodiments, a polypeptide or protein is chemically modified. In some embodiments, a polypeptide or protein is conjugated to a drug. In some embodiments, an antibody or an antibody fragment is conjugated to a payload drug, forming an antibody-drug conjugate. In some embodiments, a payload drug is cytotoxic. Exemplary antibody-drug conjugates include but are not limited to gemtuzumab ozogamicin, brentuximab vedotin, and ado-trastuzumab emtansine. In some embodiments, a cancer treatment comprises the use of a vaccine. Exemplary vaccines for cancer treatment are well known in the art, for example but not limited to sipuleucel-T.

A provided compound may be combined with an anti-hormonal compound; e.g., an anti-estrogen compound such as tamoxifen; an anti-progesterone such as onapristone (EP 616812); or an anti-androgen such as flutamide, in dosages known for such molecules. Where the cancer to be treated is hormone independent cancer, the patient may previously have been subjected to anti-hormonal therapy and, after the cancer becomes hormone independent, a provided compound (and optionally other agents as described herein) may be administered to the patient. In some embodiments, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy.

With respect to radiation, any radiation therapy protocol can be used depending upon the type of cancer to be treated. For example, but not by way of limitation, X-ray radiation can be administered; in some embodiments, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

In some embodiments, methods of treatment of cancer with a provided compound or composition are provided as an alternative to chemotherapy or radiation therapy where the chemotherapy or the radiation therapy has proven or can prove too toxic, e.g., results in unacceptable or unbearable side effects, for a subject being treated. A subject being treated can, optionally, be treated with another cancer treatment such as surgery, radiation therapy or chemotherapy, depending on which treatment is found to be acceptable or bearable.

In some embodiments, a provided compound or composition can be used in an in vitro or ex vivo fashion, such as for the treatment of certain cancers, including, but not limited to leukemias and lymphomas. In some embodiments, such a treatment involves autologous stem cell transplants. In some embodiments, this can involve a multi-step process in which a subject's autologous hematopoietic stem cells are harvested and purged of all cancer cells, a subject's remaining bone-marrow cell population is then eradicated via the administration of a high dose of a provided compound or composition with or without accompanying high dose radiation therapy, and the stem cell graft is infused back into the animal. Supportive care is then provided while bone marrow function is restored and a subject recovers.

In some embodiments, the present disclosure provides methods for treating an infectious disease, comprising administering to a subject suffering therefrom or susceptible thereto an effective amount of a provided compound or a pharmaceutically acceptable salt, tautomer, or stereoisomer thereof. In some embodiments, a provided compound or composition is useful for killing or inhibiting the multiplication of a cell that produces an infectious disease or for treating an infectious disease. A provided compound can be used in a variety of settings for the treatment of an infectious disease in a subject. In one embodiment, a provided compound kills or inhibits the multiplication of cells that produce a particular infection.

In some embodiments, the present disclosure provides methods for treating a bacterial infection, comprising administering to a subject suffering therefrom or susceptible thereto an effective amount of a provided compound or a pharmaceutically acceptable salt, tautomer, or stereoisomer thereof. In some embodiments, a provided compound or composition is useful for killing or inhibiting the multiplication of bacteria. In some embodiments, a provided compound or composition is useful for killing or inhibiting the multiplication of a bacteria that produces an infectious disease. In some embodiments, a provided compound or composition is useful for reating an infectious disease. A provided compound can be used in a variety of settings for the treatment of a bacterial infection in a subject. In one embodiment, a provided compound kills or inhibits the multiplication of bacteria that produce a particular infection or infectious disease.

In certain embodiments, the bacterial infection is an infection of Escherichia coli, Klebsiella pneumoniae, Pseudomonas aeruginosa, Acinetobacter baumanii, Neisseria gonorrhoeae, or Bacillus subtilis. In some embodiments, the disease is caused by Escherichia coli, Klebsiella pneumoniae, Pseudomonas aeruginosa, Acinetobacter baumanii, Neisseria gonorrhoeae, or Bacillus subtilis.

In certain embodiments, the bacterial infection is an infection caused by Gram-positive bacteria. In certain, embodiments, the bacterial infection is an infection caused by Gram-negative bacteria.

Exemplary bacterial infections include, but are not limited to, infections with a Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/ChlorobiBacteroidetes (FCB), phylum Fusobacteria, phylum Gemmatimonadest, phylum Ntrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the Gram negative bacteria is a bacteria of the phylum Proteobacteria and the genus Escherichia. i.e., the bacterial infection is an Escherichia infection. Exemplary Escherichia bacteria include, but are not limited to, E. albertii, E. blattae, E. coli, E. fergusonii, E. hermannii, and E. vulneris. In certain embodiments, the Escherichia infection is an E. coli infection.

In certain embodiments, the Gram negative-bacteria is a bacteria of the phylum Proteobacteria and the genus Acinetobacter. i.e., the bacterial infection is an Acinetobacter infection. Exemplary Acinetobacter bacteria include, but are not limited to, A. baumanii, A. haemolyticus, and A. lwoffii. In certain embodiments, the Acinetobacter infection is an A. baumanii infection.

In certain embodiments, the Gram-negative bacteria is a bacteria of the phylum Proteobacteria and the genus Klebsiella. i.e., the bacterial infection is a Klebsiella infection. Exemplary Klebsiella bacteria include, but are not limited to, K. granulomatis, K. oxytoca, K. michiganensis, K. pneumoniae, K. quasipneumoniae, and K. variicola. In certain embodiments, the Klebsiella infection is a K. pneumoniae infection.

In certain embodiments, the Gram-negative bacteria is a bacteria of the phylum Proteobacteria and the genus Pseudomonas. i.e., the bacterial infection is a Pseudomonas infection. Exemplary Pseudomonas bacteria include, but are not limited to, P. aeruginosa, P. oryzihabitans, P. plecoglissicida, P. syringae, P. putida, and P. fluoroscens. In certain embodiments, the Pseudomonas infection is a P. aeruginosa infection.

In certain embodiments, the Gram negative bacteria is a bacteria of the phylum Proteobacteria and the genus Neisseria i.e., the bacterial infection is an Neisseria infection. Exemplary Neisseria bacteria include, but are not limited to, N. gonorrhoeae and N. meningitidi. In certain embodiments, the Neisseria infection is an N. gonorrhoeae infection.

In certain embodiments, the bacterium is a member of the phylum Firmicutes and the genus Bacillus, i.e., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis. In certain embodiments, the Bacillus infection is a B. subtilis infection. In certain embodiments, the B. subtilis has an efflux (e.g., mef, msr) genotype. In certain embodiments, the B. subtilis has a methylase (e.g., erm) genotype.

In some embodiments, the present disclosure provides methods for treating a viral infection, comprising administering to a subject suffering therefrom or susceptible thereto an effective amount of a provided compound or a pharmaceutically acceptable salt, tautomer, or stereoisomer thereof. In some embodiments, a provided compound or composition is useful for killing or inhibiting the multiplication of a virus. In some embodiments, a provided compound or composition is useful for killing or inhibiting the multiplication of a virus that produces an infectious disease. In some embodiments, a provided compound or composition is useful for treating an infectious disease. A provided compound can be used in a variety of settings for the treatment of a viral infection in a subject. In one embodiment, a provided compound kills or inhibits the multiplication of a virus that produces a particular infection or infectious disease. In one embodiment, a provided compound interferes with the production of viral DNA. In one embodiment, a provided compound prevents a virus from entering a cell.

In certain embodiments, the viral infection is an infection of Herpes simplex type 1. In certain embodiments, the disease is caused by Herpes simplex type 1.

In certain embodiments, the virus is of the phylum incertae sedis and the genus simplexvirus. i.e., the viral infection is a simplexvirus infection. Exemplary simplexvirus viruses include, but are not limited to, Human alphaherpesvirus I and Human alphaherpesvirus 2. In certain embodiments, the simplexvirus infection is an Herpes simplex virus 1 infection. In certain embodiments, the simplexvirus infection is an Herpes simplex virus 2 infection.

In some embodiments, the present disclosure provides methods for treating a fungal infection, comprising administering to a subject suffering therefrom or susceptible thereto an effective amount of a provided compound or a pharmaceutically acceptable salt, tautomer, or stereoisomer thereof. In some embodiments, a provided compound or composition is useful for killing or inhibiting the growth or reproduction of fungal cells. In some embodiments, a provided compound or composition is useful for killing or inhibiting the growth or reproduction of fungal cells that produces an infectious disease. In some embodiments, a provided compound or composition is useful for treating an infectious disease. A provided compound can be used in a variety of settings for the treatment of a fungal infection in a subject. In one embodiment, a provided compound kills or inhibits the growth or reproduction of fungal cells that produce a particular infection or infectious disease. In one embodiment, a provided compound interferes with fungal cell walls.

In certain embodiments, the fungal infection is an infection of Candida albicans, Trichophyton interdigitale, or Amorphotheca resinae. In certain embodiments, the fungal infection is caused by Candida albicans, Trichophyton interdigitale, or Amorphotheca resinae.

In certain embodiments, the virus is of the division ascomycota and the genus candida, i.e., the fungal infection is a candida infection. Exemplary candida fungi include, but are not limited to, C. albicans, C. glabrata, C. rugosa, C. parapsilosis, C. tropicalis, C. dubliniensis, and C. auris. In certain embodiments, the candida infection is a C. albicans infection. In certain embodiments, the candida infection is a C. auris infection.

In certain embodiments, the virus is of the division ascomycota and the genus trichophyton. i.e., the fungal infection is a trichophyton infection. Exemplary trichophyton fungi include, but are not limited to, Trichophyton concentricum, Trichophyton rubrum. Trichophyton interdigitale, Trichophyton schoenleinii, Trichophyton mentagrophytes, and Trichophyton verrucosum. In certain embodiments, the trichophyton infection is a Trichophyton concentricum infection. In certain embodiments, the trichophyton infection is a Trichophyton mentagrophytes infection.

In certain embodiments, the fungus is of the division Ascomycota and the genus amorphothec i.e., the fungal infection is an amorphothec infection. Exemplary amorphothec fungi include, but are not limited to, Amorphotheca resinae. In certain embodiments, the infection is an Amorphotheca resinae infection.

Exemplary types of infectious diseases that can be treated with a provided compound include, but are not limited to: Bacterial Diseases such as Diphtheria, Pertussis, Occult Bacteremia, Urinary Tract Infection, Gastroenteritis, Cellulitis, Epiglottitis, Tracheitis, Adenoid Hypertrophy, Retropharyngeal Abcess, Impetigo, Ecthyma, Pneumonia, Endocarditis, Septic Arthritis, Pneumococcal, Peritonitis, Bactermia, Meningitis, Acute Purulent Meningitis, Urethritis, Cervicitis, Proctitis, Pharyngitis, Salpingitis, Epididymitis, Gonorrhea, Syphilis, Listeriosis, Anthrax, Nocardiosis, Salmonella, Typhoid Fever, Dysentery, Conjunctivitis, Sinusitis, Brucellosis, Tullaremia, Cholera, Bubonic Plague, Tetanus, Necrotizing Enteritis, Actinomycosis, Mixed Anaerobic Infections, Syphilis, Relapsing Fever, Leptospirosis, Lyme Disease, Rat Bite Fever, Tuberculosis, Lymphadenitis, Leprosy, Chlamydia, Chlamydial Pneumonia, Trachoma and Inclusion Conjunctivitis; Systemic Fungal Diseases such as Histoplamosis, Coccidiodomycosis, Blastomycosis, Sporotrichosis, Cryptococcsis, Systemic Candidiasis, Aspergillosis, Mucormycosis, Mycetoma and Chromomycosis; Rickettsial Diseases such as Typhus, Rocky Mountain Spotted Fever, Ehrlichiosis, Eastern Tick-Borne Rickettsioses, Rickettsialpox, Q Fever and Bartonellosis; Parasitic Diseases such as Malaria, Babesiosis, African Sleeping Sickness, Chagas' Disease, Leishmaniasis, Dum-Dum Fever, Toxoplasmosis, Meningoencephalitis, Keratitis, Entamebiasis, Giardiasis, Cryptosporidiasis, Isosporiasis, Cyclosporiasis, Microsporidiosis, Ascariasis, Whipworm Infection, Hookworm Infection, Threadworm Infection, Ocular Larva Migrans, Trichinosis, Guinea Worm Disease, Lymphatic Filariasis, Loiasis, River Blindness, Canine Heartworm Infection, Schistosomiasis, Swimmer's Itch, Oriental Lung Fluke, Oriental Liver Fluke, Fascioliasis, Fasciolopsiasis, Opisthorchiasis, Tapeworm Infections, Hydatid Disease and Alveolar Hydatid Disease; Viral Diseases such as Measles, Subacute sclerosing panencephalitis, Common Cold, Mumps, Rubella, Roseola, Fifth Disease, Chickenpox, Respiratory syncytial virus infection, Croup, Bronchiolitis, Infectious Mononucleosis, Poliomyelitis, Herpangina, Hand-Foot-and-Mouth Disease, Bornholm Disease, Genital Herpes, Genital Warts, Aseptic Meningitis, Myocarditis, Pericarditis, Gastroenteritis, Acquired Immunodeficiency Syndrome (AIDS), Human Immunodeficiency Virus (HIV), Reye's Syndrome, Kawasaki Syndrome, Influenza, Bronchitis, Viral “Walking” Pneumonia, Acute Febrile Respiratory Disease, Acute pharyngoconjunctival fever, Epidemic keratoconjunctivitis, Herpes Simplex Virus 1 (HSV-1), Herpes Simplex Virus 2 (HSV-2), Shingles, Cytomegalic Inclusion Disease, Rabies, Progressive Multifocal Leukoencephalopathy, Kuru, Fatal Familial Insomnia, Creutzfeldt-Jakob Disease, Gerstmann-Straussler-Scheinker Disease, Tropical Spastic Paraparesis, Western Equine Encephalitis, California Encephalitis, St. Louis Encephalitis, Yellow Fever, Dengue, Lymphocytic choriomeningitis, Lassa Fever, Hemorrhagic Fever, Hantvirus Pulmonary Syndrome, Marburg Virus Infections, Ebola Virus Infections and Smallpox.

In some embodiments, the present disclosure provides methods for treating an infectious disease, comprising administering to a subject suffering therefrom an effective amount of a provided compound or composition. In some embodiments, a provided method comprises administering an effective amount of a provided compound or composition and another therapeutic agent known for treatment of an infectious disease.

In some embodiments, a provided method for treating an infectious disease includes administering to a patient in need thereof a provided compound and another therapeutic agent that is an anti-infectious disease agent. In certain embodiments, a provided compound (e.g., a compound of Formula (I), Formula (I′)) is administered with (e.g., sequentially or concurrently) an anti-bacterial agent. In certain embodiments, a provided compound (e.g., a compound of Formula (I), Formula (I′)) is administered with (e.g., sequentially or concurrently) an anti-fungal agent. In certain embodiments, a provided comound (e.g., a compound of Formula (I), Formula (I′)) is administered with (e.g., sequentially or concurrently) an anti-viral agent. Exemplary anti-infectious disease agents are widely known in the art, including but not limited to β-Lactam Antibiotics such as Penicillin G, Penicillin V, Cloxacilliin, Dicloxacillin, Methicillin, Nafcillin, Oxacillin, Ampicillin, moxicillin, Bacampicillin, Azlocillin, Carbenicillin, Mezlocillin, Piperacillin and Ticarcillin; Aminoglycosides: Amikacin, Gentamicin, Kanamycin, Neomycin, Netilmicin, Streptomycin and Tobramycin; Macrolides such as Azithromycin, Clarithromycin, Erythromycin, Lincomycinand Clindamycin; Tetracyclines such as Demeclocycline, Doxycycline, Minocycline, Oxytetracycline and Tetracycline; Quinolones such as Cinoxacin and Nalidixic Acid; Fluoroquinolones such as Ciprofloxacin, Enoxacin, Grepafloxacin, Levofloxacin, Lomefloxacin, Norfloxacin, Ofloxacin, Sparfloxacin and Trovafloxicin; Polypeptides such as Bacitracin, Colistin and Polymyxin B; Sulfonamides such as Sulfisoxazole, Sulfamethoxazole, Sulfadiazine, Sulfamethizole and Sulfacetamide; Miscellaneous Antibacterial Agents such as Trimethoprim, Sulfamethazole, Chloramphenicol, Vancomycin, Metronidazole, Quinupristin, Dalfopristin, Rifampin, Spectinomycin, Nitrofurantoin; General Antiviral Agents such as Idoxuradine, Vidarabine, Trifluridine, Acyclovir, Famcicyclovir, Pencicyclovir, Valacyclovir, Gancicyclovir, Foscarnet, Ribavirin, Amantadine, Rimantadine, Cidofovir, Antisense Oligonucleotides, Immunoglobulins and Inteferons; Drugs for HIV infection such as Tenofovir, Emtricitabine, Zidovudine, Didanosine, Zalcitabine, Stavudine, Lamivudine, Nevirapine, Delavirdine, Saquinavir, Ritonavir and Indinavir, and Nelfinavir.

In certain aspects, the present disclosure provides methods of treating cardiac abnormalities (i.e., abnormal heart function) and cardiovascular diseases and conditions. In some embodiments, the cardiovascular disease is coronary artery disease, peripheral arterial disease, cerebrovascular disease, renal artery stenosis, aortic aneurysm, cardiomyopathy, hypertensive heart disease, heart failure, pulmonary heart disease, cardiac dysrhythmias, inflammatory heart disease, endocarditis, inflammatory cardiomegaly, myocarditis, eosinophilic myocarditis, valvular heart disease, congenital heart disease, and rheumatic heart disease.

In certain embodiments, provided herein are methods of treating cardiac dysrhythmias. In some embodiments, the dysrhythmia is tachycardia including supraventricular dysrhythmias (e.g., atrial flutter, atrial fibrillation, paroxysmal supraventricular tachycardia, Wolff-Parkinson-White syndrome) and ventricular dysrhythmias (e.g., premature ventricular contractions and long QT syndrome). In certain embodiments, the dysrhythmia is bradycardia including sinus bradycardia, conduction block, heart block, and Sick Sinus Syndrome.

In certain embodiments, the present disclosure provides methods of treating bradycardia. In certain embodiments, the present disclosure provides methods of treating bradycardia by administering to a subject in need thereof a compound or composition of the present disclosure. In some embodiments, the present disclosure provides methods of treating bradycardia by administering to a subject in need thereof a compound or composition of the present disclosure, in addition to administering a second therapeutic agent.

In certain aspects, a compound or composition of the present disclosure is administered with (e.g., concurrently or sequentially) a second therapeutic agent (e.g., a cardiac agent). Exemplary cardiac and cardiovascular agents include, but are not limited to, anticoagulants, antiplatelet agents, dual antiplatelet therapy, ACE inhibitors, angiotensin II receptor blockers, angiotensin-receptor neprilysin inhibitors, beta blockers, calcium channel blockers, cholesterol-lowering medications, digitalis preparations, diuretics, and vasodilators.

In certain embodiments, the present disclosure provides methods of treating or preventing an insect infestation. In some embodiments, the method comprises contacting an insect with an effective amount (e.g., an amount effective to kill the insect, an amount effective to prevent reproduction of the insect) of a compound as disclosed herein (e.g., Formula (I), Formula (I′)), or a salt, tautomer, or stereoisomer thereof.

In certain embodiments, the insect is a termite, fly, moth, ant, beetle, mosquito, or silk worm. In certain embodiments, the insect is a silk worm. In certain embodiments, the insect is a silk worm in the 2^{nd} instar larval stage. In certain embodiments, the insect is a silk worm in the 3^{rd} instar larval stage. In certain embodiments, the insect is a silk worm the 4^{th} instar larval stage. In certain embodiments, the insect is a silk worm the 5^{th} instar larval stage.

Additional exemplary insects include, but are not limited to, brown planthopper, small brown planthopper, green leafhopper, rice leafhopper, white-backed planthopper, chinch bug, rice blackbug, green stink bug, rice skipper, rice striped stem borer, gold-fringed stem borer, dark-headed stem borer, rice stalk borer, pink rice borer, white rice borer, yellow rice borer, rice leafroller, leafminer, corn blot leafminer, sugarcane borer, southwestern corn borer, green rice caterpillar, green caterpillar, fall armyworm, beet armyworm, Oriental leafworm, climbing cutworm, western yellowstriped armyworm, armyworm, corn earworm, grape colaspis, rice water weevil, rice plant weevil, rice hispa, leaf beetle, rice weevil, rice gall midge, small rice leafminer, rice stem maggot, stem maggot, western corn rootworm, northern corn rootworm, southern corn rootworm, Mexican corn rootworm, banded cucumber beetle, European corn borer, black cutworm, lesser cornstalk borer, wireworms, northern masked chafer, southern masked chafer, mustard leaf beetle, Mexican bean beetle, Japanese beetle, corn flea beetle, maize billbug, corn leaf aphid, corn root aphid, redlegged grasshopper, differential grasshopper, migratory grasshopper, seedcorn maggot, grass thrips, thief ant, twospotted spider mite, carmine spider mite, corn earworm, cotton bollworm, pink bollworm, spotted bollworm, tobacco budworm, boll weevil, cotton fleahopper, banded-winged whitefly, greenhouse whitefly, silverleaf whitefly, cotton aphid, tarnished plant bug, western tarnished plant bug, consperse stink bug, Say stinkbug, southern green stinkbug, onion thrips, tobacco thrips, western flower thrips, Colorado potato beetle, false potato beetle, Texan false potato beetle, three-lined potato beetle, potato flea beetle, flea beetle, tuber flea beetle, striped blister beetle, potato leafhopper, green peach aphid, psyllid, southern potato wireworm, tobacco wireworm, potato tuberworm, potato aphid, redshouldered stinkbug, potato tuberworm, tomato pinworm, wireworms, tobacco hornworm, tomato hornworm, leafminer, fruitflies, shoot fly, cat flea, root weevil, pine engraver, red floor beetle, tsetse fly, malaria mosquito, pea aphid, honey bee, glassy-winged sharpshooter, yellow fever mosquito, silkworm, migratory locust, cattle tick, red-haired chololate bird eater, pacific beetle cockroach, red passion flower butterfly, postman butterfly, diamontback moth, biting midge, skin beetle, caddisflies, sharpshooter, swallowtail butterfly, japanese oak silkmoth, cabbage looper, cowpea weevil, fungus gnat, minute bog beatle, black-legged tick, asian citrus psyllid, Black Predacious Diving Beetle, brown ear tick, lone star tick, brown citrus aphid, cell spider, caterpillar, and house mosquito.

In certain embodiments, the methods can be used to control pests, including insects, such as termites, flies, moths, ants, beetles, mosquitoes, and silk worms, in particular for the protection of plants, wood, seeds (e.g., stored seeds), grain (e.g., stored grain) and/or manmade structures from infestation and/or damage by such pests. As used herein, “manmade structure” refers to any structure made by man that can be damaged by pests.

The pest, soil, plant, wood, seeds (e.g., stored seeds), grain (e.g., stored grain), or manmade structure can be contacted with the compounds or compositions provided herein in any suitable manner. For example, the pest, soil, plant, wood, seeds (e.g., stored seeds), grain (e.g., stored grain), or manmade structure can be contacted with the compounds or compositions in pure or substantially pure form, for example, an aqueous solution. In this embodiment, the pest, soil, plant, wood, seeds (e.g., stored seeds), grain (e.g., stored grain), or manmade structure may be simply “soaked” with an aqueous solution comprising the compound or composition. In a further embodiment, the pest, soil, plant, wood, seeds (e.g., stored seeds), grain (e.g., stored grain), or manmade structure can be contacted by spraying the pest, soil, plant, wood, seeds (e.g., stored seeds), grain (e.g., stored grain), or manmade structure with a liquid composition. Additional methods will be known to the skilled person.

Alternatively, the compounds or compositions provided may be linked to a food component of the pests in order to increase uptake of the compound or composition by the pest.

The compounds or compositions provided may also be incorporated in the medium in which the pest grows in or on, on a material or item that is infested by the pest, or impregnated in a item or material susceptible to infestation by the pest.

In another embodiment, the compounds or compositions may be, or be used in, a coating that can be applied to a item in order to protect the item from infestation by a pest and/or to prevent, arrest or reduce pest growth on the item and thereby prevent damage caused by the pest. In this embodiment, the composition can be used to protect any item or material that is susceptible to infestation by or damage caused by a pest, for example, wood.

The nature of the excipients and the physical form of the composition may vary depending upon the nature of the item or material that is desired to treat. For example, the composition may be a liquid that is brushed or sprayed onto or imprinted into the material or item to be treated, or a coating that is applied to the material or item to be treated. Provided herein are also methods for treating and/or preventing pest infestation on a item or material comprising applying an effective amount of any of the compositions described herein to said item.

In another embodiment, the compounds or compositions are used as a pesticide or insecticide for a plant or for propagation or reproductive material of a plant, such as on seeds. As an example, the composition can be used as a pesticide or insecticide by spraying or applying it on plant tissue or spraying or mixing it on the soil before or after emergence of the plantlets.

Any of the compositions provided herein may be formulated to include the active ingredient(s) and all inert ingredients (such as solvents, diluents, and various adjuvants).

Spray adjuvants (additives) can be added to pesticides to enhance the performance or handling of those pesticides. Adjuvant may include surfactants, crop oils, antifoaming agents, stickers, and spreaders. Adjuvants may also include: surfactants (surface-active agent), such as emulsifiers (e.g. to disperse oil in water), wetting agents (e.g. to reduce interfacial tensions between normally repelling substances), stickers (e.g. to cause the pesticide to adhere to the plant foliage and also to resist wash-off), and spreader-stickers (e.g. combined products that provide better spray coverage and adhesion). Crop oils and crop oil concentrates are light, petroleum-based oils that contain surfactant. Antifoam agents (foam suppressants) may be used to suppress foam formed when pesticides are agitated in the spray tank.

Carriers may serve as the diluent for any of the formulations provided herein. The carrier is the material to which a formulated pesticide is added, e.g. for field applications. A carrier may be used to enable uniform distribution of a small amount of formulated pesticide to a large area. Carriers may include liquid, dry and foam carriers. Liquid carriers, e.g. for spray applications, may include water, liquid fertilizers, vegetable oils, and diesel oil. Dry carriers may be used to apply pesticides without further dilution and may include attapulgite, kaolinite, vermiculite, starch polymers, corn cob, and others. Dry fertilizers can also be carriers.

The compositions provided herein can be a sprayable formulation. Sprayable Formulations (with liquid carrier) include: water-soluble liquids (designated S or SL or SC: form true solutions when mixed with water); Water-soluble powders (designated SP or WSP: are finely divided solids that dissolve completely in water); emulsifiable concentrates (designated E or EC: are oil-soluble emulsifiers that form emulsions when mixed with water); wettable powders (designated W or WP: are finely ground solids consisting of a dry carrier (a finely ground hydrophilic clay), pesticide, and dispersing agents, form an unstable suspension when mixed with water); water-dispersible liquids (designated WDL, L, F, AS: are finely ground solids suspended in a liquid system and form suspension when added to water); water-dispersible granules (designated WDG or DF, also called dry flowables, are dry formulations of granular dimensions made up of finely divided solids that combine with suspending and dispersing agents). Sprayable formulations may be in the form of aerosols and may be applied as droplets.

The compositions provided herein can be a dry formulation. Dry Formulations (e.g. for direct application without dilution in a liquid carrier) include: granules (designated G: consist of dry material in which small, dry carrier particles of uniform size (e.g. clay, sand, vermiculite, or corn cob; with a granule size of e.g. less than 0.61 cubic inches) are impregnated with the active ingredient, and may be applied with granular applicators); pellets (designated P: are dry formulations of pesticide and other components in discrete particles usually larger than 0.61 cubic inches, and may be applied e.g. by hand from shaker cans or with hand spreaders for spot applications). Dry formulations may also be applied as a fine powder or dust.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### General Procedures

All reactions were performed in oven-dried or flame-dried round-bottom flasks fitted with rubber septa and were conducted under positive argon pressure using standard Schlenk techniques, unless noted otherwise. Cannulae or gas-tight syringes with stainless steel needles were used to transfer air- or moisture-sensitive liquids. Where necessary (so noted), solutions were degassed by sparging with argon for a minimum of 10 min. Flash column chromatography was performed as described by Still et al.⁴ using granular silica gel (60-Å pore size, 40.63 μm, 4-6% H₂O content, Zeochem). Analytical thin layer chromatography (TLC) was performed using glass plates pre-coated with 0.25 mm 230.400 mesh silica gel impregnated with a fluorescent indicator (254 nm). TLC plates were visualized by exposure to short wave ultraviolet light (254 nm) and irreversibly stained by treatment with an aqueous solution of ceric ammonium molybdate (CAM) or an aqueous solution of potassium permanganate (KMnO₄) followed by heating (˜1 min) on a hot plate (˜250° C.). Organic solutions were concentrated at 30-35° C. on rotary evaporators capable of achieving a minimum pressure of ˜10 Torr. Diazene photolysis was accomplished by irradiation in a Rayonet RMR-200 photochemical reactor (Southern New England Ultraviolet Company, Branford, Conn., USA) equipped with 14 radially distributed (r=12.7 cm) 25 W lamps.

### Materials

Commercial reagents and solvents were used as received with the following exceptions: acetonitrile, dichloromethane, N,N-dimethylformamide, methanol, tetrahydrofuran, toluene, and triethylamine were purchased from EMD Millipore (ReCycler™) or Sigma-Aldrich (Pure-Pac™) and were purified by the method of Grubbs et al. under positive argon pressure. Benzene, 1,2-dichloroethane, and N,N-diisopropylethylamine were dried by distillation over calcium hydride under an inert dinitrogen atmosphere. Deuterated solvents used for nuclear magnetic resonance (NMR) spectroscopy were purchased from Cambridge Isotope Laboratories, Inc. and were used as received with the exception of chloroform-d, which was stored over activated molecular sieves (Linde type 3 Å, 1/16″ pellets) and granular anhydrous potassium carbonate. Titanium(IV) ethoxide (containing 5-15% isopropanol) was purchased from Strem Chemicals Inc.; 2,6-di-tert-butyl-4-methylpyridine was purchased from Matrix Scientific and was further purified by flash column chromatography on silica gel (eluent: hexanes); (−)-diacetone-D-glucose was purchased from Chem-Impex International, Inc. and was further purified by flash chromatography on silica gel (eluent: 30% acetone in hexanes) or from Sigma-Aldrich and was used as received; hexafluoroisopropanol was purchased from Oakwood Products, Inc. and was stored under an argon atmosphere over activated 4 Å molecular sieves; tetra-n-butylammonium hydrogen sulfate, tert-butyl hypochlorite, and N-carbobenzoxy-2-nitrobenzenesulfonamide were purchased from TCI America; (S)-4-benzylthiazolidine-2-thione and tryptamine were purchased from AK Scientific, Inc.; calcium trifluoromethanesulfonate, cesium carbonate, lithium hydroxide monohydrate, thiophenol, and triphenylphosphine were purchased from Alfa Aesar. All other solvents and chemicals were purchased from Sigma-Aldrich.

### Instrumentation

Nuclear magnetic resonance (¹H, ¹³C, and ¹⁹F NMR) spectra were recorded with Bruker AVANCE NEO 600, Bruker AVANCE 600, Bruker AVANCE NEO 500, Varian inverse probe INOVA-500, Varian INOVA-500, JEOL ECZR 500, or Bruker AVANCE III 400 spectrometers and are reported in parts per million on the 6 scale. Spectra were processed with MestReNova 12.0.2 using the automatic phasing and third-order polynomial baseline correction capabilities. Splitting was determined using the automatic multiplet analysis function with manual intervention as necessary. Proton NMR spectra are referenced from the residual protium in the NMR solvent (CHCl₃: δ 7.26, CD₂HCN: δ 1.94, CD₂HOD: δ 3.31, DMSO-d₅: 2.50, C₆D₅H: δ 7.16).⁶ Carbon-13 NMR spectra are referenced from the carbon resonances of the deuterated solvent (CDCl₃: δ 77.16, CD₃CN: δ 118.26, CD₃OD: δ 49.00, DMSO-d₆: 39.52, C₆D₆: δ 128.06).⁶ Fluorine-19 NMR are referenced internally from the fluorine resonances of α,α,α-trifluorotoluene (CF₃C₆H₅ δ −63.72). Data are reported as follows: chemical shift (multiplicity [s=singlet, d=doublet, t=triplet, q=quartet, p=pentet, m=multiplet, br=broad, app=apparent], coupling constant(s) in Hertz, integration, assignment).

Infrared spectroscopic data were obtained with a Perkin-Elmer 2000 FTIR spectrometer or a Bruker ALPHA II FTIR spectrometer equipped with a diamond ATR sampling module and are reported as follows: frequency of absorption (cm⁻¹) [intensity of absorption (s=strong, m=medium, w=weak, br=broad)]. Optical rotations were measured on a Jasco P-1010 polarimeter with a sodium lamp and are reported as follows: [α]_{λ}^{T ° C.} (c=g/100 mL, solvent). Chiral HPLC analysis was performed on an Agilent Technologies 1100 Series instrument equipped with a diode array detector and columns with chiral stationary phases from Daicel Chemical Industries (CHIRALPAK® IA, Lot#IA00CE-PD046 and CHIRALCEL® OD-H, Lot#ODHOCE-KF021). Single crystal X-ray diffraction was carried out at the X-ray crystallography laboratory of the Department of Chemistry, Massachusetts Institute of Technology. High-resolution mass spectra (HRMS) were recorded on a Bruker Daltonics APEXIV 4.7 Tesla FT-ICR-MS using an electrospray (ESI) (m/z) ionization source or a direct analysis in real time (DART) ionization source, on an Agilent 6510 QToF with a Dual ESI spray ionization source, or on a JEOL AccuTOF LC-plus 4G API-HRTOFMS equipped with an IonSense DART ionization source.

### Positional Numbering System

In assigning the ¹H and ¹³C NMR data of all intermediates en route to the communesin alkaloids, a uniform numbering system illustrated below using (−)-Communesin B was employed.

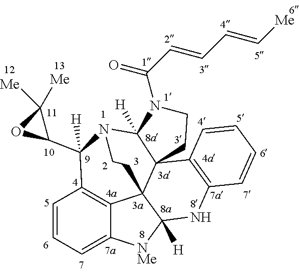

### Cell Culture Information

Cells were grown in media supplemented with fetal bovine serum (FBS) and antibiotics (100 g/mL penicillin and 100 U/mL streptomycin). Specifically, experiments were performed using the following cell lines and media compositions: HeLa (cervical adenocarcimona) and A549 (lung carcinoma) were grown in RPMI-1640+10% FBS; HCT-116 (colorectal carcinoma) was grown in DMEM+10% FBS; DU-145 (prostate carcinoma) and MCF7 (breast adenocarcinoma) were grown in EMEM+10% FBS. Cells were incubated at 37° C. in a 5% CO₂, 95% humidity atmosphere.

### Cell Viability Assays

Cells were plated at 2000 cells/well into duplicate assay plates in 50 L media into 384-well white, opaque, tissue-culture treated plates and allowed to adhere overnight at 37° C./5% CO₂. Compounds were solubilized in DMSO as 1000× stocks and 100 nl was pin-transferred to cells (V&P pin tool mounted on Tecan Freedom Evo MCA96). Compounds were tested in 10-pt, 2-fold dilution with concentrations tested between 1 nM-20 μM for most compounds, except where indicated. DMSO (32 wells of 384-wells) was used as vehicle control. After 72 hours of incubation at 37° C./5% CO₂, 10 L Cell Titer-Glo (Promega) was added to each well and plates were incubated at room temperature for 10 minutes before the luminescence was read on a Tecan M1000 plate reader. Cell Titer-Glo measures ATP levels of cells as a surrogate for cell viability. All compound-treated wells was normalized to the DMSO control averages and expressed as a % of DMSO viability. IC₅₀ values were determined from the dose curves using Spotfire (Perkin Elmer).

### DETAILED DESCRIPTION OF EXAMPLES

The retrosynthetic analysis (Scheme 2) describes the synthetic route undertaken herein for the synthesis of (−)-communesin A (2) and related epoxy-communesins via a late-stage biomimetic aminal reorganization of epoxy-heterodimer 18 followed by N1′ acylation. Consistent with a prior reported diazene-directed strategy for complex fragment assembly,^{2b,7} the C3a-C3a′ linkage in 18 could be assembled via photoextrustion of dinitrogen from unsymmetrical diazene 21 and recombination of the resulting radical fragments 19 and 20.

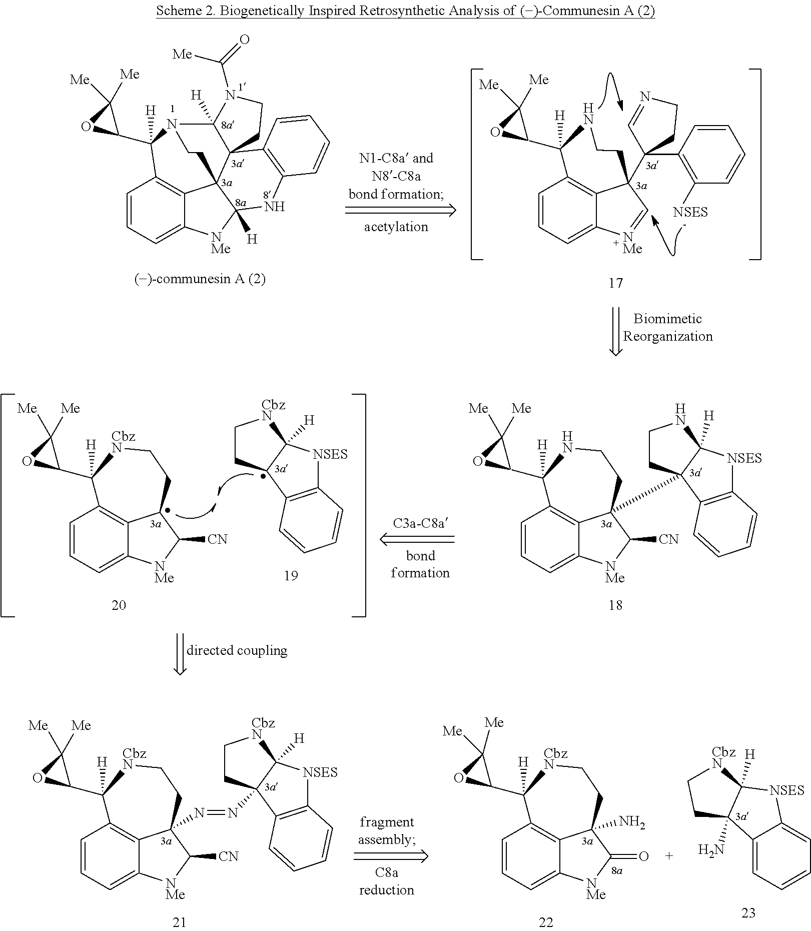

The synthesis of (−)-communesin A (2) began with the preparation of 22 and 23, the amine fragments required for the synthesis of complex diazene 21. The application of silver(I)-mediated substitution chemistry enabled rapid and scalable access to 23 and related sulfamate (+)-25. (Scheme 3). Electrophilic activation of readily available enantioenriched C3a-bromo-cyclotryptamine (+)-24^{2b,7d,8} with silver(I) trifluoromethane-sulfonate in the presence of 2,6-difluorophenylsulfamate and 2,6-di-tert-butyl-4-methylpyridine (DTBMP) afforded the corresponding sulfamate ester (+)-25 in 69% yield on a gram scale.

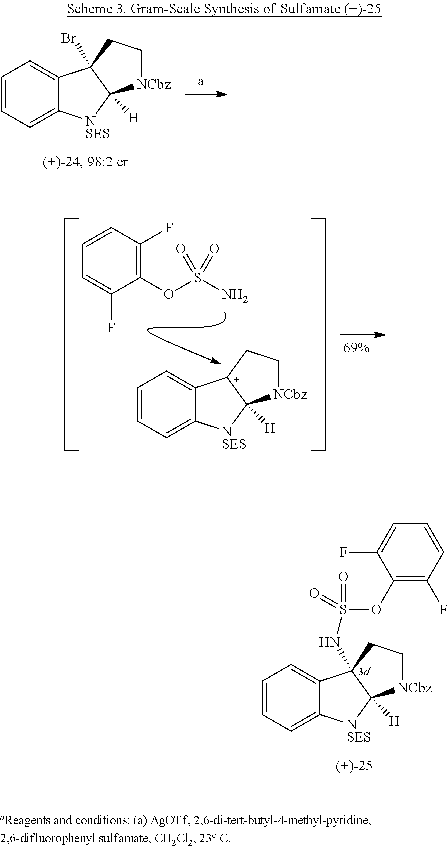

Having secured a scalable and robust synthesis of cyclotryptamine (+)-25, the preparation of amino azepane fragment 22 which contains a (10R)-configured epoxide, a critical structural feature found in communesins 2.10 was undertaken. Initial efforts directed towards the synthesis of this intermediate and related derivatives revealed a pronounced acid-sensitivity of the C10-epoxide, which stems from facile intramolecular opening of the protonated epoxide with the N1-carbamate to form stable oxazolidinone products.⁹ This precluded the use of Ellman's tert-butanesulfinamide chiral auxiliary,¹⁰ which was previously employed en route to (−)-communesin F (1).^{2b} Specifically, epoxidation of intermediates containing Ellman's auxiliary resulted in rapid concomitant oxidation of the sulfinamide to the corresponding tert-butanesulfonamide (Bus), which can only be removed by the action of anhydrous trifluoromethanesulfonic acid.¹¹ This unforeseen incompatibility prompted the design of 2-(trimethylsilyl)ethane sulfinamide (26), a new sulfinamide auxiliary whose oxidation product, 2-(trimethylsilyl)ethane sulfonamide (SES), can be removed under non-acidic and non-reducing conditions,^{1a} requirement for the preservation of the sensitive C10-epoxide (Scheme 4).

Multi-gram quantities of enantiopure (S)-sulfinamide (−)-26 were prepared using readily available (−)-diacetone-D-glucose¹³ as a chiral controller.¹⁴ Condensation of (−)-26 with N-methyl-4-bromoisatin in the presence of titanium(IV) ethoxide then afforded the corresponding sulfinyl imine (+)-27 in 80% yield. Subsequent allylation with allylmagnesium bromide afforded the corresponding addition product (+)-28 in 74% yield as a single diastereomer on a multi-gram scale after flash column chromatography. The inherent diastereoselectivity imparted by this new auxiliary (84:16 dr) was remarkably similar to that observed with Ellman's tert-butanesulfinamide (87:13 dr) under identical reaction conditions,^{2b} thereby validating the aptitude of (−)-26 in stereoselective synthesis.

Ozonolysis of alkene (+)-28 followed by in situ ozonide reduction with sodium borohydride furnished primary alcohol (+)-29 in 85% yield. Mitsunobu displacement of the alcohol with N-carbobenzoxy-2-nitrobenzenesulfonamide (o-NsNHCbz) and in situ desulfonylation then afforded benzyl carbamate (+)-30 in 76% overall yield. A palladium-catalyzed Mizoroki-Heck reaction with 1,1-dimethylallyl alcohol and silver(I) carbonate as the base then proceeded to furnish allylic alcohol (−)-31 in 92% yield.¹⁵ Unexpectedly, subjecting (−)-31 to previously employed palladium-catalyzed allylic amination conditions (PdC₂MeCN₂, MeCN, 80° C.)^{2b,16} resulted in complex mixtures containing only trace amounts of azepane (−)-32. The major side products were derived from sulfinamide epimerization and desulfinylation. It was hypothesized that the transiently generated hydrochloric acid necessary for catalyst turnover¹⁷ resulted in sulfinamide cleavage and release of the free amine and the corresponding sulfinyl chloride, which is expected to be configurationally unstable.¹⁸ Recombination of the amine and the racemized sulfinyl chloride would then afford the observed diastereomeric sulfinamide.

After extensive experimentation, calcium(II) trifluoromethanesulfonate and related Lewis acids¹⁹ could promote a highly efficient allylic amination without concomitant sulfinamide degradation. Indeed, under optimal conditions, gram scale synthesis of (−)-32 was achieved in 90% yield.

Having secured a scalable and robust synthesis of alkene (−)-32, the formation of the C10-epoxide was pursued. Mild, efficient, and stereoselective epoxidation of this intermediate could be achieved using in situ generated methyl(trifluoromethyl)dioxirane (TFDO).^{20,21} Exposure of an acetonitrile solution of (−)-32 to aqueous potassium carbonate and 30% aqueous hydrogen peroxide in the presence of 1,1,1-trifluoroacetone at 0° C. furnished the desired (R)-configured epoxide (−)-33 in 81% yield in addition to the unnatural (S)-configured epoxide (−)-34 in 8% yield with concomitant oxidation of the alkane sulfinamide to the corresponding 2-(trimethylsilyl) ethane sulfonamide (SES).²²

The relative configuration at C10 of these epimeric epoxides was determined by nuclear Overhauser effect analysis on free amines (−)-35 and (−)-36 after hydrogenolytic removal of the benzyl carbamates (Scheme 4). According to Murata's J_{H-H}-based method²³ as employed by Proksch,^{2b} Christophersen,^{2e} and Chen^{2f} for communesins 4-9, the large coupling constant between C9H and C10H (J≈9.0 Hz) in both (−)-35 and (−)-36 indicates an approximately 1800 dihedral angle between C9H-C9-C10-C10H, leaving two possible diastereomeric anti configurations. In (−)-35, the nOe enhancement at C5H observed when irradiating the geminal methyl groups C12H₃ and C13H₃ suggests a syn orientation between the epoxide oxygen and N1 as depicted in the Newman projection in Scheme 4, therefore implying an (R) configuration at C10. Conversely, the nOe enhancements observed at C2H in (−)-36 when irradiating the geminal methyl groups suggests an anti orientation between the epoxide oxygen and N1 and thus an (S) configuration at C10. The assignment in the latter case was unambiguously confirmed by single-crystal X-ray diffraction.¹⁴

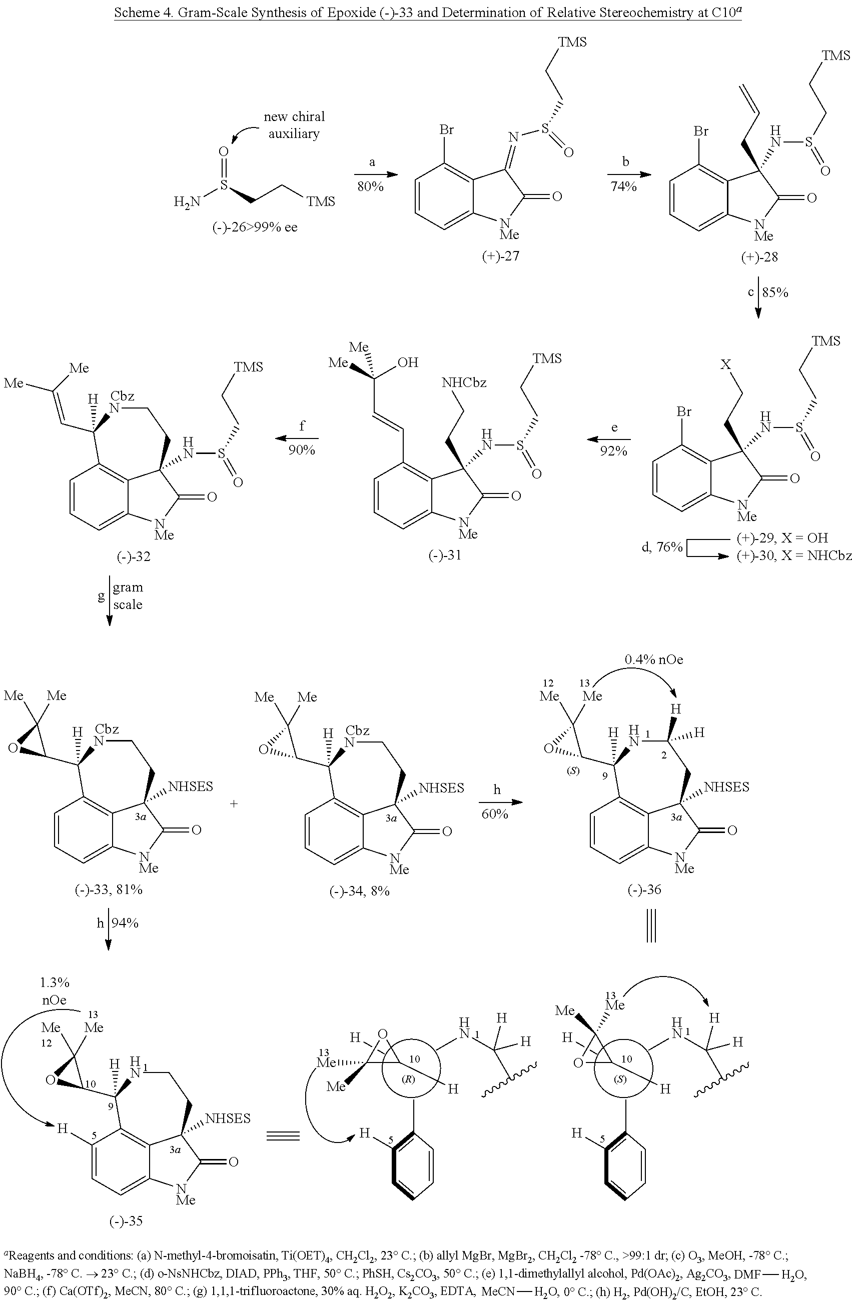

With a practical and stereoselective synthesis of epoxide (−)-33 in hand, the C8a reduction and unveiling of the C3a amine was investigated. The installation of a C8a-nitrile was investigated, which has been shown to be a trigger for late-stage C8a-iminiun ion formation while providing adequate stability during the fragment assembly steps (Scheme 5).^{2b} To this end, partial reduction of oxindole (−)-33 with lithium borohydride afforded the corresponding C8a-hemiaminal as a mixture of diastereomers. Treatment of the crude hemiaminal with trimethylsilyl cyanide in wet hexafluoroisopropanol (HFIP) afforded aminonitrile (+)-37 in 57% yield.²⁴ Fluoride-mediated C3a-N desulfonylation with tris(dimethylamino)sulfonium difluorotrimethylsilicate (TASF)²⁵ in anhydrous N,N-dimethylformamide (DMF) at 100° C. then provided benzylic amine (+)-38 in 39% yield, with the C8a-epimer and the C8a-cyanoindole elimination product comprising the remainder of the mass balance. Attempts to attenuate the basicity of the reagent by the addition of water or other acidic additives proved unsuccessful in reducing the propensity of the substrate to undergo elimination or epimerization. In addition, the efficiency of the reaction was capricious and isolated yields of (+)-38 diminished notably on scale up. Therefore, in order to circumvent these problematic side reactions, directed desulfonylation of oxindole (−)-33 was studied, along with investigations into C8a reduction after the fragment assembly. Gratifyingly, treating (−)-33 with TASF in wet DMF at 100° C. furnished amino-oxindole (−)-22 in 69% yield on a gram scale.

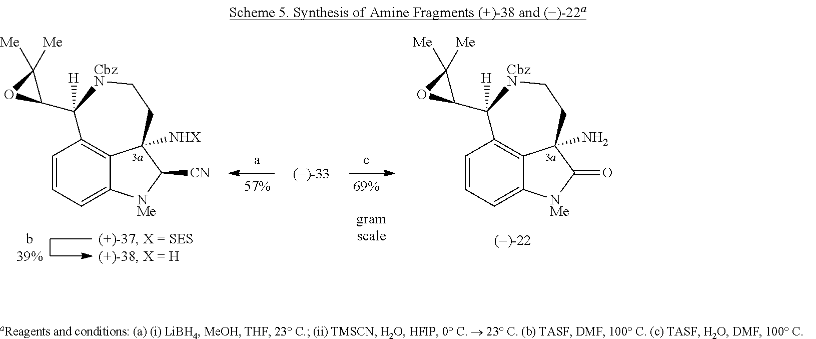

Having developed versatile and scalable syntheses of both amine fragments, the union of the fragments and the construction of the C3a-C3a′ linkage was pursued. Simply stirring a tetrahydrofuran solution of amine (−)-22 and sulfamate (+)-25 in the presence of 4-(dimethylamino)pyridine (DMAP) afforded oxindole sulfamide (−)-39 in 84% yield on a gram scale (Scheme 6). Partial reduction of the oxindole with excess lithium borohydride and treatment of the resulting crude hemiaminal with trimethylsilyl cyanide in wet²⁶ hexafluoroisopropanol then afforded aminonitrile sulfamide (+)-40 as a single diastereomer in 84% overall yield on a gram scale. Formation of the C8a-nitrile after fragment assembly proved to be much more efficient and diastereoselective, which was attributed to the steric bulk of the cyclotryptamine moiety that more effectively shields the bottom face of the C8a-iminium.

Exposure of (+)-40 to N-chloro-N-methylbenzamide in the presence of polystyrene-bound 2-tert-butylimino-2-diethylamino-1,3-dimethylperhydro-1,3,2-diazaphosphorine (BEMP) in methanol then afforded sensitive diazene 21 in 45% yield, without competitive oxidation of the electron-rich arene.^{2b} Photoexcitation and expulsion of dinitrogen from a thin film of diazene 21 followed by combination of the resulting radical fragments 19 and 20 afforded C3a-C3a′-linked heterodimer (+)-41 in 50% yield as a single diastereomer.²⁷ Hydrogenolysis of the benzyl carbamates then furnished heterodimeric diamine (+)-18 in 77% yield, setting the stage for biomimetic aminal reorganization. Consistent with the design principles underpinning this synthetic strategy, the position of the electron withdrawing group on the cyclotryptamine moiety enables selective cleavage of either aminal linkage, thereby controlling the regiochemical outcome of the rearrangement. In diamine (+)-18, the N8′-sulfonamide enables a guided fragmentation of the C8a-N8′ bond under basic conditions, which leads to the heptacyclic core of the communesin alkaloids after formation of the C8a-N8′ and C8a-N1 aminal linkages.

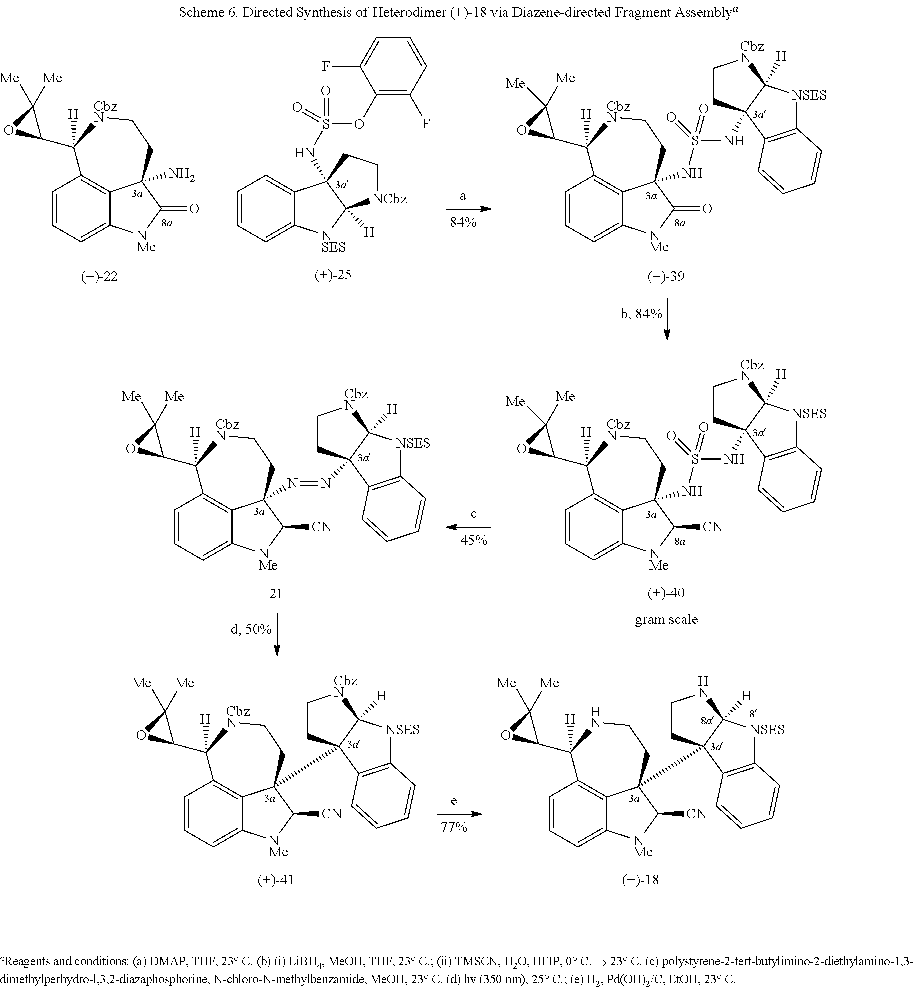

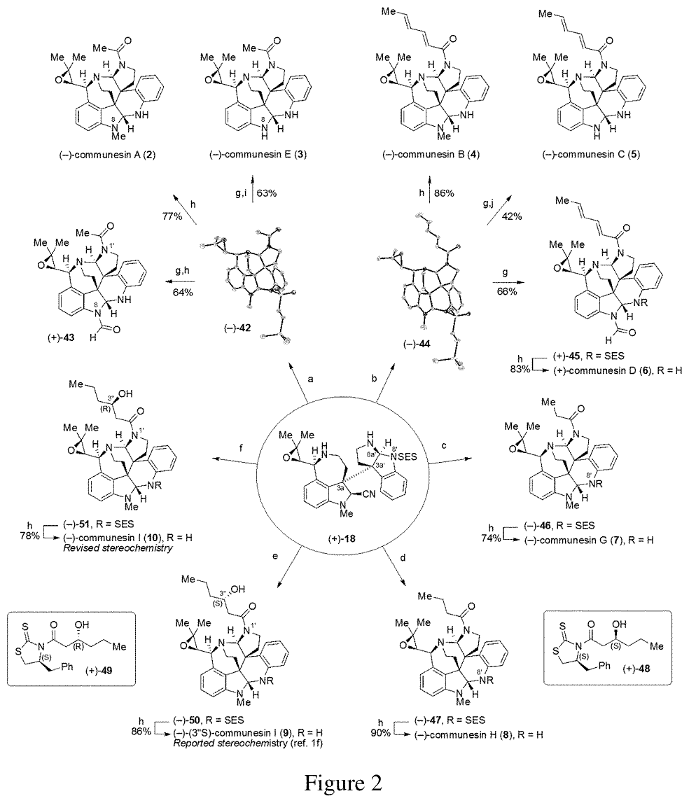

Having achieved a robust solution for the preparation of heterodimer (+)-18, efforts were undertaken to prepare all known epoxide-containing members of the communesin family, beginning with N1′-acetyl communesins (−)-2 and (−)-3, respectively. Clean and complete rearrangement to the epoxide-appended communesin core could be achieved by exposing (+)-18 to ethanolic lithium tert-butoxide at 60° C. (FIG. 2).²⁸ In situ neutralization of excess alkoxide with pyridinium p-toluenesulfonate (PPTS) followed by acetylation of the resulting sensitive heptacycle with acetic anhydride then furnished (−)-42, which upon mild N8′-desulfonylation with TASF in degassed²⁹ DMF provided (−)-communesin A (2) in 63% overall yield from (+)-18. All ¹H and ¹³C NMR data as well as optical rotation (observed [α]_{D}²⁴=−165 (c=0.39, CHCl₃); lit: [α]_{D}²²=−58 (c=0.14, CHCl₃),^{30a} [α]_{D}²⁰=−174 (c=1.34, CHCl₃),^{30c} [α]_{D}³⁰=−163.5 (c=0.14, CHCl₃)³) for synthetic (−)-2 were consistent with previously reported literature values. Oxidation of (−)-42 with pyridinium dichromate (PDC, 10 equiv) and potassium carbonate (40 equiv) in 1,2-dichloroethane at 60° C. then furnished the corresponding N8-formyl derivative,³¹ which, after desulfonylation, afforded (+)-N8-formyl communesin E (43) in 64% overall yield. Interestingly, this analogue has not yet been isolated in nature, which is notable given that natural samples of (+)-communesin D (6), the N1′-sorbyl derivative, have been repeatedly and independently isolated.^{30b,c} Following a similar sequence from (−)-42 with an additional provision for mild formamide hydrolysis with potassium hydroxide in wet dimethyl sulfoxide (DMSO) afforded (−)-communesin E (3) in 63% overall yield. All spectral data and optical rotation (observed [α]_{D}²³=−191 (c=0.31, CHCl₃); lit: [α]_{D}²⁰=−156 (c=0.11, CHCl₃)^{30c}) for alkaloid (−)-3 were in agreement with the isolation report. In addition, analysis of (−)-42 by single-crystal X-ray diffraction unambiguously confirms the stereochemical configuration of the C10-epoxide in (−)-2 and (−)-3 for the first time.

Having successfully completed the synthesis of all known N1′-acetyl communesin alkaloids and a related complex derivative, synthesis of N1′-sorbyl alkaloids was studied. Subjecting (+)-18 to the standard rearrangement conditions followed by acylation with sorbic anhydride afforded (−)-N8′-(trimethylsilyl)ethanesulfonyl communesin B (44) in 82% yield (FIG. 2). Mild N8′-desulfonylation with TASF then furnished (−)-communesin B (4) in 86% yield, whose spectroscopic data as well as optical rotation (observed [α]_{D}²³=−64 (c=0.46, CHCl₃); lit: [α]_{D}²²=+8.7 (c=0.23, CHCl₃),^{30a} [α]_{D}=−58 (c=0.10, MeOH),^{30b} [α]_{D}²⁰=−74.9 (c=1.50, CHCl₃),^{30c} [α]_{D}³⁰=−51.3 (c=0.30, CHCl₃)₃) were consistent with previously reported values, with the exception of the anomalous positive value described in Numata's 1993 isolation report.^{30a} Next, PDC-mediated oxidation of (−)-44 provided sensitive N8-formamide (−)-45 in 66% yield which was then desulfonylated to provide (+)-communesin D (6) in 83% yield. All ¹H and ¹³C data as well as optical rotation (observed [α]_{D}²³=+151 (c=0.23, CHCl₃); lit: [α]_{D}²⁰=+150 (c=0.14, CHCl₃)^{30c}) of (+)-6 were fully consistent with literature values. To complete the synthesis of all N1′-sorbyl analogues, deformylation of the N8-formamide followed by desulfonylation of the resulting crude amine produced (−)-communesin C (5) in 42% overall yield from (−)-44. The spectral data and optical rotation of alkaloid (−)-5 (observed [α]_{D}²³=−108 (c=0.28, MeOH); lit: [α]_{D}=−30 (c=0.038, MeOH)^{30b}) were in agreement with literature values. Importantly, analysis of the common precursor (−)-44 by single-crystal X-ray diffraction unambiguously confirms the relative and absolute stereochemical configuration of all known N1′-sorbyl communesin alkaloids (−)-4, (−)-5, and (+)-6 for the first time.

Also, (−)-communesin G (7) and H (8) were prepared (FIG. 2). Rearrangement of (+)-18 under the standard conditions followed by acylation with propionic anhydride efficiently furnished N1′-propionyl communesin G (−)-46 in 86% yield. Subsequent desulfonylation with TASF then afforded (−)-communesin G (7) in 74% yield, with spectral data and optical rotation (observed [α]_{D}²³=−163 (c=0.20, MeOH); lit: [α]_{D}²⁵=−157 (c=0.021, MeOH^{30e}) fully consistent with those reported in the isolation report. Similarly, rearrangement of (+)-18, acylation with butyric anhydride, and desulfonylation of the intermediate heptacycle (−)-47 efficiently furnished (−)-communesin H (8) in 76% overall yield, with all spectral data and optical rotation (observed [α]_{D}²³=−168 (c=0.38, MeOH); lit: [α]_{D}²⁵=−167 (c=0.024, MeOH)^{30e}) identical to those previously reported.

Based on the reported structure, (−)-communesin I (9), the most recently isolated member of the communesin family was synthesized using the methods described herein (FIG. 2). In order to install the (3″S)- hydroxy amide at N1′, (S′),(S′)-aldol adduct (+)-48 was used as the acyl donor¹⁴ after the key aminal reorganization. To this end, standard rearrangement of (+)-18 followed by acylation of the communesin core with excess (+)-48 furnished alkaloid (−)-50 in 84% yield. Desulfonylation with TASF then afforded (−)-(3″S)-communesin I (9) in 86% yield, which enabled careful analysis of all spectral data and conclusive comparisons with the isolation data originally reported by Fan and co-workers^{30f} for natural (−)-communesin I. The ¹H and ¹³C signals associated with the core of the alkaloid were in good agreement with the isolation report (≤0.5 ppm difference between ¹³C NMR signals), however several key ¹H and ¹³C signals on the acyl chain deviated notably from the expected values. Specifically, the ¹³C chemical shifts of C2″ (41.1¹⁴ vs. 42.1^{30f} ppm), C3″ (68.1¹⁴ vs. 69.0³⁰f ppm), and C4″ (38.8¹⁴ vs. 39.5^{30f} ppm) were found to be the most divergent.

It was hypothesized that the stereochemical configuration at C3″ had been incorrectly assigned in the isolation report. Given the ease with which the diastereomeric (S),(R)-aldol adduct (+)-49 could be prepared, the corresponding (3″R) derivative (10) was synthesized to test this hypothesis. Standard reorganization of (+)-18 followed by acylation with (+)-49 furnished (3″R) analogue (−)-51 in 48% yield, which upon N8′-desulfonylation afforded (−)-(3″R)-communesin I (10) in 78% yield. All ¹H and ¹³C spectroscopic data of this alkaloid were in excellent agreement with those reported in Fan's isolation report^{30f} of (−)-communesin I. The sign of the optical rotation was also consistent with the reported data, albeit with a somewhat higher absolute value (observed [α]_{D}²³=−137 (c=0.22, MeOH); lit: [α]_{D}²⁰=−59 (c=0.1, MeOH)^{30f}). As a result, the stereochemical configuration at C3″ of this new communesin alkaloid is not (S), but rather (R). This important finding decisively validates the importance of this strategic late stage N1′ acylation, which enables the rapid diversification and functional derivatization of the communesin core.

Finally, having completed the total synthesis of all known naturally-occurring communesin alkaloids, the construction of inaccessible complex derivative was pursued using the synthetic strategies described and developed herein. In order to further demonstrate the modularity and versatility of this convergent approach, the iso-communesin core, an unnatural constitutional isomer of the communesin skeleton, was synthesized. It was hypothesized that the core of these derivatives would be easily accessible via an analogous aminal reorganization of a C3a-C3a′ linked heterodimer containing a cyclotryptamine fragment with an inverted N1′/N8′ substitution pattern. Treatment of this hypothetical substrate under the same basic conditions required to reorganize (+)-18 should result in the selective cleavage of the C8a-N1′ aminal, thereby resulting in the elements of the iso-communesin core after formation of the C8a-N1′ and C8a-N1 aminal linkages.

As depicted in Scheme 7, fragment assembly of aminonitrile (+)-38³² and the appropriately substituted C3a′-sulfamate (+)-52¹⁴ afforded aminonitrile sulfamide (+)-53 in 75% yield. Oxidation of (+)-53 under the same conditions employed for (+)-40 afforded sensitive diazene 54 in 57% yield. Photochemical irradiation of the diazene as a neat thin film at 350 nm then furnished heterodimer (+)-55 in 53% yield, which was then subjected to the standard conditions for benzyl carbamate hydrogenolysis. These conditions furnished a mixture of two new compounds with the same molecular weight in an approximately 3:1 ratio. The major component of the mixture was identified as the expected heterodimeric diamine 56. Interestingly, the ¹H NMR spectrum of the second compound in CDCl₃ was found to contain an apparent broad triplet at δ 4.77 ppm, which was coupling to a set of adjacent methylene protons. Furthermore, no such resonance was detected in CD₃OD solvent. These spectral features suggest the presence of an untethered ethylamino group containing an electron-withdrawing group at N1′ and are consistent with partially rearranged structure 57. Indeed, when a pure sample of 56 was treated with lithium tert-butoxide (10 equiv) in CD₃OD at 23° C., rapid and complete conversion to 57 was observed by ¹H NMR, thereby corroborating this hypothesis. Evidently, the lower pKa of indoline N8′H in heterodimer 56 relative to pyrrolidine N1′H in (+)-18 enables cyclotryptamine fragmentation even under the mildly basic conditions of the carbamate hydrogenolysis.

Treatment of the crude mixture of 56 and 57 with lithium tert-butoxide in ethanol at 60° C. resulted in clean conversion to iso-communesin derivative (+)-58.²⁸ Analysis of two-dimensional NMR spectra provided decisive HMBC correlations in support of the structural assignment of this new alkaloid. Specifically, observed correlations between C8aH-C2′ and C8a′H—C9 conclusively establish the presence of the C8a-N1′ and C8a-N1 aminal linkages, respectively. The successful implementation of this synthetic strategy for the preparation of an iso-communesin derivative demonstrates the modularity and versatility of this approach and enables the exploration of previously unexplored chemical space for the treatment of human disease.

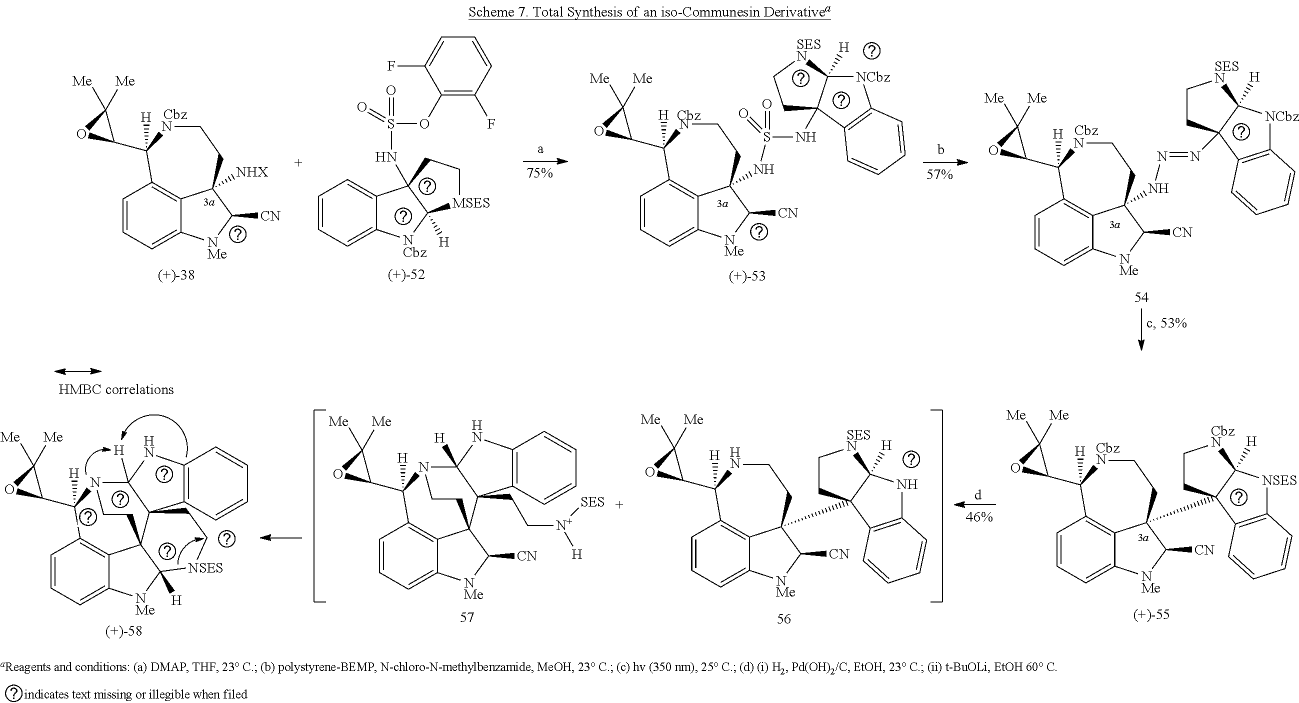

With samples of all known communesin alkaloids and a selection of unnatural derivatives in hand, the anticancer activity for this entire class of natural products was explored. While previous isolation reports have evaluated the activity of selected natural communesins, no comprehensive comparison of the entire class of alkaloids across multiple cell lines has been performed. To this end, all nine naturally occurring communesins, a selection of N8′-sulfonylated communesin derivatives, and N8′-sulfonylated iso-communesin (+)-58 were examined for cytotoxicity against human lung carcinoma (A549), prostate carcinoma (DU-145), colorectal carcinoma (HCT116), cervical adenocarcinoma (HeLa), and breast adenocarcinoma (MCF7) cell lines.¹⁴ As depicted in Table 1, (−)-communesin B (4) exhibited the highest potency of all the natural products tested across all cell lines, which is generally consistent with limited assays performed in early isolation reports.^{30a,b} The next most active natural alkaloid, (−)-communesin C (5), exhibited an approximately twofold decrease in potency, whereas compounds (−)-2, (−)-3, (−)-7, (−)-9, and (+)-43 were principally inactive across all cell lines.

More notably, however, complex derivatives containing an N8′-SES substituent generally exhibited a dramatic increase in potency relative to the N8′ unsubstituted congeners. For example, N8′-SES-communesin G, (−)-46, was found to exhibit an approximately 10-fold increase in potency relative to (−)-communesin G (7). This increase in activity was found to hold irrespective of N8 substitution (e.g. (−)-45 vs. (+)-6) or N1′ substitution (e.g., (−)-44 vs. (−)-4). To complete this preliminary structure-activity relationship (S.A.R.) study, it was noted that the N8 substituent exerts a small but measurable influence on potency. For example, a two- to threefold decrease in activity was observed moving from N8-methyl (−)-4 to either N8-H (−)-5 or N8-formyl (+)-6. The same trend was observed in the N8′-SES substituted series, but the relative variation was lower. Next, there was a general correlation between the size of the N1′ substituent and the potency of the compound. This was particularly evident in the natural series, where the activity follows the trend N1′-sorbyl>pentan-3R-ol>butyryl>propionyl>acetyl. As noted with the N8 substituent, the N8′-SES derivatives also followed the same general trend, but they were less sensitive to variation at this position. Finally, the unprecedented iso-communesin derivative(+)-58 was not impressively potent, with activity inferior to all N8′-SES communesin analogues tested as well as a number of more modestly active N8′-unsubstituted natural products.

Taken together, these preliminary data allow for the first side-by-side comparative analysis of all naturally occurring communesin alkaloids and suggest primarily that (a) substitution at N8′ can have a dramatic effect on potency; (b) N8-methyl derivatives exhibit improved activity relative to their N8-formyl or N8-unsubstituted counterparts; and (c) activity is nominally proportional to the size of the N substituent.

TABLE 1
Assessment of All Known Communesin Alkaloids and a Selection of
Unnatural Derivatives for Cytotoxicity against Lung (A549), Prostate (DU-145), Colorectal
(HCT-116), Cervical (HeLa), and Breast (MCF7) Carcinoma Cell Lines^{a}

| | | | | | IC₅₀ (μM) | | | | |
|---|---|---|---|---|---|---|---|---|---|
| Compound | Communesin | N1′ | N8 | N8′ | A549 | DU-145 | HCT-116 | HeLa | MCF7 |

SES Derivatives

| | | | | | | | | | |
|---|---|---|---|---|---|---|---|---|---|
| (−)-44 | N8′-SES-B | Sorbyl | Me | SES | 19 | 24 | >250 | >125 | 5 |
| (−)-46 | N8′-SES-G | Propionyl | Me | SES | 19 | 17 | 18 | 16 | 8 |
| (−)-50 | (3″S)-N8′- | (S)-3- | Me | SES | 24 | 36 | 32 | 17 | 15 |
| | SES-I | pentanol | | | | | | | |
| (−)-45 | N8′-SES-D | Sorbyl | CHO | SES | 39 | 29 | 27 | 15 | 24 |
| (−)-42 | N8′-SES-A | Acetyl | Me | SES | 34 | 53 | 31 | 35 | 30 |
| (+)-58 | iso- | SES | Me | H | 335 | >125 | >62.5 | 64 | 58 |
| | communesin | | | | | | | | |

N8′-H Natural Products and Derivatives

| | | | | | | | | | |
|---|---|---|---|---|---|---|---|---|---|
| (−)-4 | B | Sorbyl | Me | H | 56 | 45 | 60 | 47 | 34 |
| (−)-5 | C | Sorbyl | H | H | 115 | 82 | 106 | 94 | 65 |
| (−)-1 | F | Acetyl | Me | H | 117 | 119 | >125 | 115 | 84 |
| (−)-10 | C3″-(R)-I | (R)-3- | Me | H | 120 | >125 | >125 | 120 | 90 |
| | (natural) | pentanol | | | | | | | |
| (−)-8 | H | Butyryl | Me | H | >125 | >125 | 109 | 82 | 90 |
| (+)-6 | D | Sorbyl | CHO | H | >125 | 84 | 111 | 63 | >125 |
| (−)-9 | C3″-(S)-I | (S)-3- | Me | H | >125 | >125 | >125 | >125 | >250 |
| | (unnatural) | pentanol | | | | | | | |
| (−)-7 | G | Propionyl | Me | H | >125 | >125 | >125 | >125 | >250 |
| (−)-2 | A | Acetyl | Me | H | >125 | >250 | >250 | >125 | >250 |
| (+)-43 | N8-formyl-E | Acetyl | CHO | H | >250 | >250 | >250 | >125 | >250 |
| (−)-3 | E | Acetyl | H | H | >500 | >500 | >500 | >500 | >500 |

^{a}Cytotoxicity IC₅₀ values (in μM) after 72 h of compound treatment as determined by Cell Titer-Glo (Promega) which measures ATP levels as a surrogate for cell viability. Error is standard deviation of the mean, n ≥ 2; IC₅₀ = half maximal inhibitory concentration.

In summary, detailed herein is a unified enantioselective total synthesis of all known epoxide-containing communesin alkaloids and related complex derivatives from a common synthetic intermediate. This synthesis is predicated on the convergent and modular diazene-directed assembly of two advanced fragments to secure the C3a-C3a′ linkage followed by a guided biomimetic aminal reorganization to deliver the heptacyclic core of these alkaloids.

Concise enantioselective syntheses of the fragments were devised, with highlights including the use of a new, rationally-designed sulfinamide chiral auxiliary enabling a highly efficient stereoselective epoxidation and the application of a silver-mediated cyclotryptamine-C3a′-sulfamate synthesis from a readily-available enantioenriched C3a-bromocyclotryptamine.

The modularity of this convergent approach enabled the stereochemical revision of (−)-communesin I, the most recently isolated communesin analogue. Furthermore, the generality of the biomimetic reorganization was conclusively demonstrated in the first total synthesis of an iso-communesin derivative, an unnatural constitutional isomer of the communesin skeleton. Finally, reported herein is the first side-by-side anticancer profiling of all naturally occurring communesin alkaloids and nine complex derivatives for their ability to induce apoptosis in A549 (non-small-cell lung carcinoma), DU-145 (prostate carcinoma), HCT116 (colorectal carcinoma), HeLa (cervical adenocarcinoma), and MCF7 (breast adenocarcinoma) human cancer cell lines. From these data, (−)-communesin B was identified as the most potent natural isolate and discovered that derivatives containing an N8′-SES substituent exhibit up to a ten-fold increase in potency over the natural products. Indeed, these new synthetic analogues are among the most potent communesin alkaloids discovered to date.

This synthetic strategy sets the stage for further diversification and functional derivatization of the communesin core, which may culminate in the preparation of unnatural derivatives to enhance potency and further refine this preliminary structure-activity relationship (S.A.R.) study. In addition, the late-stage acylation at the N1′-position of the communesin core as described herein may be useful to prepare functional variants to probe the yet unknown molecular mode of action of these alkaloids.

### Synthesis of Exemplary Compounds

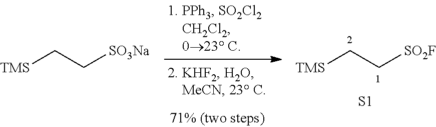

### Example 1: Synthesis of Sulfonyl Fluoride S1

Sulfuryl chloride (4.60 mL, 56.7 mmol, 2.20 equiv) was added dropwise via syringe over 6 min to a solution of triphenylphosphine (13.5 g, 51.6 mmol, 2.00 equiv) in dichloromethane (20.6 mL) at 0° C. After stirring at this temperature for 10 min, a sample of sodium 2-(trimethylsilyl)ethanesulfonate (95% purity, 5.60 g, 25.8 mmol, 1 equiv) was added as a solid in 12 portions over 6 min. After an additional 20 min at 0° C., the ice bath was removed and the resulting yellow suspension was allowed to stir vigorously at 23° C. After 24 h, the mixture was added dropwise via Pasteur pipette to a 500-mL round-bottom flask containing pentane (100 mL) with vigorous stirring over 15 min. After stirring for an additional 35 min, the suspension was diluted with pentane (100 mL) and was filtered through a 5.5-cm pad of silica gel, pre-packed with pentane in a 7.3-cm diameter column. The filter cake was washed with a solution of 5% diethyl ether in pentane (800 mL)³³ and the filtrate was concentrated under reduced pressure to yield crude 2-(trimethylsilyl)ethanesulfonyl chloride as a pale-yellow oil, which was used directly in the next step without further purification.³⁴

Under an air atmosphere, a 50-mL polypropylene Falcon tube containing a solution of potassium bifluoride (4.03 g, 51-6 mmol, 2.00 equiv) in deionized water (12.0 mL) at 23° C. was charged with a solution of crude 2-(trimethylsilyl)ethanesulfonyl chloride in HPLC-grade acetonitrile (10.0 mL). The transfer was quantitated with additional acetonitrile (2×3.0 mL). After vigorous stirring for 16 h, the layers were separated and the aqueous layer was extracted with diethyl ether (3×30 mL). The combined organic extracts were washed successively with a 10% aqueous sodium chloride solution (2×50 mL) and a saturated aqueous sodium chloride solution (50 mL), were dried over anhydrous sodium sulfate, were filtered, and were concentrated under reduced pressure. The residue was purified by flash column chromatography on silica gel (eluent: 2→4% diethyl ether in pentane) to afford sulfonyl fluoride S1 (3.38 g, 71.1%) as a colourless oil. ¹H NMR (500 MHz, CDCl₃, 20° C.): δ 3.34-3.24 (m, 2H, C1H₂), 1.23-1.13 (m, 2H, C2H₂), 0.10 (s, 9H, Si(CH₃)₃). ¹³C NMR (125.8 MHz, CDCl₃, 20° C.): δ 48.1 (d, J_{C,F}=16.4 Hz, C1), 10.6 (C2), −2.0 (Si(CH₃)₃). ¹⁹F NMR (470.9 MHz, CDCl₃, 20° C.): δ 47.1 (s, SO₂F). FTIR (thin film) cm⁻¹: 2958 (m), 2903 (w), 1399 (s), 1255 (s), 1206 (s), 1175 (w), 1125 (w), 1021 (w), 900 (m), 838 (s), 812 (s), 764 (s), 739 (m), 696 (m), 548 (s). HRMS (DART) (m/z): calc'd for C₅H₁₇FNO₂SSi [M+NH₄]: 202.0733, found: 202.0729. TLC (4% diethyl ether in pentane), Rf: 0.41 (KMnO₄).

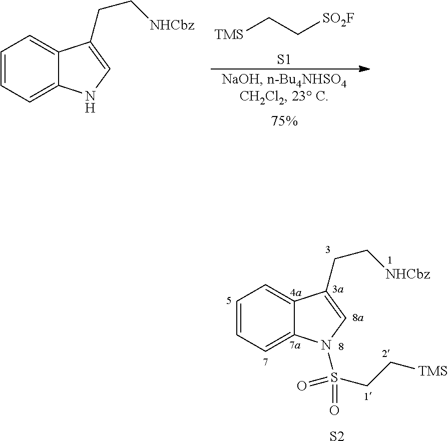

### Example 2: Tryptamine S2

2-(Trimethylsilyl)ethanesulfonyl fluoride³⁵ (S1, 1.87 g, 10.2 mmol, 1.30 equiv) was added dropwise via syringe to a suspension of benzyl (2-(1H-indol-3-yl)ethyl)carbamate³⁶ (2.30 g, 7.81 mmol, 1 equiv), freshly crushed sodium hydroxide (937 mg, 23.4 mmol, 3.00 equiv), and tetra-n-butylammonium hydrogen sulfate (265 mg, 0.781 mmol, 0.100 equiv) in dichloromethane (31 mL) at 23° C. After vigorous stirring for 8 h, the suspension was cooled to 0° C. and was acidified by portionwise addition of an aqueous hydrogen chloride solution (1 N, 31 mL). After warming to 23° C., the biphasic mixture was diluted with deionized water (30 mL) and the layers were separated. The aqueous phase was extracted with dichloromethane (3×30 mL) and the combined organic extracts were washed successively with water (2×100 mL) and a saturated aqueous sodium chloride solution (100 mL), were dried over anhydrous sodium sulfate, were filtered, and were concentrated under reduced pressure. The resulting residue was purified by flash column chromatography on silica gel (eluent: 20%→25% ethyl acetate in hexanes) to afford tryptamine S2 (2.69 g, 75.1%) as a colorless and viscous syrup. Structural assignments were made using additional information from gCOSY, gHSQC, and gHMBC experiments. ¹H NMR (500 MHz, CDCl₃, 20° C.): δ 7.88 (d, J=8.2 Hz, 1H, C7H), 7.61 (d, J=7.8 Hz, 1H, C4H), 7.40-7.29 (m, 7H, C5H, C6H, Ar_{Cbz}H), 7.28 (s, 1H, C8aH), 5.11 (s, 2H, N1CO₂CH₂Ph), 4.92 (br-t, J=6.1 Hz, 1H, HN1CO₂CH₂Ph), 3.53 (app-q, J=6.8 Hz, 2H, C2H), 3.20-3.10 (m, 2H, C1′H), 2.95 (t, J=7.0 Hz, 2H, C3H), 0.94-0.80 (m, 2H, C2′H), −0.05 (s, 9H, Si(CH₃)₃). ¹³C NMR (125.8 MHz, CDCl₃, 20° C.): δ 156.5 (N1CO₂CH₂Ph), 136.6 (Ar_{Cbz}), 135.6 (C7a), 130.5 (C4a), 128.7 (Ar_{Cbz}), 128.3 (Ar_{Cbz}), 128.2 (Ar_{Cbz}), 125.0 (C6), 124.1 (C8a), 123.2 (C5), 119.7 (C4), 118.4 (C3a), 113.3 (C7), 66.8 (N1CO₂CH₂Ph), 50.7 (C1′), 40.7 (C2), 25.7 (C3), 10.1 (C2′), −2.0 (Si(CH₃)₃). FTIR (thin film) cm⁻¹: 2954 (m), 2098 (br-m), 1699 (s), 1645 (s), 1523 (m), 1449 (m), 1361 (m). HRMS (ESI) (m/z): calc'd for C₂₃H₃₀N₂NaO₄SSi [M+Na]⁺: 481.1588, found: 481.1588. TLC (20% ethyl acetate in hexanes), Rf: 0.28 (UV, CAM).

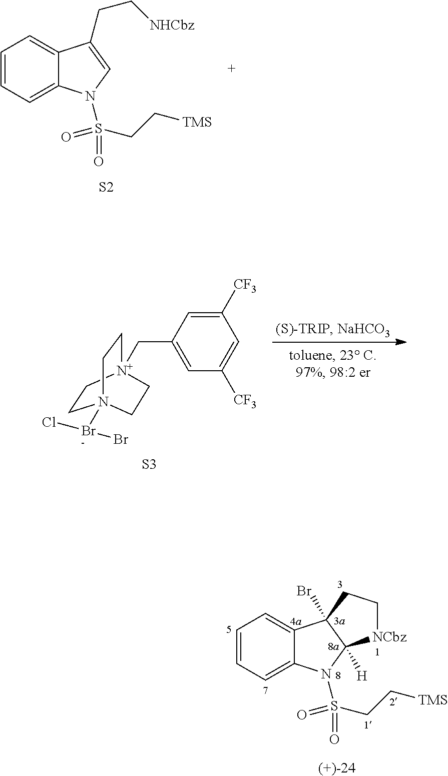

### Example 3: Bromocyclotryptamine (+)-24

A sample of bromine salt S3³⁷ (5.63 g, 10.5 mmol, 1.30 equiv) was added to a suspension of tryptamine S2 (3.72 g, 8.10 mmol, 1 equiv), (S)-3,3′-bis(2,4,6-triisopropylphenyl)-1,1′-binaphthyl-2,2′-diyl hydrogenphosphate³⁸ ((S)-TRIP, 610 mg, 0.810 mmol, 0.100 equiv), and crushed sodium hydrogen carbonate (2.72 g, 32.4 mmol, 4.00 equiv) in toluene (162 mL) at 23° C. After stirring for 24 h, the orange suspension was diluted with a saturated aqueous sodium thiosulfate solution (160 mL) and deionized water (320 mL) and was stirred vigorously for 15 min. The layers were separated and the aqueous layer was extracted with ethyl acetate (3×160 mL). The combined organic extracts were washed successively with an aqueous sodium thiosulfate solution (1 M, 320 mL) and a saturated aqueous sodium chloride solution (200 mL), were dried over anhydrous sodium sulfate, were filtered, and were concentrated under reduced pressure. The resulting residue was purified by flash column chromatography on silica gel (eluent: 14%→15% ethyl acetate in hexanes) to afford bromocyclotryptamine (+)-24 (4.21 g, 96.6%, 98:2 er) as a white foam.³⁹ The enantiomeric ratio was determined by chiral HPLC analysis (CHIRALPAK® IA, 10% iPrOH in hexanes, 1.0 mL/min, 210 nm, t_{R} (major)=10.3 min, t_{R} (minor)=12.7 min). As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (400 MHz, CD₃CN, 60° C.): δ 7.52 (ddd, J=7.7, 1.4, 0.6 Hz, 1H, C4H), 7.45-7.28 (m, 7H, C6H, C7H, Ar_{Cbz}H), 7.23 (ddd, J=7.7, 7.1, 1.3 Hz, 1H, C5H), 6.34 (s, 1H, C8aH), 5.25-5.11 (m, 2H, N1CO₂CH₂Ph), 3.86-3.75 (m, 1H, C2Ha), 3.56 (td, J=13.4, 4.7 Hz, 1H, C1′Hₐ), 3.35 (td, J=13.5, 4.8 Hz, 1H, C1′H_{b}), 3.01-2.90 (m, 1H, C3Hₐ), 2.89-2.78 (m, 2H, C2H_{b}, C3H_{b}), 1.10 (m, 2H, C2′H₂), 0.04 (s, 9H, Si(CH₃)₃). ¹³C NMR (100.6 MHz, CD₃CN, 60° C.): δ 155.5 (N1CO₂CH₂Ph), 143.3 (C7a), 138.0 (Ar_{Cbz}), 134.7 (C4a), 131.8 (C6), 129.8 (Ar_{Cbz}), 129.4 (Ar_{Cbz}), 129.2 (Ar_{Cbz}), 126.6 (C5), 125.6 (C4), 118.9 (C7), 88.3 (C8a), 68.6 (N1CO₂CH₂Ph), 64.5 (C3a), 51.9 (C1′), 47.4 (C2), 42.0 (C3), 11.3 (C2′), −1-6 (Si(CH₃)₃). FTIR (thin film) cm⁻¹: 2954 (w), 2869 (w), 2359 (m), 1707 (s), 1410 (s). HRMS (ESI) (m/z): calc'd for C₂₃H₃₀BrN₂O₄SSi [M+H]⁺: 537.0873, found: 537.0878. [α]_{D}²³: +198 (c=0.37, CH₂Cl₂). TLC (20% ethyl acetate in hexanes), Rf: 0.36 (UV, CAM).

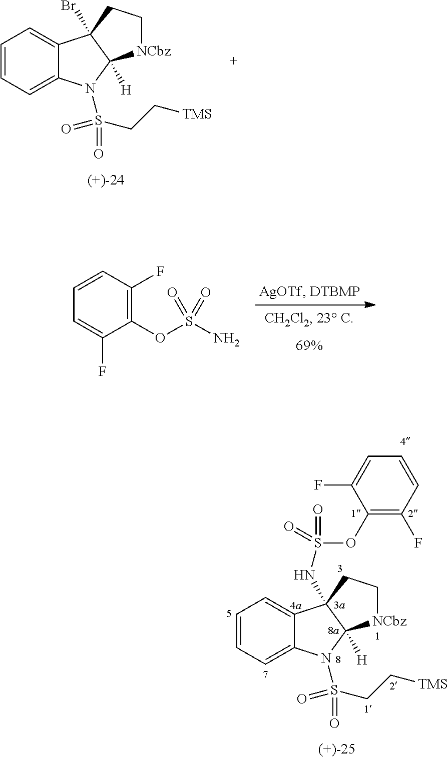

### Example 4: Sulfamate Ester (+)-25

A sample of silver trifluoromethanesulfonate (2.72 g, 10.6 mmol, 2.00 equiv) was added to a solution of bromocyclotryptamine (+)-24 (2.85 g, 5.30 mmol, 1 equiv), 2,6-difluorophenyl sulfamate⁴⁰ (2.22 g, 10.6 mmol, 2.00 equiv), and 2,6-di-tert-butyl-4-methylpyridine (DTBMP, 2.72 g, 13.2 mmol, 2.50 equiv) in dichloromethane (132 mL) at 23° C. in the dark. After 1.5 h, the off-white milky suspension was diluted with ethyl acetate (265 mL) and was filtered through a pad of silica gel covered with a pad of Celite. The filter cake was washed with ethyl acetate (500 mL) and the colorless filtrate was concentrated under reduced pressure. The residue was purified by flash column chromatography on silica gel (eluent: 50%60% diethyl ether in hexanes) to yield a white foam, which was further purified by flash column chromatography on silica gel (eluent: 1%→4% acetonitrile in dichloromethane) to afford pure sulfamate ester (+)-25 (2.42 g, 68.5%) as a white foam. As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (400 MHz, CD₃CN, 60° C.): δ 7.51 (dt, J=7.7, 1.0 Hz, 1H, C4H), 7.44-7.28 (m, 8H, C6H, C7H, C4″H, Ar_{Cbz}H), 7.25-7.17 (m, 1H, C5H), 7.16-7.08 (m, 2H, C3″H), 6.98 (br-s, 1H, NHSO₃Ar), 6.55 (s, 1H, C8aH), 5.28-5.09 (m, 2H, N1CO₂CH₂Ph), 4.08-3.94 (m, 1H, C2Hₐ), 3.41 (br-t, J=11.2 Hz, 1H, C1′Hₐ), 3.25 (td, J=13.4, 4.5 Hz, 1H, C1′H_{b}), 2.94-2.78 (m, 2H, C2H_{b}, C3Hₐ), 2.57-2.44 (m, 1H, C3H_{b}), 1.18-0.97 (m, 2H, C2′H₂), 0.03 (s, 9H, Si(CH₃)₃). ¹³C NMR (100.6 MHz, CD₃CN, 60° C.): δ 157.3 (dd, J=252, 3.5 Hz, C2″), 155.7 (N1CO₂CH₂Ph), 144.7 (C7a), 138.0 (Ar_{Cbz}), 132.0 (C6), 131.1 (C4a), 129.74 (Ar_{Cbz}), 129.66 (t, J=9.4 Hz, C4″), 129.3 (Ar_{Cbz}), 129.2 (Ar_{Cbz}), 127.9 (t, J=15.7 Hz, C₁″), 126.3 (C4), 125.7 (C5), 117.7 (C7), 114.2-113.9 (m, C3″), 84.1 (C8a), 74.1 (C3a), 68.6 (N1CO₂CH₂Ph), 51.3 (C1′), 46.2 (C2), 36.8 (C3), 11.1 (C2′), −1.6 (Si(CH₃)₃). ¹⁹F NMR (376.4 MHz, CD₃CN, 25° C.): δ −126.2 (s, C₆H₃F₂). FTIR (thin film) cm⁻¹: 3210 (br-w), 2952 (w), 1708 (s), 1604 (m), 1480 (s). HRMS (ESI) (m/z): calc'd for C₂₉H₃₃F₂N₃NaO₇S₂Si [M+Na]⁺: 688.1389, found: 688.1367. [α]_{D}²²: +84 (c=0.33, CH₂Cl₂). TLC (2% acetonitrile in dichloromethane), Rf: 0.26 (UV, CAM).

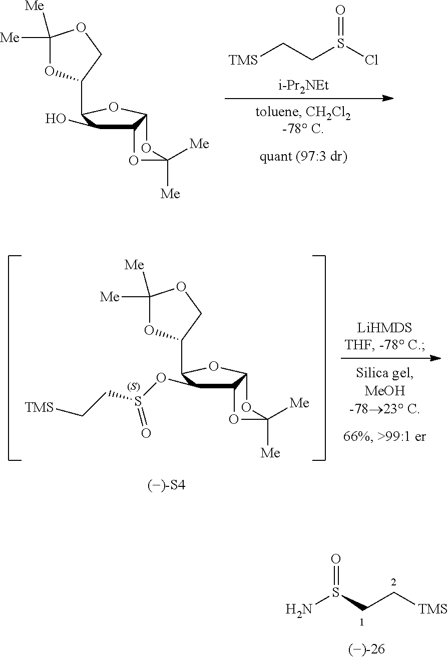

### Example 5: (−)-(S)-2-(Trimethylsilyl)ethanesulfinamide (26)

A solution of freshly prepared (±)-2-(trimethylsilyl)ethanesulfinyl chloride⁴¹ (11.8 g, 64.3 mmol, 1.25 equiv) in toluene (75 mL) was added dropwise from a pressure-equalizing addition funnel over 75 min⁴² to a solution of (−)-diacetone-d-glucose⁴³ (13.4 g, 51.4 mmol, 1 equiv) and N,N-diisopropylethyl-amine (13.0 mL, 74.6 mmol, 1.45 equiv) in toluene (440 mL) and dichloromethane (70 mL) at −78° C. After 2 h, the viscous milky solution was diluted with a saturated aqueous ammonium chloride solution (500 mL) and deionized water (50 mL) and the mixture was allowed to stir in a 23° C. water bath. After 75 min, the layers were separated and the aqueous layer was extracted with diethyl ether (3×300 mL). The combined organic extracts were washed successively with an aqueous hydrogen chloride solution (1 M, 500 mL), a saturated aqueous sodium bicarbonate solution (400 mL), and a saturated aqueous sodium chloride solution (400 mL), were dried over anhydrous sodium sulfate, were filtered, and were concentrated to yield crude (S_{S})-alkanesulfinate (−)-S4 (21.8 g, quantitative yield, 97:3 dr) 44 as a light yellow viscous oil. This inseparable mixture of diastereomers was used directly in the next step without further purification.⁴⁵

The sample of crude (Ss)-alkanesulfinate (−)-S4 was azeotropically dried by concentration from benzene (3×100 mL). The flask was then charged with a stir bar, capped with a rubber septum, and placed under high vacuum (˜0.1 Torr). After 25 min, the flask was refilled with argon and the residue was dissolved in tetrahydrofuran (206 mL). The rubber-septum was replaced with an oven-dried pressure-equalizing addition funnel and the resulting solution was cooled to −78° C. Subsequently, a solution of lithium bis(trimethylsilyl)amide (1.0 M in tetrahydrofuran, 54.0 mL, 54.0 mmol, 1.05 equiv) was added dropwise over 42 min, after which the addition funnel was rinsed with tetrahydrofuran (2.0 mL). After stirring at −78° C. for an additional 90 min, methanol (83.0 mL, 2.06 mol, 40.0 equiv) and silica gel (51.4 g) were added sequentially and the suspension was allowed to stir in a 23° C. water bath. After 1 h, the suspension was concentrated under reduced pressure and the resulting silica-adsorbed crude mixture was purified by flash column chromatography on silica gel (eluent: 10%→40% acetone in dichloromethane) to yield (−)-(S)-2-(trimethylsilyl)ethanesulfinamide⁴⁶ (26, 8.34 g, 98.1%, 88:12 er) as a light-yellow viscous oil, which solidified to an off-white waxy solid on concentration from n-heptane (3×30 mL) and standing for 7 h under high vacuum (˜0.1 Torr).⁴⁷ The enantiomeric ratio was determined by chiral HPLC analysis of a 3 mg/mL solution of (−)-26 in hexanes (CHIRALCEL® OD-H, 4% iPrOH in hexanes, 1.0 mL/min, 210 nm, t_{R} (minor)=16.3 min, t_{R} (major)=18.2 min).

To enrich the enantiomeric ratio, the product was transferred to a 100-mL round-bottom flask and was crushed with a Teflon rod. n-Heptane (30 mL) was added and the resulting suspension was sonicated for 1 h at 23° C. under an atmosphere of argon. A stir bar and an additional portion of n-heptane (10 mL) were added and the suspension was stirred vigorously at 0° C. for 30 min. The solid was then collected by filtration and was washed with cold (−20° C.) n-heptane (35 mL). Drying under vacuum (˜10 Torr) for 14 h provided (−)-26 (5.60 g, 65.9%, >99:1 er) as a flocculent white solid. The enantiomeric ratio was determined by chiral HPLC analysis of a 3 mg/mL solution of (−)-26 in hexanes (CHIRALCEL® OD-H, 4% iPrOH in hexanes, 1.0 mL/min, 210 nm, t_{R} (minor)=16.6 min, t_{R} (major)=18.2 min). ¹H NMR (500 MHz, CDCl₃, 20° C.): δ 4.14 (s, 2H, NH₂), 2.78-2.56 (m, 2H, C1H₂), 0.96-0.79 (m, 2H, C2H₂), 0.04 (s, 9H, Si(CH₃)₃). ¹³C NMR (125.8 MHz, CDCl₃, 20° C.): δ 52.9 (C1), 8.4 (C2), −1.8 (Si(CH₃)₃). FTIR (thin film) cm⁻¹: 3222 (br-m), 2954 (m), 2897 (m), 2809 (w), 1653 (m), 1577 (m), 1419 (m), 1249 (m), 1162 (m), 1036 (br-m). HRMS (DART) (m/z): calc'd for C₅H₁₆NOSSi [M+H]⁺: 166.0716, found: 166.0719. [α]_{D}²³: −22 (c=1.47, CH₂Cl₂). TLC (40% acetone in dichloromethane), Rf: 0.38 (UV, CAM).

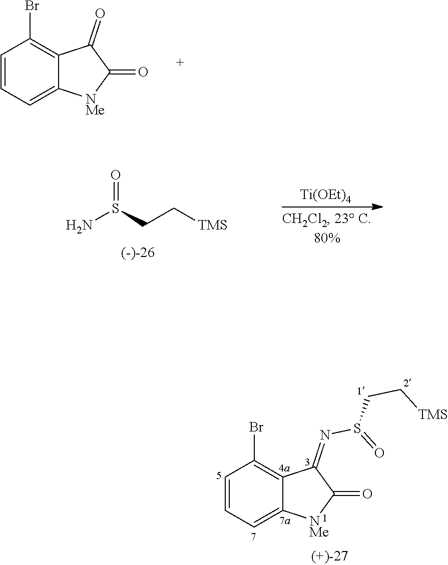

### Example 6: Alkanesulfinyl Imine (+)-27

Titanium ethoxide⁴⁸ (16.3 mL, 67.1 mmol, 2.20 equiv) was added via syringe to a stirred solution of (−)-(S)-2-(trimethylsilyl)ethanesulfinamide (26, 5.55 g, 33.6 mmol, 1.10 equiv) and 4-bromo-1-methylisatin⁴⁹ (7.33 g, 30.5 mmol, 1 equiv) in dichloromethane (61.0 mL) at 23° C. After 20 h, the reaction mixture was diluted with dichloromethane (61 mL) and deionized water (2.40 mL, 133 mmol, 4.40 equiv) was then added dropwise over 4 min with vigorous stirring. The resulting thick red slurry was diluted with an additional portion of dichloromethane (120 mL) and was stirred vigorously. After 10 min, oven-dried Celite (24 g) was added and the suspension was concentrated under reduced pressure. The Celite-adsorbed crude mixture was purified by flash column chromatography on silica gel (eluent: 5%→20% ethyl acetate in dichloromethane) to yield alkanesulfinyl imine (+)-27 (9.47 g, 80.1%) as a dark orange solid. Structural assignments were made using additional information from gCOSY, gHSQC, gHMBC, and 1D selective NOESY experiments. ¹H NMR (500 MHz, CDCl₃, 20° C.): δ 7.34-7.27 (m, 2H, C5H, C6H), 6.83 (app-d, J=7.2 Hz, 1H, C7H), 3.25 (s, 3H, N1CH₃), 3.06-2.96 (m, 1H, C1′Hₐ), 2.95-2.86 (m, 1H, C1′H_{b}), 1.19-1.06 (m, 2H, C2′H), 0.04 (s, 9H, Si(CH₃)₃). ¹³C NMR (125.8 MHz, CDCl₃, 20° C.): δ 157.3 (C2), 155.3 (C3), 149.5 (C7a), 135.3 (C6), 129.1 (C5), 121.1 (C4), 117.7 (C4a), 108.3 (C7), 53.4 (C1′), 26.6 (N1CH₃), 9.3 (C2′), −1.6 (Si(CH₃)₃). FTIR (thin film) cm⁻¹: 3080 (w), 2952 (m), 2894 (w), 1723 (s), 1596 (s), 1456 (m), 1355 (m), 1322 (m), 1249 (m), 1109 (s). HRMS (ESI) (m/z): calc'd for C₁₄H₂₀BrN₂O₂SSi [M+H]⁺: 387.0193, found: 387.0185. [α]_{D}²³: +447 (c=0.31, CH₂Cl₂). TLC (10% ethyl acetate in dichloromethane), Rf: 0.29 (UV, CAM).

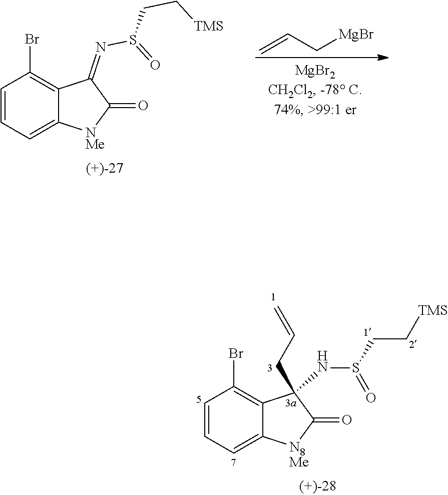

### Example 7: Allyl Oxindole (+)-28

A sample of alkanesulfinyl imine (+)-27 (9.45 g, 24.4 mmol, 1 equiv) was azeotropically dried by concentration from benzene (3×80 mL). The flask was then charged with a stir bar, capped with a rubber septum, and placed under high vacuum (˜0.1 Torr) for 14 h. A sample of magnesium bromide (8.98 g, 48.8 mmol, 2.00 equiv) and dichloromethane (160 mL) were added and the rubber septum was then replaced with an oven-dried pressure-equalizing addition funnel. The resulting dark orange suspension was cooled to −78° C. and subsequently a solution of allylmagnesium bromide (1.28 M in diethyl ether, 19.8 mL, 25.3 mmol, 1.04 equiv) was added dropwise over 30 min. After stirring for an additional 45 min, the bright yellow suspension was diluted with a saturated aqueous ammonium chloride solution (160 mL) and deionized water (160 mL). The cold-bath was removed and the mixture was allowed to warm to 23° C. with vigorous stirring. The layers were separated and the aqueous layer was extracted with dichloromethane (3×100 mL). The combined organic extracts were washed with a saturated aqueous sodium chloride solution (300 mL), were dried over anhydrous sodium sulfate, were filtered, and were concentrated under reduced pressure. The resulting residue was purified by flash column chromatography on silica gel (eluent: 6→20% acetone in dichloromethane) to afford allyl oxindole (+)-28 (7.76 g, 74.1%, >99:1 er) as a yellow solid. The enantiomeric ratio was determined by chiral HPLC analysis (CHIRALCEL® OD-H, 30% i-PrOH in hexanes, 0.7 mL/min, 220 nm, t_{R} (major)=10.4 min, t_{R} (minor)=6.6 min). Structural assignments were made using additional information from gCOSY, gHSQC, gHMBC, and 1D selective NOESY experiments. ¹H NMR (500 MHz, CDCl₃, 20° C.): δ 7.22-7.15 (m, 2H, C5H, C6H), 6.82-6.75 (m, 1H, C7H), 5.23 (app-ddt, J=17.1, 9.9, 7.3 Hz, 1H, C2H), 5.07 (app-dq, J=17.0, 1.3 Hz, 1H, C1Hₐ), 4.91 (dd, J=10.1, 1.8 Hz, 1H, C1H_{b}), 4.55 (s, 1H, NH), 3.21 (s, 3H, N8CH₃), 3.15 (dd, J=12.9, 6.9 Hz, 1H, C3Hₐ), 2.81 (dd, J=12.9, 7.7 Hz, 1H, C3H_{b}), 2.78-2.69 (m, 1H, C1′Hₐ), 2.69-2.58 (m, 1H, C1′H_{b}), 0.92-0.81 (m, 2H, C2′H), 0.02 (s, 9H, Si(CH₃)₃). ¹³C NMR (125.8 MHz, CDCl₃, 20° C.): δ 174.5 (C8a), 145.1 (C7a), 131.1 (C6), 129.4 (C2), 127.28 (C4a), 127.25 (C5), 120.7 (C1), 119.9 (C4), 107.8 (C7), 66.6 (C3a), 53.3 (C1′), 39.9 (C3), 26.6 (N8CH₃), 8.6 (C2′), −1.7 (Si(CH₃)₃). FTIR (thin film) cm⁻¹: 3249 (m), 2954 (m), 1716 (s), 1602 (s), 1583 (m), 1456 (m), 1343 (m), 1292 (m), 1074 (m). HRMS (ESI) (m/z): calc'd for C₁₇H₂₆BrN₂₀O₂SSi [M+H]⁺: 429.0662, found: 429.0675. [α]_{D}²³: +15 (c=0.29, CH₂Cl₂). TLC (10% acetone in dichloromethane), Rf: 0.27 (UV, CAM, KMnO₄).

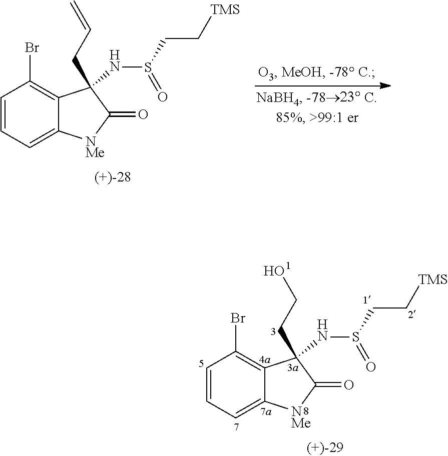 [SMILES: C=CC[C@@]1(N[S@@](=O)CC[Si](C)(C)C)C(=O)N(C)c2cccc(Br)c21 ; CN1C(=O)[C@@](CCO)(N[S@@](=O)CC[Si](C)(C)C)c2c(Br)cccc21]

### Example 8: Alcohol (+)-29

Ozone-enriched dioxygen was bubbled through a solution of allyl oxindole (+)-28 (6.94 g, 16.2 mmol, 1 equiv) in methanol (81 mL) at −78° C. After 1.5 h, ozone bubbling was suspended and the solution was sparged with dinitrogen for 40 min. A sample of sodium borohydride⁵⁰ (2.12 g, 56.0 mmol, 3.46 equiv) was then added in 16 portions over 16 min. The cold bath was removed and the mixture was allowed to warm to 23° C. After 1 h, the solution was concentrated under reduced pressure and the resulting slurry was diluted with a saturated aqueous ammonium chloride solution (80 mL) and deionized water (80 mL). The mixture was extracted with ethyl acetate (3×100 mL) and the combined organic extracts were washed with a saturated aqueous sodium chloride solution (200 mL), were dried over anhydrous sodium sulfate, were filtered, and were concentrated under reduced pressure. The resulting residue was purified by flash column chromatography on silica gel (eluent: 20%→40% acetone in dichloromethane) to afford alcohol (+)-29 (5.94 g, 84.7%, >99:1 er) as an off-white foam. The enantiomeric ratio was determined by chiral HPLC analysis (CHIRALCEL® OD-H, 30% i-PrOH in hexanes, 0.7 mL/min, 220 nm, t_{R} (major)=12.7 min, t_{R} (minor)=7.7 min). Structural assignments were made using additional information from gCOSY, gHSQC, gHMBC, and 1D selective NOESY experiments. ¹H NMR (500 MHz, CDCl₃, 20° C.): δ 7.23-7.17 (m, 2H, C5H, C6H), 6.86-6.79 (m, 1H, C7H), 4.76 (s, 1H, NH), 3.75-3.63 (m, 1H, C2Hₐ), 3.46 (app-dtd, J=11.6, 7.8, 4.5 Hz, 1H, C2H_{b}), 3.22 (s, 3H, N8CH₃), 2.79-2.68 (m, 1H, C1′Hₐ), 2.68-2.56 (m, 2H, C1′H_{b}, C3Hₐ) 2.37 (dt, J=14.2, 4.8 Hz, 1H, C3H_{b}), 2.18 (dd, J=7.4, 4.2 Hz, 1H, O1H), 0.91-0.79 (m, 2H, C2′H₂), 0.02 (s, 9H, Si(CH₃)₃). ¹³C NMR (125.8 MHz, CDCl₃, 20° C.): δ 175.7 (C8a), 145.0 (C7a), 131.3 (C6), 127.7 (C4a), 127.4 (C5), 119.9 (C4), 108.1 (C7), 65.4 (C3a), 58.2 (C2), 53.2 (C1′), 38.3 (C3), 26.9 (N8CH₃), 8.7 (C2′), −1.7 (Si(CH₃)₃). FTIR (thin film) cm⁻¹:3403 (br-m), 3242 (br-m), 2953 (m), 2893 (m), 1721 (s), 1605 (s), 1458 (s). HRMS (ESI) (m/z): calc'd for C₁₆H₂₆BrN₂₃SSi [M+H]⁺: 433.0611, found: 433.0615. [α]_{D}²³: +9 (c=0.29, CH₂Cl₂). TLC (30% acetone in dichloromethane), Rf: 0.24 (UV, CAM).

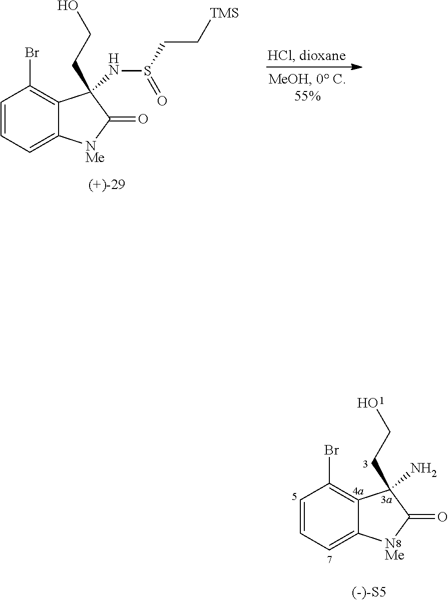 [SMILES: CN1C(=O)[C@@](CCO)(N[S@@](=O)CC[Si](C)(C)C)c2c(Br)cccc21 ; CN1C(=O)[C@](N)(CCO)c2c(Br)cccc21]

### Example 9: Amino Alcohol (−)-SS

A solution of hydrogen chloride in 1,4-dioxane (4.0 M, 58.0 μL, 232 μmol, 2.01 equiv) was added dropwise via syringe to a solution of alcohol (+)-29 (50.0 mg, 115 μmol, 1 equiv) in methanol (2.30 mL) at 0° C. After 1 h, a saturated aqueous sodium bicarbonate solution (23 mL) and an aqueous sodium hydroxide solution (1 M, 1 mL) were added and the mixture was extracted with ethyl acetate (8×10 mL). The combined organic extracts were dried over anhydrous sodium sulfate, were filtered, and were concentrated under reduced pressure. The resulting residue was purified by flash column chromatography on silica gel (eluent: 3%-5% methanol in dichloromethane) to afford amino alcohol (−)-S5 (18.1 mg, 55.1%) as a colourless film. Structural assignments were made using additional information from gCOSY, gHSQC, and gHMBC experiments. ¹H NMR (500 MHz, CDCl₃, 20° C.): δ 7.22-7.13 (m, 2H, C5H, C6H), 6.88-6.65 (m, 1H, C7H), 3.71 (app-dt, J=11.6, 5.2 Hz, 1H, C2Hₐ), 3.50 (ddd, J=11.6, 8.4, 4.4 Hz, 1H, C2H_{b}), 3.19 (s, 3H, N8CH₃), 2.46 (ddd, J=14.2, 8.4, 4.7 Hz, 1H, C3Hₐ), 2.32 (ddd, J=14.3, 5.7, 4.4 Hz, 1H, C3H_{b}), 2.04 (br-s, 3H, C20H, C3aNH₂). ¹³C NMR (125.8 MHz, CDCl₃, 20° C.): δ 179.1 (C8a), 145.2 (C7a), 130.6 (C6), 129.9 (C4a), 127.2 (C5), 119.0 (C4), 107.7 (C7), 62.0 (C3a), 58.9 (C2), 39.2 (C3), 26.6 (N8CH₃). FTIR (thin film) cm⁻¹: 3357 (br-m), 3284 (br-m), 2924 (w), 2883 (w), 1720 (s), 1605 (s), 1581 (m), 1456 (s), 1366 (m), 1288 (m), 1189 (w), 1101 (m), 1055 (m), 763 (m). HRMS (ESI) (m/z): calc'd for C₁₁H₁₄BrN₂O₂ [M+H]⁺: 285.0233, found: 285.0222. [α]_{D}²³: −10 (c=0.91, CH₂Cl₂).⁵¹ TLC (3% methanol in dichloromethane), Rf: 0.27 (UV, CAM).

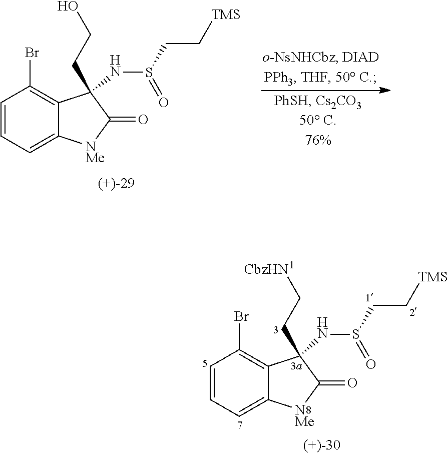 [SMILES: CN1C(=O)[C@@](CCO)(N[S@@](=O)CC[Si](C)(C)C)c2c(Br)cccc21 ; CN1C(=O)[C@@](CCNC(=O)OCc2ccccc2)(N[S@@](=O)CC[Si](C)(C)C)c2c(Br)cccc21]

### Example 10: Carbamate (+)-30

Diisopropyl azodicarboxylate (DIAD, 3.10 mL, 15.7 mmol, 1.15 equiv) was added dropwise via syringe to a solution of alcohol (+)-29 (5.93 g, 13.7 mmol, 1 equiv), triphenylphosphine (4.13 g, 15.7 mmol, 1.15 equiv), and N-carbobenzoxy-2-nitrobenzenesulfonamide (5.29 g, 13.4 mmol, 1.15 equiv) in tetrahydrofuran (91 mL) at 23° C. The flask was fitted with a reflux condenser and was immersed in a preheated oil bath at 50° C. After stirring for 1 h, the mixture was cooled to 23° C. and samples of cesium carbonate (17.8 g, 54.7 mmol, 4.00 equiv) and thiophenol (2.81 mL, 27.4 mmol, 2.00 equiv) were added. The flask was immersed in a preheated oil bath at 50° C. and the mixture was stirred vigorously for 1.5 h. The bright yellow suspension was then cooled to 23° C., was diluted with deionized water (360 mL) and a saturated aqueous sodium chloride solution (90 mL), and was extracted with diethyl ether (3×230 mL). The combined organic extracts were washed successively with an aqueous sodium hydroxide solution (0.1 M, 350 mL) and a saturated aqueous sodium chloride solution (350 mL), were dried over anhydrous sodium sulfate, were filtered, and were concentrated under reduced pressure. The resulting residue was purified by flash column chromatography on silica gel (eluent: 10%→20% isopropanol in hexanes) to afford carbamate (+)-30 (5.90 g, 76.1%) as a white foam. Structural assignments were made using additional information from gCOSY, gHSQC, and gHMBC experiments. ¹H NMR (500 MHz, CDCl₃, 20° C.): δ 7.37-7.27 (m, 5H, Ar_{Cbz}H), 7.23-7.13 (m, 2H, C5H, C6H), 6.81-6.73 (m, 1H, C7H), 5.01 (d, J=12.2 Hz, 1H, N1CO₂CHₐPh), 4.96 (d, J=12.3 Hz, 1H, N1CO₂CH_{b}Ph), 4.77-4.63 (m, 1H, N1H), 4.63-4.47 (m, 1H, NHSOAlk) 3.16 (s, 3H, N8CH₃), 3.12-2.93 (m, 2H, C2H₂), 2.76-2.66 (m, 1H, C1′Hₐ), 2.66-2.57 (m, 1H, C1′H_{b}), 2.56-2.39 (m, 2H, C3H₂), 0.91-0.78 (m, 2H, C2′H₂), 0.02 (s, 9H, Si(CH₃)₃). ¹³C NMR (125.8 MHz, CDCl₃, 20° C.): δ 174.6 (C8a), 155.9 (N1CO₂CH₂Ar), 144.9 (C7a), 136.5 (Ar_{Cbz}), 131.4 (C6), 128.6 (Ar_{Cbz}), 128.29 (Ar_{Cbz}), 128.26 (Ar_{Cbz}), 127.5 (C5), 127.2 (C4a), 120.0 (C4), 108.1 (C7), 66.7 (N1CO₂CH₂Ar), 65.4 (C3a), 53.3 (C1′), 36.1 (C2), 35.5 (C3), 26.8 (N8CH₃), 8.6 (C2′), −1.7 (Si(CH₃)₃). FTIR (thin film) cm⁻¹: 3396 (s), 3246 (s), 3032 (m), 2952 (s), 2890 (s), 1724 (br-s), 1604 (s), 1520 (s), 1457 (s), 1251 (s). HRMS (DART) (m/z): calc'd for C₂₄H₃₃BrN₃O₄SSi [M+H]⁺: 566.1139, found: 566.1148. [α]_{D}²³: +25 (c=0.33, CH₂Cl₂). TLC (15% isopropanol in hexanes), Rf: 0.19 (UV, CAM).

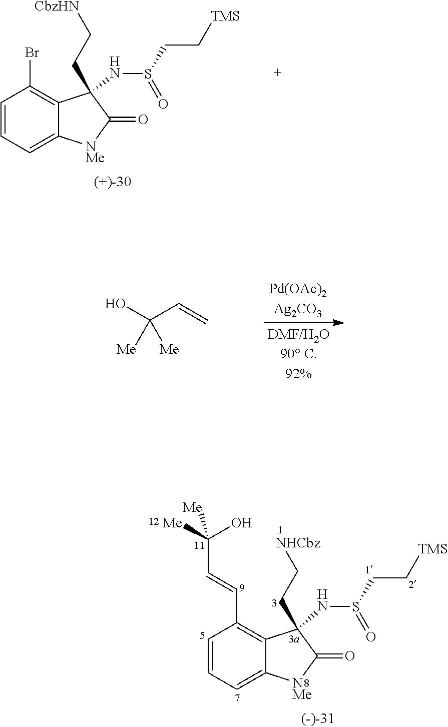

### Example 11: Allylic Alcohol (−)-31

A pressure vessel was charged sequentially with carbamate (+)-30 (5.89 g, 10.4 mmol, 1 equiv), silver(I) carbonate (5.73 g, 20.8 mmol, 2.00 equiv), palladium(II) acetate (350 mg, 1.56 mmol, 0.150 equiv), 1,1-dimethylallyl alcohol (21.7 mL, 208 mmol, 20.0 equiv), N,N-dimethylformamide (52 mL), and deionized water (52 mL). The resulting suspension was then degassed by vigorously sparging with argon for 15 min. The vessel was sealed with a Teflon screwcap and was immersed in a preheated oil bath at 90° C. After vigorous stirring for 2 h, the black suspension was cooled to 23° C. and was diluted with diethyl ether (100 mL). The mixture was filtered through a pad of Celite and the filter cake was washed with diethyl ether (400 mL). The filtrate was washed with a saturated aqueous sodium chloride solution (450 mL) and the layers were separated. The aqueous layer was extracted with diethyl ether (3×180 mL) and the combined organic extracts were washed with a saturated aqueous sodium chloride solution (2×250 mL), were dried over anhydrous sodium sulfate, were filtered, and were concentrated under reduced pressure. The resulting residue was purified by flash column chromatography on silica gel (eluent: 30→80% acetonitrile in dichloromethane) to afford allylic alcohol (−)-31 (5.43 g, 91.5%) as a pale-yellow foam. Structural assignments were made using additional information from gCOSY, gHSQC, gHMBC, and 1D selective NOESY experiments. ¹H NMR (500 MHz, CDCl₃, 20° C.): δ 7.37-7.27 (m, 6H, C6H, Ar_{Cbz}H), 7.23 (d, J=8.0 Hz, 1H, C5H), 7.12 (d, J=15.9 Hz, 1H, C9H), 6.71 (d, J=7.7 Hz, 1H, C7H), 6.36 (d, J=15.9 Hz, 1H, C10H), 5.00 (d, J=12.2 Hz, 1H, N1CO₂CHₐAr), 4.97 (d, J=12.2 Hz, 1H, N1CO₂CH_{b}Ar), 4.64 (br-t, J=5.8 Hz, 1H, N1H), 4.35 (s, 1H, NHSOAlk), 3.35 (s, 1H, OH), 3.17 (s, 3H, N8CH₃), 2.98-2.86 (m, 1H, C2Hₐ), 2.78-2.69 (m, 1H, C2H_{b}), 2.68-2.54 (m, 3H, C3Hₐ, C1′H₂), 2.42-2.32 (m, 1H, C3H_{b}), 1.44 (s, 3H, C12H₃), 1.41 (s, 3H, C12H₃), 0.86-0.73 (m, 2H, C2′H₂), 0.00 (s, 9H, Si(CH₃)₃). ¹³C NMR (125.8 MHz, CDCl₃, 20° C.): δ 176.4 (C8a), 156.1 (N1CO₂CH₂Ar), 143.7 (C7a), 142.4 (C10), 136.6 (C4), 136.4 (Ar_{Cbz}), 130.4 (C6), 128.6 (Ar_{Cbz}), 128.23 (Ar_{Cbz}), 128.21 (Ar_{Cbz}), 123.5 (C4a), 122.3 (C9), 120.8 (C5), 107.5 (C7), 70.9 (C11), 66.7 (N1CO₂CH₂Ar), 63.9 (C3a), 53.0 (C1′), 36.9 (C3), 36.4 (C2), 29.7 (C12), 29.6 (C12), 26.7 (N8CH₃), 9.3 (C2′), −1.8 (Si(CH₃)₃). FTIR (thin film) cm⁻¹: 3319 (br-m), 3066 (w), 2969 (m), 1714 (br-s), 1590 (m), 1532 (br-m), 1465 (m), 1368 (m), 1251(s). HRMS (ESI) (m/z): calc'd for C₂₉H₄₁N₃NaO₅SSi [M+Na]⁺: 594.2428, found: 594.2422. [α]_{D}²³: −53 (c=0.6, CH₂Cl₂). TLC (50% acetonitrile in dichloromethane), Rf: 0.33 (UV, CAM).

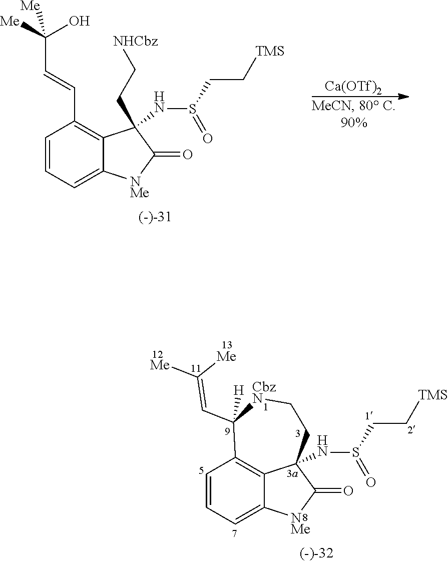

### Example 12: Tricyclic Oxindole (−)-32

A sample of calcium trifluoromethanesulfonate (3.10 g, 9.18 mmol, 1.15 equiv) was added to a solution of allylic alcohol (−)-31 (4.56 g, 7.98 mmol, 1 equiv) in acetonitrile (160 mL) at 23° C. The reaction flask was fitted with a reflux condenser and was immersed in a preheated oil bath at 80° C. After stirring for 36 h, the homogeneous yellow solution was cooled to 23° C. and was concentrated under reduced pressure. The residue was diluted with a saturated aqueous sodium bicarbonate solution (160 mL) and deionized water (40 mL) and the mixture was extracted with ethyl acetate (3×100 mL). The combined organic extracts were washed with a saturated aqueous sodium chloride solution (200 mL), were dried over anhydrous sodium sulfate, were filtered, and were concentrated under reduced pressure. The resulting residue was purified by flash column chromatography on silica gel (eluent: 18→30% acetonitrile in dichloromethane) to afford tricyclic oxindole (−)-32 (3.97 g, 89.9%) as a white foam. As a result of slow conformational equilibration at ambient temperature, NMR spectra were collected at elevated temperature. Structural assignments were made using additional information from gCOSY, gHSQC, and gHMBC experiments collected at elevated temperature. ¹H NMR (400 MHz, DMSO-d₆, 100° C.):⁵² δ 7.40-7.17 (m, 6H, Ar_{Cbz}H₅, C₆H), 6.88 (app-d, J=7.8 Hz, 2H, C5H, C7H), 6.19 (br-s, 1H, NH), 5.88 (br-d, J=8.6 Hz, 1H, C9H), 5.77 (br-s, 1H, C10H), 5.02 (d, J=12.6 Hz, 1H, N1CO₂CHₐPh), 4.98 (d, J=12.6 Hz, 1H, N1CO₂CH_{b}Ph), 3.88 (ddd, J=14.6, 7.7, 3.9 Hz, 1H, C2Hₐ), 3.68 (br-s, 1H, C2H_{b}), 3.12 (s, 3H, N8CH₃), 2.71-2.59 (m, 1H, C1′Hₐ), 2.59-2.50 (m, 1H, C1′H_{b}), 2.33-2.18 (m, 1H, C3Hₐ), 1.80 (s, 3H, C12/13H₃), 1.68 (s, 3H, C12/13H₃), 1.59 (ddd, J=14.5, 7.6, 4.8 Hz, 1H, C3H_{b}), 0.77-0.65 (m, 2H, C2′H₂), 0.00 (s, 9H, Si(CH₃)₃). ¹³C NMR (100.6 MHz, DMSO-d₆, 90° C.):⁵² δ 175.7 (C8a), 154.2 (N1CO₂CH₂Ph), 143.2 (C7a), 136.3 (2C, C4, Ar_{Cbz}), 134.2 (C11), 128.6 (C6), 127.5 (Ar_{Cbz}), 127.0 (Ar_{Cbz}), 126.9 (Ar_{Cbz}), 124.9 (C4a), 121.2 (C10), 119.9 (C), 106.8 (C₇), 65.6 (N₁CO₂CH₂Ph), 61.8 (C₃a), 57.1 (C9), 50.9 (C1′), 39.8 (C2), 31.4 (C3), 25.5 (N8CH₃), 24.7 (C12/13), 17.5 (C12/13), 8.5 (C2′), −2.5 (Si(CH₃)₃). FTIR (thin film) cm⁻¹: 3229 (br-w), 2953 (m), 1706 (s), 1610 (m), 1599 (m), 1468 (m), 1418 (m), 1251 (m), 1050 (m). HRMS (ESI) (m/z): calc'd for C₂₉H₄₀N₃O₄SSi [M+H]⁺: 554.2503, found: 554.2497. [c]_{D}²³: −64 (c=0.22, CH₂Cl₂). TLC (20% acetonitrile in dichloromethane), Rf: 0.23 (UV, CAM).

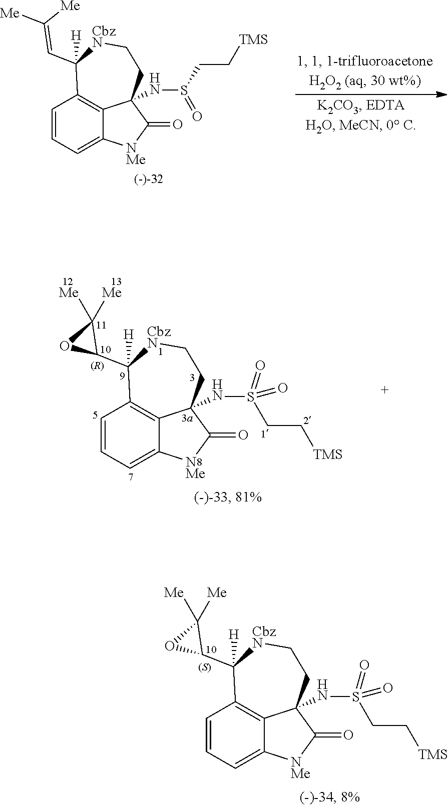

### Example 13: (10R)-Tricyclic Epoxide (−)-33⁵³

An aqueous potassium carbonate⁵⁴ solution (1.50 M in 4.00×10⁻⁴ M aqueous EDTA,⁵⁵ 4.50 mL) and an aqueous hydrogen peroxide solution⁵⁶ (30 wt %, 3.40 mL, 30.0 mmol, 10.0 equiv) were added successively to a solution of tricyclic oxindole (−)-32 (1.66 g, 3 mmol, 1 equiv) and 1,1,1-trifluoroacetone (282 μL, 3.00 mmol, 1.00 equiv) in acetonitrile (4.50 mL) at 0° C. After vigorous stirring at 0° C. for 7 h, an aqueous sodium thiosulfate solution (1 M, 90 mL) was added and the mixture was allowed to warm to 23° C. The resulting suspension was extracted with ethyl acetate (3×90 mL) and the combined organic extracts were washed with a saturated aqueous sodium chloride solution (180 mL), were dried over anhydrous sodium sulfate, were filtered, and were concentrated under reduced pressure. The residue was purified by flash column chromatography (eluent: 38%→45% ethyl acetate in hexanes) to afford (10R)-tricyclic epoxide (−)-33 (1.43 g, 81.1%) as a white foam and the C10-epimer (−)-34 (140 mg, 7.95%) as a light yellow film. As a result of slow conformational equilibration at ambient temperature, NMR spectra were collected at elevated temperature. Structural assignments were made using additional information from gCOSY, gHSQC, and gHMBC experiments collected at elevated temperature.

(10R)-Tricyclic epoxide (−)-33: ¹H NMR (400 MHz, DMSO-d₆, 130° C.):⁵⁷ δ 7.37-7.22 (m, 6H, C6H, Ar_{Cbz}H), 7.02 (br-s, 1H, NH), 6.93-6.85 (m, 2H, C5H, C7H), 5.09 (d, J=12.5 Hz, 1H, N1CO₂CHₐPh), 5.02 (d, J=12.5 Hz, 1H, N1CO₂CH_{b}Ph), 4.96 (br-d, J=8.3 Hz, 1H, C9H), 3.91 (ddd, J=14.9, 7.6, 4.3 Hz, 1H, C2Hₐ), 3.85-3.67 (br-m, 1H, C2H_{b}), 3.64-3.50 (br-m, 1H, C10H), 3.13 (s, 3H, N8CH₃), 2.88-2.78 (m, 1H, C1′Hₐ), 2.68-2.56 (m, 1H, C1′H_{b}), 2.34 (app-dt, J=14.3, 4.9 Hz, 1H, C3Hₐ), 1.62 (ddd, J=14.6, 7.5, 5.3 Hz, 1H, C3H_{b}), 1.41 (s, 3H, C12/13H₃), 1.35 (s, 3H, C12/13H₃), 0.90-0.77 (m, 2H, C2′H), −0.02 (s, 9H, Si(CH₃)₃). ¹³C NMR (100.6 MHz, DMSO-d₆, 130° C.): δ 174.5 (C8a), 154.5 (N1CO₂CH₂Ph), 143.3 (C7a), 136.6 (C4), 136.0 (Ar_{Cbz}), 128.6 (C6), 127.4 (Ar_{Cbz}), 126.8 (Ar_{Cbz}), 126.7 (Ar_{Cbz}), 124.9 (C4a), 118.8 (C5), 107.1 (C7), 65.9 (N1CO₂CH₂Ph), 61.2 (2C: C10, C3a), 59.1 (C9), 58.1 (C11), 49.1 (C1′), 43.8 (br-s, C2), 32.0 (C3), 25.5 (N8CH₃), 23.6 (C12/13), 18.0 (C12/13), 8.9 (C2′), −2.9 (Si(CH₃)₃). FTIR (thin film) cm⁻¹: 3218 (br-m), 2957 (m), 2899 (m), 1717 (s), 1702 (s), 1601 (m), 1467 (s), 1420 (s), 1368 (m), 1327 (s). HRMS (ESI) (m/z): calc'd for C₂₉H₃₉N₃NaO₆SSi [M+Na]⁺: 608.2221, found: 608.2217. [α]_{D}²³: −87 (c=0.22, CH₂Cl₂). TLC (50% ethyl acetate in hexanes), Rf: 0.32 (UV, CAM).

(10S)-Tricyclic epoxide (−)-34: ¹H NMR (400 MHz, CD₃CN, 70° C.): δ 7.44-7.15 (m, 7H), 6.97-6.83 (m, 1H), 5.75 (br-s, 1H), 5.08 (d, J=12.2 Hz, 1H), 5.00 (d, J=12.6 Hz, 1H), 4.75 (br-s, 1H), 4.02 (br-s, 1H), 3.84 (ddd, J=14.4, 9.4, 2.8 Hz, 2H), 3.15 (s, 3H), 2.77 (td, J=13.5, 4.7 Hz, 1H), 2.45 (td, J=13.6, 4.2 Hz, 1H), 2.18 (br-d, J=14.2 Hz, 1H), 1.68 (ddd, J=14.5, 9.8, 4.9 Hz, 1H), 1.45 (s, 3H), 1.33 (s, 3H), 0.85 (td, J=13.6, 4.6 Hz, 1H), 0.74 (td, J=13.7, 4.2 Hz, 1H), −0.03 (s, 9H). ¹³C NMR (100.6 MHz, CD₃CN, 70° C.): δ 177.5, 156.9, 146.3, 140.6, 138.4, 131.2, 129.9, 129.4 (2C), 126.3, 121.3, 109.6, 68.5, 63.9, 63.0 (br), 61.5, 60.1, 52.5, 48.1 (br), 34.3, 27.7, 25.5, 20.4, 11.0, −1.5. FTIR (thin film) cm⁻¹: 3208 (br-w), 2956 (w), 2900 (w), 1709 (s), 1606 (m), 1474 (m), 1416 (m), 1367 (m), 1323 (s), 1251 (s). HRMS (ESI) (m/z): calc'd for C₂₉H₄₀N₃O₆SSi [M+H]⁺: 586.2402, found: 586.2403. [α]_{D}²³: −70 (c=0.83, CH₂Cl₂). TLC (50% ethyl acetate in hexanes), Rf: 0.44 (UV, CAM).

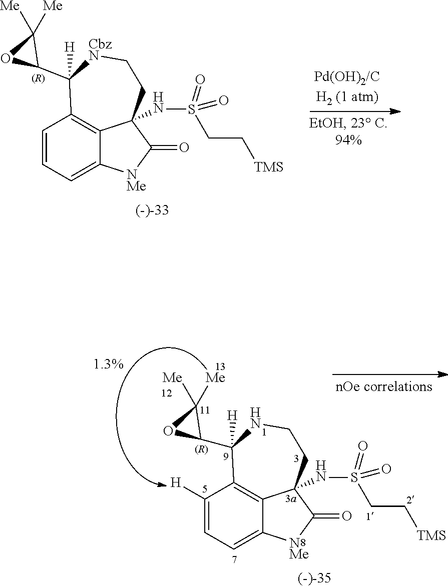

### Example 14: (10R)-Tetracyclic Amine (−)-35

A sample of palladium(II) hydroxide on carbon (15.7 wt % on wet support, 12.0 mg, 13.4 μmol, 0.0785 equiv) was added to a solution of (10R)-tricyclic epoxide (−)-33 (100 mg, 171 mol, 1 equiv) in anhydrous ethanol (200 proof, 3.40 mL) at 23° C. The resulting suspension was sparged with dihydrogen for 5 min by discharge of a balloon equipped with a needle extending into the reaction mixture. After stirring for 2 h under an atmosphere of dihydrogen, the suspension was sparged with dinitrogen for 5 min and was diluted with ethyl acetate (7 mL). The mixture was then filtered through a plug of Celite and the filter cake was washed with ethyl acetate (15 mL). The colourless filtrate was concentrated under reduced pressure and the residue was purified by flash column chromatography on silica gel (eluent: 40-50% acetone in dichloromethane) to afford (10R)-tetracyclic amine (−)-35 (72.4 mg, 93.9%) as a white foam. As a result of slow conformational equilibration at ambient temperature, NMR spectra were collected at elevated temperature. Structural assignments were made using additional information from gCOSY, gHSQC, gHMBC, and 1D selective NOESY experiments also collected at elevated temperature. ¹H NMR (400 MHz, C₆D₆, 70° C.): δ 7.07 (t, J=7.9 Hz, 1H, C6H), 6.68 (d, J=7.9 Hz, 1H, C5H), 6.41 (d, J=7.8 Hz, 1H, C7H), 4.22 (d, J=8.7 Hz, 1H, C9H), 3.06-2.95 (m, 3H, C2Hₐ, Cl′H₂), 2.92 (s, 3H, N8CH₃), 2.87 (d, J=8.8 Hz, 1H, C10H), 2.83 (app-dt, J=14.3, 6.0 Hz, 1H, C2H_{b}), 2.14 (app-dt, J=14.1, 6.2 Hz, 1H, C3Hₐ), 1.36 (app-dt, J=14.1, 5.9 Hz, 1H, C3H_{b}), 1.25 (s, 3H, C12/13H₃), 1.24 (s, 3H, C12/13H₃), 1.17-1.10 (m, 2H, C2′H₂), −0.09 (s, 9H, Si(CH₃)₃). ¹³C NMR (100.6 MHz, C₆D₆, 70° C.): δ 175.3 (C8a), 144.3 (C7a), 138.7 (C4), 129.6 (C6), 128.8 (C4a), 119.5 (C5), 107.4 (C7), 65.4 (C10), 63.2 (C3a), 60.2 (C11), 59.5 (C9), 52.1 (C1′), 42.3 (C2), 36.2 (C3), 26.4 (N8CH₃), 24.9 (C12/13), 19.4 (C12/13), 10.9 (C2′), −2.0 (Si(CH₃)₃). FTIR (thin film) cm⁻¹: 3228 (br-w), 2955 (m), 1721 (s), 1606 (m), 1470 (m), 1327 (s), 1251 (m), 1148 (m). HRMS (DART) (m/z): calc'd for C₂₁H₃₄N₃O₄SSi [M+H]⁺: 452.2034, found: 452.2036. [α]_{D}²³: −66 (c=0.49, CH₂Cl₂). TLC (50% acetone in dichloromethane), Rf: 0.35 (UV, CAM).

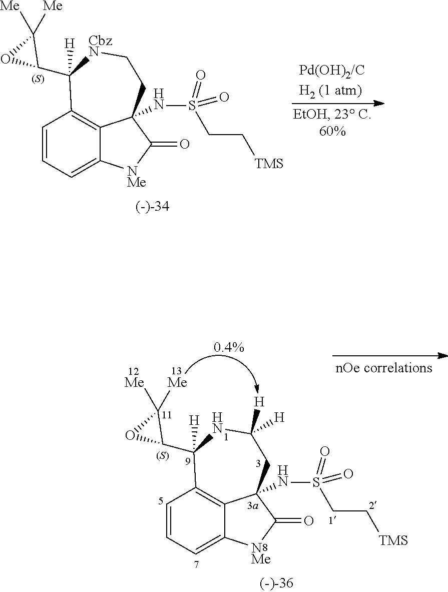

### Example 15: (10S)-Tetracyclic Amine (−)-36

A sample of palladium(II) hydroxide on carbon (15.7 wt % on wet support, 3.2 mg, 3.6 μmol, 0.080 equiv) was added to a solution of (10S)-tricyclic epoxide (−)-34 (26.6 mg, 45.4 mol, 1 equiv) in anhydrous ethanol (200 proof, 900 μL) at 23° C. The resulting suspension was sparged with dihydrogen for 5 min by discharge of a balloon equipped with a needle extending into the reaction mixture. After stirring for 2 h under an atmosphere of dihydrogen, the suspension was sparged with dinitrogen for 5 min and was diluted with ethyl acetate (3 mL). The mixture was then filtered through a plug of Celite and the filter cake was washed with ethyl acetate (10 mL). The colourless filtrate was concentrated under reduced pressure and the resulting residue was purified by flash column chromatography on silica gel (eluent: 50% acetone in dichloromethane) to afford (10S)-tetracyclic amine (−)-36 (12.3 mg, 60.1%) as a colourless film. Crystals suitable for X-ray diffraction were obtained by layer diffusion of n-heptane into a solution of (−)-36 in dichloromethane at 0° C.

As a result of slow conformational equilibration at ambient temperature, NMR spectra were collected at elevated temperature. Structural assignments were made using additional information from gCOSY, gHSQC, gHMBC, and 1D selective NOESY experiments also collected at elevated temperature. ¹H NMR (400 MHz, C₆D₆, 60° C.): δ 7.28 (app-dt, J=7.9, 0.9 Hz, 1H, C5H), 7.08 (t, J=7.9 Hz, 1H, C6H), 6.38 (d, J=7.8 Hz, 1H, C7H), 6.28 (br-s, 1H, NHSO₂), 4.18 (d, J=9.2 Hz, 1H, C9H), 3.52 (ddd, J=14.5, 11-6, 2.7 Hz, 1H, C2Hₐ), 3.02 (ddd, J=14.9, 4.2, 3.3 Hz, 1H, C2H_{b}), 2.95 (app-td, J=13.3, 4.8 Hz, 1H, C1′Hₐ), 2.92 (s, 3H, N8CH₃), 2.84 (d, J=9.2 Hz, 1H, C10H), 2.60 (app-td, J=13.4, 4.0 Hz, 1H, C1′H_{b}), 2.21 (app-dt, J=14.3, 2.9 Hz, 1H, C3Hₐ), 1.34 (s, 3H, C13H₃), 1.23 (s, 3H, C12H₃), 1.17 (ddd, J=14.4, 11.7, 4.2 Hz, 1H, C3H_{b}), 1.01 (app-td, J=13.8, 4.7 Hz, 1H, C2′Hₐ), 0.92 (app-td, J=13.7, 4.0 Hz, 1H, C2′H_{b}), −0.12 (s, 9H, Si(CH₃)₃). ¹³C NMR (100.6 MHz, C₆D₆, 60° C.): δ 177.2 (C8a), 145.1 (C7a), 144.0 (C4), 129.9 (C6), 126.6 (C4a), 119.0 (C5), 107.6 (C7), 64.5 (C10), 63.3 (C3a), 58.5 (2C, C11, C9), 51.7 (C1), 46.8 (C2), 35.7 (C3), 26.7 (N8CH₃), 25.1 (C12), 19.4 (C13), 10.1 (C2′), −2.1 (Si(CH₃)₃). FTIR (thin film) cm⁻¹: 3209 (br-m), 2954 (m), 1714 (s), 1605 (s), 1471 (m), 1422 (m), 1367 (m), 1327 (s), 1252 (m), 1151 (m). HRMS (ESI) (m/z): calc'd for C₂₁H₃₄N₃O₄SSi [M+H]⁺: 452.2034, found: 452.2032. [α]_{D}²⁴: −67 (c=0.62, CH₂Cl₂). TLC (50% acetone in dichloromethane), Rf: 0.24 (UV, CAM).

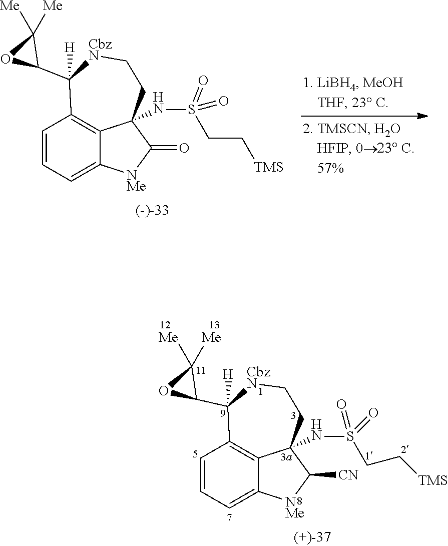

### Example 16: Aminonitrile (+)-37

A solution of lithium borohydride (2.0 M in tetrahydrofuran, 3.8 mL, 7.6 mmol, 15 equiv) was added dropwise via syringe over 2 min to a solution of oxindole (−)-33 (293 mg, 0.500 mmol, 1 equiv) in tetrahydrofuran (6.70 mL) at 23° C. Methanol (1.21 mL, 30.0 mmol, 60.0 equiv) was then added dropwise over 3 h by syringe pump and the resulting white suspension was allowed to stir vigorously at 23° C. After 17 h, the mixture was cooled to 0° C. and was diluted with a saturated aqueous ammonium chloride solution (50 mL) and water (20 mL). After vigorous stirring at 23° C. for 10 min, the mixture was extracted with ethyl acetate (3×30 mL). The combined organic extracts were washed with a saturated aqueous sodium chloride solution (60 mL), were dried over anhydrous sodium sulfate, were filtered, and were concentrated under reduced pressure to provide the crude hemiaminal as a white foam, which was used directly in the next step without further purification.

Trimethylsilyl cyanide (375 μL, 3.00 mmol, 6.00 equiv) was added dropwise over 2 min to a solution of the crude hemiaminal and water (81.0 μL, 4.50 mmol, 9.00 equiv) in hexafluoroisopropanol (HFIP, 3.30 mL) at 0° C. After 10 min, the reaction flask was sealed under an argon atmosphere with a Teflon-lined glass stopper and the ice bath was removed. After 20 h, the solution was cooled to 0° C. and was diluted with an aqueous sodium hydroxide solution (0.5 M, 20 mL). After warming to 23° C., the mixture was extracted with dichloromethane (3×20 mL). The combined organic extracts were washed with a saturated aqueous sodium chloride solution (40 mL), were dried over anhydrous sodium sulfate, were filtered, and were concentrated under reduced pressure. The resulting residue was purified by flash column chromatography on silica gel (eluent: 20→24% ethyl acetate in hexanes) to afford aminonitrile (+)-37 (171 mg, 57.3%) as a white foam. As a result of slow conformational equilibration at ambient temperature, NMR spectra were collected at elevated temperature. ¹H NMR (500 MHz, CD₃CN, 70° C.): δ 7.58-7.06 (m, 6H), 6.71 (br-d, J=7.0 Hz, 1H), 6.68 (d, J=7.9 Hz, 1H), 5.40-4.82 (m, 5H), 4.16 (app br-s, 1H), 3.94-3.77 (m, 1H), 3.40 (app br-s, 1H), 3.07-2.97 (m, 1H), 2.95 (s, 3H), 2.90 (ddd, J=14.0, 10.4, 7.5 Hz, 1H), 2.86-2.78 (m, 1H), 2.58 (ddd, J=15.0, 9.9, 5.8 Hz, 1H), 1.41 (s, 3H), 1.37 (s, 3H), 1.01-0.90 (m, 2H), 0.05 (s, 9H). ¹H NMR (125.8 MHz, CD₃CN, 70° C.): δ 157.3, 153.1, 139.8, 138.4, 132.5, 129.8, 129.2, 129.0, 127.7, 119.2, 117.0, 109.7, 68.6 (2C), 68.5, 64.3, 61.2, 61.0, 53.1, 44.6, 35.8, 34.0, 25.1, 20.2, 11.4, −1.5. FTIR (thin film) cm⁻¹: 3248 (br-w), 2956 (w), 1694 (m), 1596 (m), 1453 (m), 1419 (s), 1324 (s), 1250 (s), 1143 (s), 1119 (s), 991 (m), 840 (s), 744 (s), 697 (s), 540 (m). HRMS (DART) (m/z): calc'd for C₃₀H₄₁N₄OSSi [M+H]: 597.2567, found: 597.2565. [α]_{D}²⁴: +60 (c=0.98, CH₂Cl₂). TLC (22% ethyl acetate in hexanes), Rf: 0.17 (UV, CAM).

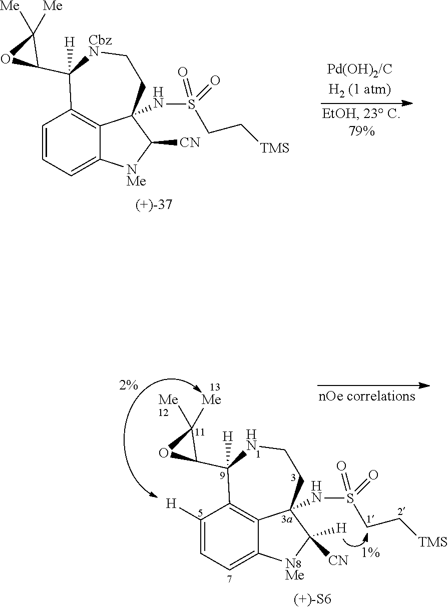

### Example 17: Azepane (+)-S6

A sample of palladium(II) hydroxide on carbon (15.7 wt % on wet support, 2.0 mg, 1.7 μmol, 0.10 equiv) was added to a solution of aminonitrile (+)-37 (10 mg, 16.8 μmol, 1 equiv) in anhydrous ethanol (200 proof, 0.84 mL) at 23° C. The resulting black suspension was sparged with dihydrogen for 5 min by discharge of a balloon equipped with a needle extending into the reaction mixture. After stirring for 2 h under an atmosphere of dihydrogen, the suspension was sparged with argon for 5 min and was diluted with ethyl acetate (5 mL). The mixture was then filtered through a plug of Celite and the filter cake was washed with ethyl acetate (15 mL). The colourless filtrate was concentrated under reduced pressure and the resulting residue was purified by flash column chromatography on silica gel (eluent: 40→45% ethyl acetate in hexanes) to afford azepane (+)-S6 (6.2 mg, 79.4%) as a colourless oil. Structural assignments were made using additional information from gCOSY, gHSQC, gHMBC, and 1D selective NOESY experiments. ¹H NMR (500 MHz, CDCl₃, 20° C.): δ 7.20 (t, J=7.8 Hz, 1H, C6H), 6.53 (d, J=7.8 Hz, 1H, C7H), 6.50 (app-dt, J=7.7, 0.7 Hz, 1H, C5H), 5.33 (s, 3H, C8aH), 3.86 (d, J=9.9 Hz, 1H, C9H), 3.60 (ddd, J=13.3, 7.4, 2.2 Hz, 1H, C2Hₐ), 3.39 (ddd, J=13.3, 10.5, 7.1 Hz, 1H, C2H_{b}), 3.02 (d, J=9.4 Hz, 1H, C10H), 2.92 (s, 3H, N8CH₃), 2.91-2.83 (m, 2H, C1′H₂), 2.75 (ddd, J=14.3, 7.1, 2.1 Hz, 1H, C3Hₐ), 2.48 (ddd, J=14.2, 10.5, 7.3 Hz, 1H, C3Hₐ), 1.49 (s, 3H, C13H₃), 1.41 (s, 3H, C12H₃), 1.07-0.96 (m, 1H, C2′Hₐ), 0.92-0.79 (m, 1H, C2′H_{b}), −0.02 (s, 9H, Si(CH₃)₃ ¹³C NMR (125.8 MHz, CDCl₃, 25° C.): δ 150.8 (C7a), 139.2 (C4), 130.7 (C6), 129.5 (C4a), 117.9 (C5), 115.5 (CN), 107.9 (C7), 67.2 (C3a), 64.9 (C10), 64.7 (C8a), 60.6 (C11), 59.4 (C9), 52.2 (C1), 40.8 (C2), 36.4 (C3), 33.1 (N8CH₃), 25.0 (C12), 20.0 (C13), 10.5 (C2′), −1.9 (Si(CH₃)₃). FTIR (thin film) cm⁻¹: 3248 (br-w), 2955 (m), 1595 (m), 1451 (m), 1329 (s), 1251 (s), 1143 (s), 968 (w), 905 (m), 843 (s). HRMS (ESI) (m/z): calc'd for C₂₂H₃₅N₄O₃SSi [M+H]⁺: 463.2194, found: 463.2195. [α]_{D}²⁴: +141 (c=0.31, CH₂Cl₂). TLC (40% ethyl acetate in hexanes), Rf: 0.15 (UV, CAM).

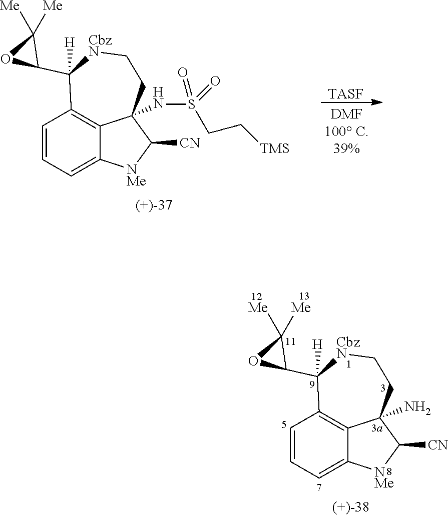

### Example 18: Benzylic Aminonitrile (+)-38

A sample of aminonitrile (+)-37 (45.0 mg, 75.4 μmol, 1 equiv) contained in a 5-mL Schlenk flask (Kjeldahl shape) was azeotropically dried by concentration from anhydrous benzene (3×1 mL). After drying under high vacuum for 2.5 h, the flask was refilled with argon and was charged with a sample of tris(dimethylamino)sulfonium difluorotrimethylsilicate (TASF, 83.1 mg, 302 μmol, 4.00 equiv). The mixture was dissolved in N,N-dimethylformamide (1.00 mL) and the resulting homogeneous solution was stirred at 23° C. for 10 min. The flask was then sealed and was immersed in a preheated oil bath at 100° C. After stirring at this temperature for 9 h, the light-brown solution was cooled to 23° C. and was diluted with a saturated aqueous sodium chloride solution (20 mL) and water (5 mL) and the yellow suspension was extracted with ethyl acetate (3×10 mL). The combined organic extracts were washed with a saturated aqueous sodium chloride solution (2×20 mL), were dried over anhydrous sodium sulfate, were filtered, and were concentrated under reduced pressure. The resulting residue was purified by flash column chromatography on silica gel (eluent: 50→65% ethyl acetate in hexanes) to afford benzylic aminonitrile (+)-38 (12.8 mg, 39.2%) as a pale-yellow film. As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (400 MHz, CD₃CN, 70° C.): δ 7.40-7.22 (br-m, 5H, Ar_{Cbz}H), 7.20 (t, J=7.8 Hz, 1H, C6H), 6.67 (d, J=7.3 Hz, 1H, C5H), 6.60 (d, J=7.9 Hz, 1H, C7H), 5.10 (d, J=12.6 Hz, 1H, N1CO₂CHₐPh), 5.08 (d, J=12.6 Hz, 1H, N1CO₂CH_{b}Ph), 4.94 (d, J=8.8 Hz, 1H, C9H), 4.29 (s, 1H, C8aH), 4.06 (ddd, J=14.8, 10.5, 4.6 Hz, 1H, C2Hₐ), 3.92-3.81 (m, 1H, C2H_{b}), 3.60-3.40 (br-m, 1H, C10H), 2.91 (s, 3H, N8CH₃), 2.47 (ddd, J=14.1, 10.3, 5.6 Hz, 1H, C3Hₐ), 2.21 (ddd, J=14.1, 4.8, 3.7 Hz, 1H, C3H_{b}), 1.37 (s, 3H, C12/13H₃), 1.33 (s, 3H, C12/13H₃). ¹³C NMR (100.6 MHz, CD₃CN, 70° C.): δ 157.4 (N1CO₂CH₂Ph), 151.8 (C7a), 139.0 (C4), 138.7 (Ar_{Cbz}), 132.2 (C4a), 131.1 (C6), 129.8 (Ar_{Cbz}), 129.2 (Ar_{Cbz}), 129.0 (Ar_{Cbz}), 118.8 (C5), 117.6 (CN), 109.2 (C7), 72.9 (C8a), 68.3 (N1CO₂CH₂Ph), 65.2 (C3a), 64.0 (C10), 61.7 (C9), 60.7 (C11), 45.4 (C2), 36.9 (C3), 34.1 (N8CH₃), 25.2 (C12/13), 19.9 (C12/13). FTIR (thin film) cm⁻¹: 3371 (br-w), 3309 (br-w), 2960 (w), 1698 (s), 1597 (m), 1465 (m), 1419 (m), 1329 (m), 1259 (m), 747 (m). HRMS (DART) (m/z): calc'd for C₂₅H₂₉N₄O₃ [M+H]: 433.2240, found: 433.2244. [α]_{D}²³: +65 (c=0.55, CH₂Cl₂). TLC (60% ethyl acetate in hexanes), Rf: 0.13 (UV, CAM).

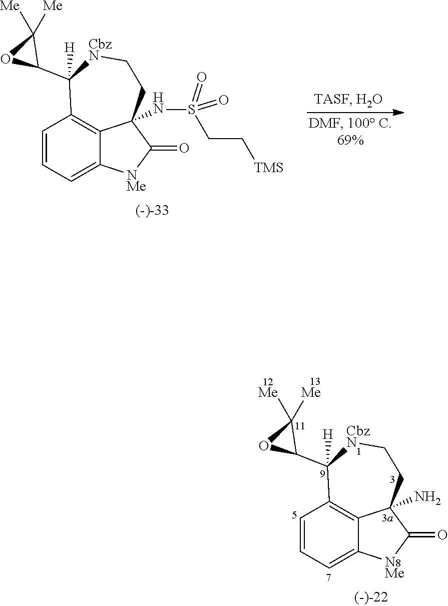

### Example 19: Benzylic Amine (−)-22

A sample of tris(dimethylamino)sulfonium difluorotrimethylsilicate (TASF, 3.99 g, 14.5 mmol, 3.20 equiv) was added as a solid to a pressure flask containing a solution of tricyclic epoxide (−)-33 (2.65 g, 4.52 mmol, 1 equiv) and deionized water (82.0 μL, 4.52 mmol, 1.00 equiv) in N,N-dimethylformamide (30.0 mL) at 23° C. The reaction vessel was sealed with a Teflon screwcap under an argon atmosphere and was immersed in a preheated oil bath at 100° C. After 19 h, the reaction mixture was cooled to 23° C., was diluted with a saturated aqueous sodium chloride solution (300 mL) and deionized water (50 mL) and was extracted with diethyl ether (5×200 mL). The combined organic extracts were washed with a saturated aqueous sodium chloride solution (3×400 mL), were dried over anhydrous sodium sulfate, were filtered, and were concentrated under reduced pressure. The resulting residue was purified by flash column chromatography on silica gel (eluent: 40% ethyl acetate, 4% triethylamine, 56% hexanes→44% ethyl acetate, 4% triethylamine, 52% hexanes) to afford benzylic amine (−)-22 (1.31 g, 68.7%) as a light-yellow foam. As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (400 MHz, DMSO-d₆, 130° C.): δ 7.35-7.19 (m, 6H, C6H, Ar_{Cbz}H), 6.93-6.82 (m, 2H, C5H, C7H), 5.09-4.95 (m, 3H, N1CO₂CH₂Ph, C9H), 3.94 (ddd, J=14.4, 8.8, 3.9 Hz, 1H, C2Hₐ), 3.83 (app-dt, J=14.5, 5.0 Hz, 1H, C2H_{b}), 3.66 (d, J=8.6 Hz, 1H, C10H), 3.13 (s, 3H, N8CH₃), 1.96 (ddd, J=14.1, 5.2, 3.9 Hz, 1H, C3Hₐ), 1.85 (br-s, 2H, C3aNH₂), 1.54 (ddd, J=13.9, 8.7, 4.8 Hz, 1H, C3H_{b}), 1.40 (s, 3H, C12/13H₃), 1.27 (s, 3H, C12/13H₃). ¹³C NMR (100.6 MHz, DMSO-d₆, 130° C.): δ 178.5 (C8a), 154.5 (N1C₂CH₂Ph), 142.6 (C7a), 136.7 (C4), 136.1 (Ar_{Cbz}), 128.6 (C4a), 127.7 (C6), 127.3 (Ar_{Cbz}), 126.8 (Ar_{Cbz}), 126.7 (Ar_{Cbz}), 118.6 (C5), 106.7 (C7), 65.6 (N1CO₂CH₂Ph), 60.9 (C10), 59.3 (C9), 58.6 (C3a), 57.7 (C11), 44.7 (C2), 32.2 (C3), 25.1 (N8CH₃), 23.6 (C12/13), 17.8 (C12/13). FTIR (thin film) cm⁻¹: 3360 (w), 3288 (w), 2962 (m), 1710 (s), 1608 (s), 1473 (s), 1417 (m), 1368 (m), 1301 (m), 1260 (m). HRMS (ESI) (m/z): calc'd for C₂₄H₂₈N₃O₄ [M+H]⁺: 422.2074, found: 422.2079. [α]_{D}²³: −111 (c=1.14, CH₂Cl₂). TLC (60% ethyl acetate in hexanes), Rf: 0.20 (UV, CAM).

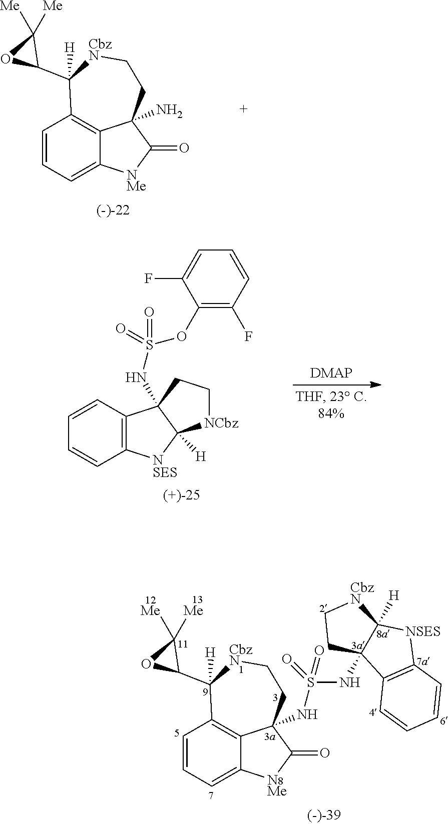

### Example 20: Sulfamide (−)-39

A sample of 4-(dimethylamino)pyridine (DMAP, 386 mg, 3.16 mmol, 1.10 equiv) was added to a solution of benzylic amine (−)-22 (1.21 g, 2.88 mmol, 1 equiv) and sulfamate ester (+)-25 (2.87 g, 4.31 mmol, 1.50 equiv) in tetrahydrofuran (11.5 mL) at 23° C. After 24 h, the homogeneous solution was diluted with a saturated aqueous ammonium sulfate solution (80 mL) and deionized water (20 mL) and the resulting mixture was extracted with ethyl acetate (3×100 mL). The combined organic extracts were washed with a saturated aqueous sodium chloride solution (200 mL), were dried over anhydrous sodium sulfate, were filtered, and were concentrated under reduced pressure. The residue was purified by flash column chromatography on silica gel (eluent: 45%→50% ethyl acetate in hexanes) to afford sulfamide (−)-39 (2.32 g, 84.3%) as a white foam. As a result of the slow conformational equilibration at ambient temperature, NMR spectra were collected at elevated temperature. ¹H NMR (400 MHz, C₆D₆, 70° C.): δ 7.59-7.24 (m, 6H), 7.13-6.99 (m, 6H), 6.94 (t, J=7.8 Hz, 2H), 6.80 (t, J=7.6 Hz, 1H), 6.55 (s, 1H), 6.50-6.20 (m, 2H), 5.16 (d, J=12.2 Hz, 1H), 5.08 (d, J=12.1 Hz, 1H), 4.97 (br-s, 3H), 3.77 (br-s, 1H), 3.69-3.46 (m, 2H), 3.38-3.21 (m, 1H), 3.15 (s, 3H), 2.52 (td, J=11.6, 5.4 Hz, 1H), 2.33-1.80 (m, 3H), 1.61-0.99 (m, 10H), −0.10 (s, 9H). ¹³C NMR (100.6 MHz, C₆D₆, 70° C.):^{58 δ 176.6, 156.2} (br), 155.0, 144.9, 143.6, 137.6, 137.0, 132.1, 130.1, 129.7 (br), 128.8 (2C), 128.6, 128.5 (2C), 127.9, 125.3, 124.5, 120.8, 117.2, 108.1, 83.3, 73.1, 67.8, 67.7, 62.9 (br), 59.1 (br), 51.5, 45.6, 36.5, 32.9, 26.8, 24.9, 19.4, 10.6, −2.0. FTIR (thin film) cm⁻¹: 3415 (br-w), 3222 (br-w), 2956 (w), 1707 (s), 1602 (m), 1465 (m), 1412 (m), 1346 (m), 1317 (m), 1253 (m), 1198 (m), 1150 (m), 1102 (m), 753 (m). HRMS (ESI) (m/z): calc'd for C₄₇H₅₇N₆O₁₀S₂Si [M+H]⁺: 957.3341, found: 957.3353. [α]_{D}²³: −37 (c=0.37, CH₂Cl₂). TLC (50% ethyl acetate in hexanes), Rf: 0.30 (UV, CAM).

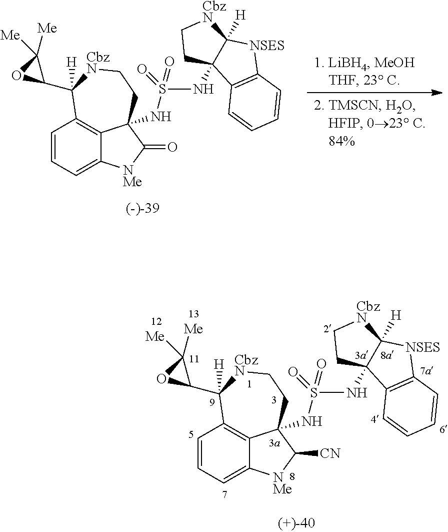

### Example 21: Aminonitrile Sulfamide (+)-40

A solution of lithium borohydride (2.0 M in tetrahydrofuran, 15 mL, 30 mmol, 15 equiv) was added over 5 min to a solution of sulfamide (−)-39 (1.91 g, 2.00 mmol, 1 equiv) in tetrahydrofuran (27.0 mL) at 23° C. Methanol (4.85 mL, 120 mmol, 60.0 equiv) was then added dropwise by syringe pump over 3.5 h and the resulting white suspension was allowed to stir vigorously at 23° C. After 17 h, the mixture was cooled to 0° C. and was diluted with a saturated aqueous ammonium chloride solution (200 mL) and deionized water (50 mL). After vigorous stirring at 23° C. for 10 min, the mixture was extracted with ethyl acetate (3×150 mL). The combined organic extracts were washed with a saturated aqueous sodium chloride solution (200 mL), were dried over anhydrous sodium sulfate, were filtered, and were concentrated under reduced pressure to provide the crude hemiaminal as a white foam, which was used directly in the next step without further purification.

Trimethylsilyl cyanide (1.50 mL, 12.0 mmol, 6.00 equiv) was added dropwise to a solution of the crude hemiaminal and deionized water (324 μL, 18.0 mmol, 9.00 equiv) in hexafluoroisopropanol (HFIP, 13.3 mL) at 0° C. After 5 min, the reaction flask was sealed under an argon atmosphere with a Teflon-lined glass stopper and the ice-bath was removed. After 20 h, the solution was cooled to 0° C. and was diluted with an aqueous sodium hydroxide solution (0.1 M, 133 mL) and a saturated aqueous sodium chloride solution (133 mL). After warming to 23° C., the mixture was extracted with dichloromethane (3×130 mL). The combined organic extracts were dried over anhydrous sodium sulfate, were filtered, and were concentrated under reduced pressure. The resulting residue was purified by flash column chromatography on silica gel (eluent: 16% ethyl acetate, 42% hexanes, 42% dichloromethane→20% ethyl acetate, 40% hexanes, 40% dichloromethane) to afford aminonitrile sulfamide (+)-40 (1.62 g, 83.6%) as a white foam. As a result of the slow conformational equilibration at ambient temperature, NMR spectra were collected at elevated temperature. ¹H NMR (500 MHz, C₆D₆, 70° C.): δ 7.49 (d, J=8.2 Hz, 1H), 7.35 (br-d, J=5.7 Hz, 1H), 7.26 (d, J=7.3 Hz, 2H), 7.23-7.16 (br-m, 2H), 7.15-6.98 (m, 8H), 6.93 (t, J=7.5 Hz, 1H), 6.85-6.59 (br-m, 1H), 6.50 (br-s, 1H), 6.26 (d, J=7.9 Hz, 1H), 5.36 (br-s, 1H), 5.20 (s, 1H), 5.09 (s, 2H), 5.04 (s, 2H), 4.96-3.99 (br-m, 2H), 3.82-3.52 (m, 3H), 3.43 (td, J=14.1, 13.6, 4.6 Hz, 1H), 3.32-2.75 (br-m, 2H), 2.74-2.42 (m, 6H), 2.25-2.14 (m, 1H), 1.38-1.09 (m, 5H), 1.02 (s, 3H), −0.07 (s, 9H). ¹³C NMR (100.6 MHz, C₆D₆, 70° C.): δ 157.4, 154.9, 152.1, 143.8, 138.8, 137.3, 137.0, 131.9 (br), 131.4, 130.5, 128.8, 128.7, 128.4, 128.3, 128.2, 127.9 (br), 127.2, 125.0, 124.9 (br), 118.4 (br), 118.2, 115.6, 108.7, 83.4, 72.6, 68.2, 67.8, 67.6, 66.6, 63.4, 59.8, 59.4 (br), 50.9, 45.3, 41.1 (br), 36.5 (br), 33.6 (br), 33.0, 24.4, 19.9, 10.6, −1.9. FTIR (thin film) cm-1:3246 (br-w), 2956 (w), 2896 (w), 1708 (m), 1600 (m), 1414 (m), 1357 (m), 1252 (m), 1149 (m). HRMS (ESI) (m/z): calc'd for C₄₈H₅₇N₇NaO₉S₂Si [M+Na]⁺: 990.3321, found: 990.3312. [α]_{D}²⁴: +69 (c=0.47, CH₂Cl₂). TLC (19% ethyl acetate, 41% hexanes, 41% dichloromethane), Rf: 0.29 (UV, CAM).

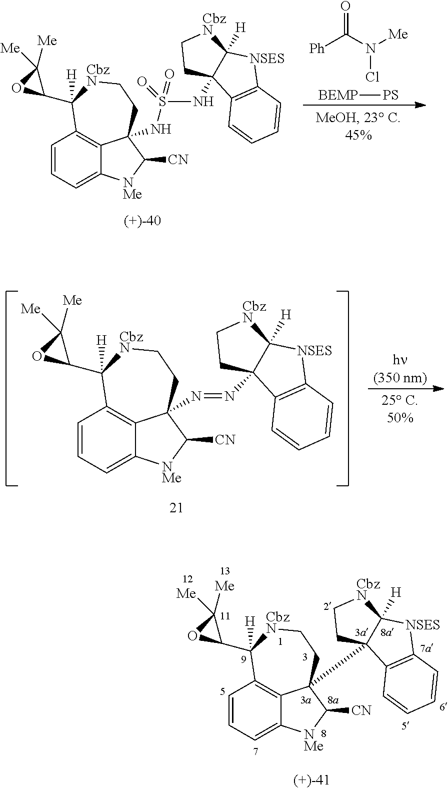

### Example 22: Heterodimer (+)-41

N-Chloro-N-methylbenzamide⁵⁹ (672 mg, 3.96 mmol, 6.00 equiv) and resin-bound 2-tert-butylimino-2-diethylamino-1,3-dimethylperhydro-1,3,2-diazaphosphorine (BEMP, 3.62 g, 2.19 mmol/g on 200.400 mesh polystyrene resin, 7.92 mmol, 12.0 equiv) were added in rapid succession to a solution of aminonitrile sulfamide (+)-40 (639 mg, 660 μmol, 1 equiv) in methanol (66.0 mL) at 23° C. in the dark. After 15 min, the suspension was filtered through a pad of Celite, and the filter cake was washed sequentially with dichloromethane (70 mL) and ethyl acetate (70 mL). The light yellow filtrate was concentrated under reduced pressure and the resulting residue was purified by flash column chromatography on silica gel (eluent: 6% ethyl acetate, 47% hexanes, 47% dichloromethane→10% ethyl acetate, 45% hexanes, 45% dichloromethane) to afford unsymmetrical diazene 21 (270 mg, 45.3%) as a light yellow foam,⁶⁰ which was used directly in the next step without further purification.

A solution of diazene 21 (268 mg, 297 μmol, 1 equiv) in dichloromethane (20 mL) was concentrated under reduced pressure in a 500-mL round-bottom flask to provide a thin film of the diazene coating the flask. The flask was evacuated and backfilled with argon (three cycles) and was then irradiated in a Rayonet photoreactor equipped with 14 radially distributed (r=12.7 cm) 25 W lamps (X=350 nm) at 25° C. After irradiating for 3 h, the lamps were turned off and the resulting residue was purified by flash column chromatography on silica gel (eluent: 10% ethyl acetate, 60% hexanes, 30% dichloromethane→10% ethyl acetate, 45% hexanes, 45% dichloromethane) to afford heterodimer (+)-41 (129 mg, 49.6%) as an off-white film. As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (500 MHz, CD₃CN, 70° C.): δ 7.56-7.45 (br-m, 1H, C4′H), 7.43-7.18 (m, 13H, C6H, C6′H, C7′H, Ar_{Cbz}H), 7.10 (br-t, J=7.4 Hz, 1H, C5′H), 6.65 (app-dt, J=7.8, 0.9 Hz, 1H, C7), 6.59 (app-dt, J=7.9, 1.0 Hz, 1H, C5H), 5.61 (s, 1H, C8a′H), 5.43 (d, J=8.7 Hz, 1H, C9H), 5.36-4.97 (br-m, 2H, N1CO₂CH₂), 4.92 (d, J=12.6 Hz, 1H, N1′CO₂CHa), 4.89 (d, J=12.6 Hz, 1H, N1′CO₂CH_{b}), 4.06 (dddd, J=14.4, 3.8, 2.6, 1.0 Hz, 1H, C2Hₐ), 3.79 (s, 1H, C8aH), 3.50-3.38 (br-m, 1H, C2′Hₐ), 3.36 (ddd, J=14.6, 12.6, 2.3 Hz, 1H, C2H_{b}), 3.22-3.09 (m, 2H, N8′SO₂CH₂), 3.09-2.90 (br-m, 1H, C3Hₐ), 2.86 (d, J=8.7 Hz, 1H, C10H), 2.74 (s, 3H, N8CH₃), 2.44-2.24 (br-m, 1H, C2′H_{b}), 2.12 (app-dt, J=15.5, 2.5 Hz, 1H, C3H_{b}), 1.89-1.62, (br-m, 2H, C3′H₂), 1.56 (br-s, 3H, C12/13H₃), 1.38 (s, 3H, C12/13H₃), 1.10-0.99 (m, 1H, N8′SO₂CH₂CHa), 0.93 (app-td, J=13.6, 5.0 Hz, 1H, N8′SO₂CH₂CH_{b}), −0.01 (s, 9H, Si(CH₃)₃). ¹³C NMR (125.8 MHz, CD₃CN, 70° C.): δ 156.8 (N1CO₂), 155.3 (N1′CO₂), 154.0 (C7a), 145.0 (C7a′), 138.7 (C4), 138.4 (Ar_{Cbz}), 138.0 (Ar_{Cbz}), 132.0 (C6), 131.4 (C4a′), 130.9 (C6′), 130.3 (C4′), 129.9 (Ar_{Cbz}), 129.7 (Ar_{Cbz}), 129.6 (Ar_{Cbz}), 129.2 (Ar_{Cbz}), 129.1 (Ar_{Cbz}), 126.3 (C4a), 126.0 (Ar_{Cbz}), 124.8 (C5′), 120.1 (C5), 117.7 (CN), 116.1 (C7′), 109.5 (C7), 82.1 (C8a′), 70.7 (C8a), 68.4 (N1CO₂CH₂), 68.1 (N1′CO₂CH₂), 67.1 (C10), 67.0 (C3a′), 62.2 (C11), 59.3 (C9), 58.8 (C3a), 52.7 (N8′SO₂CH₂), 45.7 (C2′), 43.0 (C2), 36.1 (C3′), 35.1 (br, C3), 34.9 (N8CH₃), 24.9 (C12/13), 20.0 (C12/13), 10.9 (N8′SO₂CH₂CH₂), −1.6 (Si(CH₃)₃). FTIR (thin film) cm⁻¹: 3065 (m), 2958 (s), 2895 (m), 1706 (s), 1586 (s), 1459 (s), 1417 (s), 1356 (s), 1153 (s), 1051 (s). HRMS (ESI) (m/z): calc'd for C₄₈H₅₆N₅O₇SSi [M+H]⁺: 874.3664, found: 874.3661. [α]_{D}²³: +204 (c=0.34, CH₂Cl₂). TLC (10% ethyl acetate, 60% hexanes, 30% dichloromethane), Rf: 0.13 (UV, CAM).

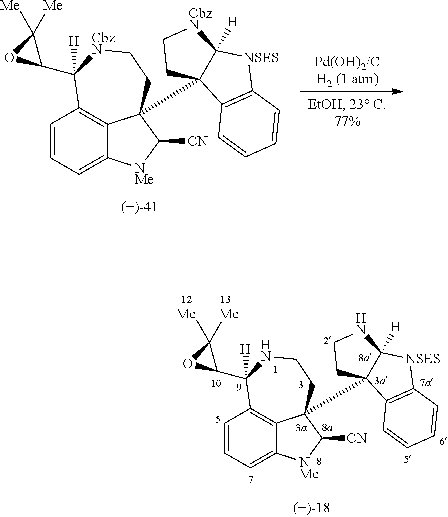

### Example 23: Heterodimeric Diamine (+)-18

A sample of palladium(II) hydroxide on carbon (15.7 wt % on wet support, 79.1 mg, 88.5 mol, 0.600 equiv) was added to a solution of heterodimer (+)-41 (129 mg, 148 mol, 1 equiv) in anhydrous ethanol (200 proof, 5.90 mL) at 23° C. The resulting suspension was sparged with dihydrogen for 5 min by discharge of a balloon equipped with a needle extending into the reaction mixture. After stirring for 6 h under an atmosphere of dihydrogen, the suspension was sparged with argon for 5 min, was diluted with ethanol (6 mL), and was filtered through a pad of Celite. The filter cake was washed with ethanol (60 mL) and the colourless filtrate was concentrated under reduced pressure. The resulting residue was purified by flash column chromatography on silica gel (eluent: 40→60% acetonitrile in dichloromethane) to afford heterodimeric diamine (+)-18 (68.9 mg, 77.1%) as a white film. Structural assignments were made using additional information from gCOSY, gHSQC, gHMBC, and 1D selective NOESY experiments. ¹H NMR (500 MHz, CD₃OD, 20° C.): δ 7.47 (d, J=7.6 Hz, 1H, C4′H), 7.38-7.30 (m, 3H, C6H, C6′H, C7′H), 7.09 (ddd, J=8.2, 6.2, 2.3 Hz, 1H, C5′H), 6.79 (d, J=7.7 Hz, 1H, C5H), 6.70 (d, J=7.9 Hz, 1H, C7H), 5.65 (s, 1H, C8a′H), 4.15-4.09 (m, 2H, C8aH, C9H), 3.42-3.32 (m, 2H, N8′SO₂CH₂), 3.18 (d, J=8.1 Hz, 1H, C10H), 3.16-3.02 (m, 2H, C2H₂), 2.84 (s, 3H, N8CH₃), 2.82-2.78 (m, 1H, C2′Hₐ), 2.74-2.64 (m, 1H, C3Hₐ), 2.54 (dd, J=10.9, 3.9 Hz, 1H, C3′Hₐ), 2.23 (app-td, J=11.5, 3.9 Hz, 1H, C2′H_{b}), 2.17-2.04 (m, 2H, C3H_{b}, C3′H_{b}), 1.49 (s, 3H, C12/13H₃), 1.33 (s, 3H, C12/13H₃), 1.14 (ddd, J=13.9, 10.9, 6.6 Hz, 1H, N8′SO₂CH₂CHa), 1.01 (ddd, J=13.9, 11.2, 6.7 Hz, 1H, N8′SO₂CH₂CH_{b}), 0.01 (s, 9H, Si(CH₃)₃). ¹³C NMR (125.8 MHz, CD₃OD, 20° C.): δ 153.2 (C7a), 144.9 (C7a′), 136.3 (C4), 132.0 (C6), 131.9 (C4a′), 130.8 (C6′), 128.5 (C4a), 127.5 (C4′), 123.9 (C5′), 117.7 (C5), 117.2 (CN), 113.7 (C7′), 109.3 (C7), 85.6 (C8a′), 67.9 (C8a), 67.2 (C10), 66.5 (C3a′), 60.2 (C11), 58.2 (C3a), 57.1 (C9), 51.6 (N8′SO₂CH₂), 46.9 (C2′), 43.9 (C2), 39.0 (C3′), 34.3 (C3), 32.8 (N8CH₃), 24.9 (C12/13), 19.9 (C12/13), 10.3 (N8′SO₂CH₂CH₂), −2.1 (Si(CH₃)₃). FTIR (thin film) cm⁻¹: 3346 (br-w), 2955 (m), 2878 (w), 1588 (m), 1476 (m), 1459 (m), 1347 (m), 1250 (m), 1149 (m). HRMS (ESI) (m/z): calc'd for C₃₂H₄₄N₅O₃SSi [M+H]⁺: 606.2929, found: 606.2929. [α]_{D}²³: +262 (c=0.13, CH₂Cl₂). TLC (50% acetonitrile in dichloromethane), Rf: 0.36 (UV, CAM).

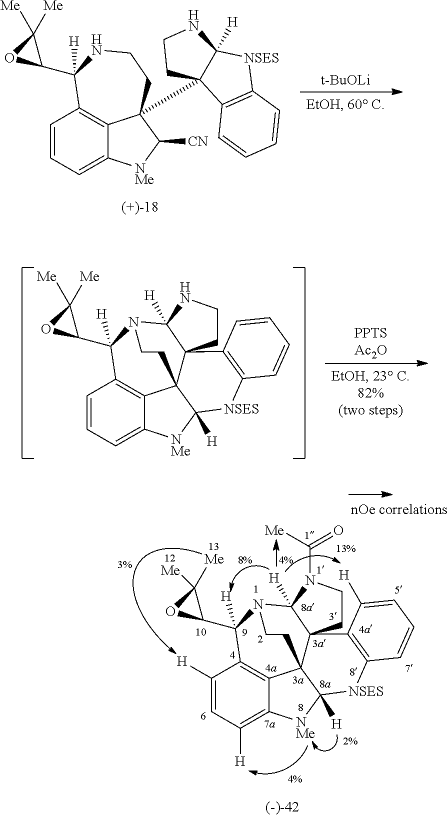

### Example 24: (−)-N8′-(Trimethylsilyl)ethanesulfonyl communesin A (42)

A sample of lithium tert-butoxide (25.0 mg, 312 μmol, 10.0 equiv) was added to a solution of heterodimer (+)-18 (18.9 mg, 31.2 μmol, 1 equiv) in anhydrous ethanol (200 proof, 820 μL) at 23° C. The flask was sealed with a Teflon-lined glass stopper under an argon atmosphere and was immersed in a preheated oil bath at 60° C. After 22 h, the reaction mixture was cooled to 23° C. and samples of pyridinium p-toluenesulfonate (PPTS, 62.8 mg, 250 μmol, 8.00 equiv) and acetic anhydride (12.0 μL, 125 μmol, 4.00 equiv) were added sequentially. After 40 min, a saturated aqueous sodium bicarbonate solution (10 mL) and deionized water (4 mL) were added and the resulting mixture was extracted with ethyl acetate (3×8 mL). The combined organic extracts were washed with a saturated aqueous sodium chloride solution (15 mL), were dried over anhydrous sodium sulfate, were filtered, and were concentrated under reduced pressure. The residue was purified by flash column chromatography on silica gel (eluent: 30% acetone in hexanes) to afford (−)-N8′-(trimethylsilyl)ethanesulfonyl communesin A (42, 15.9 mg, 82.0%) as a white solid. Crystals suitable for X-ray diffraction were obtained by slow evaporation of a solution of (−)-42 in 15% water in methanol at −20° C. Structural assignments were made using additional information from gCOSY, gHSQC, gHMBC, and 1D selective NOESY experiments. ¹H NMR (500 MHz, CDCl₃, 20° C., 5.9:1 mixture of atropisomers, *denotes minor atropisomer): δ 7.56 (dd, J=8.0, 1.3 Hz, 1H, C7′H), 7.41 (dd, J=8.0, 1.3 Hz, 1H, C7′H*), 7.20 (app-td, J=7.9, 1.5 Hz, 2H, C6′H, C6′H*), 7.06 (app-td, J=7.6, 1.3 Hz, 2H, C5′H, C5′H*), 6.91 (t, J=7.8 Hz, 1H, C6H), 6.89 (t, J=7.8 Hz, 1H, C6H*), 6.84 (dd, J=7.9, 1.5 Hz, 1H, C4′H), 6.78 (dd, J=7.9, 1.4 Hz, 1H, C4′H*), 6.14 (d, J=7.9 Hz, 1H, C5H*), 6.10 (d, J=7.6 Hz, 1H, C5H), 5.98 (d, J=7.6 Hz, 1H, C7H), 5.94 (d, J=7.6 Hz, 1H, C7H*), 5.71 (s, 1H, C8aH), 5.63 (s, 1H, C8aH*), 5.43 (app-s, 1H, C8a′H*), 5.04 (d, J=1.5 Hz, 1H, C8a′H), 4.49 (d, J=9.0 Hz, 1H, C9H*), 4.09 (d, J=9.2 Hz, 1H, C9H), 3.93 (app-dd, J=11.5, 9.1 Hz, 1H, C2′Hₐ), 3.75 (app-t, J=9.3 Hz, 1H, C2′Hₐ*), 3.55 (app-dd, J=16.0, 10.2 Hz, 1H, C2Hₐ*), 3.49 (app-dd, J=15.9, 9.8 Hz, 1H, C2Hₐ), 3.40-3.29 (m, 3H, C2H_{b}, C2H_{b}*, N8′SO₂CHa*), 3.25 (app-td, J=13.4, 5.0 Hz, 3H, N8′SO₂CHa, N8′SO₂CH_{b}*, C2′H_{b}*), 3.17 (app-td, J=13.4, 4.9 Hz, 1H, N8′SO₂CH_{b}), 3.14-3.04 (m, 1H, C2′H_{b}) 3.07-2.96 (m, 1H, C3′Hₐ*), 2.91 (s, 3H, N8CH₃), 2.87 (s, 3H, N8CH₃*), 2.85-2.75 (m, 1H, C3′Hₐ) 2.84 (d, J=9.0 Hz, 1H, C10H), 2.77 (d, J=8.7 Hz, 1H, C10H*), 2.55-2.43 (m, 2H, C3Hₐ, C3Hₐ*), 2.32-2.25 (m, 2H, C3H_{b}, C3H_{b}*), 2.32 (s, 3H, C2″H₃), 2.11 (dd, J=13.1, 7.5 Hz, 1H, C3′H_{b}*), 2.09 (s, 3H, C2″H₃*), 1.93 (dd, J=13.1, 6.4 Hz, 1H, C3′H_{b}), 1.57 (s, 3H, C13H₃*), 1.53 (s, 3H, C13H₃), 1.38 (s, 3H, C12H₃), 1.36 (s, 3H, C12H₃*), 1.31-1.13 (m, 4H, N8′SO₂CH₂CH₂, N8′SO₂CH₂CH₂*), 0.10 (s, 9H, Si(CH₃)₃*), 0.06 (s, 9H, Si(CH₃)₃). ¹³C NMR (125.8 MHz, CDCl₃, 21° C., 5.9:1 mixture of atropisomers, *denotes minor atropisomer): δ 171.8 (C1″), 170.5 (C1″), 149.9 (C7a), 149.5 (C7a*), 139.2 (C4a′*), 139.0 (C4*), 138.3 (C4a′), 137.2 (C4), 136.1 (2C, C7a′*, C7a′), 131.2 (2C, C4a, C4a*), 129.4 (C6), 129.2 (C6*), 127.1 (C6′), 127.7 (C6′*), 126.8 (C5′*), 126.4 (C5′), 125.4 (C7′*), 124.9 (C7′), 124.3 (C4′*), 123.9 (C4′), 114.6 (C5*), 113.9 (C5), 102.6 (C7), 102.3 (C7*), 85.3 (C8a*), 84.7 (C8a), 80.0 (C8a′), 78.2 (C8a′*), 65.5 (C9), 65.2 (C9*), 63.9 (2C, C10, C10*), 59.9 (2C, C11, C11*), 54.2 (2C, C3a, C3a*), 52.5 (2C, C3a′, N8′SO₂CH₂*), 51.8 (N8′SO₂CH₂), 50.3 (C3a′*), 45.9 (C2′*), 44.2 (C2′), 38.0 (C3*), 37.9 (C2*), 37.7 (C3), 36.6 (C2), 33.3 (C3′*), 31.7 (C3′), 31.0 (N8CH₃*), 30.9 (N8CH₃), 24.9 (C12), 24.8 (C12*), 23.1 (C2″*), 22.8 (C2″), 20.6 (C13), 20.5 (C13*), 10.8 (N8′SO₂CH₂CH₂*), 10.7 (N8′SO₂CH₂CH₂), −1.7 (Si(CH₃)₃*), −1.8 (Si(CH₃)₃). FTIR (thin film) cm⁻¹: 3055 (w), 2954 (m), 2928 (m), 2880 (m), 1651 (s), 1598 (m), 1487 (m), 1454 (m), 1400 (s), 1341 (s), 1281 (m), 1250 (m), 1207 (m), 1155 (s), 1081 (m), 1054 (m), 859 (m), 843 (m), 740 (m), 568 (m). HRMS (ESI) (m/z): calc'd for C₃₃H₄₅N₄O₄SSi [M+H]⁺: 621.2925, found: 621.2916. [α]_{D}²⁴: −144 (c=0.81, CH₂Cl₂). TLC (30% acetone in hexanes), Rf: 0.13 (UV, CAM).

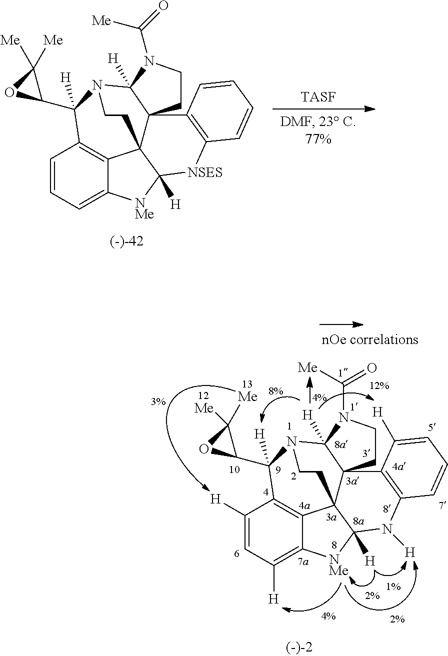

### Example 25: (−)-Communesin A (2)

A degassed solution of tris(dimethylamino)sulfonium difluorotrimethylsilicate (TASF, 24.3 mg, 88.0 μmol, 4.00 equiv) in N,N-dimethylformamide (235 μL) was added to a degassed solution of (−)-N8′-(trimethylsilyl)ethanesulfonyl communesin A (42, 13.6 mg, 22.0 mol, 1 equiv) in N,N-dimethylform-amide (500 L) at 23° C. After 2.7 h, a saturated aqueous sodium chloride solution (10 mL) and deionized water (5 mL) were added and the mixture was extracted with ethyl acetate (3×8 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 40% acetone in hexanes) to afford (−)-communesin A (2, 7.74 mg, 77.0%) as a white solid. Structural assignments were made using additional information from gCOSY, gHSQC, gHMBC, and 1D selective NOESY experiments. ¹H NMR (600 MHz, CDCl₃, 20° C., 11:1 mixture of atropisomers, *denotes minor atropisomer): δ 7.01 (app-td, J=7.4, 1.9 Hz, 2H, C6′H, C6′H*), 6.88 (t, J=7.7 Hz, 2H, C6H, C6H*), 6.74-6.66 (m, 5H, C4′H, C5′H, C5′H*, C7′H, C7′H*), 6.63 (d, J=7.4 Hz, 1H, C4′H*), 6.10 (d, J=7.4 Hz, 1H, C5H*), 6.06 (d, J=7.7 Hz, 1H, C5H), 5.95 (d, J=7.7 Hz, 1H, C7H), 5.91 (d, J=7.4 Hz, 1H, C₇H*), 5.41 (app-s, 1H, C8a′H*), 5.02 (d, J=1.5 Hz, 1H, C8a′H), 4.69 (s, 1H, C8aH), 4.67 (s, 1H, C8aH*), 4.59 (br-s, 1H, N8′H), 4.53-4.47 (m, 1H, C9H*), 4.08 (d, J=9.1 Hz, 1H, C9H), 3.89 (app-dd, J=11.8, 8.4 Hz, 1H, C2′Hₐ), 3.77-3.66 (m, 1H, C2′Hₐ*), 3.60-3.51 (m, 1H, C2Hₐ*), 3.47 (app-dd, J=15.7, 9.6 Hz, 1H, C2Hₐ), 3.36 (app-dt, J=16.3, 8.8 Hz, 2H, C2H_{b}, C2H_{b}*), 3.13 (app-q, J=9.9 Hz, 1H, C2′H_{b}*), 3.01 (app-td, J=11.6, 7.2 Hz, 1H, C2′H_{b}), 2.97-2.90 (m, 1H, C3′Hₐ*), 2.87 (d, J=8.9 Hz, 1H, C10H), 2.84 (s, 3H, N8CH₃), 2.82 (s, 3H, N8CH₃*), 2.80 (d, J=8.5 Hz, 1H, C10H*), 2.78-2.70 (m, 1H, C3′Hₐ), 2.37 (app-dd, J=12.5, 7.7 Hz, 2H, C3Hₐ, C3Hₐ*), 2.33 (s, 3H, C2″H₃), 2.28 (app-dt, J=13.0, 9.3 Hz, 2H, C3H_{b}, C3H_{b}*), 2.09(s, 3H, C2H₃*), 2.07-2.02n, 1H, C3H_{b}*), 1.97 (app-dd, J=13.3, 7.1 Hz, 1H, C3′H_{b}), 1.58 (s, 3H, C13H₃*), 1.53 (s, 3H, C13H₃), 1.38 (s, 3H, C12H₃), 1.37 (s, 3H, C12H₃*). ¹³C NMR (150.9 MHz, CDCl₃, 20C, 11:1mixture of atropisomers, *denotes minor atropisomer): δ 172.1 (C1″), 150.7 (C7a), 150.6 (C7a*), 142.8 (C7a), 137.0 (C4), 132.6 (C4a′), 132.4(C4a), 129.0 (C6), 127.6 (C6′), 127.4(C6*), 123.6 (C4*), 123.4 (C4′*), 121.0(C5*), 120.7 (C5′), 117.3 (C7*), 117.1(C7), 114.0 (C5*), 113.4 (C5), 102.0(C7), 101.6 (C7*), 83.0(C8a*), 82.6 (C8a), 79.8(C8a′), 77.9 (C8a*), 65.5(C9), 64.2 (d), 59.9(C), 52.1 (C3a′), 51-6(3a), 46.1(C2′*), 44.2(C), 38.2 (C3), 36.5 (C2), 32.7 (C3*), 31.0(C3′), 29.9(N8CH₃*), 29.8 (N8CH₃), 24.9 (C12), 24.8 (C12*), 23.2(C2*), 22.8(C2″), 20.6(C13), 20.5(C13*). FTIR (thin film) cm⁻¹: 3320 (br-w), 3051 (w), 2961 (m), 2926(m), 2880 (m), 1638 (s), 1605(s), 1595(s), 1493(s), 1402 (s), 1347(m), 1254(i), 1083(m), 1007(m), 2738 (s). HRMS (ESI) (m/z): calc'd for C₂₈H₃₃N₄O₂ [M+H]⁺: 457.2598, found: 457.2590. [α]_{D}²⁴: −165 (c=0.39, CHCl₃).⁶¹ TLC (40 acetone in hexanes), Rf: 0.19 (UV, CAM).

TABLE 2
Comparison of ¹H NMR data for (−)-communesin A (2) with literature data
(CDCl₃, major atropisomer):

| | Numata's Isolation | | | |
|---|---|---|---|---|
| | Report^{62,63} | Hayashi's Isolation | Ma's Report⁶⁶ | This Work |
| | ¹H NMR, 300 | Report^{64,65} | ¹H NMR, | ¹H NMR, |
| Assignment | MHz, CDCl₃ | ¹H NMR, CDCl₃ | 400 MHz, CDCl₃ | 600 MHz, CDCl₃ |
| C2 | 3.47 (dd, J = 16.0, | 3.32 (dd, J = 15.5, | 3.47 (dd, J = 16.0, | 3.47 (app-dd, J = 15.7, |
| | 9.0 Hz, 1H) | 9.5 Hz, 1H) | 9.2 Hz, 1H) | 9.6 Hz, 1H) |
| | 3.35 (dt, J = 16.0, | 3.18 (dt, J = 16.0, | 3.36 (dt, J = 16.0, | 3.36 (app-dt, J = 16.3, |
| | 9.2 Hz, 1H) | 9.2 Hz, 1H) | 9.2 Hz, 1H) | 8.8 Hz, 1H) |
| C3 | 2.35 (m, 1H) | 2.27 (dd, J = 12.5 | 2.39-2.25 (m, 2H) | 2.37 (app-dd, J = 12.5, |
| | 2.26 (dd, J = 12.4, | 9.5 Hz, 1H) | | 7.7 Hz, 1H) |
| | 9.2 Hz, 1H) | 2.12 (ddd, J = 12.5, | | 2.28 (app-dt, J = 13.0, |
| | | 9.5, 8.5 Hz, 1H) | | 9.3 Hz, 1H) |
| C3a | — | — | — | — |
| C4a | — | — | — | — |
| C4 | — | — | — | — |
| C5 | 6.07 (d, J = 7.8 Hz, | 6.03 (d, J = 7.5 Hz, | 6.06 (d, J = 7.6 Hz, | 6.06 (d, J = 7.7 Hz, 1H) |
| C6 | 6.89 (t, J = 7.8 Hz, | 6.79 (t, J = 7.5 Hz, | 6.88 (t, J = 7.6 Hz, | 6.88 (t, J = 7.7 Hz, 1H) |
| C7 | 5.95 (d, J = 7.8 Hz, | 5.93 (d, J = 7.5 Hz, | 5.95 (d, J = 7.6 Hz, | 5.95 (d, J = 7.7 Hz, 1H) |
| C7a | — | — | — | — |
| N8CH₃ | 2.85 (s, 3H) | 2.79 (s, 3H) | 2.84 (s, 3H) | 2.84 (s, 3H) |
| C8a | 4.70 (s, 1H) | 4.63 (d, J = 1.0 Hz, | 4.69 (s, 1H) | 4.69 (s, 1H) |
| C9 | 4.08 (d, J = 9.0 Hz, | 4.13 (d, J = 9.0 Hz, | 4.08 (d, J = 9.2 Hz, | 4.08 (d, J = 9.1 Hz, 1H) |
| C10 | 2.87 (d, J = 9.0 Hz, | 2.89 (d, J = 9.0 Hz, | 2.86 (d, J = 9.6 Hz, | 2.87 (d, J = 8.9 Hz, 1H) |
| C11 | — | | | |
| C12 | 1.39 (s, 3H) | 1.31 (s, 3H) | 1.38 (s, 3H) | 1.38 (s, 3H) |
| C13 | 1.54 (s, 3H) | 1.48 (s, 3H) | 1.53 (s, 3H) | 1.53 (s, 3H) |
| C2′ | 3.89 (dd, J = 12.0, | 3.67 (dt, J = 15.0, | 3.89 (dd, J = 12.0, | 3.89 (app-dd, J = 11.8, |
| | 8.8 Hz, 1H) | 7.0 Hz, 1H) | 8.8 Hz, 1H) | 8.4 Hz, 1H) |
| | 3.01 (td, J = 12.0, | 2.70 (m, 1H) | 3.01 (td, J = 11.6, | 3.01 (app-td, J = 11.6, |
| | 7.2 Hz, 1H) | | 8.0 Hz, 1H) | 7.2 Hz, 1H) |
| C3′ | 2.74 (td, J = 12.0, | 2.67 (m, 1H) | 2.78-2.70 (m, 1H) | 2.78-2.70 (m, 1H) |
| | 8.8 Hz, 1H) | 1.72 (dt, J = 12.5, | 1.97 (dd, J = 12.8, | 1.97 (app-dd, J = 13.3, |
| | 1.98 (td, J = 12.0, | 6.0 Hz, 1H) | 7.2 Hz, 1H) | 7.1 Hz, 1H) |
| | 7.2 Hz, 1H) | | | |
| C3a′ | — | — | — | — |
| C4a′ | — | — | — | — |
| C4′ | 6.70 (d, J = 2.8 Hz, 1H) | 6.73 (dd, J = 7.5, | 6.73-6.67 (m, 3H) | 6.74-6.66 (m, 3H) |
| | | 1.5 Hz, 1H) | | |
| C5′ | 6.71 (d, J = 7.5 Hz, 1H) | 6.61 (td, J = 7.5, | 6.73-6.67 (m, 3H) | 6.74-6.66 (m, 3H) |
| | | 1.5 Hz, 1H) | | |
| C6′ | 7.01 (td, J = 7.5, | 6.94 (td, J = 7.5, | 7.01 (td, J = 7.6, | 7.01 (app-td, J = 7.4, |
| | 2.8 Hz, 1H) | 1.5 Hz, 1H) | 1.6 Hz, 1H) | 1.9 Hz, 1H) |
| C7′ | 6.69 (d, J = 7.5 Hz, 1H) | 6.80 (dd, J = 7.5, | 6.73-6.67 (m, 3H) | 6.74-6.66 (m, 3H) |
| | | 1.5 Hz, 1H) | | |
| C7a′ | — | — | — | — |
| C8a′ | 5.03 (s, 1H) | 5.11 (s, 1H) | 5.02 (s, 1H) | 5.02 (d, J = 1.5 Hz, 1H) |
| C1″ | — | — | — | — |
| C2″ | 2.34 (s, 3H) | 2.23 (s, 3H) | 2.33 (s, 3H) | 2.33 (s, 3H) |
| N8'H | 4.62 (br-s, 1H) | 6.51 (d, 1.0 Hz) | — | 4.59 (br-s, 1H) |

TABLE 3
Comparison of ¹³C NMR data for (−)-communesin A (2) with literature
data (CDCl₃, major atropisomer):

| | Numata's Isolation | Hayashi's Isolation | Ma's Report⁶⁶ | This Work | Chemical Shift |
|---|---|---|---|---|---|
| | Report^{62,63} | Report^{64,65} | ¹³C NMR, | ¹³C NMR | Difference |
| | ¹³C NMR, | ¹³C NMR, | 100 MHz, | 150.9 MHz, | Δδ = δ (this work) - |
| Assignment | 75.4 MHz, CDCl₃ | CDCl₃ | CDCl₃ | CDCl₃ | δ (Numata's report) |
| C2 | 36.03 | 35.7 | 36.3 | 36.45 | 0.42⁶⁷ |
| C3 | 38.03 | 37.8 | 38.0 | 38.19 | 0.16 |
| C3a | 51.42 | 50.7 | 51.4 | 51.59 | 0.17 |
| C4a | 132.23 | 132.5 | 132.3 | 132.40 | 0.17 |
| C4 | 136.78 | 137.0 | 136.8 | 136.96 | 0.18 |
| C5 | 113.19 | 112.7 | 113.2 | 113.36 | 0.17 |
| C6 | 128.89 | 128.2 | 128.9 | 129.04 | 0.15 |
| C7 | 101.81 | 101.3 | 101.8 | 101.97 | 0.16 |
| C7a | 150.55 | 150.6 | 150.6 | 150.71 | 0.16 |
| N8CH₃ | 29.63 | 29.6 | 29.6 | 29.78 | 0.15 |
| C8a | 82.44 | 81.4 | 82.5 | 82.62 | 0.18 |
| C9 | 65.38 | 64.5 | 65.4 | 65.54 | 0.16 |
| C10 | 64.01 | 63.1 | 64.0 | 64.16 | 0.15 |
| C11 | 59.79 | 59.2 | 59.8 | 59.90 | 0.11 |
| C12 | 24.80 | 24.5 | 24.8 | 24.94 | 0.14 |
| C13 | 20.50 | 20.1 | 20.5 | 20.64 | 0.14 |
| C2′ | 44.08 | 43.5 | 44.1 | 44.21 | 0.13 |
| C3′ | 30.81 | 30.4 | 30.8 | 30.97 | 0.16 |
| C3a′ | 51.92 | 51.4 | 52.0 | 52.10 | 0.18 |
| C4a′ | 132.38 | 132.1 | 132.4 | 132.56 | 0.18 |
| C4′ | 123.21 | 123.3 | 123.2 | 123.38 | 0.17 |
| C5′ | 120.54 | 118.7 | 120.6 | 120.71 | 0.17 |
| C6′ | 127.43 | 126.9 | 127.4 | 127.56 | 0.13 |
| C7′ | 116.97 | 116.5 | 117.0 | 117.10 | 0.13 |
| C7a′ | 142.69 | 144.1 | 142.7 | 142.80 | 0.11 |
| C8a′ | 79.65 | 78.4 | 79.7 | 79.79 | 0.14 |
| C1″ | 170.02 | 170.9 | 172.1 | 172.12 | 2.10⁶⁷ |
| C2″ | 22.65 | 22.2 | 22.6 | 22.80 | 0.15 |

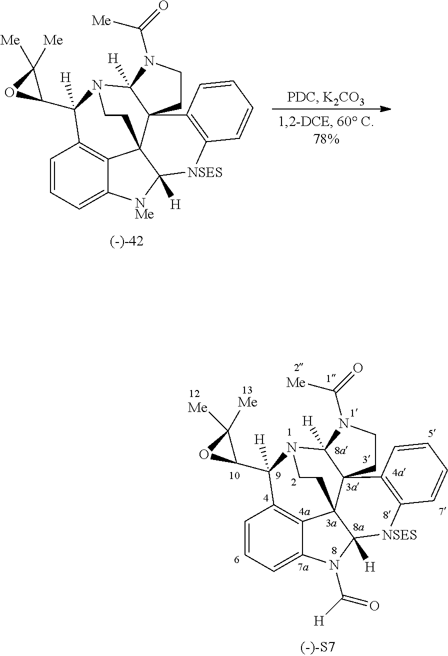

### Example 26: Heptacyclic Formamide (−)-S7

Samples of crushed potassium carbonate (123 mg, 891 μmol, 40.0 equiv) and pyridinium dichromate (PDC, 83.8 mg, 223 μmol, 10.0 equiv) were added sequentially to a solution of (−)-N8′-(trimethylsilyl)ethanesulfonyl communesin A (42, 13.8 mg, 22.3 μmol, 1 equiv) in 1,2-dichloroethane (1.50 mL) at 23° C. The flask was sealed with a Teflon-lined glass stopper under an argon atmosphere and was immersed in a preheated oil bath at 60° C. After stirring for 8 h, the brown suspension was cooled to 23° C., was diluted with dichloromethane (5 mL), and was filtered through a pad of silica gel covered with a pad of celite. The filter cake was washed with acetone-dichloromethane (1:1, 70 mL) and the filtrate was concentrated under reduced pressure. The resulting residue was purified by flash column chromatography on silica gel (eluent: 20% isopropanol in hexanes) to afford heptacyclic formamide (−)-S7 (11.0 mg, 77.6%) as a white solid. ¹H NMR (600 MHz, CDCl₃, 20° C., 23:9.6*:2.0:1.0 mixture of atropisomers, *denotes minor atropisomer): δ 8.83 (s, 1H), 8.81* (s, 1H), 7.25-7.15 (m, 2H), 7.12-7.00 (m, 2H), 6.81 (dd, J=11.3, 7.4 Hz, 1H), 6.75 (app-t, J=7.3 Hz, 1H), 6.64* (d, J=8.1 Hz, 1H), 6.62 (d, J=7.7 Hz, 1H), 6.43 (s, 1H), 6.38* (s, 1H), 5.41* (s, 1H), 5.01 (s, 1H), 4.59* (d, J=8.1 Hz, 1H), 4.18 (d, J=8.9 Hz, 1H), 4.11 (td, J=13.8, 3.9 Hz, 1H), 3.95 (dd, J=12.3, 8.7 Hz, 1H), 3.77* (app-t, J=9.4 Hz, 1H), 3.59-3.45 (m, 2H), 3.44-3.35 (m, 1H), 3.30-3.21* (m, 1H), 3.10 (app-td, J=11.4, 7.4 Hz, 1H), 3.06-2.97* (m, 1H), 2.88-2.81 (m, 1H), 2.79 (d, J=8.9 Hz, 1H), 2.71* (d, J=8.5 Hz, 1H), 2.67 (dd, J=14.1, 8.2 Hz, 1H), 2.44* (dd, J=14.1, 7.3 Hz, 1H), 2.32 (s, 3H), 2.37-2.25 (m, 2H), 2.10* (s, 3H), 1.57* (s, 3H), 1.54 (s, 3H), 1.37 (s, 3H), 1.35* (s, 3H), 1.31-1.15 (m, 2H), 0.16 (s, 9H). ¹³C NMR (150.9 MHz, CDCl₃, 20° C., mixture of atropisomers):⁶⁸ δ 171.5, 170.7, 159.3, 141.2, 140.2, 139.9, 139.6, 139.2, 138.8, 135.6 (2C), 134.4, 134.2, 129.5, 129.3, 128.1, 127.9, 127.3, 127.2, 127.1, 126.6, 124.2, 123.8, 122.6, 122.1, 107.1, 106.7, 79.9, 78.5, 78.1, 76.9, 76.6, 65.2, 65.0, 63.8, 59.9, 59.8, 54.8, 54.6, 52.8, 52.7, 52.3, 50.0, 46.0, 44.3, 37.8, 37.1, 36.8, 36.4, 32.5, 30.9, 24.8 (2C), 23.1, 22.9, 20.6, 20.4, 10.9, 10.8, −1.5, −1.7, −1.8, (2C). FTIR (thin film) cm⁻¹: 2954 (w), 2895 (w), 1682 (s), 1651 (s), 1592 (m), 1486 (m), 1468 (m), 1400 (m), 1342 (s), 1250 (m), 1072 (m), 893 (m), 842 (m), 701 (m). HRMS (ESI) (m/z): calc'd for C₃₃H₄₃N₄O₅SSi [M+H]⁺: 635.2718, found: 635.2715. [α]_{D}²³: −56 (c=0.49, CH₂Cl₂). TLC (20% isopropanol in hexanes), Rf: 0.21 (UV, CAM).

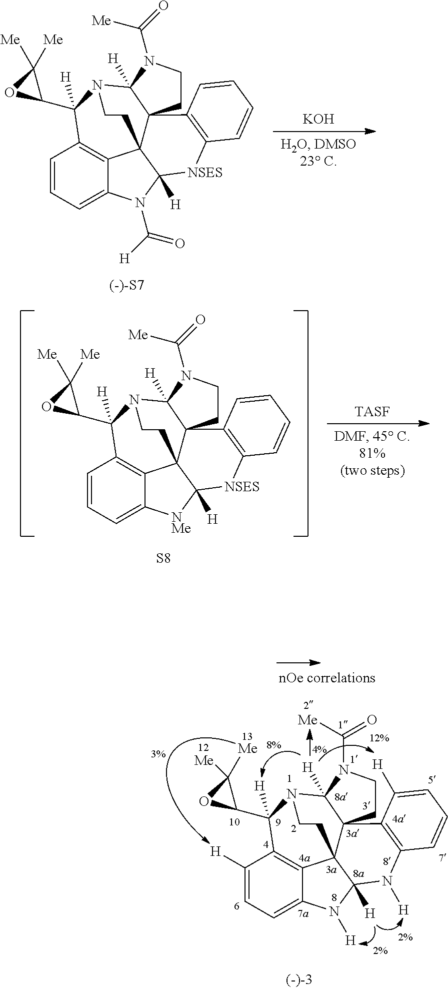

### Example 27: (−)-Communesin E (3)

An aqueous potassium hydroxide solution (0.5 M, 172 μL, 86.0 μmol, 5.00 equiv) was added rapidly to a solution of heptacyclic formamide (−)-S7 (10.9 mg, 17.2 μmol, 1 equiv) in dimethyl sulfoxide (1.72 mL) and deionized water (172 L) at 23° C. After 25 min, the light-yellow homogeneous solution was diluted with a saturated aqueous sodium chloride solution (20 mL) and the mixture was extracted with ethyl acetate (3×10 mL). The combined organic extracts were washed with a saturated aqueous sodium chloride solution (2×20 mL), were dried over anhydrous sodium sulfate, were filtered, and were concentrated under reduced pressure. The resulting residue was purified by flash column chromatography on silica gel (eluent: 40% acetone in hexanes) to afford sulfonamide S8 as a colourless film, contaminated with a trace amount of (−)-3. This mixture was used directly in the next step without further purification.

A degassed solution of tris(dimethylamino)sulfonium difluorotrimethylsilicate (TASF, 19.0 mg, 68.8 μmol, 4.00 equiv) in N,N-dimethylformamide (175 μL) was added to a degassed solution of sulfonamide S8 (1 equiv) in N,N-dimethylformamide (400 μL) at 23° C. The flask was then immersed in a preheated oil bath at 45° C. After 2 h, the solution was cooled to 23° C. and a saturated aqueous sodium chloride solution (10 mL) and deionized water (5 mL) were added and the mixture was extracted with ethyl acetate (3×10 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 50→60% ethyl acetate in dichloromethane) to afford (−)-communesin E (3, 6.14 mg, 80.9% over two steps) as a off-white film. Structural assignments were made using additional information from gCOSY, gHSQC, gHMBC, and 1D selective NOESY experiments. ¹H NMR (500 MHz, CDCl₃, 21° C., 11:1 mixture of atropisomers, *denotes minor atropisomer): δ 7.02 (ddd, J=7.6, 6.4, 2.7 Hz, 2H, C6′H, C6′H*), 6.85 (app-t, J=7.6 Hz, 1H, C6H), 6.84 (app-t, J=7.6 Hz, 1H, C6H*), 6.75-6.66 (m, 5H, C4′H, C5′H, C5′H*, C7′H, C7′H*), 6.64 (d, J=7.5 Hz, 1H, C4′H*), 6.22 (d, J=7.6 Hz, 1H, C7H), 6.21 (d, J=7.9 Hz, 1H, C5H*), 6.19 (d, J=7.1 Hz, 1H, C7H*), 6.17 (d, J=7.6 Hz, 1H, C5H), 5.41 (s, 1H, C8a′H*), 5.03 (d, J=1.8 Hz, 1H, C8a′H), 5.01 (s, 1H, C8aH), 4.52 (d, J=8.5 Hz, 1H, C9H*), 4.28 (br-s, 1H, N8H/N8′H), 4.20 (br-s, 1H, N8H/N8′H), 4.09 (d, J=9.2 Hz, 1H, C9H), 3.89 (app-dd, J=11.9, 8.2 Hz, 1H, C2′Hₐ), 3.76-3.67 (m, 1H, C2′Hₐ*), 3.60-3.50 (m, 1H, C2Hₐ*), 3.50-3.42 (m, 1H, C2Hₐ), 3.36 (app-dt, J=15.9, 8.9 Hz, 2H, C2H_{b}, C2H_{b}*), 3.19-3.08 (m, 1H, C2′H_{b}*), 3.03 (app-td, J=11.6, 7.3 Hz, 1H, C2′H_{b}), 2.98-2.89 (m, 1H, C3′Hₐ*), 2.86 (d, J=9.2 Hz, 1H, C10H), 2.80 (d, J=8.9 Hz, 1H, C10H*), 2.74 (ddd, J=13.4, 11-6, 8.9 Hz, 1H, C3′Hₐ), 2.42-2.34 (m, 1H, C3Hₐ), 2.34 (s, 3H, C2″H₃), 2.33-2.25 (m, 1H, C3H_{b}), 2.08 (s, 3H, C2″H₃*), 2.02 (app-dd, J=12.8, 7.6 Hz, 1H, C3′H_{b}*), 1.94 (app-dd, J=13.1, 6.7 Hz, 1H, C3′H_{b}), 1.58 (s, 3H, C13H₃*), 1.53 (s, 3H, C13H₃), 1.39 (s, 3H, C12H₃), 1.37 (s, 3H, C12H₃*). ¹³C NMR (150.9 MHz, CDCl₃, 20° C., 11:1 mixture of atropisomers, *denotes minor atropisomer): δ 172.2 (C1″), 171.5 (C1*), 149.8 (C7a), 142.7 (C7a′*), 142.6 (C7a′), 137.6 (C4), 133.0 (C4a), 131.9 (C4a′), 128.8 (C6), 128.7 (C6*), 127.5 (C6′), 127.3 (C6′*), 123.6 (C4′*), 123.3 (C4′), 120.9 (C5′*), 120.5 (C5′), 117.3 (C7′*), 117.0 (C7′), 116.1 (C5*), 115.5 (C5), 106.3 (C7), 106.0 (C7*), 80.0 (C8a′), 78.1 (C8a′*), 77.2 (C8a), 65.6 (C9), 65.2 (C9*), 64.1 (C10), 59.9 (C11), 52.5 (C3a), 52.0 (C3a′), 46.1 (C2′*), 44.2 (C2′), 38.3 (C3), 37.9 (C2*), 36.3 (C2), 32.6 (C3′*), 30.9 (C3′), 25.0 (C12), 24.9 (C12*), 23.2 (C2″*), 22.8 (C2″), 20.6 (C13), 20.5 (C13*). FTIR (thin film) cm⁻¹: 3341 (br-m), 3051 (w), 3028 (w), 2962 (m), 2926 (m), 2879 (m), 1631 (s), 1605 (s), 1481 (m), 1460 (m), 1403 (s), 1348 (m), 1250 (m), 1166 (m), 1084 (m), 1062 (m), 1015 (m), 747 (m). HRMS (ESI) (m/z): calc'd for C27H₃₁N₄O₂ [M+H]⁺: 443.2442, found: 443.2439. [α]_{D}²³: −191 (c=0.31, CHCl₃).⁶⁹ TLC (50% ethyl acetate in dichloromethane), Rf: 0.11 (UV, CAM).

TABLE 4
Comparison of ¹H NMR data for (−)-communesin E (3) with literature data
(CDCl₃, major atropisomer):

| | Hayashi's Isolation Report^{64,65} | This Work |
|---|---|---|
| Assignment | ¹H NMR, CDCl₃ | ¹H NMR, 500 MHz, CDCl₃ |
| C2 | 3.46 (ddd, J = 15.9, 8.9, 1.8 Hz, 1H) | 3.50-3.42 (m, 1H) |
| | 3.36 (dt, J = 15.9, 9.2 Hz, 1H) | 3.36 (app-dt, J = 15.9, 8.9 Hz, 1H) |
| C3 | 2.37 (ddd, J = 12.8, 9.2, 1.8 Hz, 1H) | 2.42-2.34 (m, 1H) |
| | 2.30 (ddd, J = 12.8, 9.2, 8.9 Hz, 1H) | 2.33-2.25 (m, 1H) |
| C3a | — | — |
| C4a | — | — |
| C4 | — | — |
| C5 | 6.17 (d, J = 7.6 Hz, 1H) | 6.17 (d, J = 7.6 Hz, 1H) |
| C6 | 6.85 (d, J = 7.6 Hz, 1H) | 6.85 (d, J = 7.6 Hz, 1H) |
| C7 | 6.22 (d, J = 7.6 Hz, 1H) | 6.22 (d, J = 7.6 Hz, 1H) |
| C7a | — | — |
| C8a | 5.02 (s, 1H) | 5.01 (s, 1H) |
| C9 | 4.10 (d, J = 9.1 Hz, 1H) | 4.09 (d, J = 9.2 Hz, 1H) |
| C10 | 2.87 (d, J = 9.1 Hz, 1H) | 2.86 (d, J = 9.2 Hz, 1H) |
| C11 | — | — |
| C12 | 1.39 (s, 3H) | 1.39 (s, 3H) |
| C13 | 1.54 (s, 3H) | 1.53 (s, 3H) |
| C2′ | 3.90 (dd, J = 11.6, 8.6 Hz, 1H) | 3.89 (app-dd, J = 11.9, 8.2 Hz, 1H) |
| | 3.03 (td, J = 11.6, 7.3 Hz, 1H) | 3.03 (app-td, J = 11.6, 7.3 Hz, 1H) |
| C3′ | 2.74 (ddd, J = 13.4, 11.6, 8.6 Hz, 1H) | 2.74 (ddd, J = 13.4, 11.6, 8.9 Hz, 1H) |
| | 1.95 (dd, J = 13.4, 7.3 Hz, 1H) | 1.94 (app-dd, J = 13.1, 6.7 Hz, 1H) |
| C3a′ | — | — |
| C4a′ | — | — |
| C4′ | 6.71 (m, 3H) | 6.75-6.66 (m, 3H) |
| C5′ | 6.71 (m, 3H) | 6.75-6.66 (m, 3H) |
| C6′ | 7.02 (ddd, J = 7.9, 6.7, 2.1 Hz, 1H) | 7.02 (ddd, J = 7.6, 6.4, 2.7 Hz, 1H) |
| C7′ | 6.71 (m, 3H) | 6.75-6.66 (m, 3H) |
| C7a′ | — | — |
| C8a′ | 5.04 (s, 1H) | 5.03 (d, J = 1.8 Hz, 1H) |
| C1″ | — | — |
| C2″ | 2.35 (s, 3H) | 2.34 (s, 3H) |
| N8H / N8′H | — | 4.28 (br-s, 1H) |
| N8H / N8′H | — | 4.20 (br-s, 1H) |

TABLE 5
Comparison of ¹³C NMR data for (−)-communesin E (3) with
literature data (CDCl₃, major atropisomer):

| | Hayashi's | This Work | Chemical Shift |
|---|---|---|---|
| | Isolation | ¹³C NMR, | Difference |
| | Report^{64,65} | 150.9 | Δδ = δ (this work) - |
| Assignment | ¹³C NMR, CDCl₃ | MHz, CDCl₃ | δ (Hayashi report) |
| C2 | 36.2 | 36.3 | 0.1 |
| C3 | 38.1 | 38.3 | 0.2 |
| C3a | 51.8 | 52.5 | 0.7⁷⁰ |
| C4a | 132.8 | 133.0 | 0.2 |
| C4 | 137.4 | 137.6 | 0.2 |
| C5 | 115.2 | 115.4 | 0.2 |
| C6 | 128.7 | 128.8 | 0.1 |
| C7 | 106.1 | 106.3 | 0.2 |
| C7a | 149.6 | 149.8 | 0.2 |
| C8a | 77.0 | 77.2 | 0.2 |
| C9 | 65.4 | 65.6 | 0.2 |
| C10 | 64.0 | 64.1 | 0.1 |
| C11 | 59.8 | 59.9 | 0.1 |
| C12 | 24.8 | 25.0 | 0.2 |
| C13 | 20.5 | 20.6 | 0.1 |
| C2′ | 44.1 | 44.2 | 0.1 |
| C3′ | 30.7 | 30.9 | 0.2 |
| C3a′ | 52.8 | 52.0 | −0.8⁷⁰ |
| C4a′ | 131.6 | 131.9 | 0.3 |
| C4′ | 123.1 | 123.3 | 0.2 |
| C5′ | 120.3 | 120.5 | 0.2 |
| C6′ | 127.3 | 127.5 | 0.2 |
| C7′ | 116.9 | 117.1 | 0.2 |
| C7a′ | 142.5 | 142.6 | 0.1 |
| C8a′ | 79.8 | 80.0 | 0.2 |
| C1″ | 172.1 | 172.2 | 0.1 |
| C2″ | 22.7 | 22.8 | 0.1 |

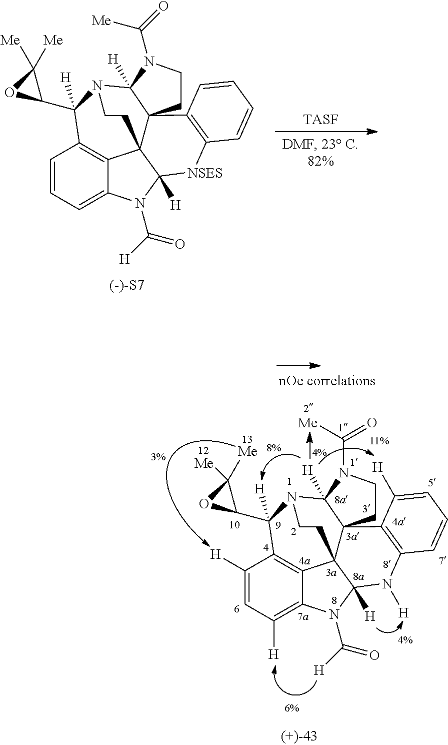

### Example 28: (+)-N8-Formyl Communesin E(43)

A solution of tris(dimethylamino)sulfonium difluorotrimethyl silicate (TASF, 16.7 mg, 60.5 μmol, 4.00 equiv) in N,N-dimethylformamide (200 μL) was added to a solution of heptacyclic formamide (−)-S7 (9.60 mg, 15.1 μmol, 1equiv) in N,N-dimethylformamide (300 μL) at 23° C. After 2 h, a saturated aqueous sodium chloride solution (10 mL) and deionized water (5 mL) were added and the mixture was extracted with ethyl acetate (3×8 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 40%→50% acetone in hexanes) to afford (+)-N8-formyl communesin E (43, 5.83 mg, 81.9%) as a white solid. Structural assignments were made using additional information from gCOSY, gHSQC, gHMBC, and 1D selective NOESY experiments. ¹H NMR (500 MHz, CDCl₃, 20° C., 48:8.9*:1** mixture of atropisomers, * and ** denote minor atropisomers): δ 6 8.91 (d, J=0.8 Hz, 1H, N8CHO), 8.89 (s, 1H, N8CHO*), 8.70 (s, 1H, N8CHO**), 7.09-6.99 (m, 4H, C6H, C6H*, C6′H, C6′H*), 6.81 (d, J=7.9 Hz, 1H, C7H), 6.76 (d, J=8.1 Hz, 1H, C7H*), 6.74 (td, J=7.5, 1.4 Hz, 2H, C5′H, C5′H*), 6.72-6.67 (m, 3H, C4′H, C7′H, C7′H*), 6.63 (d, J=7.6 Hz, 1H, C4′H*), 6.62 (d, J=8.1 Hz, 1H, C5H*), 6.59 (d, J=7.8 Hz, 1H, C5H), 5.55 (app-t, J=1.4 Hz, 1H, C8aH), 5.52 (d, J=2.3 Hz, 1H, C8aH*), 5.43 (d, J=2.0 Hz, 1H, C8aH**), 5.42-5.36 (m, 2H, C8a′H*, N8′H), 5.05 (s, 1H, C8a′H**), 5.02 (d, J=1.7 Hz, 1H, C8a′H), 4.61-4.52 (m, 1H, C9H*), 4.16 (d, J=8.9 Hz, 1H, C9H), 3.91 (app-dd, J=11.9, 8.4 Hz, 1H, C2′Hₐ), 3.72 (app-t, J=8.9 Hz, 1H, C2′Hₐ*), 3.57-3.51 (m, 1H, C2Hₐ*), 3.48 (app-dd, J=15.9, 9.6 Hz, 1H, C2Hₐ), 3.44-3.34 (m, 2H, C2H_{b}, C2H_{b}*), 3.14 (app-td, J=10.8, 7.5 Hz, 1H, C2′H_{b}*), 3.00 (app-td, J=11.6, 7.2 Hz, 1H, C2′H_{b}), 2.98-2.90 (m 1H, C3′Hₐ*), 2.84 (d, J=8.9 Hz, 1H, C10H), 2.80-2.70 (m, 2H, C10H*, C3′Hₐ), 2.52-2.45 (m, 1H, C3Hₐ*), 2.49 (ddd, J=13.3, 8.7, 2.0 Hz, 1H, C3Hₐ), 2.36-2.25 (m, 2H, C3H_{b}, C3H_{b}*), 2.33 (s, 3H, C2″H₃), 2.12 (dd, J=13.4, 7.0 Hz, 1H, C3′H_{b}*), 2.10 (s, 3H, C2″H₃*), 2.06 (dd, J=13.4, 6.8 Hz, 1H, C3′H_{b}), 1.58 (br-s, 3H, C13H₃*), 1.55 (s, 3H, C13H₃), 1.40 (s, 3H, C12H₃), 1.38 (s, 3H, C12H₃*). ¹³C NMR (150.9 MHz, CDCl₃, 20° C., 48:8.9*:1 mixture of atropisomers, * denotes minor atropisomer): δ 171.9 (C1″), 170.9 (C1″*), 158.2 (2C, N8CHO*, N8CHO), 141.7 (C7a′*), 141.5 (C7a′), 140.5 (C7a), 140.3 (C7a*), 139.4 (C4), 135.2 (C4a), 135.0 (C4a*), 131.6 (C4a′*), 131.2 (C4a′), 129.1 (C6), 128.9 (C6*), 128.0 (C6′), 127.8 (C6′*), 123.4 (C4′*), 123.2 (C4′), 122.5 (C5*), 121.9 (C5), 121.4 (C5′*), 121.1 (C5′), 117.4 (2C, C7′*, C7′), 106.5 (C7), 106.1 (C7*), 79.3 (C8a′), 77.8 (C8a*), 77.5 (C8a′*), 77.4 (C8a), 65.3 (C9), 64.9 (C9*), 63.9 (2C, C10, C10*), 60.0 (C11), 59.9 (C11*), 51.9 (C3a′), 50.9 (C3a), 50.8 (C3a*), 49.6 (C3a′*), 45.9 (C2′*), 44.1 (C2′), 38.0 (C3*), 37.8 (C3), 37.6 (C2*), 36.3 (C2), 32.5 (C3′*), 30.8 (C3′), 24.9 (C12), 24.8 (C12*), 23.1 (C2″*), 22.8 (C2″), 20.6 (C13), 20.4 (C13*). FTIR (thin film) cm⁻¹: 3292 (br-w), 3056 (w), 2978 (w), 2959 (w), 2926 (w), 2876 (w), 1662 (s), 1640 (s), 1585 (m), 1495 (m), 1466 (m), 1415 (s), 1346 (m), 1254 (m), 750 (m). HRMS (ESI) (m/z): calc'd for C₂₈H₃₁N₄O₃ [M+H]⁺: 471.2391, found: 471.2392. [α]_{D}²³: +60 (c=0.29, CHCl₃). TLC (40% acetone in hexanes), Rf: 0.11 (UV, CAM).

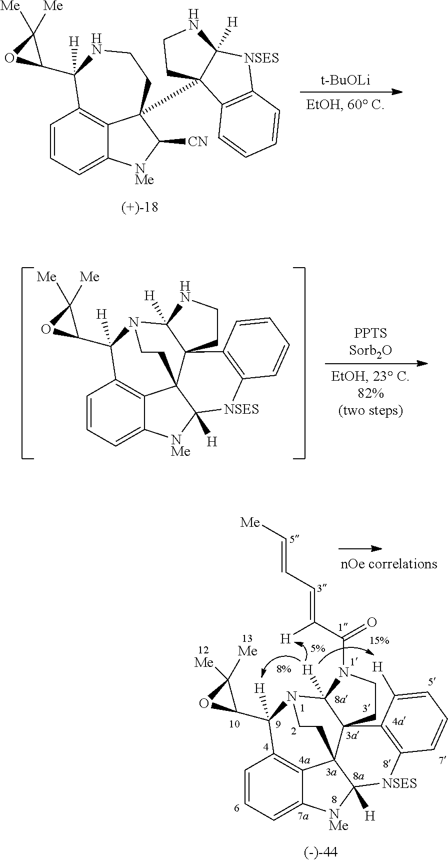

### Example 29: (−)-N8′-(Trimethylsilyl)Ethanesulfonyl Communesin B (44)

A sample of lithium tert-butoxide (21.5 mg, 268 μmol, 10.0 equiv) was added to a solution of heterodimer (+)-18 (16.3 mg, 26.8 μmol, 1 equiv) in anhydrous ethanol (200 proof, 700 μL) at 23° C. The flask was sealed with a Teflon-lined glass stopper under an argon atmosphere and was immersed in a preheated oil bath at 60° C. After 20 h, the reaction mixture was cooled to 23° C. and samples of pyridinium p-toluenesulfonate (PPTS, 54.0 mg, 215 μmol, 8.00 equiv) and sorbic anhydride⁷¹ (22.1 mg, 107 μmol, 4.00 equiv) were added sequentially. After 30 min, a saturated aqueous sodium bicarbonate solution (10 mL) and deionized water (10 mL) were added and the resulting mixture was extracted with ethyl acetate (3×10 mL). The combined organic extracts were washed with a saturated aqueous sodium chloride solution (15 mL), were dried over anhydrous sodium sulfate, were filtered, and were concentrated under reduced pressure. The residue was purified by flash column chromatography on silica gel (eluent: 35%→40% ethyl acetate in hexanes) to afford (−)-N8′-(trimethylsilyl)ethanesulfonyl communesin B (44, 14.8 mg, 82.1%) as a white solid. Crystals suitable for X-ray diffraction were obtained by slow evaporation a solution of (−)-39 in ethanol at 0° C. The thermal ellipsoid representation of (−)-39 is depicted later in this document. Structural assignments were made using additional information from gCOSY, gHSQC, gHMBC, and 1D selective NOESY experiments. ¹H NMR (500 MHz, CDCl₃, 20° C., 6.8:1 mixture of atropisomers, *denotes minor atropisomer): δ 7.57 (dd, J=8.0, 1.2 Hz, 1H, C7′H), 7.41 (d, J=8.0 Hz, 1H, C7′H*), 7.32 (dd, J=15.1, 10.5 Hz, 2H, C3″H, C3″H*), 7.17 (app-td, J=7.7, 1.4 Hz, 2H, C6′H, C6′H*), 7.02 (app-td, J=7.6, 1.3 Hz, 2H, C5′H, C5′H*), 6.91 (t, J=7.7 Hz, 1H, C6H), 6.90 (t, J=7.4 Hz, 1H, C6H*), 6.80 (dd, J=7.9, 1.4 Hz, 1H, C4′H), 6.77 (app-d, J=7.8 Hz, 1H, C4′H*), 6.48 (d, J=15.1 Hz, 1H, C2″H), 6.24-6.09 (m, 6H, C4″H, C4″H*, C5″H, C5″H*, C5H, C5H*), 6.08 (d, J=16.2 Hz, 1H, C2″H*), 5.98 (d, J=7.8 Hz, 1H, C7H), 5.94 (d, J=7.7 Hz, 1H, C7H*), 5.73 (s, 1H, C8aH), 5.65 (s, 1H, C8aH*), 5.54 (app-s, 1H, C8a′H*), 5.13 (app-s, 1H, C8a′H), 4.58-4.50 (m, 1H, C9H*), 4.18 (d, J=9.0 Hz, 1H, C9H), 3.92 (app-dd, J=12.2, 8.5 Hz, 2H, C2′Hₐ, C2′Hₐ*), 3.61-3.54 (m, 1H, C2Hₐ*), 3.50 (app-dd, J=16.0, 9.6 Hz, 1H, C2Hₐ), 3.41 (app-dt, J=16.2, 8.6 Hz, 2H, C2H_{b}, C₂H_{b}*), 3.33-3.24 (m, 1H, C2′H_{b}*), 3.23 (app-td, J=13.5, 4.6 Hz, 2H, N8′SO₂CHa, N8′SO₂CHa*), 3.19-3.10 (m, 3H, C2′H_{b}, N8′SO₂CH_{b}, N8′SO₂CH_{b}*), 3.08-2.98 (m, 1H, C3′Hₐ*), 2.93 (s, 3H, N8CH₃), 2.88 (d, J=9.0 Hz, 1H, C10H), 2.87 (s, 3H, N8CH₃*), 2.81 (app-td, J=12.0, 8.6 Hz, 2H, C10H*, C3′Hₐ), 2.56-2.44 (m, 2H, C3Hₐ, C3Hₐ*), 2.30 (app-dt, J=13.1, 9.2 Hz, 2H, C3H_{b}, C3H_{b}*), 2.12 (app-dd, J=13.3, 7.5 Hz, 1H, C3′H_{b}*), 1.93 (app-dd, J=13.0, 7.2 Hz, 1H, C3′H_{b}), 1.85 (d, J=6.4 Hz, 3H, C6″H₃), 1.83 (d, J=6.8 Hz, 3H, C6″H₃*), 1.64 (s, 3H, C12/13H₃), 1.60 (s, 3H, C12/13H₃*), 1.41 (s, 3H, C12/13H₃), 1.35 (s, 3H, C12/13H₃*), 1.32-1.20 (m, 2H, N8′SO₂CH₂CH₂*), 1.23 (td, J=13.8, 4.4 Hz, 1H, N8′SO₂CH₂CHa), 1.16 (td, J=13.7, 4.5 Hz, 1H, N8′SO₂CH₂CH_{b}), 0.10 (s, 9H, Si(CH₃)₃*), 0.06 (s, 9H, Si(CH₃)₃). ¹³C NMR (150.9 MHz, CDCl₃, 20° C., 6.8:1 mixture of atropisomers, *denotes minor atropisomer): δ 168.2 (C1″), 166.5 (C1″*), 149.9 (C7a), 149.5 (C7a*), 143.3 (C3″*), 142.3 (C3″), 139.0 (2C, C4*, C4a′*), 138.4 (C5″*), 138.1 (C4a′), 137.7 (C5″), 137.0 (C4), 136.0 (2C, C7a′, C7a′*), 131.2 (2C, C4a, C4a*), 130.7 (C4″), 130.3 (C4″*), 129.4 (C6), 129.2 (C6*), 127.9 (C6′), 127.7 (C6′*), 126.8 (C5′*), 126.3 (C5′), 125.2 (C7′*), 124.6 (C7′), 124.4 (C4′*), 124.2 (C4′), 120.9 (C2′), 119.6 (C2″*), 114.6 (C5*), 113.8 (C5), 102.6 (C7), 102.3 (C7*), 85.2 (C8a*), 84.7 (C8a), 79.3 (C8a′), 78.6 (C8a′*), 65.7 (C9), 65.3 (C9*), 64.0 (C10*), 63.9 (C10), 59.9 (2C, C11*, C11), 54.2 (C3a), 54.1 (C3a*), 52.7 (C3a′), 52.4 (N8′SO₂CH₂*), 51.7 (N8′SO₂CH₂), 50.1 (C3a′*), 45.2 (C2′*), 44.2 (C2′), 38.1 (C2*), 37.9 (C3*), 37.5 (C3), 36.3 (C2), 33.3 (C3′*), 31.4 (C3′), 31.0 (N8CH₃*), 30.9 (N8CH₃), 25.0 (C12/13), 24.9 (C12/13*), 20.7 (C12/13), 20.6 (C12/13*), 18.9 (C6″), 18.8 (C6″*), 10.8 (N8′SO₂CH₂CH₂*), 10.7 (N8′SO₂CH₂CH₂), −1.7 (Si(CH₃)₃*), −1.8 (Si(CH₃)₃). FTIR (thin film) cm⁻¹: 3055 (w), 2956 (m), 1708 (w), 1654 (m), 1627 (m), 1599 (s), 1487 (m), 1391 (s), 1338 (s), 1284 (m), 1250 (m), 1156 (s), 1052 (m), 1000 (m), 859 (m), 760 (m), 564 (m). HRMS (ESI) (m/z): calc'd for C₃₇H₄₉N₄O₄SSi [M+H]⁺: 673.3238, found: 673.3216. [α]_{D}²²: −60 (c=0.74, CH₂Cl₂). TLC (40% ethyl acetate in hexanes), Rf: 0.23 (UV, CAM).

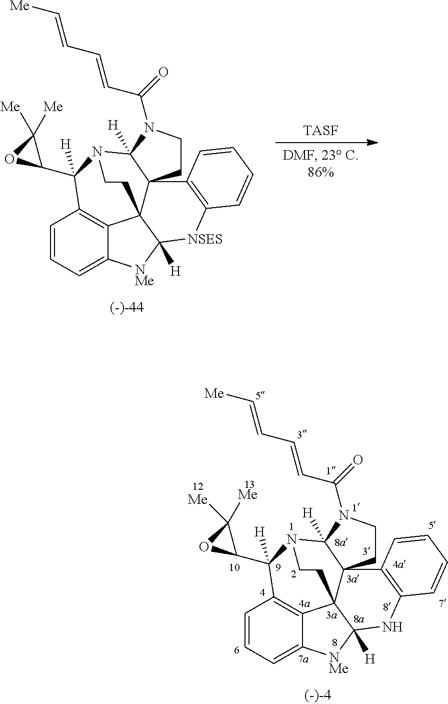

### Example 30: (−)-Communesin B (4)

A solution of tris(dimethylamino)sulfonium difluorotrimethylsilicate (TASF, 23.3 mg, 84.6 μmol, 4.00 equiv) in N,N-dimethylformamide (200 μL) was added to a solution of (−)-N8′-(trimethylsilyl)ethane-sulfonyl communesin B (44, 14.2 mg, 21.2 μmol, 1 equiv) in N,N-dimethylformamide (500 L) at 23° C. After 2 h, a saturated aqueous sodium chloride solution (10 mL) and deionized water (5 mL) were added and the mixture was extracted with ethyl acetate (3×10 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 30% acetone in hexanes) to afford (−)-communesin B (4, 9.27 mg, 86.2%) as a white solid. Structural assignments were made using additional information from gCOSY, gHSQC, gHMBC, and 1D selective NOESY experiments. ¹H NMR (500 MHz, CDCl₃, 20° C., 11:1 mixture of atropisomers, *denotes minor atropisomer): δ 7.31 (dd, J=15.1, 10.6 Hz, 1H, C3″H), 6.98 (ddd, J=7.7, 5.9, 3.0 Hz, 1H, C6′H), 6.88 (t, J=7.7 Hz, 1H, C6H), 6.74-6.59 (m, 3H, C4′H, C5′H, C7′H), 6.54 (d, J=15.1 Hz, 1H, C2″H), 6.19 (ddd, J=14.9, 10.7, 1.6 Hz, 1H, C4″H), 6.14-6.04 (m, 2H, C5H, C5″H), 5.95 (d, J=7.8 Hz, 1H, C7H), 5.54 (s, 1H, C8a′H*), 5.10 (d, J=1.5 Hz, 1H, C8a′H), 4.70 (s, 1H, C8aH), 4.62 (br-s, 1H, N8′H), 4.17 (d, J=9.0 Hz, 1H, C9H), 3.87 (app-dd, J=12.1, 8.3 Hz, 1H, C2′Hₐ), 3.53-3.36 (m, 2H, C2H₂), 3.07 (app-td, J=11.8, 7.0 Hz, 1H, C2′H_{b}), 2.90 (d, J=9.0 Hz, 1H, C10H), 2.85 (s, 3H, N8CH₃), 2.72 (app-td, J=12.4, 8.4 Hz, 1H, C3′Hₐ), 2.37 (ddd, J=13.0, 8.5, 2.2 Hz, 1H, C3Hₐ), 2.28 (app-dt, J=12.9, 9.2 Hz, 1H, C3′H_{b}), 1.99 (dd, J=13.1, 6.9 Hz, 1H, C3′H_{b}), 1.85 (dd, J=6.6, 1.3 Hz, 3H, C6″H₃), 1.65 (s, 3H, C13H₃), 1.42 (s, 3H, C12H₃), 1.37 (s, 3H, C12H₃*). ¹³C NMR (150.9 z, CDCl₃, 20° C., 11:1 mixture of atropisomers, * denotes minor atropisomer): δ 168.5 (C1″), 150.6 (C7a), 142.7 (C7a′), 141.9 (C3″), 137.3 (C5″), 136.7 (C4), 132.4 (2C, C4a, C4a′), 130.8 (C4″), 129.0 (C6), 127.5 (C6′), 123.5 (C4′), 121.4 (C2″), 120.7 (C5′), 116.9 (C7′), 113.4 (C5), 102.0 (C7), 82.5 (C8a), 79.1 (C8a′), 65.7 (C9), 64.1 (C10), 59.9 (C11), 52.3 (C3a′), 51.50 (C3a), 44.3 (C), 38.0 (C3), 36.2 (C2), 30.6 (C3′), 29.7 (N8CH₃), 25.0 (C12), 24.9 (C12*), 20.7 (C13), 18.9 (C6″), 18.8 (C6*). FTR (thin film) cm: 3319 (br-w), 3052 (w), 2960 (d), 2926 (m), 2877 (m), 1652 (m), 1625 (m), 1594 (s), 1493 (m), 1474 (m), 1389 (s), 1152 (m), 1000 (s), 737 (m). HRMS (ESI) (m/z): calc'd for C₃₂H₃₇N₄O₂ [M+H]⁺: 509.2911, found: 509.2906. [α]_{D}²³: −64 (c=0.46, CHCl₃).⁷² TLC (30 acetone in hexanes), Rf 0.20 (UV, CAM).

TABLE 6
Comparison of 1H NMR data for (−)-communesin B (4) with literature data
(CDCl3, major atropisomer):

| | Numata's | Hayashi's solation | | |
|---|---|---|---|---|
| | Isolation Report^{73,74} | Report^{75,76} | Ma's Report⁷⁷ | This Work |
| | (−)-Communesin | (−)-Communesin | (−)-Communesin | (−)-Communesin |
| | B (4) ¹H NMR, | B (4) ¹H NMR, | B(4) 1H NMR, | B(4) ¹H NMR, |
| Assignment | 300 MHz, CDCl₃ | CDCl₃ | 400 MHz, CDCl₃ | 500 MHz, CDCl₃ |
| C2 | 3.48 (dd, J = 16.0, | 3.47 (ddt, J = 15.9, | 3.51-3.38 (m, 2H) | 3.53-3.36 (m, 2H) |
| | 9.2 Hz, 1H) | 9.0, 1.0 Hz, 1H) | | |
| | 3.40 (dt, J = 16.0, | 3.41 (ddd, J = 15.9, | | |
| | 8.5 Hz, 1H) | 9.8, 8.0 Hz, 1H) | | |
| C3 | 2.34 (ddd, J = 12.8, | 2.37 (ddd, J = 12.8, | 2.39-2.33 (m, 1H) | 2.37 (ddd, J = 13.0, |
| | 9.2, 8.5 Hz, 1H) | 8.0, 1.0 Hz, 1H) | 2.31-224 (m, 1H) | 8.5, 2.2 Hz, 1H) |
| | 2.25 (dd, J = 12.8, | 2.28 (dt, J = 128 | | 2.28 (app-dt, J = |
| | 8.5 Hz, 1H) | 9.0 Hz, 1H) | | 12.9, 9.2 Hz, 1H) |
| C3a | — | — | — | — |
| C4a | — | — | — | — |
| C4 | — | — | — | — |
| C5 | 6.08 (d, J = 7.8 Hz, | 6.08 (d, J = 7.6 Hz, | 6.14-6.08 (m, | 6.14-6.04 (m, |
| C6 | 6.87 (t, J = 7.8 Hz, | 6.88 (t, J = 7.6 Hz, | 6.89 (t, J = 7.5 | 6.88 (t, J = 7.7 |
| C7 | 5.95 (d, J = 7.8 Hz, | 5.95 (d, J = 7.6 Hz, | 5.95 (d, J = 8.0 | 5.95 (d, J = 7.8 |
| C7a | — | — | — | |
| N8CH₃ | 2.85 (s, 3H) | 2.85 (s, 3H) | 2.85 (s, 3H) | 2.85 (s, 3H) |
| C8a | 4.70 (s, 1H) | 4.70 (s, 1H) | 4.70 (s, 1H) | 4.70 (s, 1H) |
| C9 | 4.18 (d, J = 9.0 Hz, | 4.18 (d, J = 9.0 Hz, | 4.18 (d, J = 9.0 | 4.17 (d, J = 9.0 |
| C10 | 2.90 (d, J = 9.0 Hz, | 2.90 (d, J = 9.0 Hz, | 2.90 (d, J = 9.0 | 2.90 (d, J = 9.0 |
| C11 | — | — | — | — |
| C12 | 1.42 (s, 3H) | 1.42 (s, 3H) | 1.42 (s, 3H) | 1.42 (s, 3H) |
| C13 | 1.65 (s, 3H) | 1.65 (s, 3H) | 1.65 (s, 3H) | 1.65 (s, 3H) |
| C2′ | 3.87 (dd, J = 12.5, | 3.87 (dd, J = 11.9, | 3.88 (dd, J = 12.0, | 3.87 (dd, J = 12.1, |
| | 8.4 Hz, 1H) | 8.5 Hz, 1H) | 9.0 Hz, 1H) | 8.3 Hz, 1H) |
| | 3.07 (td, J = 12.5, | 3.07 (dt, J = 11.9, | 3.07 (td, J = 11.5, | 3.07 (app-td, J = |
| | 7.0 Hz, 1H) | 7.0 Hz, 1H) | 7.0 Hz, 1H) | 11.8, 7.0 Hz, 1H) |
| C3′ | 2.71 (td, J = 12.5, | 2.72 (ddd, J = 13.1, | 2.76-2.68 (m, 1H) | 2.72 (app-td, J = |
| | 8.4 Hz, 1H) | 11.9, 8.5 Hz, 1H) | 2.00 (dd, J = 13.2, | 12.4, 8.4 Hz, 1H) |
| | 2.00 (dd, J = 12.5, | 2.00 (dd, J = 13.1, | 6.8 Hz, 1H) | 1.99 (dd, J = 13.1, |
| | 7.0 Hz, 1H) | 7.0 Hz, 1H) | | 6.9 Hz, 1H) |
| C3a′ | — | — | — | — |
| C4a′ | — | — | — | — |
| C4′ | 6.66 (d, J = 3.5 Hz, 1H) | 6.65 (m, 3H) | 6.68-6.66 (m, 3H) | 6.74-6.59 (m, 3H) |
| C5′ | 6.67 (d, J = 7.8 Hz, 1H) | 6.65 (m, 3H) | 6.68-6.66 (m, 3H) | 6.74-6.59 (m, 3H) |
| C6′ | 6.98 (ddd, J = 7.8, | 6.98 (ddd, J = 7.6, | 7.00-6.97 (m, 1H) | 6.98 (ddd, J = 7.7, |
| | 5.2, 3.5 Hz, 1H) | 5.8, 3.3 Hz, 1H) | | 5.9, 3.0 Hz, 1H) |
| C7′ | 6.66 (d, J = 5.2 Hz, 1H) | 6.65 (m, 3H) | 6.68-6.66 (m, 3H) | 6.74-6.59 (m, 3H) |
| C7a′ | — | — | — | — |
| C8a′ | 5.11 (s, 1H) | 5.10 (s, 1H) | 5.11 (s, 1H) | 5.10 (d, J = 1.5 Hz, 1H) |
| C1″ | — | — | — | — |
| C2″ | 6.55 (d, J = 15.2 | 6.55 (d, J = 15.0 | 6.55 (d, J = 15.0 | 6.54 (d, J = 15.1 |
| | Hz, 1H) | Hz, 1H) | Hz, 1H) | Hz, 1H) |
| C3″ | 7.32 (dd, J = 15.2, | 7.32 (dd, J = 15.0, | 7.31 (dd, J = 15.5, | 7.31 (dd, J = 15.1, |
| | 10.1 Hz, 1H) | 10.4 Hz, 1H) | 10.5 Hz, 1H) | 10.6 Hz, 1H) |
| C4″ | 6.18 (dd, J = 15.5, | 6.19 (dd, J = 15.0, | 6.19 (dd, J = 15.0, | 6.19 (ddd, J = 14.9, |
| | 10.1 Hz, 1H) | 10.4 Hz, 1H) | 9.0 Hz, 1H) | 10.7, 1.6 Hz, 1H) |
| C5″ | 6.12 (dd, J = 15.5, | 6.12 (dq, J = 15.0, | 6.14-6.08 (m, 2H) | 6.14-6.04 (m, 2H) |
| | 5.8 Hz, 1H) | 6.7 Hz, 1H) | | |
| C6″ | 1.85 (d, J = 5.8 Hz, | 1.85 (d, J = 6.7 Hz, | 1.85 (d, J = 6.4 | 1.85 (dd, J = 6.6, |
| | 1H) | 1H) | Hz, 1H) | 1.3 Hz, 3H) |
| N8′H | 4.60 (br-s, 1H) | 4.62 (br-s, 1H) | — | 4.62 (br-s, 1H) |

TABLE 7
Comparison of ¹³C NMR data for (−)-communesin B (4) with literature data
(CDCl₃, major atropisomer):

| | Numata's | | | | Chemical |
|---|---|---|---|---|---|
| | Isolation | Hayashi's | | This Work | Shift |
| | Report^{73,74} | Isolation | Ma's Report⁷⁷ | (−)-Communesin | Difference |
| | (−)-Communesin | Report^{75,76} | (−)-Communesin | B (4) | Δδ = δ |
| | B (4) | (−)-Communesin | B (4) | ¹³C NMR, | (this work) - δ |
| | ¹³C NMR, 75.4 | B (4) | ¹³C NMR, 100 | 150. 9 MHz, | (Numata's |
| Assignment | MHz, CDCl₃ | ¹H NMR, CDCl₃ | MHz, CDCl₃ | CDCl₃ | report) |
| C2 | 36.03 | 35.9 | 36.0 | 36.16 | 0.13 |
| C3 | 37.82 | 37.7 | 37.8 | 37.96 | 0.14 |
| C3a | 51.40 | 51.3 | 51.4 | 51.52 | 0.12 |
| C4a | 132.35 | 132.1 | 132.2 | 132.45 | 0.10 |
| C4 | 136.57 | 136.4 | 136.6 | 136.69 | 0.12 |
| C5 | 113.23 | 113.1 | 113.2 | 113.35 | 0.12 |
| C6 | 128.87 | 128.8 | 128.9 | 128.98 | 0.11 |
| C7 | 101.85 | 101.7 | 101.9 | 101.97 | 0.12 |
| C7a | 150.53 | 150.4 | 150.5 | 150.62 | 0.09 |
| N8CH₃ | 29.60 | 29.5 | 29.6 | 29.74 | 0.14 |
| C8a | 82.39 | 82.3 | 82.4 | 82.52 | 0.13 |
| C9 | 65.55 | 65.4 | 65.6 | 65.67 | 0.12 |
| C10 | 63.95 | 63.8 | 64.0 | 64.07 | 0.12 |
| C11 | 59.75 | 59.7 | 59.8 | 59.87 | 0.12 |
| C12 | 24.89 | 24.8 | 24.9 | 25.02 | 0.13 |
| C13 | 20.54 | 20.5 | 20.5 | 20.68 | 0.14 |
| C2′ | 44.21 | 44.1 | 44.2 | 44.33 | 0.12 |
| C3′ | 30.46 | 30.3 | 30.5 | 30.58 | 0.12 |
| C3a′ | 52.14 | 52.0 | 52.1 | 52.26 | 0.12 |
| C4a′ | 132.32 | 132.2 | 132.2 | 132.36 | 0.04 |
| C4' | 123.41 | 123.3 | 123.4 | 123.55 | 0.14 |
| C5′ | 120.52 | 120.4 | 120.5 | 120.65 | 0.13 |
| C6′ | 127.38 | 127.3 | 127.4 | 127.49 | 0.11 |
| C7′ | 116.82 | 116.7 | 116.8 | 116.93 | 0.11 |
| C7a′ | 142.65 | 142.5 | 142.7 | 142.75 | 0.10 |
| C8a′ | 79.00 | 78.9 | 79.0 | 79.11 | 0.11 |
| C1″ | 168.43 | 168.3 | 168.4 | 168.52 | 0.09 |
| C2″ | 121.27 | 121.1 | 121.3 | 121.39 | 0.12 |
| C3″ | 141.83 | 141.7 | 141.8 | 141.93 | 0.10 |
| C4″ | 130.72 | 130.6 | 130.7 | 130.84 | 0.12 |
| C5″ | 137.13 | 137.1 | 137.2 | 137.26 | 0.13 |
| C6″ | 18.71 | 18.7 | 18.7 | 18.85 | 0.14 |

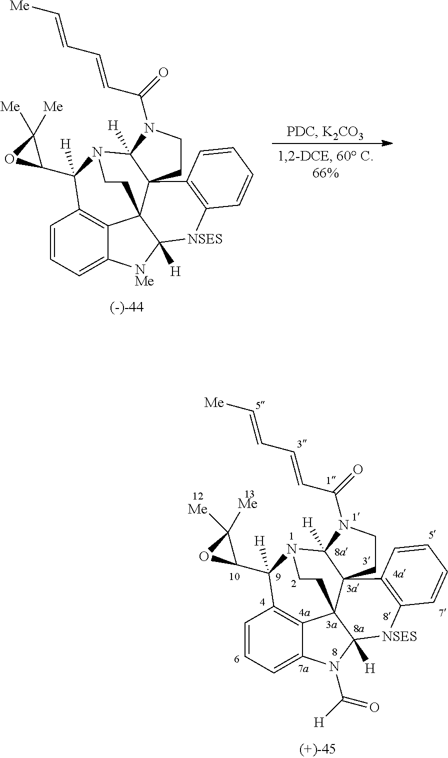

### Example 31: (+)-N8′-(Trimethylsilyl)ethanesulfonyl communesin D (45)

Samples of crushed potassium carbonate (161 mg, 1.17 mmol, 40.0 equiv) and pyridinium dichromate (PDC, 87.7 mg, 233 μmol, 8.00 equiv) were added sequentially to a solution of (−)-N8′-(trimethylsilyl)ethanesulfonyl communesin B (44, 19.6 mg, 29.1 μmol, 1 equiv) in 1,2-dichloroethane (1.90 mL) at 23° C. The flask was sealed with a Teflon-lined glass stopper under an atmosphere of argon and was immersed in a preheated oil bath at 60° C. After stirring for 7 h, the brown suspension was cooled to 23° C., was diluted with dichloromethane (5 mL), and was filtered through a pad of silica gel covered with a pad of celite. The filter cake was washed with acetone-hexanes (1:1, 65 mL) and the colourless filtrate was concentrated under reduced pressure. The resulting residue was purified by flash column chromatography on silica gel (eluent: 40% ethyl acetate in hexanes) to afford (+)-N8′-(trimethylsilyl)ethanesulfonyl communesin D (45, 13.2 mg, 66.0%) as an off-white solid. ¹H NMR (500 MHz, CDCl₃, 20° C., mixture of atropisomers, *denotes minor atropisomers):⁷⁸ δ 8.87* (s, 1H), 8.83* (s, 1H), 8.82 (s, 1H), 8.81* (s, 1H), 7.33 (dd, J=15.1, 10.1 Hz, 1H), 7.23-7.13 (m, 2H), 7.11-7.00 (m, 2H), 6.88-6.72 (m, 2H), 6.68-6.59 (m, 1H), 6.51-6.36 (m, 2H), 6.25-6.04 (m, 2H), 5.54* (s, 1H), 5.51* (s, 1H), 5.28* (s, 1H), 5.18* (s, 1H), 5.15* (s, 1H), 5.09 (s, 1H), 4.63* (d, J=6.6 Hz, 1H), 4.27 (d, J=8.2 Hz, 1H), 4.12 (td, J=13.7, 3.7 Hz, 1H), 3.98-3.83 (m, 1H), 3.60-3.35 (m, 3H), 3.30* (q, J=10.1 Hz, 1H), 3.23* (dd, J=10.2, 7.6 Hz, 1H), 3.14 (td, J=12.0, 7.3 Hz, 1H), 3.03* (dt, J=12.7, 9.5 Hz, 1H), 2.82 (d, J=9.0 Hz, 1H), 2.80-2.75* (m, 1H), 2.72* (d, J=8.7 Hz, 1H), 2.71-2.63 (m, 1H), 2.48-2.40* (m, 1H), 2.39-2.24 (m, 2H), 1.85 (d, J=5.6 Hz, 3H), 1.84* (d, J=6.1 Hz, 3H), 1.65 (s, 3H), 1.58* (s, 3H), 1.41 (s, 3H), 1.34* (s, 3H), 1.32-1.13 (m, 2H), 0.15 (s, 9H), 0.08* (s, 9H*). ¹³C NMR (150.9 MHz, CDCl₃, 20° C., mixture of atropisomers):⁷⁹ δ 168.0, 166.5, 160.6, 159.3, 143.3, 142.7, 142.6, 142.4, 140.2, 139.9, 139.4, 139.2, 138.6, 138.4, 137.8, 135.6, 135.5, 134.5, 134.3, 130.7, 130.4, 129.5, 129.4, 129.2, 128.5, 128.0, 127.9, 127.3, 127.1 (2C), 126.4, 124.7, 124.4, 124.2, 124.0, 123.7, 122.7, 122.1, 120.9, 120.7, 119.6, 114.5, 107.1, 106.6, 79.1, 78.5, 77.4, 76.9, 76.6, 65.4, 65.1, 63.9, 63.7, 59.8, 59.7, 54.8, 54.6, 52.9, 52.7, 52.5, 49.8, 45.2, 44.4, 37.8, 37.1, 36.7, 36.1, 32.5, 31.0, 30.6, 29.8, 24.9 (2C), 24.8, 20.7, 20.6 (2C), 18.8, 18.7, 10.9, 10.8, −1.7, −1.8. FTIR (thin film) cm⁻¹: 2956 (m), 2899 (m), 1685 (s), 1656 (s), 1629 (s), 1595 (s), 1486 (s), 1469 (s), 1390 (s), 1343 (s), 1295 (m), 1251 (m), 1158 (s), 1093 (m), 1002 (m), 898 (m), 860 (s). HRMS (ESI) (m/z): calc'd for C₃₇H₄₇N₄OSSi [M+H]⁺: 687.3031, found: 687.3029. [α]_{D}²⁴: +30 (c=0.66, CH₂Cl₂). TLC (50% ethyl acetate in hexanes), Rf: 0.26 (UV, CAM).

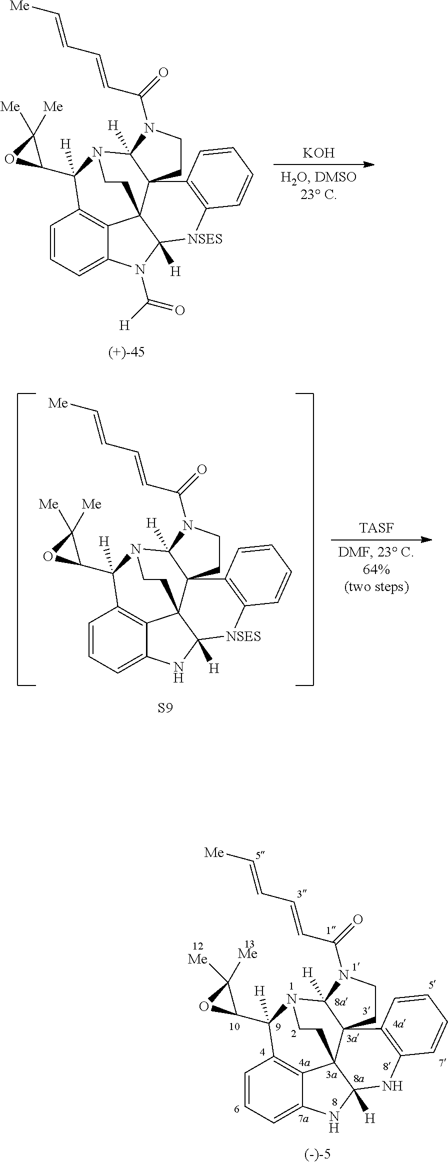

### Example 32: (−)-Communesin C (5)

An aqueous potassium hydroxide solution (0.5 M, 175 μL, 87.3 μmol, 5.00 equiv) was added rapidly to a solution of (+)-N8′-(trimethylsilyl)ethanesulfonyl communesin D (45, 12.0 mg, 17.5 μmol, 1 equiv) in dimethyl sulfoxide (1.75 mL) and deionized water (175 L) at 23° C. After 21 min, the light-orange homogeneous solution was diluted with a saturated aqueous sodium chloride solution (20 mL) and the mixture was extracted with ethyl acetate (3×10 mL). The combined organic extracts were washed with a saturated aqueous sodium chloride solution (2×20 mL), were dried over anhydrous sodium sulfate, were filtered, and were concentrated under reduced pressure. The resulting residue was filtered through a plug of silica gel (eluent: ethyl acetate) to afford crude sulfonamide S9 as a pale-yellow solid, which was used directly in the next step without further purification.

A degassed solution of tris(dimethylamino)sulfonium difluorotrimethylsilicate (TASF, 19.3 mg, 70.0 μmol, 4.00 equiv) in N,N-dimethylformamide (180 μL) was added to a degassed solution of crude sulfonamide S9 (1 equiv) in N,N-dimethylformamide (400 L) at 23° C. After 2 h, an additional portion of TASF (9.6 mg, 35 μmol, 2.0 equiv) in N,N-dimethylformamide (90 μL) was added. After 1 h, a saturated aqueous sodium chloride solution (10 mL) and deionized water (5 mL) were added and the mixture was extracted with ethyl acetate (3×10 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 30%→40% acetone in hexanes) to afford (−)-communesin C (5, 5.53 mg, 63.8% over two steps) as a white solid. Structural assignments were made using additional information from gCOSY, gHSQC, gHMBC, and 1D selective NOESY experiments. ¹H NMR (500 MHz, DMSO-d₆, 20° C., 11:1 mixture of atropisomers, major atropisomer): δ 7.07 (dd, J=15.2, 9.7 Hz, 1H), 6.92 (ddd, J=7.8, 6.2, 2.6 Hz, 1H), 6.74 (t, J=7.6 Hz, 1H), 6.70 (d, J=7.9 Hz, 1H), 6.61-6.55 (m, 2H), 6.53 (d, J=15.1 Hz, 1H), 6.21-6.12 (m, 2H), 6.11 (d, J=7.5 Hz, 1H), 6.05 (d, J=7.6 Hz, 1H), 6.03 (d, J=1.5 Hz, 1H), 5.85 (d, J=1.4 Hz, 1H), 5.14 (d, J=1.4 Hz, 1H), 4.84 (s, 1H), 4.19 (d, J=9.0 Hz, 1H), 3.72 (dd, J=11.2, 7.7 Hz, 1H), 3.30 (dd, J=15.5, 9.8 Hz, 1H), 3.19 (dt, J=15.4, 8.6 Hz, 1H), 2.90 (d, J=9.2 Hz, 1H), 2.79 (td, J=11.3, 6.6 Hz, 1H), 2.70 (td, J=12.3, 8.1 Hz, 1H), 2.28 (td, J=8.9, 4.2 Hz, 1H), 2.10 (dt, J=12.8, 9.8 Hz, 1H), 1.82 (d, J=5.3 Hz, 3H), 1.73-1.64 (m, 1H), 1.58 (s, 3H), 1.33 (s, 3H). ¹H NMR (600 MHz, CDCl₃, 20° C., 15:1 mixture of atropisomers, *denotes minor atropisomer): δ 7.32 (dd, J=15.1, 10.8 Hz, 2H, C3″, C3″H*), 6.99 (ddd, J=7.6, 5.4, 3.6 Hz, 2H, C6′H, C6′H*), 6.85 (app-t, J=7.7 Hz, 1H, C6H), 6.70-6.64 (m, 6H, C4′H, C4′H*, C5′H, C5′H*, C7′H, C7′H*), 6.57 (d, J=14.8 Hz, 1H, C2″H), 6.25-6.15 (m, 6H, C5H, C5H*, C7H, C7H*, C4″H, C5″H*), 6.14-6.04 (m, 3H, C2″H*, C4″H*, C5″H), 5.50 (s, 1H, C8a′H*), 5.12 (s, 1H, C8a′H), 5.01 (s, 1H, C8aH), 4.57-4.51 (m, 1H, C9H*), 4.28 (br-s, 1H, N8H/N8′H), 4.19 (br-s, 1H, N8H/N8′H), 4.18 (d, J=9.1 Hz, 1H, C9H), 3.88 (app-dd, J=12.3, 8.2 Hz, 1H, C2′Hₐ), 3.83 (app-t, J=9.2 Hz, 1H, C2′Hₐ*), 3.57-3.49 (m, 1H, C2Hₐ*), 3.50-3.38 (m, 2H, C2H₂), 3.42-3.33 (m, 1H, C2H_{b}*), 3.22-3.15 (m, 1H, C2′H_{b}*), 3.09 (app-td, J=12.0, 7.2 Hz, 1H, C2′H_{b}), 2.97-2.91 (m, 1H, C3′Hₐ*), 2.89 (d, J=9.1 Hz, 1H, C10H), 2.80 (d, J=8.7 Hz, 1H, C10H*), 2.72 (app-td, J=12.9, 8.7 Hz, 1H, C3′Hₐ), 2.41-2.35 (m, 2H, C3Hₐ, C3Hₐ*), 2.29 (app-dt, J=13.0, 9.3 Hz, 2H, C3H_{b}, C3H_{b}*), 2.03 (app-dd, J=13.4, 6.8 Hz, 1H, C3′H_{b}*), 1.97 (app-dd, J=12.8, 6.7 Hz, 1H, C3′H_{b}), 1.85 (d, J=6.8 Hz, 3H, C6″H₃), 1.83 (d, J=6.6 Hz, 3H, C6″H₃*), 1.65 (s, 3H, C13H₃), 1.62 (s, 3H, C13H₃*), 1.42 (s, 3H, C12H₃), 1.37 (s, 3H, C12H₃*). ¹³C NMR (150.9 MHz, CDCl₃, 20° C., 15:1 mixture of atropisomers, *denotes minor atropisomer): δ 168.6 (C₁″), 149.8 (C7a), 142.9 (C3″*), 142.6 (C7a′), 142.0 (C3″), 137.4 (C4), 137.3 (C5″), 137.2 (C5″*), 133.0 (C4a), 131.7 (C4a′), 130.9 (2C, C4″, C4″*), 128.8 (C6), 127.4 (2C, C6′, C6′*), 123.8 (C4′*), 123.5 (C4′), 121.4 (C2″), 120.5 (C5′), 120.0 (C2″*), 117.2 (C7′*), 116.9 (C7′), 116.1 (C5*), 115.4 (C5), 106.3 (C7), 105.9 (C7*), 79.3 (C8a′), 78.4 (C8a′*), 77.1 (C8a), 65.7 (C9), 65.2 (C9*), 64.3 (C10*), 64.1 (C10), 59.9 (C11), 52.5 (C3a), 52.2 (C3a′), 45.3 (C2′*), 44.4 (C2′), 38.7 (C3*), 38.0 (C3), 37.8 (C2*), 36.0 (C2), 32.5 (C3′*), 30.5 (C3′), 25.0 (C12), 24.9 (C12*), 20.6 (2C, C13, C13*), 18.8 (2C, C6″, C6″*). FTIR (thin film) cm⁻¹: 3341 (br-m), 3054 (w), 3022 (w), 2963 (m), 2927 (m), 2878 (m), 1651 (s), 1624 (s), 1596 (s), 1482 (m), 1461 (m), 1400 (s), 1338 (m), 1250 (m), 1165 (m), 1062 (m), 1002 (m), 748 (m). HRMS (ESI) (m/z): calc'd for C₃₁H₃₅N₄O₂ [M+H]⁺: 495.2755, found: 495.2760. [α]_{D}²³: −108 (c=0.28, MeOH).⁸⁰ TLC (40% acetone in hexanes), Rf: 0.26 (UV, CAM).

TABLE 8
Comparison of ¹H NMR data for (−)-communesin C (5) with literature data
(DMSO-d₆, major atropisomer):

| | Proksch Isolation Report^{81,82} | This Work |
|---|---|---|
| | (−)-Communesin C (5) | (−)-Communesin C (5) |
| Assignment | ¹H NMR, 500 MHz, DMSO-d₆ | ¹H NMR, 500 MHz, DMSO-d6 |
| C2 | —⁸³ | 3.30 (dd, J = 15.5, 9.8 Hz, 1H) |
| | | 3.19 (dt, J = 15.4, 8.6 Hz, 1H) |
| C3 | 2.27 (m, 1H) | 2.28 (td, J = 8.9, 4.2 Hz, 1H) |
| | 2.09 (m, 1H) | 2.10 (dt, J = 12.8, 9.8 Hz, 1H) |
| C3a | — | — |
| C4a | — | — |
| C4 | — | — |
| C5 | 6.10 (d, J = 7.7 Hz, 1H) | 6.11 (d, J = 7.5 Hz, 1H) |
| C6 | 6.74 (dd, J = 8.2, 7.7 Hz, 1H) | 6.74 (t, J = 7.6 Hz, 1H) |
| C7 | 6.03 (d, J = 8.2 Hz, 1H) | 6.05 (d, J = 7.6 Hz, 1H) |
| C7a | — | — |
| C8a | 4.83 (s, 1H) | 4.84 (s, 1H) |
| C9 | 4.18 (d, J = 9.2 Hz, 1H) | 4.19 (d, J = 9.0 Hz, 1H) |
| C10 | 2.88 (d, J = 9.2 Hz, 1H) | 2.90 (d, J = 9.2 Hz, 1H) |
| C11 | — | — |
| C12 | 1.55 (s, 3H) | 1.58 (s, 3H) |
| C13 | 1.32 (s, 3H) | 1.33 (s, 3H) |
| C2′ | 3.73 (m, 1H) | 3.72 (dd, J = 11.2, 7.7 Hz, 1H) |
| | 2.78 (m, 2H) | 2.79 (td, J = 11.3, 6.6 Hz, 1H) |
| C3′ | 2.78 (m, 2H) | 2.70 (td, J = 12.3, 8.1 Hz, 1H) |
| | 1.78 (m, 1H) | 1.73-1.64 (m, 1H) |
| C3a′ | — | — |
| C4a′ | — | — |
| C4′ | 6.57 (br-m, 2H) | 6.61-6.55 (m, 2H) |
| C5′ | 6.57 (br-m, 2H) | 6.61-6.55 (m, 2H) |
| C6′ | 6.90 (m, 1H) | 6.92 (ddd, J = 7.8, 6.2, 2.6 Hz, 1H) |
| C7′ | 6.79 (br-d, J = 7.7 Hz, 1H) | 6.70 (d, J = 7.9 Hz, 1H) |
| C7a′ | — | — |
| C8a′ | 5.12 (s, 1H) | 5.14 (d, J = 1.4 Hz, 1H) |
| C1″ | — | — |
| C2″ | 6.53 (d, J = 15.1 Hz, 1H) | 6.53 (d, J = 15.1 Hz, 1H) |
| C3″ | 7.07 (dd, J = 15.1, 10.1 Hz, 1H) | 7.07 (dd, J = 15.2, 9.7 Hz, 1H), |
| C4″ | 6.16 (m, 2H) | 6.21-6.12 (m, 2H) |
| C5″ | 6.16 (m, 2H) | 6.21-6.12 (m, 2H) |
| C6″ | 1.81 (d, J = 5.4 Hz, 3H) | 1.82 (d, J = 5.3 Hz, 3H) |
| N8H / N8′H | — | 6.03 (d, J = 1.5 Hz, 1H) |
| N8H / N8′H | — | 5.85d, J = 1.4 Hz, 1H) |

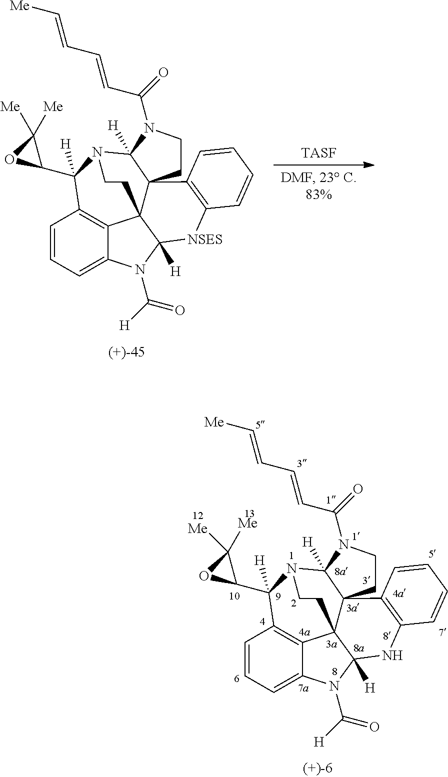

### Example 33: (+)-Communesin D (6)

A solution of tris(dimethylamino)sulfonium difluorotrimethylsilicate (TASF, 11.9 mg, 43.1 μmol, 4.00 equiv) in N,N-dimethylformamide (120 μL) was added to a solution of (+)-N8′-(trimethylsilyl)ethanesulfonyl communesin D (45, 7.40 mg, 10.8 μmol, 1 equiv) in N,N-dimethylformamide (260 L) at 23° C. After 2 h, a saturated aqueous sodium chloride solution (5 mL) and deionized water (3 mL) were added and the mixture was extracted with ethyl acetate (3×5 mL). The combined organic extracts were washed with a saturated aqueous sodium chloride solution (2×10 mL), were dried over anhydrous sodium sulfate, were filtered, and were concentrated under reduced pressure. The resulting residue was purified by flash column chromatography on silica gel (eluent: 40%→50% acetone in hexanes) to afford (+)-communesin D (6, 4.66 mg, 82.8%) as a white solid. Structural assignments were made using additional information from gCOSY, gHSQC, gHMBC, and 1D selective NOESY experiments. ¹H NMR (500 MHz, CDCl₃, 20° C., 40:7.3*:1** mixture of atropisomers, * and ** denote minor atropisomers): δ 8.92 (d, J=0.9 Hz, 1H, N8CHO), 8.89 (s, 1H, N8CHO*), 8.70 (s, 1H, N8CHO**), 7.33 (dd, J=15.1, 10.1 Hz, 1H, C3″H), 7.04 (app-t, J=7.8 Hz, 1H, C6H), 7.03-6.99 (m, 2H, C6′H, C6′H*), 6.81 (d, J=7.9 Hz, 1H, C7H), 6.77 (d, J=7.9 Hz, 1H, C7H*), 6.73-6.67 (m, 3H, C5′H, C7′H, C7′H*), 6.66 (dd, J=7.7, 1.5 Hz, 1H, C4′H), 6.61 (d, J=7.8 Hz, 1H, C5H), 6.49 (d, J=15.1 Hz, 1H, C2″H), 6.24-6.04 (m, 4H, C2″H*, C4″H, C4″H*, C5″H), 5.57-5.54 (m, 1H, C8aH), 5.53 (s, C8aH*), 5.50 (s, 1H, C8a′H*), 5.43 (s, 1H, C8aH**), 5.40-5.38 (m, 1H, N8′H*), 5.37 (d, J=2.0 Hz, 1H, N8′H), 5.13 (s, 1H, C8a′H**), 5.10 (s, 1H, C8a′H), 4.68-4.57 (m, 1H, C9H*), 4.26 (d, J=8.8 Hz, 1H, C9H), 3.89 (app-dd, J=12.1, 8.3 Hz, 2H, C2′Hₐ, C2′Hₐ*), 3.59-3.39 (m, 2H, C2H₂), 3.19 (app-q, J=9.2, 1H, C2′H_{b}*), 3.06 (app-td, J=11.9, 6.9 Hz, 1H, C2′H_{b}), 2.96 (app-q, J=11.1 Hz, 1H, C3′Hₐ*), 2.88 (d, J=8.9 Hz, 1H, C10H), 2.80-2.74 (m, 1H, C10H*), 2.73 (app-td, J=12.5, 8.5 Hz, 1H, C3′Hₐ), 2.49 (d, J=13.4, 8.0, 2.7 Hz, 1H, C3Hₐ), 2.29 (app-dt, J=13.2, 9.3 Hz, 1H, C3H_{b}), 2.14 (app-dd, J=13.4, 7.3 Hz, 1H, C3′H_{b}*), 2.08 (app-dd, J=13.1, 6.9 Hz, 1H, C3′H_{b}), 1.85 (d, J=6.1 Hz, 3H, C6″H), 1.84 (d, J=6.2 Hz, 3H, C6″H*), 1.66 (s, 3H, C13H₃), 1.60 (s, 3H, C13H₃*), 1.44 (s, 3H, C12H₃), 1.37 (s, 3H, C12H₃*). ¹³C NMR (150.9 MHz, CDCl₃, 20° C., 40:7.3*:1 mixture of atropisomers, * denotes minor atropisomer): δ 168.4 (C1″), 166.9 (C1″*), 158.2 (N8CHO), 143.3 (C3″*), 142.3 (C3″), 141.6 (C7a′*), 141.5 (C7a′), 140.5 (C7a), 140.3 (C7a*), 139.2 (C4), 138.5 (C5″*), 137.7 (C5″), 135.3 (C4a), 135.0 (C4a*), 131.6 (C4a′*), 131.0 (C4a′), 130.7 (C4″), 130.3 (C4″*), 129.1 (C6), 129.0 (C6*), 128.0 (C6′), 127.8 (C6′*), 123.6 (C4′*), 123.4 (C4′), 122.5 (C5*), 121.9 (C5), 121.4 (C5′*), 121.1 (C5′), 121.0 (C2″), 119.6 (C2″*), 117.4 (C7′*), 117.2 (C7′), 106.5 (C7), 106.1 (C7*), 78.7 (C8a′), 77.9 (C8a′*), 77.8 (C8a*), 77.4 (C8a), 65.4 (C9), 65.0 (C9*), 64.0 (C10*), 63.8 (C10), 59.9 (C11), 52.1 (C3a′), 50.9 (C3a), 50.8 (C3a*), 49.5 (C3a′*), 45.1 (C2′*), 44.2 (C2′), 37.7 (C3), 36.0 (C2), 32.4 (C3′*), 30.4 (C3′), 25.0 (C12), 24.9 (C12*), 20.7 (C13), 20.5 (C13*), 18.9 (C6″), 18.8 (C6″*). FTIR (thin film) cm⁻¹: 3378 (br-w), 2961 (w), 2934 (w), 2881 (w), 1651 (s), 1626 (m), 1605 (m), 1590 (m), 1488 (m), 1472 (m), 1403 (m), 1306 (m), 1170 (m), 996 (m), 752 (m). HRMS (ESI) (m/z): calc'd for C₃₂H₃₅N₄O₃ [M+H]+: 523.2704, found: 523.2702. [α]_{D}²³: +151 (c=0.23, CHCl₃).⁸⁴ TLC (40% acetone in hexanes), Rf: 0.24 (UV, CAM).

TABLE 9
Comparison of ¹H NMR data for (+)-communesin D (6) with literature
data (CDCl₃, major atropisomer):

| | Hayashi's Isolation | |
|---|---|---|
| | Report^{85,86} | This Work |
| | (+)-Communesin D (6) | (+)-Communesin D (6) |
| Assignment | ¹H NMR, CDCl₃ | ¹H NMR, 500 MHz, CDCl₃ |
| C2 | 3.47 (m, 1H) | 3.59-3.39 (m, 2H) |
| | 3.43 (m, 1H) | |
| C3 | 2.50 (ddd, J = 13.0, 8.0, 2.5 | 2.49 (ddd, J = 13.4, 8.0, 2.7 Hz, |
| | Hz, 1H) | 1H) |
| | 2.30 (dt, J = 13.0, 9.0 Hz, 1H) | 2.29 (app-dt, J = 13.2, 9.3 Hz, 1H) |
| C3a | — | — |
| C4a | — | — |
| C4 | — | — |
| C5 | 6.62 (d, J = 8.0 Hz, 1H) | 6.61 (d, J = 7.8 Hz, 1H) |
| C6 | 7.04 (t, J = 8.0 Hz, 1H) | 7.04 (app-t, J = 7.8 Hz, 1H) |
| C7 | 6.81 (d, J= 8.0 Hz, 1H) | 6.81 (d, J = 7.9 Hz, 1H) |
| C7a | — | — |
| N8CHO | 8.91 (d, J = 0.5 Hz, 1H) | 8.92 (d, J = 0.9 Hz, 1H) |
| C8a | 5.55 (br-d, J = 1.0 Hz, 1H) | 5.57-5.54 (m, 1H) |
| C9 | 4.26 (d, J = 9.0 Hz, 1H) | 4.26 (d, J = 8.8 Hz, 1H) |
| C10 | 2.88 (d, J = 9.0 Hz, 1H) | 2.88 (d, J = 8.9 Hz, 1H) |
| C11 | — | — |
| C12 | 1.43 (s, 3H) | 1.44 (s, 3H) |
| C13 | 1.67 (s, 3H) | 1.66 (s, 3H) |
| C2′ | 3.89 (dd, J = 12.0, 8.5 Hz, 1H) | 3.89 (app-dd, J = 12.1, 8.3 Hz, 1H) |
| | 3.06 (dt, J = 12.0, 7.0 Hz, 1H) | 3.06 (app-td, J = 11.9, 6.9 Hz, 1H) |
| C3′ | 2.73 (ddd, J = 13.0, 8.0, 2.5 | 2.73 (app-td, J = 12.5, 8.5 Hz, 1H) |
| | Hz, 1H) | 2.08 (app-dd, J = 13.1, 6.9 Hz, 1H) |
| | 2.09 (dd, J = 12.5, 7.0 Hz, 1H) | |
| C3a′ | — | — |
| C4a′ | — | — |
| C4′ | 6.69 (dd, J = 6.5, 2.0 Hz, 1H) | 6.66 (dd, J = 7.7, 1.5 Hz, 1H) |
| C5′ | 6.70 (td, J = 6.5, 2.0 Hz, 1H) | 6.73-6.67 (m, 2H) |
| C6′ | 7.01 (td, J = 6.5, 2.0 Hz, 1H) | 7.03-6.99 (m, 1H) |
| C7′ | 6.65 (dd, J = 6.5, 2.0 Hz, 1H) | 6.73-6.67 (m, 2H) |
| C7a′ | — | — |
| C8a′ | 5.10 (s, 1H) | 5.10 (s, 1H) |
| C1″ | — | — |
| C2″ | 6.49 (d, J = 15.0 Hz, 1H) | 6.49 (d, J = 15.1 Hz, 1H) |
| C3″ | 7.34 (dd, J = 15.0, 10.5 Hz) | 7.33 (dd, J = 15.1, 10.1 Hz, 1H) |
| C4″ | 6.19 (dd, J = 16.0, 10.5 Hz, | 6.24-6.04 (m, 2H) |
| | 1H) | |
| C5″ | 6.14 (dq, J = 16.0, 6.0 Hz, 1H) | 6.24-6.04 (m, 2H) |
| C6″ | 1.86 (d, J = 6.0 Hz, 3H) | 1.85 (d, J = 6.1 Hz, 3H) |
| N8′H | 5.37 (d, J = 1.5 Hz, 1H) | 5.40-5.38 (m, 1H) |

TABLE 10
Comparison of ¹³C NMR data for (+)-communesin D (6) with
literature data (CDCl₃, major atropisomer):

| | Hayashi's Isolation | This Work | Chemical Shift |
|---|---|---|---|
| | Report^{85,86} | (+)-Communesin | Difference |
| | (+)-Communesin | D (6) ¹³C NMR, | Δδ = δ |
| | D (6) | 150.9 MHz, | (this work) - δ |
| Assignment | ¹³C NMR, CDCl₃ | CDCl₃ | (Hayashi's report) |
| C2 | 35.8 | 36.0 | 0.2 |
| C3 | 37.5 | 37.7 | 0.2 |
| C3a | 50.7 | 50.9 | 0.2 |
| C4a | 135.1 | 135.3 | 0.2 |
| C4 | 139.0 | 139.2 | 0.2 |
| C5 | 121.8 | 121.9 | 0.1 |
| C6 | 128.9 | 129.0 | 0.2 |
| C7 | 106.3 | 106.5 | 0.2 |
| C7a | 140.3 | 140.5 | 0.2 |
| N8CHO | 158.0 | 158.2 | 0.2 |
| C8a | 77.2 | 77.4 | 0.2 |
| C9 | 65.2 | 65.4 | 0.2 |
| C10 | 63.6 | 63.8 | 0.2 |
| C11 | 59.8 | 59.9 | 0.1 |
| C12 | 24.8 | 25.0 | 0.2 |
| C13 | 20.5 | 20.7 | 0.2 |
| C2′ | 44.0 | 44.2 | 0.2 |
| C3′ | 30.2 | 30.4 | 0.2 |
| C3a′ | 51.9 | 52.1 | 0.2 |
| C4a′ | 130.8 | 131.0 | 0.2 |
| C4′ | 123.2 | 123.4 | 0.2 |
| C5′ | 120.9 | 121.1 | 0.2 |
| C6′ | 127.8 | 128.0 | 0.2 |
| C7′ | 117.0 | 117.2 | 0.2 |
| C7a′ | 141.3 | 141.5 | 0.2 |
| C8a′ | 78.4 | 78.7 | 0.3 |
| C1″ | 168.2 | 168.4 | 0.2 |
| C2″ | 120.8 | 121.1 | 0.3 |
| C3″ | 142.1 | 142.3 | 0.2 |
| C4″ | 130.5 | 130.7 | 0.2 |
| C5″ | 137.5 | 137.7 | 0.2 |
| C6″ | 18.7 | 18.9 | 0.2 |

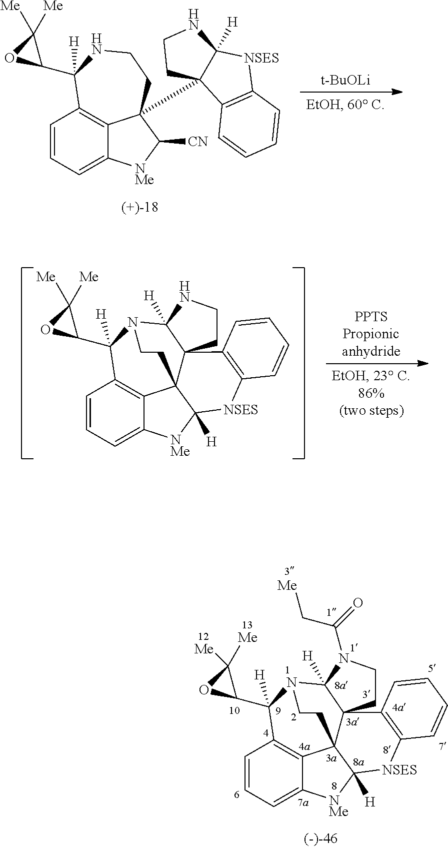

### Example 34: (−)-N8′-(Trimethylsilyl)ethanesulfonyl communesin G(46)

A sample of lithium tert-butoxide (14.4 mg, 180 μmol, 10.0 equiv) was added to a solution of heterodimer (+)-18 (10.9 mg, 18.0 μmol, 1equiv) in anhydrous ethanol (200 proof, 475 L) at 23° C. The flask was sealed with a Teflon-lined glass stopper under an argon atmosphere and was immersed in a preheated oil bath at 60° C. After 21 h, the reaction mixture was cooled to 23° C. and samples of pyridinium p-toluenesulfonate (PPTS, 36.3 mg, 144 μmol, 8.00 equiv) and propionic anhydride (9.5 μL, 74 μmol, 4.1 equiv) were added sequentially. The resulting viscous suspension was diluted with anhydrous ethanol (200 proof, 500 μL). After 30 min, a saturated aqueous sodium bicarbonate solution (8 mL) and deionized water (8 mL) were added and the resulting mixture was extracted with ethyl acetate (3×8 mL). The combined organic extracts were washed with a saturated aqueous sodium chloride solution (12 mL), were dried over anhydrous sodium sulfate, were filtered, and were concentrated under reduced pressure. The residue was purified by flash column chromatography on silica gel (eluent: 50% ethyl acetate in hexanes) to afford (−)-N8′-(trimethylsilyl)ethanesulfonyl communesin G (46, 9.80 mg, 85.5%) as a white solid. Structural assignments were made using additional information from gCOSY, gHSQC, gHMBC, and 1D selective NOESY experiments. ¹H NMR (500 MHz, CDCl₃, 20° C., 4.6:1 mixture of atropisomers, *denotes minor atropisomer): δ 7.56 (dd, J=7.9, 1.2 Hz, 1H, C7′H), 7.41 (d, J=8.0, 1H, C7′H*), 7.19 (app-td, J=7.9, 1.5 Hz, 1H, C6′H), 7.18 (app-t, J=7.8 Hz, 1H, C6′H*), 7.05 (app-td, J=7.6, 1.3 Hz, 1H, C5′H), 7.04 (app-t, J=7.6 Hz, 1H, C5′H*), 6.91 (t, J=7.7 Hz, 2H, C6H, C6H*), 6.81 (dd, J=7.8, 1.5 Hz, 1H, C4′H), 6.73 (d, J=7.6 Hz, 1H, C4′H*), 6.14 (d, J=8.1 Hz, 1H, C5H*), 6.10 (d, J=7.6 Hz, 1H, C5H), 5.97 (d, J=7.8 Hz, 1H, C7H), 5.94 (d, J=7.8 Hz, 1H, C7H*), 5.71 (s, 1H, C8aH), 5.63 (s, 1H, C8aH*), 5.43 (s, 1H, C8a′H*), 5.07 (s, 1H, C8a′H), 4.51 (d, J=8.9 Hz, 1H, C9H*), 4.10 (d, J=9.0 Hz, 1H, C9H), 3.94 (app-dd, J=11.2, 8.8 Hz, 1H, C2′Hₐ), 3.72 (app-t, J=9.3 Hz, 1H, C2′Hₐ*), 3.56 (app-dd, J=15.6, 9.9 Hz, 1H, C2Hₐ*), 3.48 (app-dd, J=16.1, 10.0 Hz, 2H, C2Hₐ, C2H_{b}*), 3.41-3.29 (m, 2H, C2H_{b}, N8′SO₂CHa*), 3.28-3.20 (m, 1H, N8′SO₂CH_{b}*), 3.24 (app-td, J=13.4, 5.0 Hz, 1H, N8′SO₂CHa), 3.22-3.15 (m, 1H, C2′H_{b}*), 3.16 (app-td, J=13.3, 4.7 Hz, 1H, N8′SO₂CH_{b}), 3.10 (app-td, J=11.4, 7.6 Hz, 1H, C2′H_{b}), 3.05-2.96 (m, 1H, C3′Hₐ*), 2.92 (s, 3H, N8CH₃), 2.91-2.75 (m, 7H, N8CH₃*, C10H, C10H*, C3′Hₐ, C2″Hₐ), 2.55-2.44 (m, 2H, C3Hₐ, C₃Hₐ*), 2.39 (app-dq, J=14.6, 7.4 Hz, 1H, C2″H_{b}), 2.34-2.24 (m, 4H, C3H_{b}, C3H_{b}*, C2″H₂*), 2.11 (app-dd, J=13.4, 7.4 Hz, 1H, C3′H_{b}*), 1.91 (app-dd, J=13.2, 6.6 Hz, 1H, C3′H_{b}), 1.58 (s, 3H, C13H₃*), 1.53 (s, 3H, C13H₃), 1.38 (s, 3H, C12H₃), 1.35 (s, 3H, C12H₃*), 1.30-1.19 (m, 5H, C3″H₃, N8′SO₂CH₂CH₂), 1.19-1.13 (m, 5H, C3″H₃*, N8′SO₂CH₂CH₂*), 0.10 (s, 9H, Si(CH₃)₃*), 0.06 (s, 9H, Si(CH₃)₃). ¹³C NMR (150.9 MHz, CDCl₃, 20° C., 4.6:1 mixture of atropisomers, *denotes minor atropisomer): δ 175.0 (C1″), 173.6 (C1″*), 150.0 (C7a), 149.5 (C7a*), 139.3 (C4a′*), 138.4 (C4a′), 137.4 (C4), 136.2 (C7a′*), 136.1 (C7a′), 131.3 (C4a), 129.4 (C6), 129.2 (C6*), 127.9 (C6′), 127.7 (C6′*), 126.7 (C5′*), 126.4 (C5′), 125.4 (C7′*), 124.8 (C7′), 124.3 (C4′*), 124.0 (C4′), 114.6 (C5*), 113.9 (C5), 102.6 (C7), 102.3 (C7*), 85.3 (C8a*), 84.8 (C8a), 79.4 (C8a′), 78.4 (C8a′*), 65.4 (C9), 65.2 (C9*), 64.0 (C10*), 63.9 (C10), 59.8 (2C, C11, C11*), 54.2 (2C, C3a, C3a*), 52.6 (C3a′), 52.5 (N8′SO₂CH₂*), 51.8 (N8′SO₂CH₂), 50.2 (C3a′*), 45.1 (C2′*), 44.3 (C2′), 38.0 (2C, C3*, C2*), 37.8 (C3), 36.6 (C2), 33.3 (C3′*), 31.6 (C3′), 31.0 (N8CH₃*), 30.9 (N8CH₃), 28.2 (C2″*), 27.8 (C2″), 24.9 (2C, C12, C12*), 20.6 (2C, C13*, C13), 10.8 (N8′SO₂CH₂CH₂*), 10.7 (N8′SO₂CH₂CH₂), 9.3 (C3″), 8.7 (C3″*), −1.7 (Si(CH₃)₃*), −1.8 (Si(CH₃)₃). FTIR (thin film) cm⁻¹: 3055 (w), 2955 (m), 2879 (w), 1650 (m), 1599 (s), 1487 (m), 1408 (m), 1341 (m), 1250 (m), 1157 (m), 1056 (m). HRMS (ESI) (m/z): calc'd for C₃₄H₄₇N₄O₄SSi [M+H]⁺: 635.3082, found: 635.3085. [α]_{D}²²: −129 (c=0.49, CH₂Cl₂). TLC (50% ethyl acetate in hexanes), Rf: 0.19 (UV, CAM).

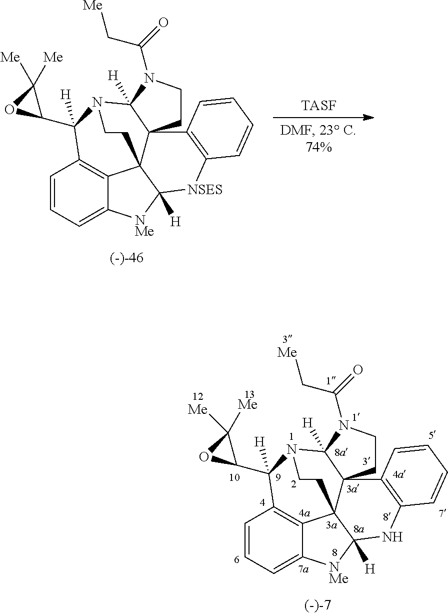

### Example 35: (−)-Communesin G (7)

A solution of tris(dimethylamino)sulfonium difluorotrimethylsilicate (TASF, 12.7 mg, 46.0 μmol, 4.00 equiv) in N,N-dimethylformamide (180 μL) was added to a suspension of (−)-N8′-(trimethylsilyl)ethanesulfonyl communesin G (46, 7.30 mg, 11.5 μmol, 1 equiv) in N,N-dimethylformamide (200 L) at 23° C. After 4 h, a saturated aqueous sodium chloride solution (5 mL) and deionized water (3 mL) were added and the mixture was extracted with ethyl acetate (3×8 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 30% acetone in hexanes) to afford (−)-communesin G (7, 3.99 mg, 73.8%) as a white solid. Structural assignments were made using additional information from gCOSY, gHSQC, gHMBC, and 1D selective NOESY experiments. ¹H NMR (500 MHz, CDCl₃, 20° C., 8.7:1 mixture of atropisomers, *denotes minor atropisomer): δ 7.00 (app-td, J=7.6, 1.6 Hz, 2H, C6′H, C6′H*), 6.88 (t, J=7.7 Hz, 2H, C6H, C6H*), 6.73-6.64 (m, 5H, C4′H, C5′H, C5′H*, C7′H, C7′H*), 6.60 (d, J=7.0 Hz, 1H, C4′H*), 6.09 (d, J=7.2 Hz, 1H, C5H*), 6.06 (d, J=7.6 Hz, 1H, C5H), 5.95 (d, J=7.8 Hz, 1H, C7H), 5.91 (d, J=7.8 Hz, 1H, C7H*), 5.30 (app-s, 1H, C8a′H*), 5.05 (d, J=1.4 Hz, 1H, C8a′H), 4.69 (s, 1H, C8aH), 4.67 (s, 1H, C8aH*), 4.58 (br-s, 1H, N8′H), 4.50 (d, J=8.5 Hz, 1H, C9H*), 4.10 (d, J=9.2 Hz, 1H, C9H), 3.89 (app-dd, J=11.9, 8.4 Hz, 1H, C2′Hₐ), 3.70-3.63 (m, 1H, C2′Hₐ*), 3.54 (app-dd, J=15.4, 9.2 Hz, 1H, C2Hₐ*), 3.45 (app-dd, J=15.7, 9.5 Hz, 1H, C2Hₐ), 3.41-3.30 (m, 2H, C2H_{b}, C2H_{b}*), 3.12-3.06 (m, 1H, C2′H_{b}*), 3.02 (app-td, J=11.6, 7.3 Hz, 1H, C2′H_{b}), 2.96-2.83 (m, 3H, C10H, C3′Hₐ*, C2″Hₐ), 2.84 (s, 3H, N8CH₃), 2.82 (s, 3H, N8CH₃*), 2.80 (d, J=9.2 Hz, 1H, C10H*), 2.73 (app-td, J=12.3, 11.3, 8.6 Hz, 1H, C3′Hₐ), 2.46-2.32 (m, 3H, C3Hₐ, C3Hₐ*, C2″H_{b}), 2.32-2.22 (m, 3H, C3H_{b}, C3H_{b}*, C2″H_{b}*), 2.03 (app-dd, J=13.0, 7.2 Hz, 1H, C3′H_{b}*), 1.96 (app-dd, J=13.3, 6.7 Hz, 1H, C3′H_{b}), 1.59 (s, 3H, C13H₃*), 1.54 (s, 3H, C13H₃), 1.38 (s, 3H, C12H₃), 1.36 (s, 3H, C12H₃*), 1.22 (t, J=7.4 Hz, 3H, C3″H₃), 1.16 (t, J=7.3 Hz, 3H, C3″H₃*).¹³C NMR (125.8 MHz, CDCl₃, 20° C., 8.7:1 mixture of atropisomers, *denotes minor atropisomer): δ 175.3 (C₁″), 150.7 (C7a), 150.6 (C7a*), 142.9 (C7a′*), 142.8 (C7a′), 137.1 (C4), 132.7 (C4a′), 132.4 (C4a), 132.2 (C4a*), 129.0 (C6), 128.8 (C6*), 127.5 (C6′), 127.3 (C6′*), 123.6 (C4′*), 123.4 (C4′), 121.0 (C5′*), 120.7 (C5′), 117.2 (C7′*), 117.1 (C7′), 114.0 (C5*), 113.4 (C5), 101.9 (C7), 101.5 (C7*), 83.1 (C8a*), 82.7 (C8a), 79.1 (C8a′), 78.1 (C8a′*), 65.4 (C9), 65.1 (C9*), 64.2 (C10*), 64.1 (C10), 59.8 (C11), 52.1 (C3a′), 51.6 (C3a), 49.8 (C3a′*), 45.2 (C2′*), 44.3 (C2′), 38.5 (C3*), 38.2 (C3), 37.8 (C2*), 36.5 (C2), 32.6 (C3′*), 30.8 (C3′), 29.9 (N8CH₃*), 29.8 (N8CH₃), 28.3 (C2″*), 27.8 (C2″), 24.9 (2C, C12, C12*), 20.6 (2C, C13*, C13), 9.4 (C3″), 8.7 (C3″*). FTIR (thin film) cm⁻¹: 3321 (br-m), 3052 (w), 2963 (m), 2921 (m), 2880 (m), 1641 (m), 1596 (m), 1494 (m), 1409 (m), 1280 (m), 1086 (m), 739 (m). HRMS (ESI) (m/z): calc'd for C29H₃₅N402 [M+H]⁺: 471.2755, found: 471.2754. [α]_{D}²³: −163 (c=0.20, MeOH).⁸⁷ TLC (30% acetone in hexanes), Rf: 0.16 (UV, CAM).

TABLE 11
Comparison of ¹H NMR data for (−)-communesin G (7) with literature
data (CDCl₃, major atropisomer):

| | Christophersen's Isolation | |
|---|---|---|
| | Report^{88,89} | This Work |
| | (−)-Communesin G (7) | (−)-Communesin G (7) |
| Assignment | ¹H NMR, 500 MHz, CDCl₃ | ¹H NMR, 500 MHz, CDCl₃ |
| C2 | 3.44 (m, 1H) | 3.45 (app-dd, J = 15.7, |
| | 3.35 (m, 1H) | 9.5 Hz, 1H) |
| | | 3.41-3.30 (m, 1H) |
| C3 | 2.35 (m, 1H) | 2.46-2.32 (m, 2H) |
| | 2.27 (m, 1H) | 2.32-2.22 (m, 2H) |
| C3a | — | — |
| C4a | — | — |
| C4 | — | — |
| C5 | 6.06 (d, J = 7.5 Hz, 1H) | 6.06 (d, J = 7.6 Hz, 1H) |
| C6 | 6.87 (t, J = 7.5 Hz, 1H) | 6.88 (t, J = 7.7 Hz, 1H) |
| C7 | 5.95 (d, J = 7.5 Hz, 1H) | 5.95 (d, J = 7.8 Hz, 1H) |
| C7a | — | — |
| N8CH₃ | 2.82 (s, 3H) | 2.84 (s, 3H) |
| C8a | 4.69 (s, 1H) | 4.69 (s, 1H) |
| C9 | 4.10 (d, J = 9.5 Hz, 1H) | 4.10 (d, J = 9.2 Hz, 1H) |
| C10 | 2.85 (m, 1H) | 2.96-2.83 (m, 2H) |
| C11 | — | — |
| C12 | 1.37 (s, 3H) | 1.38 (s, 3H) |
| C13 | 1.54 (s, 3H) | 1.54 (s, 3H) |
| C2' | 3.88 (dd, J = 12.0, 8.8 Hz, 1H) | 3.89 (app-dd, J = 11.9, |
| | 3.01 (ddd, J = 12.0, 11.7, | 8.4 Hz, 1H) |
| | 6.5 Hz, 1H) | 3.02 (app-td, J = 11.6, |
| | | 7.3 Hz, 1H) |
| C3' | 2.73 (ddd, J = 13.0, 11.7, | 2.73 (app-td, J = 12.3, 11.3, |
| | 8.8 Hz, 1H) | 8.6 Hz, 1H) |
| | 1.95 (dd, J = 13.0, 6.5 Hz, 1H) | 1.96 (app-dd, J = 13.3, |
| | | 6.7 Hz, 1H) |
| C3a' | — | — |
| C4a' | — | — |
| C4' | 6.66 (m, 1H) | 6.73-6.64 (m, 3H) |
| C5' | 6.68 (m, 1H) | 6.73-6.64 (m, 3H) |
| C6' | 6.99 (td, J = 8.0, 1.5 Hz, 1H) | 7.00 (app-td, J = 7.6, |
| | | 1.6 Hz, 1H) |
| C7' | 6.69 (m, 1H) | 6.73-6.64 (m, 3H) |
| C7a′ | | |
| C8a′ | 5.05 (s, 1H) | 5.05 (d, J = 1.4 Hz, 1H) |
| C1″ | — | — |
| C2″ | 2.88 (m, 1H) | 2.96-2.83 (m, 2H) |
| | 2.42 (m, 1H) | 2.46-2.32 (m, 2H) |
| C3″ | 1.22 (t, J = 7.5 Hz, 3H) | 1.22 (t, J = 7.4 Hz, 3H) |
| N8′H | — | 4.58 (br-s, 1H) |

TABLE 12
Comparison of ¹³C NMR data for (−)-communesin G (7) with
literature data (CDCl₃, major atropisomer):

| | Christophersen's | | |
|---|---|---|---|
| | Isolation Report^{88,90} | This Work | Chemical Shift |
| | (−)-Communesin | (−)-Communesin | Difference |
| | G (7) | G (7) ¹³C NMR, | Δδ = δ (this work) - |
| | ¹³C NMR, 75 MHz | 150.9 MHz, | δ (Christophersen |
| Assignment | CDCl₃ | CDCl₃ | report) |
| C2 | 36.3 | 36.5 | 0.2 |
| C3 | 38.0 | 38.2 | 0.2 |
| C3a | 51.7 | 51.6 | −0.1⁹¹ |
| C4a | 132.4 | 132.4 | 0.0 |
| C4 | 136.7 | 137.1 | 0.4 |
| C5 | 113.2 | 113.4 | 0.2 |
| C6 | 128.8 | 129.0 | 0.2 |
| C7 | 101.7 | 101.9 | 0.2 |
| C7a | 150.5 | 150.7 | 0.2 |
| N8CH₃ | 29.6 | 29.8 | 0.2 |
| C8a | 82.5 | 82.7 | 0.2 |
| C9 | 65.1 | 65.4 | 0.3 |
| C10 | 64.0 | 64.2 | 0.2 |
| C11 | 59.7 | 59.8 | 0.1 |
| C12 | 24.6 | 24.9 | 0.3 |
| C13 | 20.4 | 20.6 | 0.2 |
| C2′ | 44.1 | 44.3 | 0.2 |
| C3′ | 30.5 | 30.8 | 0.3 |
| C3a′ | 51.4 | 52.1 | 0.7⁹¹ |
| C4a′ | 132.5 | 132.7 | 0.2 |
| C4′ | 123.3 | 123.4 | 0.1 |
| C5′ | 117.0 | 120.7 | 3.7⁹² |
| C6′ | 127.4 | 127.5 | 0.1 |
| C7′ | 120.6 | 117.1 | −3.5⁹² |
| C7a′ | 142.6 | 142.8 | 0.2 |
| C8a′ | 78.9 | 79.1 | 0.2 |
| C1″ | 175.3 | 175.3 | 0.0 |
| C2″ | 27.6 | 27.8 | 0.2 |
| C3″ | 9.2 | 9.4 | 0.2 |

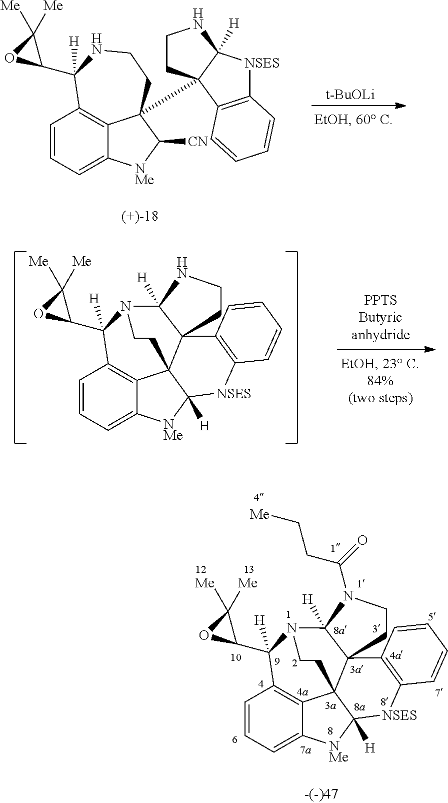

### Example 36: (−)-N8′-(Trimethylsilyl)ethanesulfonyl communesin H (47)

A sample of lithium tert-butoxide (21.6 mg, 270 μmol, 10.0 equiv) was added to a solution of heterodimer (+)-18 (16.3 mg, 27.0 μmol, 1 equiv) in anhydrous ethanol (200 proof, 710 L) at 23° C. The flask was sealed with a Teflon-lined glass stopper under an argon atmosphere and was immersed in a preheated oil bath at 60° C. After 23 h, the reaction mixture was cooled to 23° C. and samples of pyridinium p-toluenesulfonate (PPTS, 54.2 mg, 216 μmol, 8.00 equiv) and butyric anhydride (18.0 μL, 108 μmol, 4.00 equiv) were added sequentially. After 40 min, a saturated aqueous sodium bicarbonate solution (10 mL) and deionized water (10 mL) were added and the resulting mixture was extracted with ethyl acetate (3×10 mL). The combined organic extracts were washed with a saturated aqueous sodium chloride solution (15 mL), were dried over anhydrous sodium sulfate, were filtered, and were concentrated under reduced pressure. The residue was purified by flash column chromatography on silica gel (eluent: 45→50% ethyl acetate in hexanes) to afford (−)-N8′-(trimethylsilyl)ethanesulfonyl communesin H (47, 14.6 mg, 83.7%) as a white solid. Structural assignments were made using additional information from gCOSY, gHSQC, gHMBC, and 1D selective NOESY experiments. ¹H NMR (500 MHz, CDCl₃, 20° C., 5.9:1 mixture of atropisomers, *denotes minor atropisomer): δ 7.57 (dd, J=8.0, 1.1 Hz, 1H, C7′H), 7.41 (d, J=8.1, 1H, C7′H*), 7.20-7.16 (m, 1H, C6′H*), 7.19 (app-td, J=7.8, 1.4 Hz, 1H, C6′H), 7.06-7.01 (m, 1H, C5′H*), 7.05 (dd, J=8.2, 6.9 Hz, 1H, C5′H), 6.91 (t, J=7.7 Hz, 1H, C6H), 6.89 (t, J=7.8 Hz, 1H, C6H*), 6.81 (dd, J=7.9, 1.3 Hz, 1H, C4′H), 6.75 (d, J=7.9 Hz, 1H, C4′H*), 6.14 (d, J=8.1 Hz, 1H, C5H*), 6.10 (d, J=7.6 Hz, 1H, C5H), 5.98 (d, J=7.8 Hz, 1H, C7H), 5.93 (d, J=7.9 Hz, 1H, C7H*), 5.71 (s, 1H, C8aH), 5.63 (s, 1H, C8aH*), 5.44 (s, 1H, C8a′H*), 5.06 (s, 1H, C8a′H), 4.51 (d, J=8.4 Hz, 1H, C9H*), 4.11 (d, J=9.0 Hz, 1H, C9H), 3.93 (app-dd, J=12.1, 8.4 Hz, 1H, C2′Hₐ), 3.74 (app-t, J=9.6 Hz, 1H, C2′Hₐ*), 3.56 (app-dd, J=15.7, 10.1 Hz, 1H, C2Hₐ*), 3.48 (app-dd, J=16.1, 9.5 Hz, 1H, C2Hₐ), 3.36-3.20 (m, 2H, N8′SO₂CH₂*), 3.35 (app-dt, J=15.9, 8.8 Hz, 2H, C2H_{b}, C2H_{b}*), 3.24 (app-td, J=13.4, 4.8 Hz, 1H, N8′SO₂CHa), 3.23-3.19 (m, 1H, C2′H_{b}*), 3.16 (app-td, J=13.4, 4.8 Hz, 1H, N8′SO₂CH_{b}), 3.08 (app-td, J=11.4, 7.2 Hz, 1H, C2′H_{b}), 3.05-2.96 (m, 1H, C3′Hₐ*), 2.92 (s, 3H, N8CH₃), 2.87 (s, 3H, N8CH₃*), 2.86-2.75 (m, 5H, C10H, C10H*, C3′Hₐ, C2″Hₐ, C2″Hₐ*), 2.48 (app-dd, J=13.0, 8.2 Hz, 2H, C3Hₐ, C3Hₐ*), 2.38-2.21 (m, 4H, C3H_{b}, C3H_{b}*, C2″H_{b}, C2″H_{b}*), 2.10 (app-dd, J=13.3, 7.3 Hz, 1H, C3′H_{b}*), 1.90 (app-dd, J=13.2, 6.8 Hz, 1H, C3′H_{b}), 1.84-1.68 (m, 4H, C3″H₂, C3″H₂*), 1.58 (s, 3H, C13H₃*), 1.55 (s, 3H, C13H₃), 1.38 (s, 3H, C12H₃), 1.35 (s, 3H, C12H₃*), 1.33-1.13 (m, 4H, N8′SO₂CH₂CH₂, N8′SO₂CH₂CH₂*), 1.01 (t, J=7.4 Hz, 3H, C4″H₃), 0.95 (t, J=7.5 Hz, 3H, C4″H₃*), 0.10 (s, 9H, Si(CH₃)₃*), 0.06 (s, 9H, Si(CH₃)₃). ¹³C NMR (150.9 MHz, CDCl₃, 20° C., 5.9:1 mixture of atropisomers, *denotes minor atropisomer): δ 174.3 (C1″), 173.0 (C1″*), 150.0 (C7a), 149.5 (C7a*), 139.3 (C4a′*), 138.4 (C4a′), 137.3 (C4), 136.2 (C7a′*), 136.1 (C7a′), 131.3 (2C, C4a, C4a*), 129.4 (C6), 129.2 (C6*), 127.9 (C6′), 127.7 (C6′*), 126.7 (C5′*), 126.4 (C5′), 125.4 (C7′*), 124.8 (C7′), 124.3 (C4′*), 124.0 (C4′), 114.7 (C5*), 113.9 (C5), 102.6 (C7), 102.3 (C7*), 85.3 (C8a*), 84.8 (C8a), 79.4 (C8a′), 78.3 (C8a′*), 65.4 (C9), 65.2 (C9*), 64.0 (C10*), 63.9 (C10), 59.8 (2C, C11, C11*), 54.2 (2C, C3a, C3a*), 52.6 (C3a′), 52.5 (N8′SO₂CH₂*), 51.8 (N8′SO₂CH₂), 50.1 (C3a′*), 45.2 (C2′*), 44.1 (C2′), 38.1 (C3*), 38.0 (C2*), 37.8 (C3), 37.1 (C2″*), 36.8 (C2″), 36.6 (C2), 33.3 (C3′*), 31.5 (C3′), 31.0 (N8CH₃*), 30.9 (N8CH₃), 24.9 (2C, C12, C12*), 20.6 (C13*), 20.5 (C13), 18.4 (C3″), 18.1 (C3″*), 14.3 (C4″), 14.2 (C4″*), 10.8 (N8′SO₂CH₂CH₂*), 10.7 (N8′SO₂CH₂CH₂), −1.7 (Si(CH₃)₃*), −1.8 (Si(CH₃)₃). FTIR (thin film) cm⁻¹: 3055 (w), 2958 (m), 2875 (w), 1649 (m), 1599 (m), 1487 (m), 1408 (m), 1341 (m), 1251 (m), 1156 (m), 1053 (m). HRMS (ESI) (m/z): calc'd for C₃₅H₄₉N₄O₄SSi [M+H]⁺: 649.3238, found: 649.3244. [α]_{D}²²: −128 (c=0.73, CH₂Cl₂). TLC (50% ethyl acetate in hexanes), Rf: 0.35 (UV, CAM).

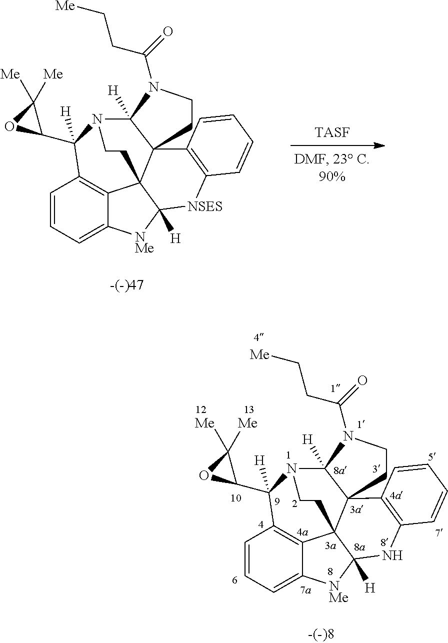

### Example 37: (−)-Communesin H (8)

A degassed solution of tris(dimethylamino)sulfonium difluorotrimethylsilicate (TASF, 19.0 mg, 69.0 μmol, 4.00 equiv) in N,N-dimethylformamide (175 μL) was added to a degassed solution of (−)-N8′-(trimethylsilyl)ethanesulfonyl communesin H (47, 11.2 mg, 17.3 mol, 1 equiv) in N,N-dimethylformamide (400 L) at 23° C. After 2 h, a saturated aqueous sodium chloride solution (10 mL) and deionized water (5 mL) were added and the mixture was extracted with ethyl acetate (3×10 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 30% acetone in hexanes) to afford (−)-communesin H (8, 7.51 mg, 89.8%) as a white solid. Structural assignments were made using additional information from gCOSY, gHSQC, gHMBC, and 1D selective NOESY experiments.

¹H NMR (500 MHz, CDCl₃, 20° C., 12:1 mixture of atropisomers, *denotes minor atropisomer): δ 7.00 (app-td, J=7.5, 1.5 Hz, 2H, C6′H, C6′H*), 6.88 (t, J=7.7 Hz, 1H, C6H), 6.85 (t, J=7.6 Hz, 1H, C6H*), 6.73-6.63 (m, 5H, C4′H, C5′H, C5′H*, C7′H, C7′H*), 6.60 (d, J=6.9 Hz, 1H, C4′H*), 6.09 (d, J=7.3 Hz, 1H, C5H*), 6.07 (d, J=7.8 Hz, 1H, C5H), 5.95 (d, J=7.8 Hz, 1H, C7H), 5.90 (d, J=7.6 Hz, 1H, C7H*), 5.40 (app-s, 1H, C8a′H*), 5.04 (d, J=1.4 Hz, 1H, C8a′H), 4.69 (s, 1H, C8aH), 4.67 (s, 1H, C8aH*), 4.59 (br-s, 1H, N8′H), 4.50 (d, J=8.2 Hz, 1H, C9H*), 4.10 (d, J=9.0 Hz, 1H, C9H), 3.88 (app-dd, J=11.6, 8.9 Hz, 1H, C2′Hₐ), 3.69 (app-t, J=9.2 Hz, 1H, C2′Hₐ*), 3.54 (app-dd, J=15.8, 9.2 Hz, 1H, C2Hₐ*), 3.46 (app-dd, J=15.8, 9.5 Hz, 1H, C2Hₐ), 3.35 (app-dt, J=16.2, 8.7 Hz, 2H, C2H_{b}, C2H_{b}*), 3.15-3.07 (m, 1H, C2′H_{b}*), 3.00 (app-td, J=11.6, 7.2 Hz, 1H, C2′H_{b}), 2.96-2.88 (m, 1H, C3′Hₐ*), 2.89-2.81 (m, 8H, N8CH₃,N8CH₃*, C10H, C2″Hₐ), 2.79 (d, J=8.9 Hz, 1H, C10H*), 2.77-2.66 (m, 1H, C3′Hₐ), 2.40-2.31 (m, 3H, C3Hₐ, C3Hₐ*, C2″H_{b}), 2.31-2.22 (m, 4H, C3H_{b}, C3H*, C2″H₂*), 2.03 (app-dd, J=13.2, 7.1 Hz, 1H, C3′H_{b}*), 1.96 (app-dd, J=13.1, 7.0 Hz, 1H, C3′H_{b}), 1.85-1.67 (m, 4H, C3″H₂, C3″H₂*), 1.58 (s, 3H, C13H₃*), 1.55 (s, 3H, C13H₃), 1.39 (s, 3H, C12H₃), 1.36 (s, 3H, C12H₃*), 1.01 (t, J=7.4 Hz, 3H, C4″H₃), 0.96 (t, J=7.5 Hz, 3H, C4″H₃*). ¹³C NMR (150.9 MHz, CDCl₃, 20° C., 12:1 mixture of atropisomers, *denotes minor atropisomer): δ 174.6 (C1″), 173.2 (C1″*), 150.8 (C7a), 150.6 (C7a*), 143.0 (C7a′*), 142.9 (C7a′), 137.2 (C4), 133.3 (C4a′*), 132.8 (C4a′), 132.5 (C4a), 132.3 (C4a*), 129.0 (C6), 128.8 (C6*), 127.5 (C6′), 127.3 (C6′*), 123.7 (C4′*), 123.4 (C4′), 121.0 (C5′*), 120.7 (C5′), 117.2 (C7′*), 117.1 (C7′), 114.1 (C5*), 113.4 (C5), 102.0 (C7), 101.6 (C7*), 83.2 (C8a*), 82.8 (C8a), 79.2 (C8a′), 78.1 (C8a′*), 65.5 (C9), 65.2 (C9*), 64.3 (C10*), 64.2 (CO), 59.8 (C₁), 52.2 (C3a′), 51.7 (C3a), 51.6 (C3a*), 49.8 (C3a′*), 45.4 (C2′*), 44.2 (C2′), 38.7 (C3*), 38.3 (C3), 37.9 (C2*), 37.2 (C2″*), 36.8 (C2″), 36.5 (C2), 32.7 (C3′*), 30.8 (C3′), 29.9 (N8CH₃*), 29.8 (N8CH₃), 25.0 (C12), 24.9 (C12*), 20.6 (2C, C13, C13*), 18.6 (C3″), 18.1 (C3″*), 14.3 (C4″), 14.2 (C4″*). FTIR (thin film) cm⁻¹: 3321 (br-m), 3052 (w), 2961 (m), 2930 (m), 2876 (m), 1639 (m), 1596 (m), 1494 (m), 1427 (m), 1408 (m), 1339 (m), 1265 (m), 1090 (m), 1003 (w), 739 (m). HRMS (ESI) (m/z): calc'd for C₃₀H₃₇N₄O₂ [M+H]⁺: 485.2911, found: 485.2913. [α]_{D}²³: −168 (c=0.38, MOH).⁹³ TLC (30% oacetone in hexanes), Rf: 0.19 (UV, CAM).

TABLE 13
Comparison of 1H NMR data for (−)-communesin H (8) with literature
data (CDCl3, major atropisomer):

| | Christophersen's Isolation | |
|---|---|---|
| | Report^{88,89} | This Work |
| | (−)-Communesin H (8) | (−)-Communesin H (8) |
| | ¹H NMR, 500 MHz, | ¹H NMR, 500 MHz, |
| Assignment | CDCl₃ | CDCl₃ |
| C2 | 3.44 (m, 1H) | 3.46 (app-dd, J = 15.8, |
| | | 9.5 Hz, 1H) |
| | 3.36 (m, 1H) | 3.35 (app-dt, J = 16.2, |
| | | 8.7 Hz, 2H) |
| C3 | 2.35 (m, 1H) | 2.40-2.31 (m, 3H) |
| | 2.27 (m, 1H) | 2.31-2.22 (m, 4H) |
| C3a | — | — |
| C4a | — | — |
| C4 | — | — |
| C5 | 6.07 (d, J = 7.7 Hz, 1H) | 6.07 (d, J = 7.8 Hz, 1H) |
| C6 | 6.89 (t, J = 7.7 Hz, 1H) | 6.88 (t, J = 7.7 Hz, 1H) |
| C7 | 5.95 (d, J = 7.7 Hz, 1H) | 5.95 (d, J = 7.8 Hz, 1H) |
| C7a | — | — |
| N8CH₃ | 2.84 (s, 3H) | 2.89-2.81 (m, 8H) |
| C8a | 4.69 (s, 1H) | 4.69 (s, 1H) |
| C9 | 4.10 (d, J = 9.0 Hz, 1H) | 4.10 (d, J = 9.0 Hz, 1H) |
| C10 | 2.86 (m, 1H) | 2.89-2.81 (m, 8H) |
| C11 | — | — |
| C12 | 1.39 (s, 3H) | 1.39 (s, 3H) |
| C13 | 1.54 (s, 3H) | 1.55 (s, 3H) |
| C2′ | 3.88 (dd, J = 11.8, | 3.88 (app-dd, J = 11.6, |
| | 8.6 Hz, 1H) | 8.9 Hz, 1H) |
| | 3.00 (ddd, J = 11.8, 11.5, | 3.00 (app-td, J = 11.6, |
| | 7.4 Hz, 1H) | 7.2 Hz, 1H) |
| C3′ | 2.72 (ddd, J = 13.2, 11.5, | 2.77-2.66 (m, 1H) |
| | 8.6 Hz, 1H) | 1.96 (app-dd, J = 13.1, |
| | 1.96 (dd, J = 13.2, 7.4 Hz, | 7.0 Hz, 1H) |
| | 1H) | |
| C3a′ | — | — |
| C4a′ | — | — |
| C4′ | 6.66 (m, 1H) | 6.73-6.63 (m, 5H) |
| C5′ | 6.68 (m, 1H) | 6.73-6.63 (m, 5H) |
| C6′ | 6.99 (td, J = 7.5, 1.5 Hz, | 7.00 (app-td, J = 7.5, |
| | 1H) | 1.5 Hz, 2H) |
| C7′ | 6.70 (m, 1H) | 6.73-6.63 (m, 5H) |
| C7a′ | — | — |
| C8a′ | 5.04 (s, 1H) | 5.04 (d, J = 1.4 Hz, 1H) |
| C1″ | — | — |
| C2″ | 2.84 (m, 1H) | 2.89-2.81 (m, 8H) |
| | 2.35 (m, 1H) | 2.40-2.31 (m, 3H) |
| C3″ | 1.75 (m, 2H) | 1.85-1.67 (m, 4H) |
| C4″ | 1.00 (t, J = 7.4 Hz, 3H) | 1.01 (t, J = 7.4 Hz, 3H) |
| N8′H | — | 4.59br-s, 1H) |

TABLE 14
Comparison of ¹³C NMR data for (−)-communesin H (8) with
literature data (CDCl₃, major atropisomer):

| | Christophersen's | This Work | Chemical Shift |
|---|---|---|---|
| | Isolation Report^{88,90} | (−)-Communesin | Difference |
| | (−)-Communesin | H (8) ¹³C NMR, | Δδ = δ (this work) - |
| | H (8) ¹³C NMR, | 150.9 MHz, | δ (Christophersen |
| Assignment | 75 MHz CDCl₃ | CDCl₃ | report) |
| C2 | 36.3 | 36.5 | 0.2 |
| C3 | 38.1 | 38.3 | 0.2 |
| C3a | 51.9 | 51.7 | −0.2⁹⁴ |
| C4a | 132.3 | 132.5 | 0.2 |
| C4 | 136.9 | 137.2 | 0.3 |
| C5 | 113.2 | 113.4 | 0.2 |
| C6 | 128.8 | 129.0 | 0.2 |
| C7 | 101.7 | 102.0 | 0.3 |
| C7a | 150.5 | 150.8 | 0.3 |
| N8CH₃ | 29.6 | 29.8 | 0.2 |
| C8a | 82.4 | 82.8 | 0.4 |
| C9 | 65.2 | 65.5 | 0.3 |
| C10 | 63.9 | 64.2 | 0.3 |
| C11 | 59.6 | 59.8 | 0.2 |
| C12 | 24.8 | 25.0 | 0.2 |
| C13 | 20.4 | 20.6 | 0.2 |
| C2′ | 44.0 | 44.2 | 0.2 |
| C3′ | 30.8 | 30.8 | 0.0 |
| C3a′ | 51.4 | 52.2 | 0.8⁹⁴ |
| C4a′ | 132.4 | 132.8 | 0.4 |
| C4′ | 123.2 | 123.4 | 0.2 |
| C5′ | 116.9 | 120.7 | 3.8⁹⁵ |
| C6′ | 127.3 | 127.5 | 0.2 |
| C7′ | 120.5 | 117.1 | −3.4⁹⁵ |
| C7a′ | 142.6 | 142.9 | 0.3 |
| C8a′ | 78.9 | 79.2 | 0.3 |
| C1″ | 174.5 | 174.6 | 0.1 |
| C2″ | 36.6 | 36.8 | 0.2 |
| C3″ | 18.4 | 18.6 | 0.2 |
| C4″ | 14.2 | 14.3 | 0.1 |

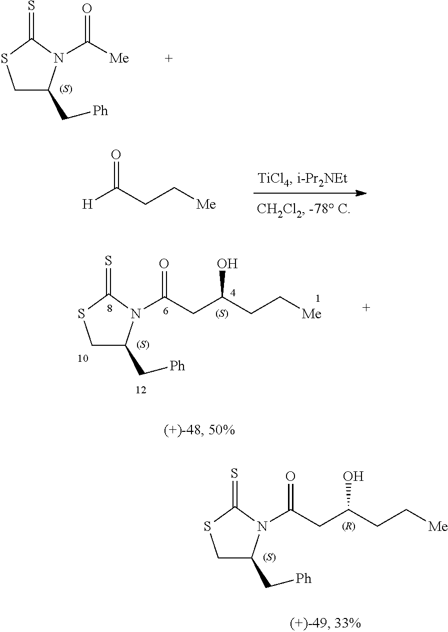

### Example 38: Aldol Adducts (+)-48 and (+)-49

A sample of (S)-1-(4-benzyl-2-thioxothiazolidin-3-yl)ethan-1-one⁹⁶ (648 mg, 2.58 mmol, 1equiv) was azeotropically dried by concentration from anhydrous benzene (2×5 mL). The residue was dissolved in dichloromethane (8.0 mL) and the resulting bright-yellow solution was cooled to −78° C. A freshly-prepared solution of titanium(IV) chloride (489 mg, 2.58 mmol, 1.00 equiv) in dichloromethane (2.0 mL) was then added dropwise over 4 min. The transfer was quantitated with additional dichloromethane (1.0 mL). After stirring at −78° C. for 10 min N,N-diisopropylethyl amine (898 μL, 5.15 mmol, 2.00 equiv) was added dropwise via syringe over 4 min to the resulting bright-orange viscous suspension causing an immediate colour change to dark burgundy. After 1 h, a solution of butyraldehyde (697 μL, 7.73 mmol, 3.00 equiv) in dichloromethane (4.0 mL) was added dropwise via syringe over 3 min and the transfer was quantitated with additional dichloromethane (1.0 mL). After 45 min, the resulting homogeneous burgundy-orange solution was diluted with a saturated aqueous ammonium chloride solution (20 mL) and deionized water (20 mL). The cooling bath was removed and the mixture was allowed to warm to 23° C. The layers were separated and the aqueous layer was extracted with dichloromethane (2×25 mL). The combined organic extracts were washed sequentially with an aqueous sodium bisulfite solution (1 M, 3×40 mL) and a saturated aqueous sodium chloride solution (40 mL), were dried over anhydrous sodium sulfate, were filtered, and were concentrated under reduced pressure. The residue was purified by flash column chromatography on silica gel (eluent: 30%→40% diethyl ether in hexanes) to afford impure aldol adducts (+)-48 (more polar) and (+)-49 (less polar) as viscous yellow oils. Each sample was independently subjected to a second chromatographic purification on silica gel (eluent: 0%→2% diethyl ether in dichloromethane) to afford pure (11S,4S)-adduct (+)-48 (420 mg, 50.3%) as a bright-yellow viscous oil and pure (11S,4R)-adduct (+)-49 (278 mg, 33.4%) as a bright-yellow solid.⁹⁷ Structural assignments were made using additional information from gCOSY, gHSQC, and gHMBC experiments.

(11S,4S)-Aldol adduct (+)-48: ¹H NMR (500 MHz, CDCl₃, 20° C.): δ 7.38-7.31 (m, 2H, Ph-m-H), 7.31-7.26 (m, 3H, Ph-o-H, Ph-p-H), 5.40 (app-ddd, J=10.8, 7.1, 4.0 Hz, 1H, C11H), 4.21-4.12 (m, 1H, C4H), 3.64 (dd, J=17.8, 2.4 Hz, 1H, C5Hₐ), 3.40 (ddd, J=11.6, 7.2, 1.0 Hz, 1H, C10Hₐ), 3.22 (dd, J=13.2, 4.0 Hz, 1H, C12Hₐ), 3.13 (dd, J=17.8, 9.5 Hz, 1H, C5H_{b}), 3.05 (dd, J=13.2, 10.5 Hz, 1H, C12H_{b}), 2.89 (d, J=11.6 Hz, 1H, C10H_{b}), 2.77 (d, 1H, J=3.9 Hz, C40H), 1.64-1.54 (m, 1H, C3Hₐ), 1.54-1.34 (m, 3H, C2H₂, C3H_{b}), 0.95 (t, J=6.9 Hz, 3H, C1H₃). ¹³C NMR (125.8 MHz, CDCl₃, 24° C.): δ 201.5 (C8), 173.4 (C6), 136.5 (Ph-ipso-C), 129.5 (Ph-o-C), 129.0 (Ph-m-C), 127.4 (Ph-p-C), 68.4 (C11), 67.7 (C4), 46.0 (C5), 38.6 (C3), 36.9 (C12), 32.2 (C10), 18.9 (C2), 14.1 (C₁). FTIR (thin film) cm⁻¹: 3448 (br-w), 2957 (m), 2930 (m), 1691 (s), 1455 (m), 1342 (s), 1267 (s), 1192 (m), 1138 (s), 1044 (s). HRMS (ESI) (m/z): calc'd for C₁₆H₂₁NNaO₂S₂ [M+Na]⁺: 346.0906, found: 346.0914. [α]_{D}²³: +234 (c=0.89, CH₂C₂). TLC (40% diethyl ether in hexanes), Rf: 0.16 (UV, CAM).

(11S,4R)-Aldol adduct (+)-49: ¹H NMR (500 MHz, CDCl₃, 25° C.): δ 7.38-7.32 (m, 2H, Ph-m-H), 7.31-7.26 (m, 3H, Ph-o-H, Ph-p-H), 5.41 (app-ddd, J=10.8, 7.1, 4.0 Hz, 1H, C11H), 4.07 (dddd, J=9.9, 7.4, 4.3, 2.5 Hz, 1H, C4H), 3.46 (dd, J=17.5, 9.4 Hz, 1H, C5Hₐ), 3.40 (dd, J=11.6, 7.2 Hz, 1H, C10Hₐ), 3.33 (dd, J=17.5, 2.6 Hz, 1H, C5H_{b}), 3.22 (dd, J=13.2, 4.0 Hz, 1H, C12Hₐ), 3.13 (br-s, 1H, C40H), 3.04 (dd, J=13.2, 10.4 Hz, 1H, C12H_{b}), 2.91 (d, J=11.6 Hz, 1H, C10H_{b}), 1.64-1.53 (m, 1H, C3Hₐ), 1.53-1.34 (m, 3H, C2H₂, C3H), 0.94 (t, J=6.9 Hz, 3H, C1H₃). ¹³C NMR (125.8 MHz, CDCl₃, 25° C.): δ 201.6 (C8), 174.0 (C6), 136.5 (Ph-ipso-C), 129.5 (Ph-o-C), 129.1 (Ph-m-C), 127.4 (Ph-p-C), 68.3 (2C, C4, C11), 45.6 (C5), 38.9 (C3), 36.9 (C12), 32.1 (C10), 18.8 (C2), 14.1 (C1). FTIR (thin film) cm⁻¹: 3435 (br-w), 2957 (m), 2930 (m), 1693 (m), 1454 (w), 1342 (s), 1293 (m), 1259 (s), 1164 (s), 1138 (s), 1041 (m). HRMS (ESI) (m/z): calc'd for C₁₆H₂₁NNaO₂S₂ [M+Na]⁺: 346.0906, found: 346.0901. [α]_{D}²³: +160 (c=0.81, CH₂Cl₂). TLC (40% diethyl ether in hexanes), Rf: 0.28 (UV, CAM).

### Example 39: Determination of the Relative Stereochemistry of Aldol Adducts (+)-48 and (+)-49

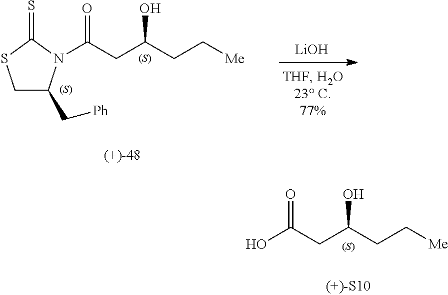

(+)-(3S)-Hydroxyhexanoic acid (S10): An aqueous lithium hydroxide solution (1.0 M, 1.7 mL, 1.7 mmol, 4.0 equiv) was added to a bright-yellow solution of (11S,4S)-aldol adduct (+)-48 (138 mg, 0.430 mmol, 1 equiv) in tetrahydrofuran (1.50 mL) at 23° C. After 30 min, the resulting off-white turbid solution was concentrated under reduced pressure to remove tetrahydrofuran. The aqueous suspension was diluted with deionized water (10 mL) and was washed with ethyl acetate (4×10 mL) to remove residual (S)-4-benzylthiazolidine-2-thione. The aqueous layer was acidified to pH 1 by adding an aqueous hydrogen chloride solution (1 M, 3 mL) and was extracted with ethyl acetate (3×10 mL). The combined organic extracts were dried over anhydrous sodium sulfate, were filtered, and were concentrated under reduced pressure to yield (+)-(3S)-hydroxyhexanoic acid (S10, 43.6 mg, 77.2%) as a colourless semi-solid. Spectral data were in agreement with those previously reported in the literature.⁹⁶ 1H NMR (500 MHz, CDCl₃, 20° C.): δ 7.62 (br-s, 1H), 4.05 (tdd, J=7.9, 4.5, 3.1 Hz, 1H), 2.55 (dd, J=16.5, 3.2 Hz, 1H), 2.46 (dd, J=16.5, 9.1 Hz, 1H), 1.60-1.28 (m, 4H), 0.92 (t, J=7.0 Hz, 3H). ¹³C NMR (100.6 MHz, CDCl₃, 25° C.): δ 178.0, 68.0, 41.3, 38.6, 18.8, 14.0. [α]_{D}²³: +25 (c=2.18, CHCl₃).⁹⁸

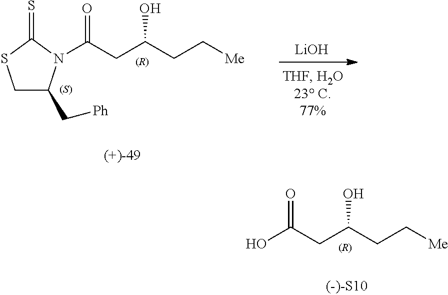

(−)-(3R)-Hydroxyhexanoic acid (S10): An aqueous lithium hydroxide solution (1.0 M, 1.8 mL, 1.8 mmol, 4.0 equiv) was added to a bright-yellow solution of (11S,4R)-aldol adduct (+)-49 (143 mg, 0.440 mmol, 1 equiv) in tetrahydrofuran (2.20 mL) at 23° C. After 30 min, the resulting off-white turbid solution was concentrated under reduced pressure to remove tetrahydrofuran. The aqueous suspension was diluted with deionized water (10 mL) and was washed with ethyl acetate (4×10 mL) to remove residual (S)-4-benzylthiazolidine-2-thione. The aqueous layer was acidified to pH 1 by adding an aqueous hydrogen chloride solution (1 M, 3 mL) and was extracted with ethyl acetate (3×10 mL). The combined organic extracts were dried over anhydrous sodium sulfate, were filtered, and were concentrated under reduced pressure to yield (−)-(3R)-hydroxyhexanoic acid (S10, 44.0 mg, 76.6%) as a colourless viscous oil. Spectral data for (−)-(3R)-S10 were identical to those obtained for (+)-(3S)-S10 as described above. [α]_{D}²³: −28 (c=2.20, CHCl₃).⁹⁹

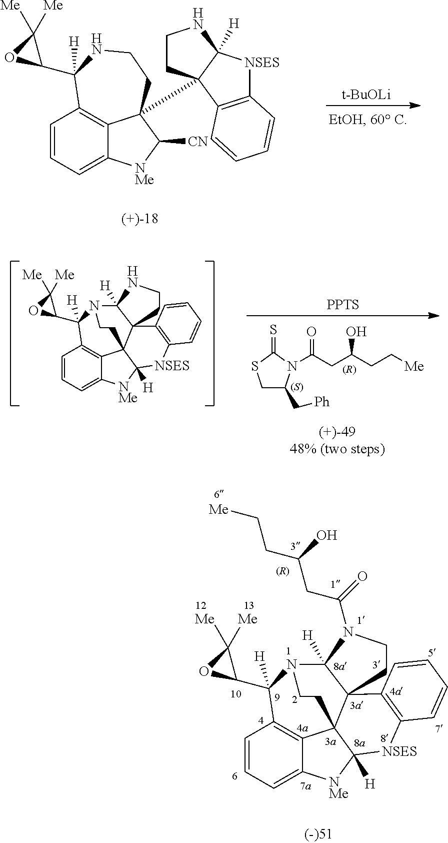

### Example 40: (−)-N8′-(Trimethylsilyl)ethanesulfonyl communesin I(51)

A sample of lithium tert-butoxide (12.8 mg, 0.160 mmol, 10.0 equiv) was added to a solution of heterodimer (+)-18 (9.67 mg, 16.0 μmol, 1equiv) in anhydrous ethanol (200 proof, 400 μL). The flask was sealed with a Teflon-lined glass stopper under an argon atmosphere and was immersed in a preheated oil bath at 60° C. After 16 h, the reaction mixture was cooled to 23° C. and samples of pyridinium p-toluenesulfonate (PPTS, 34.1 mg, 136 μmol, 8.50 equiv) and (11S,4R)-aldol adduct (+)-49 (77.7 mg, 0.240 mmol, 15.1 equiv) were added sequentially. After 23 min, an additional portion of (+)-49 (79.4 mg, 0.246 mmol, 15.4 equiv) was added. After 47 min, a final portion of (+)-49 (114 mg, 0.352 mmol, 22.1 equiv) was added. After 96 min, a saturated aqueous sodium bicarbonate solution (8 mL) was added and the resulting mixture was extracted with dichloromethane (3×5 mL). The combined organic extracts were dried over anhydrous sodium sulfate, were filtered, and were concentrated under reduced pressure. The residue was purified by flash column chromatography on silica gel (eluent: 20%→30% acetone in hexanes) to afford (−)-N8′-(trimethylsilyl)ethanesulfonyl communesin I (51, 5.27 mg, 47.7%) as a white solid. Structural assignments were made using additional information from gCOSY, gHSQC, gHMBC, and 1D selective ROESY experiments. ¹H NMR (500 MHz, CDCl₃, 20° C., 5.4:1 mixture of atropisomers, *denotes minor atropisomer): δ 7.56 (dd, J=8.0, 1.2 Hz, 1H, C7′H), 7.40 (d, J=7.9 Hz, 1H, C7′H*), 7.21 (app-td, J=7.8, 1.4 Hz, 2H, C6′H, C6′H*), 7.07 (app-td, J=7.6, 1.3 Hz, 2H, C5′H, C5′H*), 6.91 (t, J=7.8 Hz, 2H, C6H, C6H*), 6.83 (dd, J=7.8, 1.4 Hz, 1H, C4′H), 6.72 (d, J=7.6 Hz, 1H, C4′H*), 6.14 (d, J=7.7 Hz, 1H, C5H*), 6.09 (d, J=7.7 Hz, 1H, C5H), 5.98 (d, J=7.8 Hz, 1H, C7H), 5.94 (d, J=7.7 Hz, 1H, C7H*), 5.72 (s, 1H, C8aH), 5.63 (s, 1H, C8aH*), 5.45 (s, 1H, C8a′H*), 5.07 (s, 1H, C8a′H), 4.48 (d, J=8.2 Hz, 1H, C9H*), 4.20-4.14 (m, 1H, C3″H*), 4.11 (d, J=9.1 Hz, 1H, C9H), 4.09-4.03 (m, 1H, C3″H), 3.97 (app-dd, J=12.0, 8.7 Hz, 1H, C2′Hₐ), 3.70 (app-t, J=9.4 Hz, 1H, C2′Hₐ*), 3.48 (app-dd, J=16.0, 9.6 Hz, 1H, C2Hₐ), 3.41-3.31 (m, 1H, C2H_{b}), 3.25 (app-td, J=13.3, 4.9 Hz, 1H, N8′SO₂CHa), 3.17 (app-td, J=13.4, 4.9 Hz, 1H, N8′SO₂CH_{b}), 3.07 (app-td, J=11.7, 7.3 Hz, 1H, C2′H_{b}), 3.03-2.95 (m, 1H, C3′Hₐ*), 2.92 (s, 3H, N8CH₃), 2.88-2.74 (m, 3H, C10H, C3′Hₐ, C2″Hₐ), 2.87 (s, 3H, N8CH₃*), 2.56-2.43 (m, 2H, C3Hₐ, C2″H_{b}), 2.36-2.24 (m, 1H, C3H_{b}), 2.19-2.07 (m, 1H, C3′H_{b}*), 1.94 (app-dd, J=13.1, 7.2 Hz, 1H, C3′H_{b}), 1.61-1.39 (m, 7H, C12/13H₃, C4″H₂, C5″H₂), 1.38 (s, 3H, C12/13H₃), 1.36 (s, 3H, C12/13H₃*), 1.30-1.13 (m, 2H, N8'SO₂CH₂CH₂), 0.95 (t, J=6.9 Hz, 3H, C6″H₃), 0.92 (t, J=6.9 Hz, 3H, C6″H₃*), 0.11 (s, 9H, Si(CH₃)₃*), 0.07 (s, 9H, Si(CH₃)₃). ¹³C NMR (125.8 MHz, CDCl₃, 25° C., 5.4:1 mixture of atropisomers, *denotes minor atropisomer): δ 173.8 (C1″), 173.2 (C1″*), 149.9 (C7a), 149.5 (C7a*), 138.9 (C4a′*), 138.0 (C4a′), 136.6 (C4), 136.1 (C7a′), 136.0 (C7a′*), 131.1 (C4a), 129.5 (C6), 129.3 (C6*), 128.0 (C6′), 127.8 (C5′*), 126.4 (C5′), 125.5 (C7′*), 124.9 (C7′), 124.1 (C4′*), 123.9 (C4′), 114.6 (C5*), 113.8 (C5), 102.7 (C7), 102.4 (C7*), 85.2 (C8a*), 84.7 (C8a), 79.5 (C8a′), 78.2 (C8a′*), 69.0 (C3″), 67.6 (C3″*), 65.3 (2C, C9, C9*), 64.0 (C10), 60.5 (C11), 60.1 (C11*), 54.1 (2C, C3a, C3a*), 52.6 (C3a′), 51.9 (N8′SO₂CH₂), 50.1 (C3a′*), 45.2 (C2′*), 44.1 (C2′), 42.1 (C2″), 41.3 (C2″*), 39.5 (C4″), 38.7 (C4″*), 37.5 (C3), 36.3 (C2), 33.2 (C3′*), 31.5 (C3′), 31.0 (N8CH₃*), 30.9 (N8CH₃), 25.0 (C12/13), 24.8 (C12/13*), 20.6 (C12/13*), 20.5 (C12/13), 19.1 (C5″), 18.9 (C5″*), 14.3 (C6″), 14.2 (C6″*), 10.8 (N8′SO₂CH₂CH₂*), 10.7 (N8′SO₂CH₂CH₂), −1.7 (Si(CH₃)₃*), −1.8 (Si(CH₃)₃). FTIR (thin film) cm⁻¹: 3474 (br-w), 2956 (s), 1636 (s), 1600 (s), 1489 (m), 1457 (m), 1338 (s), 1251 (m), 1157 (s), 1053 (m), 860 (m), 739 (m). HRMS (ESI) (m/z): calc'd for C₃₇H₃N₄O₅SSi [M+H]⁺: 693.3500, found: 693.3482 [α]_{D}²⁷: −111 (c=0.27, CH₂Cl₂). TLC (30% acetone in hexanes), Rf: 0.25 (UV, CAM).

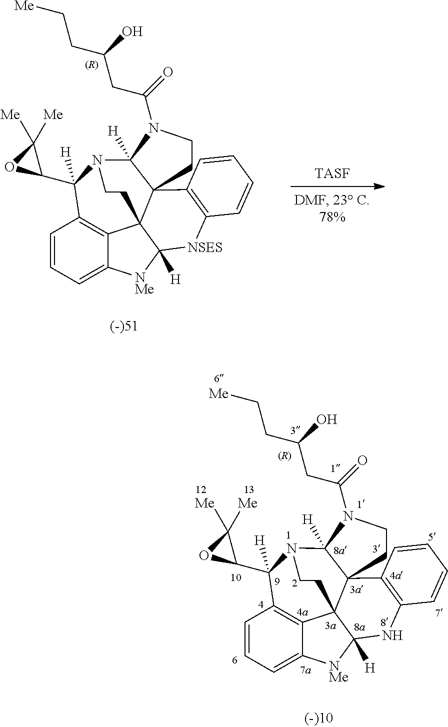

### Example 41: (−)-Communesin I (10)¹⁰⁰

A solution of tris(dimethylamino)sulfonium difluorotrimethylsilicate (TASF, 8.3 mg, 0.030 mmol, 4.0 equiv) in N,N-dimethylformamide (50 μL) was added to a solution of (−)-N8′-(trimethylsilyl)-ethanesulfonyl communesin I (51, 5.2 mg, 7.5 μmol, 1 equiv) in N,N-dimethylformamide (200 L) at 23° C. After 2 h, a saturated aqueous sodium chloride solution (5 mL) and deionized water (3 mL) were added and the mixture was extracted with ethyl acetate (3×5 mL). The combined organic extracts were washed with a saturated aqueous sodium chloride solution (2×8 mL), were dried over anhydrous sodium sulfate, were filtered, and were concentrated under reduced pressure. The resulting residue was purified by flash column chromatography on silica gel (eluent: 50%→60% ethyl acetate in dichloromethane) to afford (−)-communesin I (10, 3.11 mg, 78.4%) as a colourless film. Structural assignments were made using additional information from gCOSY, gHSQC, gHMBC, and 1D selective NOESY experiments. ¹H NMR (600 MHz, CDCl₃, 25° C., 11:1 mixture of atropisomers, *denotes minor atropisomer): δ 7.01 (app-td, J=7.6, 1.4 Hz, 2H, C6′H, C6′H*), 6.88 (t, J=7.7 Hz, 1H, C6H), 6.85 (t, J=8.2 Hz, 1H, C6H*), 6.72 (t, J=7.4 Hz, 2H, C5′H, C5′H*), 6.69 (d, J=7.7 Hz, 2H, C7′H, C7′H*), 6.68 (d, J=6.7 Hz, 1H, C4′H), 6.58 (d, J=7.5 Hz, 1H, C4′H*), 6.09 (d, J=7.6 Hz, 1H, C5H*), 6.06 (d, J=7.6 Hz, 1H, C5H), 5.95 (d, J=7.7 Hz, 1H, C7H), 5.91 (d, J=7.9 Hz, 1H, C7H*), 5.41 (app-s, 1H, C8a′H*), 5.05 (d, J=1.5 Hz, 1H, C8a′H), 4.70 (s, 1H, C8aH), 4.67 (s, 1H, C8aH*), 4.59 (br-s, 1H, N8′H), 4.47 (d, J=9.1 Hz, 1H, C9H*), 4.20-4.15 (m, 1H, C3″H*), 4.14 (d, J=5.9 Hz, 1H, C3″OH), 4.11 (d, J=9.3 Hz, 1H, C9H), 4.09-4.02 (m, 1H, C3″H), 3.92 (app-dd, J=12.0, 8.4 Hz, 1H, C2′Hₐ), 3.64 (app-t, J=9.1 Hz, 1H, C2′Hₐ*), 3.55 (app-dd, J=16.1, 8.9 Hz, 1H, C2Hₐ*), 3.46 (app-dd, J=15.9, 9.6 Hz, 1H, C2Hₐ), 3.41-3.30 (m, 2H, C2H_{b}, C2H_{b}*), 3.13-3.06 (m, 1H, C2′H_{b}*), 3.00 (app-td, J=11.8, 7.2 Hz, 1H, C2′H_{b}), 2.97-2.89 (m, 1H, C3′Hₐ*), 2.87 (d, J=9.1 Hz, 1H, C10H), 2.86-2.81 (m, 7H, N8CH₃, N8CH₃*, C2″Hₐ), 2.80 (d, J=9.1 Hz, 1H, C10H*), 2.71 (app-td, J=12.4, 8.6 Hz, 1H, C3′Hₐ), 2.49 (app-dd, J=14.5, 3.6 Hz, 1H, C2″H_{b}), 2.37 (app-dd, J=13.1, 6.9 Hz, 2H, C3Hₐ, C3Hₐ*), 2.29 (app-dt, J=13.0, 9.3 Hz, 2H, C3H_{b}, C3H_{b}*), 2.08-2.03 (m, 1H, C3′H_{b}*), 2.00 (app-dd, J=13.3, 6.9 Hz, 1H, C3′H_{b}), 1.64-1.50 (m, 9H, C13H₃, C13H₃*, C4″H₂, C5″Hₐ), 1.49-1.40 (m, 1H, C5″H_{b}), 1.39 (s, 3H, C12H₃), 1.37 (s, 3H, C12H₃*), 0.96 (t, J=7.1 Hz, 3H, C6″H₃), 0.93 (t, J=7.2 Hz, 3H, C6″H₃*). ¹³C NMR (150.9 MHz, CDCl₃, 25° C., 11:1 mixture of atropisomers, *denotes minor atropisomer): δ 174.1 (C1″), 150.7 (C7a), 142.8 (C7a′), 136.3 (C4), 132.3 (C4a), 132.2 (C4a′), 129.1 (C6), 128.9 (C6*), 127.7 (C6′), 127.5 (C6′*), 123.4 (C4′*), 123.3 (C4′), 121.2 (C5′*), 120.7 (C5′), 117.3 (C7′*), 117.2 (C7′), 113.3 (C5), 102.0 (C7), 101.6 (C7*), 83.0 (C8a*), 82.6 (C8a), 79.3 (C8a′), 77.9 (C8a′*), 69.2 (C3″), 67.6 (C3″*), 65.4 (C9), 65.2 (C9*), 64.2 (2C, C10, C10*), 60.5 (C11), 52.2 (C3a′), 51.5 (C3a), 45.3 (C2′*), 44.2 (C2′), 42.2 (C2″), 39.6 (C4″), 37.9 (C3), 36.2 (C2), 32.5 (C3′*), 30.8 (C3′), 29.8 (N8CH₃*), 29.7 (N8CH₃), 25.0 (C12), 24.9 (C12*), 20.6 (C13*), 20.5 (C13), 19.1 (C5″), 14.3 (C6″), 14.2 (C6″*). FTIR (thin film) cm⁻¹:3460 (br-w), 3325 (br-w), 3052 (w), 2957 (m), 2927 (m), 2872 (m), 1625 (s), 1606 (s), 1596 (s), 1493 (s), 1428 (s), 1338 (m), 1279 (m), 1002 (m), 908 (m), 737 (s). HRMS (ESI) (m/z): calc'd for C₃₂H₄₁N₄O₃ [M+H]⁺: 529.3173, found: 529.3167. [α]_{D}²³: −137 (c=0.22, MeOH).¹⁰¹ TLC (50% ethyl acetate in dichloromethane), Rf: 0.19 (UV, CAM).

TABLE 15
Comparison of ¹H NMR data for (−)-communesin I (10) with literature
data (CDCl₃, major atropisomer):

| | Chen's Isolation Report¹⁰² | This Work |
|---|---|---|
| | (−)-Communesin I (10) | (−)-Communesin I (10) |
| Assignment | ¹H NMR, 600 MHz, CDCl₃ | ¹H NMR, 600 MHz, CDCl₃ |
| C2 | 3.46 (dd, J = 15.8, 9.5 Hz, 1H) | 3.46 (app-dd, J = 15.9, 9.6 Hz, 1H) |
| | 3.36 (dt, J = 15.8, 8.5 Hz, 1H) | 3.41-3.30 (m, 2H) |
| C3 | 2.37 (dd, J = 12.8, 8.5 Hz, 1H) | 2.37 (app-dd, J = 13.1, 6.9 Hz, 2H) |
| | 2.28 (dt, J = 12.8, 9.5 Hz, 1H) | 2.29 (app-dt, J = 13.0, 9.3 Hz, 2H) |
| C3a | — | — |
| C4a | — | — |
| C4 | — | — |
| C5 | 6.05 (d, J = 7.7 Hz, 1H) | 6.06 (d, J = 7.6 Hz, 1H) |
| C6 | 6.88 (t, J = 7.7 Hz, 1H) | 6.88 (t, J = 7.7 Hz, 1H) |
| C7 | 5.95 (d, J = 7.7 Hz, 1H) | 5.95 (d, J = 7.7 Hz, 1H) |
| C7a | — | — |
| N8CH₃ | 2.84 (s, 3H) | 2.86-2.81 (m, 7H) |
| C8a | 4.70 (s, 1H) | 4.70 (s, 1H) |
| C9 | 4.11 (d, J = 9.0 Hz, 1H) | 4.11 (d, J = 9.3 Hz, 1H) |
| C10 | 2.87 (d, J = 9.0 Hz, 1H) | 2.87 (d, J = 9.1 Hz, 1H) |
| C11 | — | — |
| C12 | 1.39 (s, 3H) | 1.39 (s, 3H) |
| C13 | 1.57 (s, 3H) | 1.64-1.50 (m, 9H) |
| C2′ | 3.92 (dd, J = 12.2, 8.7 Hz, 1H) | 3.92 (app-dd, J = 12.0, 8.4 Hz, 1H) |
| | 3.00 (dt, J = 12.2, 7.1 Hz, 1H) | 3.00 (app-td, J = 11.8, 7.2 Hz, 1H) |
| C3′ | 2.71 (dt, J = 12.2, 8.7 Hz, 1H) | 2.71 (app-td, J = 12.4, 8.6 Hz, 1H) |
| | 2.00 (dd, J = 12.2, 7.1 Hz, 1H) | 2.00 (app-dd, J = 13.3, 6.9 Hz, 1H) |
| C3a′ | — | — |
| C4a′ | — | — |
| C4′ | 6.68 (d, J = 7.6 Hz, 1H) | 6.68 (d, J = 6.7 Hz, 1H) |
| C5′ | 6.71 (t, J = 7.6 Hz, 1H) | 6.72 (t, J = 7.4 Hz, 2H) |
| C6′ | 7.01 (t, J = 7.6 Hz, 1H) | 7.01 (app-td, J = 7.6, 1.4 Hz, 1H) |
| C7′ | 6.69 (d, J = 7.6 Hz, 1H) | 6.69 (d, J = 7.7 Hz, 2H) |
| C7a′ | — | — |
| C8a′ | 5.05 (s, 1H) | 5.05 (d, J = 1.5 Hz, 1H) |
| C1″ | — | — |
| C2″ | 2.82 (dd, J = 14.6, 3.4 Hz, 1H) | 2.86-2.81 (m, 7H) |
| | 2.48 (dd, J = 14.6, 3.4 Hz, 1H) | 2.49 (dd, J = 14.5, 3.6 Hz, 1H) |
| C3″ | 4.06 (br-s, 1H) | 4.09-4.02 (m, 1H) |
| C3″OH | 4.14 (br-s, 1H) | 4.14 (d, J = 5.9 Hz, 1H) |
| C4″ | 1.60 (m, 1H), 1.53 (m, 1H) | 1.64-1.50 (m, 9H) |
| C5″ | 1.54 (m, 1H), 1.44 (m, 1H) | 1.64-1.50 (m, 9H), 1.49-1.40 (m, |
| | | 1H) |
| C6″ | 0.96 (t, J = 7.0 Hz, 3H) | 0.96 (t, J = 7.1 Hz, 3H) |
| N8′H | — | 4.59br-s, 1H) |

TABLE 16
Comparison of ¹³C NMR data for (−)-communesin I (10) with
literature data (CDCl₃, major atropisomer):

| | Chen's Isolation | | |
|---|---|---|---|
| | Report¹⁰² | This Work | Chemical Shift |
| | (−)-Communesin | (−)-Communesin | Difference |
| | I (10) ¹³C NMR, | I (10) ¹³C NMR, | Δδ = δ |
| | 150 MHz | 150.9 MHz, | (this work) - |
| Assignment | CDCl₃ | CDCl₃ | δ (Chen report) |
| C2 | 36.1 | 36.2 | 0.1 |
| C3 | 37.7 | 37.9 | 0.2 |
| C3a | 51.3 | 51.5 | 0.2 |
| C4a | 132.1 | 132.3 | 0.2 |
| C4 | 136.1 | 136.3 | 0.2 |
| C5 | 113.1 | 113.3 | 0.2 |
| C6 | 129.0 | 129.1 | 0.1 |
| C7 | 101.9 | 102.0 | 0.1 |
| C7a | 150.5 | 150.7 | 0.2 |
| N8CH₃ | 29.6 | 29.7 | 0.1 |
| C8a | 82.4 | 82.6 | 0.2 |
| C9 | 65.2 | 65.4 | 0.2 |
| C10 | 64.1 | 64.2 | 0.1 |
| C11 | 60.4 | 60.5 | 0.1 |
| C12 | 24.9 | 25.0 | 0.1 |
| C13 | 20.3 | 20.5 | 0.2 |
| C2′ | 44.1 | 44.2 | 0.1 |
| C3′ | 30.6 | 30.8 | 0.2 |
| C3a′ | 52.1 | 52.2 | 0.1 |
| C4a′ | 132.1 | 132.3 | 0.2 |
| C4′ | 123.2 | 123.3 | 0.1 |
| C5′ | 120.6 | 120.7 | 0.1 |
| C6′ | 127.5 | 127.7 | 0.2 |
| C7′ | 117.0 | 117.2 | 0.2 |
| C7a′ | 142.6 | 142.8 | 0.2 |
| C8a′ | 79.1 | 79.3 | 0.2 |
| C1″ | 173.9 | 174.1 | 0.2 |
| C2″ | 42.1 | 42.2 | 0.1 |
| C3″ | 69.0 | 69.2 | 0.2 |
| C4″ | 39.5 | 39.6 | 0.1 |
| C5″ | 18.9 | 19.1 | 0.2 |
| C6″ | 14.1 | 14.3 | 0.2 |

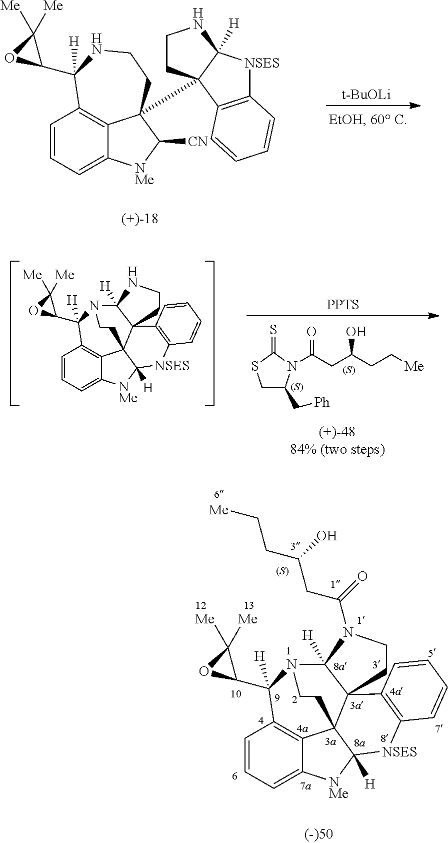

### Example 42: (−)-N8′-(Trimethylsilyl)ethanesulfonyl (C3″S)-communesin I (50)

A sample of lithium tert-butoxide (11.5 mg, 143 μmol, 10.0 equiv) was added to a solution of heterodimer (+)-18 (8.67 mg, 14.3 μmol, 1 equiv) in anhydrous ethanol (200 proof, 380 L) at 23° C. The flask was sealed with a Teflon-lined glass stopper under an argon atmosphere and was immersed in a preheated oil bath at 60° C. After 18.5 h, the reaction mixture was cooled to 23° C. and a sample of pyridinium p-toluenesulfonate (PPTS, 28.8 mg, 114 μmol, 8.00 equiv) was added as a solid followed by a solution of (11S,4S)-aldol adduct (+)-48 (253 mg, 782 μmol, 54.7 equiv) in dichloromethane (500 μL). The transfer was quantitated with additional dichloromethane (300 μL). After 50 min, the solution was diluted with a saturated aqueous sodium bicarbonate solution (5 mL) and deionized water (5 mL) and the resulting mixture was extracted with ethyl acetate (3×5 mL). The combined organic extracts were washed with a saturated aqueous sodium chloride solution (10 mL), were dried over anhydrous sodium sulfate, were filtered, and were concentrated under reduced pressure. The residue was purified by flash column chromatography on silica gel (eluent: 20%→30% acetone in hexanes) to afford (−)-N8′-(trimethylsilyl)ethanesulfonyl (C3″S)-communesin I (50, 8.27 mg, 83.5%) as a white solid. Structural assignments were made using additional information from gCOSY, gHSQC, gHMBC, and 1D selective NOESY experiments. ¹H NMR (600 MHz, CDCl₃, 20° C., 15:1 mixture of atropisomers, *denotes minor atropisomer): δ 7.54 (dd, J=8.0, 1.2 Hz, 1H, C7′H), 7.42 (d, J=8.5 Hz, 1H, C7′H*), 7.20 (app-td, J=7.7, 1.4 Hz, 2H, C6′H, C6′H*), 7.07 (app-td, J=7.6, 1.2 Hz, 2H, C5′H, C5′H*), 6.91 (app-t, J=7.7 Hz, 2H, C6H, C6H*), 6.84 (dd, J=7.7, 1.4 Hz, 1H, C4′H), 6.74-6.70 (m, 1H, C4′H*), 6.15 (d, J=7.6 Hz, 1H, C5H*), 6.10 (d, J=7.7 Hz, 1H, C5H), 5.98 (d, J=7.8 Hz, 1H, C7H), 5.96 (d, J=7.0 Hz, 1H, C7H*), 5.71 (s, 1H, C8aH), 5.64 (s, 1H, C8aH*), 5.50 (br-s, 1H, C8a′H*), 5.08 (d, J=1.6 Hz, 1H, C8a′H), 4.20-4.15 (m, 1H, C3″H), 4.06 (d, J=9.1 Hz, 1H, C9H), 4.01 (br-s, 1H, C3″OH), 3.94 (app-dd, J=11.7, 9.0 Hz, 1H, C2′Hₐ), 3.47 (app-dd, J=15.9, 9.7 Hz, 1H, C2Hₐ), 3.33 (app-dt, J=16.5, 8.8 Hz, 1H, C2H_{b}), 3.25 (app-td, J=13.5, 4.7 Hz, 1H, N8′SO₂CHa), 3.22-3.11 (m, 2H, C2″Hₐ, N8′SO₂CH_{b}), 3.08 (app-td, J=11.6, 7.3 Hz, 1H, C2′H_{b}), 2.92 (s, 3H, N8CH₃), 2.87 (s, 3H, N8CH₃*), 2.84 (d, J=9.1 Hz, 1H, C10H), 2.83-2.77 (m, 1H, C3′Hₐ), 2.49 (app-dd, J=13.0, 8.7 Hz, 1H, C3Hₐ), 2.36 (app-dd, J=16.4, 9.8 Hz, 1H, C2″H_{b}), 2.29 (app-dt, J=13.1, 9.4 Hz, 1H, C3H_{b}), 1.95 (app-dd, J=13.1, 7.2 Hz, 1H, C3′H_{b}), 1.61-1.41 (m, 7H, C13H₃, C4″H₂, C5″H₂), 1.38 (s, 3H, C12H₃), 1.30-1.15 (m, 2H, N8′SO₂CH₂CH₂), 0.96 (t, J=7.0 Hz, 3H, C6″H₃), 0.92 (t, J=7.3 Hz, 3H, C6″H₃*), 0.11 (s, 9H, Si(CH₃)₃*), 0.07 (s, 9H, Si(CH₃)₃). ¹³C NMR (150.9 MHz, CDCl₃, 20° C., 15:1 mixture of atropisomers, *denotes minor atropisomer): δ 174.1 (C₁″), 149.9 (C7a), 138.2 (C4a′), 136.9 (C4), 136.0 (C7a′), 131.2 (C4a), 129.5 (C6), 128.0 (C6′), 126.5 (C5′), 124.9 (C7′), 124.0 (C4′), 113.9 (C5), 102.7 (C7), 84.7 (C8a), 79.5 (C8a′), 68.1 (C3″), 65.6 (C9), 63.8 (C10), 60.0 (C11), 54.2 (C3a), 52.5 (C3a′), 52.0 (N8′SO₂CH₂), 44.0 (C2′), 41.3 (C2″), 38.9 (C4″), 37.7 (C3), 36.5 (C2), 31.5 (C3′), 31.0 (N8CH₃), 25.0 (C12), 20.5 (C13), 18.9 (C5″*), 18.8 (C5″), 14.3 (C6″), 14.2 (C6″*), 10.8 (N8′SO₂CH₂CH₂*), 10.7 (N8′SO₂CH₂CH₂), −1.7 (Si(CH₃)₃*), −1.8 (Si(CH₃)₃). FTIR (thin film) cm⁻¹: 3438 (br-m), 2957 (s), 1630 (s), 1600 (s), 1487 (s), 1455 (s), 1340 (s), 1251 (s), 1156 (s), 1053 (m), 859 (m), 844 (m), 740 (m). HRMS (ESI) (m/z): calc'd for C₃₇H₅₃N₄O₅SSi [M+H]⁺: 693.3500, found: 693.3503 [α]_{D}²³: −100 (c=0.41, CH₂Cl₂). TLC (30% acetone in hexanes), Rf: 0.27 (UV, CAM).

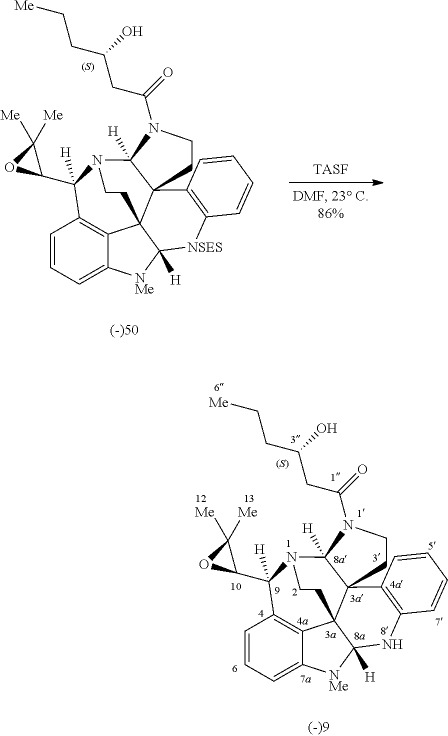

### Example 43: (−)-(C3″S)-Communesin I (9)

A degassed solution of tris(dimethylamino)sulfonium difluorotrimethylsilicate (TASF, 12.3 mg, 44.6 μmol, 4.00 equiv) in N,N-dimethylformamide (100 μL) was added to a degassed solution of (−)-N8′-(trimethylsilyl)ethanesulfonyl (C3″S)-communesin I (50, 7.73 mg, 11.2 μmol, 1 equiv) in N,N-dimethylformamide (300 L) at 23° C. After 2.2 h, a saturated aqueous sodium chloride solution (10 mL) and deionized water (5 mL) were added and the mixture was extracted with ethyl acetate (3×10 mL). The combined organic extracts were washed with a saturated aqueous sodium chloride solution (2×15 mL), were dried over anhydrous sodium sulfate, were filtered, and were concentrated under reduced pressure. The resulting residue was purified by flash column chromatography on silica gel (eluent: 50%→60% ethyl acetate in hexanes) to afford (−)-(C3″S)-Communesin I (9, 5.08 mg, 86.1%) as a white solid. Structural assignments were made using additional information from gCOSY, gHSQC, gHMBC, and 1D selective NOESY experiments. ¹H NMR (500 MHz, CDCl₃, 20° C., 34:1 mixture of atropisomers, *denotes minor atropisomer): δ 7.01 (app-td, J=7.6, 1.5 Hz, 1H, C6′H), 6.89 (t, J=7.8 Hz, 1H, C6H), 6.86 (t, J=7.9 Hz, 1H, C6H*), 6.72 (app-td, J=7.4, 1.2 Hz, 1H, C5′H), 6.70-6.64 (m, 1H, C7′H, C4′H), 6.60 (d, J=6.6 Hz, 1H, C4′H*), 6.09 (d, J=7.7 Hz, 1H, C5H*), 6.06 (d, J=7.6 Hz, 1H, C5H), 5.95 (d, J=7.6 Hz, 1H, C7H), 5.91 (d, J=8.1 Hz, 1H, C7H*), 5.41 (app-s, 1H, C8a′H*), 5.04 (app-s, 1H, C8a′H), 4.70 (s, 1H, C8aH), 4.67 (s, 1H, C8aH*), 4.59 (br-s, 1H, N8′H), 4.46 (d, J=8.7 Hz, 1H, C9H*), 4.22-4.12 (m, 1H, C3″H), 4.19 (d, J=2.0 Hz, 1H, C3″OH), 4.05 (d, J=9.2 Hz, 1H, C9H), 3.89 (app-dd, J=11.8, 8.3 Hz, 1H, C2′Hₐ), 3.45 (app-ddt, J=16.1, 9.6, 1.6 Hz, 1H, C2Hₐ), 3.34 (app-dt, J=15.9, 8.8 Hz, 1H, C2H_{b}), 3.21 (dd, J=16.4, 2.2 Hz, 1H, C2″Hₐ) 3.00 (app-td, J=11.7, 7.2 Hz, 1H, C2′H_{b}), 2.87 (d, J=9.2 Hz, 1H, C10H), 2.84 (s, 3H, N8CH₃), 2.81 (s, 3H, N8CH₃*), 2.80 (d, J=8.8 Hz, 1H, C10H*), 2.72 (ddd, J=13.3, 11-6, 8.7 Hz, 1H, C3′Hₐ), 2.40-2.31 (m, 2H, C3Hₐ, C2″H_{b}), 2.27 (app-dt, J=13.0, 9.4 Hz, 1H, C3H_{b}), 1.99 (app-dd, J=13.3, 6.7 Hz, 1H, C3′H_{b}), 1.59-1.43 (m, 10H, C13H₃, C13H₃*, C4″H₂, C5″H₂), 1.39 (s, 3H, C12H₃), 1.36 (s, 3H, C12H₃*), 0.96 (t, J=7.1 Hz, 3H, C6″H₃). ¹³C NMR (150.9 MHz, CDCl₃, 25° C., 34:1 mixture of atropisomers, *denotes minor atropisomer): δ 174.5 (C1″), 150.7 (C7a), 142.7 (C7a′), 136.6 (C4), 132.3 (2C, C4a, C4a′), 129.1 (C6), 127.6 (C6′), 123.4 (C4′), 120.9 (C5′), 117.1 (C7′), 113.4 (C5), 102.1 (C7), 82.6 (C8a), 79.2 (C8a′), 68.1 (C3″), 65.6 (C9), 64.1 (C10), 60.0 (C11), 52.1 (C3a′), 51.6 (C3a), 44.1 (C2′), 41.1 (C2″), 38.8 (C4″), 38.1 (C3), 36.4 (C2), 30.7 (C3′), 29.8 (N8CH₃), 25.0 (C12), 20.5 (C13), 18.8 (C5″), 14.3 (C6″). FTIR (thin film) cm⁻¹: 3429 (br-w), 3330 (br-w), 3052 (w), 2957 (m), 2925 (m), 2873 (m), 1620 (s), 1606 (s), 1596 (s), 1493 (m), 1427 (s), 1338 (m), 1279 (m), 1004 (m), 908 (m), 740 (m). HRMS (ESI) (m/z): calc'd for C₃₂H₄₁N₄O₃ [M+H]⁺: 529.3173, found: 529.3155. [α]_{D}²³: −147 (c=0.25, MeOH). TLC (60% ethyl acetate in hexanes), Rf: 0.15 (UV, CAM).

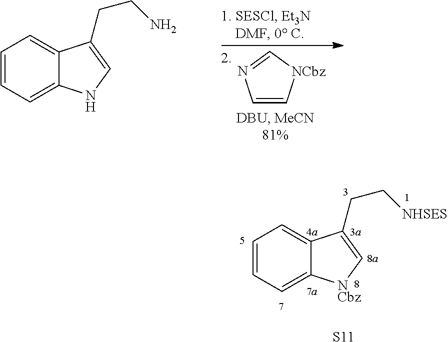

### Example 44: Tryptamine S11

2-(Trimethylsilyl)ethanesulfonyl chloride (455 μL, 2.40 mmol, 1.20 equiv) was added dropwise via syringe over 15 min to a solution of tryptamine (320 mg, 2.00 mmol, 1 equiv) and triethylamine (1.00 mL, 7.20 mmol, 3.60 equiv) in N,N-dimethylformamide (4.00 mL) at 0° C. After 30 min, the suspension was diluted with a saturated aqueous ammonium sulfate solution (45 mL) and deionized water (15 mL). After warming to 23° C., the mixture was extracted with diethyl ether (3×40 mL) and the combined organic extracts were washed successively with an aqueous hydrogen chloride solution (1 N, 80 mL) and a saturated aqueous sodium chloride solution (80 mL), were dried over anhydrous sodium sulfate, were filtered, and were concentrated under reduced pressure. The resulting residue was dissolved in acetonitrile (6.00 mL) and samples of benzyl 1H-imidazole-1-carboxylate¹⁰³ (445 mg, 2.20 mmol, 1.10 equiv) and 1,8-diazabicyclo[5.4.0]undec-7-ene (75.0 μL, 0.500 mmol, 0.250 equiv) were then added. After stirring for 21 h at 23° C., the pale-beige solution was diluted with an aqueous hydrogen chloride solution (1 N, 10 mL) and deionized water (40 mL). The mixture was extracted with ethyl acetate (3×50 mL) and the combined organic extracts were washed with a saturated aqueous sodium chloride solution (100 mL), were dried over anhydrous sodium sulfate, were filtered, and were concentrated under reduced pressure. The resulting residue was purified by flash column chromatography on silica gel (eluent: 20%→25% ethyl acetate in hexanes) to afford tryptamine S11 (747 mg, 81.5% over two steps) as white solid. Structural assignments were made using additional information from gCOSY, gHSQC, and gHMBC experiments. ¹H NMR (500 MHz, CDCl₃, 21° C.): δ 8.19 (br-s, 1H, C7H), 7.55 (d, J=7.6 Hz, 1H, C4H), 7.53-7.47 (m, 3H, C8aH, Ar_{Cbz}-o-H), 7.45-7.37 (m, 3H, Ar_{Cbz}-m-H, Ar_{Cbz}-p-H), 7.34 (app-t, J=7.6 Hz, 1H, C6H), 7.27 (app-t, J=7.1 Hz, 1H, C5H), 5.43 (s, 2H, N8CO₂CH₂Ph), 4.66 (t, J=6.3 Hz, 1H, N8HCO₂CH₂Ph), 3.43 (app-q, J=6.9 Hz, 2H, C2H₂), 2.97 (t, J=7.1 Hz, 2H, C3H₂), 2.89-2.83 (m, 2H, N1HSO₂CH₂), 0.99-0.83 (m, 2H, N1HSO₂CH₂CH₂), −0.01 (s, 9H, Si(CH₃)₃). ¹³C NMR (125.8 MHz, CDCl₃, 21° C.): δ 150.7 (N8CO₂CH₂Ph), 135.7 (C7a), 135.1 (Ar_{Cbz}-ipso-C), 130.0 (C4a), 128.8 (2C, Ar_{Cbz}-m-C, Ar_{Cbz}-p-C), 128.6 (Ar_{Cbz}-o-C), 125.1 (C6), 123.3 (C8a), 123.1 (C5), 118.9 (C4), 117.7 (C3a), 115.5 (C7), 68.8 (N8CO₂CH₂Ph), 48.8 (N1SO₂CH₂), 42.8 (C2), 26.4 (C3), 10.5 (N1SO₂CH₂CH₂), −2.0 (Si(CH₃)₃). FTIR (thin film) cm⁻¹: 3239 (w), 2955 (w), 1739 (s), 1454 (m), 1394 (s), 1355 (s), 1317 (m), 1249 (s), 1212 (m), 860 (m), 742 (s). HRMS (DART) (m/z): calc'd for C₂₃H₃₁N₂O₄SSi [M+H]: 459.1774, found: 459.1771. TLC (20% ethyl acetate in hexanes), Rf: 0.20 (UV, CAM).

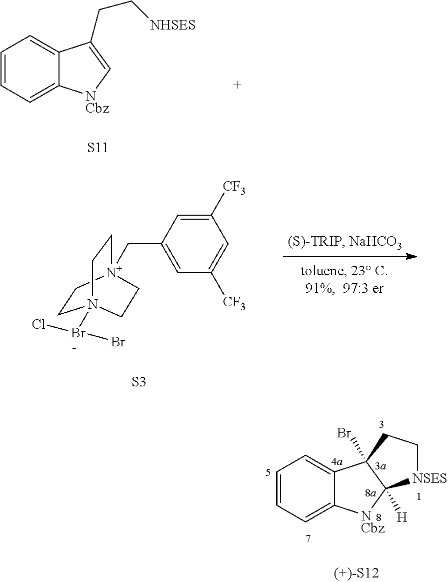

### Example 45: Bromocyclotryptamine (+)-S12

A sample of bromine salt S3³⁷ (437 mg, 817 μmol, 1.30 equiv) was added to a suspension of tryptamine S11 (288 mg, 628 μmol, 1 equiv), (S)-3,3′-bis(2,4,6-triisopropyl-phenyl)-1,1′-binaphthyl-2,2′-diyl hydrogenphosphate ((S)-TRIP, 47.3 mg, 62.8 μmol, 0.100 equiv), and crushed sodium hydrogen carbonate (211 mg, 2.51 mmol, 4.00 equiv) in toluene (12.6 mL) at 23° C. After stirring for 23 h, the orange suspension was diluted with an aqueous sodium thiosulfate solution (1M, 50 mL) and was stirred vigorously for 5 min. The mixture was extracted with ethyl acetate (3×25 mL). The combined organic extracts were washed successively with an aqueous sodium thiosulfate solution (1 M, 75 mL) and a saturated aqueous sodium chloride solution (75 mL), were dried over anhydrous sodium sulfate, were filtered, and were concentrated under reduced pressure. The residue was purified by flash column chromatography on silica gel (eluent: 10% ethyl acetate in hexanes) to afford bromocyclotryptamine (+)-S12 (307 mg, 90.9%, 97:3 er) as a sticky white foam.³⁹ The enantiomeric ratio was determined by chiral HPLC analysis (CHIRALPAK® IA, 15% iPrOH in hexanes, 1.0 mL/min, 220 nm, t_{R} (major)=7.0 min, t_{R} (minor)=9.3 min). As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (500 MHz, CD₃CN, 60° C.): δ 7.69 (d, J=8.2 Hz, 1H, C7), 7.54-7.48 (m, 3H, C4H, Ar_{Cbz}-o-H), 7.45-7.39 (m, 2H, Ar_{Cbz}-m-H), 7.39-7.32 (m, 2H, C6H, Ar_{Cbz}-p-H), 7.19 (t, J=7.6 Hz, 1H, C5H), 6.47 (s, 1H, C8aH), 5.41 (d, J=12.2 Hz, 1H, N8CO₂CHₐPh), 5.29 (d, J=12.2 Hz, 1H, N8CO₂CH_{b}Ph), 3.86 (app-dd, J=10.8, 7.2 Hz, 1H, C2Hₐ), 3.14-2.99 (m, 2H, N1SO₂CH₂), 2.93 (app-dd, J=11.9, 4.3 Hz, 1H, C3Hₐ), 2.85 (app-td, J=10.9, 4.1 Hz, 1H, C2H_{b}), 2.76 (app-td, J=11.9, 7.5 Hz, 1H, C3H_{b}), 1.01-0.78 (m, 2H, N1SO₂CH₂CH₂), 0.01 (s, 9H, Si(CH₃)₃). ¹³C NMR (125.8 MHz, CD₃CN, 60° C.): δ 154.2 (N8CO₂CH₂Ph), 142.2 (C7a), 137.2 (Ar_{Cbz}-ipso-C), 133.7 (C4a), 132.1 (C6), 129.9 (Ar_{Cbz}-m-C), 129.7 (2C, Ar_{Cbz}-o-C, Ar_{Cbz}-p-C), 126.1 (C5), 126.0 (C4), 117.6 (C7), 87.7 (C8a), 69.4 (N8CO₂CH₂Ph), 64.6 (C3a), 51.1 (N1SO₂CH₂), 50.1 (C2), 44.7 (C3), 11.3 (N1SO₂CH₂CH₂), −1.5 (Si(CH₃)₃). FTIR (thin film) cm⁻¹: 2952 (w), 2896 (w), 1710 (s), 1481 (s), 1395 (s), 1326 (s), 1249 (s), 1140 (s), 1040 (m), 832 (s). HRMS (DART) (m/z): calc'd for C₂₃H₃₀BrN₂O₄SSi [M+H]: 537.0879, found: 537.0877. [α]_{D}²²: +77 (c=1.43, CH₂Cl₂). TLC (10% ethyl acetate in hexanes), Rf: 0.25 (UV, CAM).

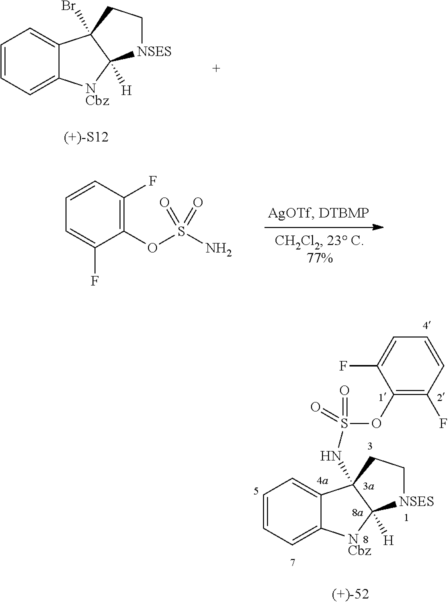

### Example 46: Sulfamate Ester (+)-52

A sample of silver trifluoromethanesulfonate (278 mg, 1.08 mmol, 2.00 equiv) was added to a solution of bromocyclotryptamine (+)-S12 (291 mg, 0.542 mmol, 1 equiv), 2,6-difluorophenyl sulfamate⁴⁰ (227 mg, 1.08 mmol, 2.00 equiv), and 2,6-di-tert-butyl-4-methylpyridine (DTBMP, 278 mg, 1.35 mmol, 2.50 equiv) in dichloromethane (13.6 mL) at 23° C. in the dark. After 1.5 h, the milky beige suspension was diluted with ethyl acetate (27 mL) and was filtered through a pad of pad of Celite. The filter cake was washed with ethyl acetate (136 mL) and the colorless filtrate was concentrated under reduced pressure. The residue was purified by flash column chromatography on silica gel (eluent: 40%→50% diethyl ether in hexanes) to afford sulfamate ester (+)-52 (278 mg, 77.0%) as a white foam. As a result of the slow conformational equilibration at ambient temperature, NMR spectra were collected at elevated temperature. Structural assignments were made using additional information from gCOSY, gHSQC, and gHMBC experiments also collected at elevated temperature. ¹H NMR (400 MHz, CD₃CN, 60° C.): δ 7.73 (br-d, J=8.3 Hz, 1H, C7H), 7.53 (ddd, J=7.7, 1.4, 0.6 Hz, 1H, C4H), 7.48-7.43 (m, 2H, Ar_{Cbz}-o-H), 7.43-7.38 (m, 1H, C6H), 7.38-7.27 (m, 4H, C4′H, Ar_{Cbz}-m-H, Ar_{Cbz}-p-H), 7.18 (app-td, J=7.6, 1.1 Hz, 1H, C₅H), 7.13 (br-s, 1H, NHSO₃Ar), 7.11-7.04 (m, 2H, C3′H), 6.56 (s, 1H, C8aH), 5.37 (d, J=12.3 Hz, 1H, N8CO₂CHₐPh), 5.23 (d, J=12.3 Hz, 1H, N8CO₂CH_{b}Ph), 3.93 (app-dd, J=11.2, 7.5 Hz, 1H, C2Hₐ), 3.15-2.94 (m, 2H, N1SO₂CH₂), 2.87 (ddd, J=12.2, 11.2, 4.6 Hz, 1H, C2H_{b}), 2.72 (app-td, J=12.2, 7.5 Hz, 1H, C3Hₐ), 2.54 (app-dd, J=12.2, 4.6 Hz, 1H, C3H_{b}), 0.99-0.79 (m, 2H, N1SO₂CH₂CH₂), 0.01 (s, 9H, Si(CH₃)₃).¹³C NMR (100.6 MHz, CD₃CN, 60° C.): δ 157.0 (dd, J=252, 3.7 Hz, C2′), 154.1 (N8CO₂CH₂Ph), 143.9 (C₇a), 137.2 (Ar_{Cbz}-ipso-C), 131.9 (C6), 130.3 (C4a), 129.6 (Ar_{Cbz}-m-C), 129.4 (t, J=9.5 Hz, C4′), 129.4 (Ar_{Cbz}-p-C), 129.4 (Ar_{Cbz}-o-C), 127.6 (t, J=15.8 Hz, C1′), 126.1 (C4), 125.3 (C5), 117.4 (C7), 114.0-113.6 (m, C3′), 82.7 (C8a), 74.0 (C3a), 68.8 (N8CO₂CH₂Ph), 50.7 (N1SO₂CH₂), 48.7 (C2), 38.8 (C3), 11.1 (N1SO₂CH₂CH₂), −1.8 (Si(CH₃)₃). ¹⁹F NMR (376.4 MHz, CD₃CN, 25° C.): 6-126.2 (s, C₆H₃F₂). FTIR (thin film) cm⁻¹: 3210 (br-w), 2952 (w), 2897 (w), 1710 (m), 1605 (m), 1480 (m), 1389 (m), 1324 (m), 1247 (m), 1138 (m), 1010 (s), 859 (s), 832 (s), 744 (s), 522 (s). HRMS (DART) (m/z): calc'd for C₂₉H₃₄F₂N₃O₇S₂Si [M+H]: 666.1575, found: 666.1567. [α]_{D}²³: +52 (c=0.51, CH₂Cl₂). TLC (50% diethyl ether in hexanes), Rf: 0.20 (UV, CAM).

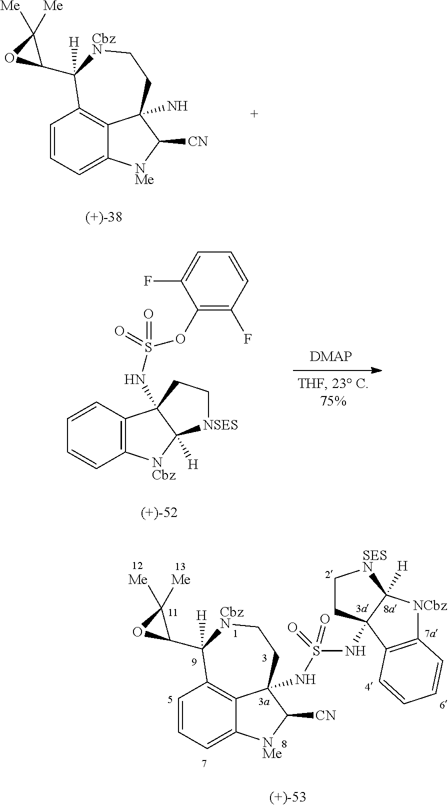

### Example 47. Sulfamide (+)-53

A 10-mL round-bottom flask was charged with samples of benzylic aminonitrile (+)-38 (37.0 mg, 85.5 μmol, 1 equiv) and sulfamate ester (+)-52 (102 mg, 154 μmol, 1.80 equiv) and the resulting mixture was azeotropically dried by concentration from anhydrous benzene (3×1.5 mL). The residue was dissolved in tetrahydrofuran (340 μL) and a sample of 4-(dimethylamino)pyridine (20.9 mg, 171 μmol, 2.00 equiv) was added as a solid at 23° C. After 23 h, the light-brown solution was diluted with a saturated aqueous ammonium sulfate solution (8 mL) and deionized water (4 mL) and the mixture was extracted with ethyl acetate (3×8 mL). The combined organic extracts were washed with a saturated aqueous sodium chloride solution (20 mL), were dried over anhydrous sodium sulfate, were filtered, and were concentrated under reduced pressure. The residue was purified by flash column chromatography on silica gel (eluent: 30%→40% ethyl acetate in hexanes) to afford sulfamide (+)-53 (62.5 mg, 75.4%) as a white film. As a result of the slow conformational equilibration at ambient temperature, NMR spectra were collected at elevated temperature. ¹H NMR (400 MHz, C₆D₆, 70° C.): 1° 4 6 7.85 (d, J=8.2 Hz, 1H), 7.36 (dd, J=7.3, 2.2 Hz, 3H), 7.26 (d, J=7.6 Hz, 2H), 7.19-7.15 (m, 3H), 7.15-7.03 (m, 6H), 7.03-6.95 (m, 1H), 6.95-6.78 (m, 2H), 6.45 (br-s, 1H), 6.20 (br-s, 1H), 5.36 (d, J=9.3 Hz, 1H), 5.29 (d, J=12.3 Hz, 1H), 5.18 (d, J=12.3 Hz, 1H), 5.17-5.08 (m, 2H), 5.08-4.88 (br-m, 1H), 4.68 (br-s, 1H), 4.49 (s, 1H), 3.98 (dd, J=11.6, 7.2 Hz, 1H), 3.56 (dd, J=14.1, 6.3 Hz, 1H), 3.20 (dd, J=13.3, 4.0 Hz, 1H), 3.09 (td, J=13.6, 4.4 Hz, 1H), 2.88 (br-s, 1H), 2.71-2.24 (m, 6H), 2.11 (d, J=8.3 Hz, 1H), 1.28 (s, 3H), 1.06-0.86 (m, 2H), 0.99 (s, 3H), −0.12 (s, 9H). ¹³C NMR (100.6 MHz, C₆D₆, 70° C.):¹⁰⁵ δ 157.7 (br), 153.9, 152.0, 143.3, 138.8, 137.2, 136.4, 131.4, 130.6, 128.9, 128.7 (2C), 128.5, 128.2, 127.9, 127.4 (br), 125.5, 124.2, 118.2, 116.6, 115.5, 108.6, 84.1, 73.1, 68.5, 68.3, 67.2, 66.9 (br), 63.4, 59.8, 59.0 (br), 50.8, 47.1, 41.5, 40.5 (br), 33.2 (br), 32.8, 24.3, 19.9, 10.8, −2.0. FTIR (thin film) cm⁻¹: 3253 (br-w), 2955 (w), 2896 (w), 1708 (s), 1600 (m), 1484 (m), 1397 (m), 1327 (s), 1263 (s), 1250 (s), 1141 (s), 838 (m), 750 (m), 698 (m). HRMS (ESI) (m/z): calc'd for C₄₈H₅₇N₇NaO₉S₂Si [M+Na]*: 990.3321, found: 990.3337. [α]_{D}²³: +86 (c=0.13, CH₂Cl₂). TLC (40% ethyl acetate in hexanes), Rf: 0.33 (UV, CAM).

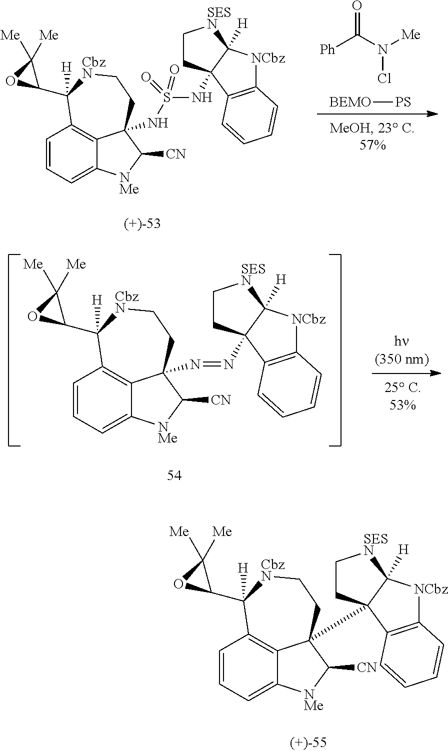

### Example 48: Heterodimer (+)-55

N-Chloro-N-methylbenzamide⁵⁹ (63.1 mg, 372 μmol, 6.00 equiv) and resin-bound 2-tert-butyl-imino-2-diethylamino-1,3-dimethyl-perhydro-1,3,2-diazaphosphorine (BEMP, 340 mg, 2.19 mmol/g on 200.400 mesh polystyrene resin, 744 μmol, 12.0 equiv) were added in rapid succession to a solution of sulfamide (+)-53 (60.0 mg, 62.0 μmol, 1 equiv) in methanol (6.2 mL) at 23° C. in the dark. After 15 min, the suspension was filtered through a pad of Celite, and the filter cake was washed sequentially with methanol (10 mL), dichloromethane (10 mL), and ethyl acetate (10 mL). The colourless filtrate was concentrated under reduced pressure and the resulting residue was purified by flash column chromatography on silica gel (eluent: 6% ethyl acetate, 47% hexanes, 47% dichloromethane→10% ethyl acetate, 45% hexanes, 45% dichloromethane) to afford diazene 54 (32.0 mg, 57.2%) as a light yellow foam,¹⁰⁶ which was used directly in the next step without further purification. A solution of diazene 54 (32.0 mg, 35.5 μmol, 1 equiv) in dichloromethane (10 mL) was concentrated under reduced pressure in a 100-mL round bottom flask to provide a thin film of the diazene coating the flask. The flask was evacuated and backfilled with argon (three cycles) and was then irradiated in a Rayonet photoreactor equipped with 14 radially distributed (r=12.7 cm) 25 W lamps (λ=350 nm) at 25° C. After irradiating for 3 h, the lamps were turned off and the resulting residue was purified by flash column chromatography on silica gel (eluent: 8% ethyl acetate, 46% hexanes, 46% dichloromethane→10% ethyl acetate, 45% hexanes, 45% dichloromethane) to afford heterodimer (+)-55 (16.4 mg, 52.8%) as a pale-yellow film. As a result of the slow conformational equilibration at ambient temperature, NMR spectra were collected at elevated temperature. ¹H NMR (400 MHz, CD₃CN, 70° C.):¹⁰⁴ δ 7.75 (d, J=8.1 Hz, 1H), 7.56-7.48 (m, 4H), 7.48-7.35 (m, 7H), 7.34-7.27 (m, 2H), 7.15 (t, J=6.8 Hz, 1H), 6.64 (dt, J=7.9, 1.0 Hz, 1H), 6.36 (d, J=7.3 Hz, 1H), 5.50 (s, 1H), 5.47 (d, J=8.8 Hz, 1H), 5.36 (d, J=12.1 Hz, 1H), 5.17 (d, J=12.0 Hz, 2H), 4.05 (dt, J=14.3, 2.7 Hz, 1H), 3.68 (s, 1H), 3.40-3.27 (m, 1H), 3.08-2.90 (m, 1H), 2.86 (d, J=8.7 Hz, 1H), 2.67-2.55 (m, 2H), 2.45 (s, 3H), 2.44-2.33 (m, 1H), 2.04 (dt, J=15.5, 2.2 Hz, 1H), 1.96 (s, 2H), 1.82 (br-s, 2H), 1.57 (s, 3H), 1.38 (s, 3H), 0.61-0.52 (m, 2H), −0.05 (s, 9H). ¹³C NMR (100.6 MHz, CD₃CN, 70° C.): δ 156.9, 153.9, 153.8, 144.2, 139.2, 138.5, 137.4, 132.2, 132.1, 131.3, 130.5, 130.4, 130.0, 129.9, 129.7, 128.5, 126.5, 126.1, 125.7, 120.7, 118.1, 117.6, 109.5, 81.8, 70.8, 69.3, 68.6, 67.5, 67.2, 62.3, 59.5, 58.5, 50.1, 48.9, 43.0, 36.0, 34.3, 28.9, 24.9, 20.1, 11.3, −1.4. FTIR (thin film) cm⁻¹: 2956 (w), 2893 (w), 1703 (s), 1585 (w), 1481 (m), 1403 (s), 1330 (s), 1141 (s), 1053 (m), 860 (m), 753 (s), 699 (s). HRMS (DART) (m/z): calc'd for C₄₈H₅₆N₅O₇SSi [M+H]: 874.3670, found: 874.3683. [α]_{D}²³: +128 (c=0.82, CH₂Cl₂). TLC (10% ethyl acetate, 45% hexanes, 45% dichloromethane), Rf: 0.22 (UV, CAM).

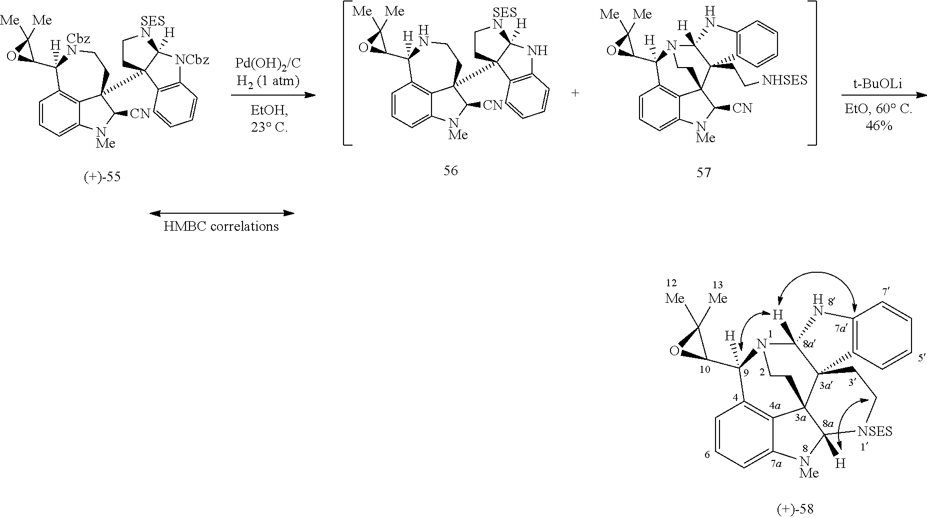

### Example 49: (+)—N1′-(Trimethylsilyl)ethanesulfonyl iso-communesin (58)

A sample of palladium(II) hydroxide on carbon (15.7 wt % on wet support, 3.2 mg, 3.6 μmol, 0.60 equiv) was added to a solution of heterodimer (+)-55 (5.3 mg, 6.0 μmol, 1 equiv) in anhydrous ethanol (200 proof, 400 μL) at 23° C. The resulting suspension was sparged with dihydrogen for 7 min by discharge of a balloon equipped with a needle extending into the reaction mixture. After vigorous stirring for 3.5 h under an atmosphere of dihydrogen, the suspension was sparged with dinitrogen for 5 min and was then diluted with ethyl acetate (6 mL) and filtered through a plug of Celite. The filter cake was washed with ethyl acetate (8 mL) and the colourless filtrate was concentrated under reduced pressure. The resulting residue was filtered through a plug of silica gel (eluent: ethyl acetate) and the filtrate was concentrated under reduced pressure to yield a mixture of heterodimeric diamine 56 (major) and hexacyclic aminonitrile 57 (minor), which was used directly in the next step without further purification.¹⁰⁷

The crude mixture of 56 and 57 was dissolved in dichloromethane (1 mL) and transferred to a pressure tube equipped with a magnetic stir bar. The transfer was quantitated with additional dichloromethane (2×1 mL) and the resulting solution was concentrated under reduced pressure. The tube was refilled with argon and was then charged with a solution of lithium tert-butoxide in anhydrous ethanol (0.20 M, 0.60 mL, 0.12 mmol, 20 equiv). The tube was sealed under an argon atmosphere with a Teflon screwcap and was immersed in a pre-heated oil bath at 60° C. After stirring at this temperature for 45 h, the homogeneous orange solution was cooled to 23° C. and was diluted with a saturated aqueous sodium bicarbonate solution (5 mL). The mixture was extracted with ethyl acetate (3×5 mL) and the combined organic extracts were washed with a saturated aqueous sodium chloride solution (10 mL), were dried over anhydrous sodium sulfate, were filtered, and were concentrated under reduced pressure. The resulting orange residue was purified by flash column chromatography on silica gel (eluent: 30%→40% ethyl acetate in hexanes) to afford (+)-N1′-(trimethylsilyl)ethanesulfonyl iso-communesin (58, 1.62 mg, 46.3%) as a white film. Structural assignments were made using additional information from gCOSY, gHSQC, gHMBC, and 1D selective NOESY experiments. ¹H NMR (600 MHz, CD₃OD, 25° C.): δ 7.19 (d, J=7.8 Hz, 1H, C4′H), 6.89 (t, J=7.8 Hz, 1H, C6H), 6.78 (td, J=7.5, 1.3 Hz, 1H, C6′H), 6.44 (d, J=7.8 Hz, 1H, C7′H), 6.42 (d, J=7.8 Hz, 1H, C7H), 6.18 (td, J=7.5, 1.3 Hz, 1H, C5′H), 6.05 (d, J=8.0 Hz, 1H, C5H), 4.88 (s, 1H, C8aH), 4.39 (s, 1H, C8a′H), 3.75 (dt, J=15.0, 3.0 Hz, 1H, C2′Hₐ), 3.69 (d, J=9.5 Hz, 1H, C9H), 3.57-3.49 (m, 2H, C2Hₐ, C2′H_{b}), 3.26-3.13 (m, 3H, C2H_{b}, N1′SO₂CH₂), 3.09 (d, J=9.3 Hz, 1H, C10H), 2.88 (s, 3H, N8CH₃), 2.48 (dt, J=13.5, 9.5 Hz, 1H, C3Hₐ), 2.09 (td, J=13.1, 3.6 Hz, 1H, C3′Hₐ), 2.00-1.94 (m, 1H, C3H_{b}), 1.40 (s, 3H, C12H₃), 1.37 (dt, J=13.5, 2.4 Hz, 1H, C3′H_{b}), 1.21 (s, 3H, C13H₃), 1.16-1.10 (m, 2H, N1′SO₂CH₂CH₂), 0.13 (s, 9H, Si(CH₃)₃). ¹³C NMR (150.9 MHz, CD₃OD, 25° C.): δ 150.5 (2C, C7a, C7a′), 137.6 (C4), 132.9 (C4a′), 132.5 (C4a), 129.8 (C6), 129.2 (C6′), 126.2 (C4′), 118.3 (C5′), 117.6 (C5), 108.5 (C7′), 108.1 (C7), 83.8 (C8a′), 82.9 (C8a), 65.0 (C10), 62.2 (C11), 59.6 (C9), 56.3 (C3a′), 50.5 (N1′SO₂CH₂), 43.4 (C3a), 41.0 (C2), 39.4 (C2′), 35.3 (C3), 33.2 (N8CH₃), 31.1 (C3′), 24.9 (C12), 19.6 (C13), 11.6 (N1′SO₂CH₂CH₂), −2.0 (Si(CH₃)₃). FTIR (thin film) cml: 3354 (br-w), 2954 (m), 2925 (m), 2867 (w), 1605 (m), 1468 (s), 1341 (m), 1324 (m), 1144 (s), 1029 (s), 860 (s), 833 (s), 742 (s), 600 (m). HRMS (DART) (m/z): calc'd for C₃₁H₄₃N₄O₃SSi [M+H]: 579.2825, found: 579.2828. [α]_{D}²²: +94 (c=0.08, MeOH). TLC (40% ethyl acetate in hexanes), Rf: 0.22 (UV, CAM).

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto; embodiments may be practiced otherwise than as specifically described and claimed. embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments can be implemented in any of numerous ways. Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In addition, those of ordinary skill in the art recognize that some functional groups can be protected/deprotected using various protecting groups before a certain reaction takes place. Suitable conditions for protecting and/or deprotecting specific functional group, and the use of protecting groups are well-known in the art.

For example, various kinds of protecting groups are described in T. W. Greene and G. M. Wuts, Protecting Groups in Organic Synthesis, Second edition, Wiley, New York, 1991, and other references cited above.

All documents cited herein are herein incorporated by reference in their entirety for all purposes.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

### REFERENCES

1. (a) Yang, J.; Wu, H.; Shen, Y.; Qin, Y. J. Am. Chem. Soc. 2007, 129, 13794. (b) Liu, P.; Seo, J. H.; Weinreb, S. M. Angew. Chem., Int. Ed. 2010, 49, 2000. (c) Seo, J. H.; Artman, G. D., III; Weinreb, S. M. J. Org. Chem. 2006, 71, 8891. (d) Belmar, J.; Funk, R. L. J. Am. Chem. Soc. 2012, 134, 16941. (f) Han, S.-J.; Vogt, F.; Krishnan, S.; May, J. A.; Gatti, M.; Virgil, S. C.; Stoltz, B. M. Org. Lett. 2014, 16, 3316. (g) Han, S.-J.; Vogt, F.; May, J. A.; Krishnan, S.; Gatti, M.; Virgil, S. C.; Stoltz, B. M. J. Org. Chem. 2015, 80, 528.
2. (a) Zuo, Z.; Xie, W.; Ma, D. J. Am. Chem. Soc. 2010, 132, 13226. (b) Lathrop, S. P.; Pompeo, M.; Chang, W.-T. T.; Movassaghi, M. J. Am. Chem. Soc. 2016, 138, 7763. (c) Liang, X.; Zhang, T.-Y.; Zeng, X.-Y.; Zheng, Y.; Wei, K.; Yang, Y.-R. J. Am. Chem. Soc. 2017, 139, 3364. (d) Park. J.; Jean, A.; Chen, D. Y.-K. Angew. Chem. Int. Ed. 2017, 56, 14237. (e) Park, J.; Jean, A.; Chen, D. Y.-K. J Org. Chem. 2018, 83, 6936.
3. Zuo, Z.; Ma, D. Angew. Chem., Int. Ed. 2011, 50, 12008.
4. Still, W. C.; Kahn, M.; Mitra, A. J. Org. Chem. 1978, 43, 2923.
5. Pangborn, A. B.; Giardello, M. A.; Grubbs, R. H.; Rosen, R. K.; Timmers, F. Organometallics 1996, 15, 1518.
6. (a) Gottlieb, H. E.; Kotlyar, V.; Nudelman, A. J. Org. Chem. 1997, 62, 7512. (b) Fulmer, G. R.; Miller, A. J. M.; Sherden, N. H.; Gottlieb, H. E.; Nudelman, A.; Stoltz, B. M.; Bercaw, J. E.; Goldberg, K. I Organometallics 2010, 29, 2176.
7. Movassaghi, M.; Ahmad, O. K.; Lathrop, S. P. J. Am. Chem. Soc. 2011, 133, 13002. (b) Lathrop, S. P.; Kim, J.; Movassaghi, M. Chimia 2012, 66, 389. (c) Lathrop, S. P.; Movassaghi, M. Chem. Sci. 2014, 5, 333. (d) Lindovska, P.; Movassaghi, M. J Am. Chem. Soc., 2017, 139, 17590.
8. Xie, X.; Jiang, G.; Liu, H.; Hu, J.; Pan, X.; Zhang, H.; Wan, X.; Lai, Y.; Ma, D. Angew. Chem., Int. Ed. 2013, 52, 12924.
9. The stereochemistry of the epoxide is also implicated in the observed rate of decomposition. For example, derivatives containing a (10S) epoxide were found to be much more susceptible to this intramolecular opening than the corresponding (10R) derivatives.
10. Ellman, J. A.; Owens, T. D.; Tang, T. P. Acc. Chem. Res. 2002 35, 984.
11. Sun, P.; Weinreb, S. M. J. Org. Chem. 1997 62, 8604.
12. (a) Weinreb, S. M.; Demko, D. M.; Lessen, T. A.; Demers, J. P. Tetrahedron Lett. 1986 27, 2099. (b) Ribiere, P.; Declerck, V.; Martinez, J.; Lamaty, F. Chem. Rev., 2006, 106, 2249.
13. For relevant studies on the use of (−)-diacetone-D-glucose (DAG) as a chiral controller for the preparation of chiral sulfoxides and sulfinamides, see: (a) Llera, J. M.; Fernández, I.; Alcudia, F. Tetrahedron Lett. 1991, 32, 7299. (b) Fernández, I.; Khiar, N.; Llera, J. M.; Alcudia, F. J. Org. Chem. 1992, 57, 6789. (c) Khiar, N.; Fernández, I.; Alcudia, F. Tetrahedron Lett. 1994, 35, 5719. (d) Fernández, I.; Valdivia, V.; Khiar, N. J Org. Chem. 2008, 73, 745. (e) Chelouan, A.; Recio, R.; Alcudia, A.; Khiar, N.; Fernández, I. Eur. J. Org. Chem. 2014, 6935.
14. See the Examples for details.
15. The use of silver(I) carbonate as base was needed to provide the desired coupled product in high yield. A Heck protocol employing potassium carbonate as the base under otherwise identical conditions resulted in 81% yield of the protodebromination product with only 12% yield of the desired styrene.
16. Harrington, P. J.; Hegedus, L. S.; McDaniel, K. F. J Am. Chem. Soc. 1987, 109, 4335.
17. Attempts to sequester the acid with mild inorganic bases such as sodium bicarbonate resulted in an equimolar amount of desired product relative to the catalyst loading, indicating that the acid is needed for catalyst turnover. For a proposed reaction mechanism, see: Banfi, L.; Basso, A.; Cerulli, V.; Guanti, G.; Riva, R. J. Org. Chem. 2008, 73, 1608.
18. Wakayama, M.; Ellman, J. A. J. Org. Chem. 2009, 74, 2646.
19. Magnesium(II) perchlorate and magnesium(II) trifluoro-methanesulfonate were also found to be competent promoters of the allylic amination, however due to safety considerations in the first case and slightly lower efficiency in the second case, calcium(II) trifluoromethanesulfonate was selected.
20. For alkene epoxidations mediated by catalytic amounts of 1,1,1-trifluoroacetone with aqueous hydrogen peroxide as the primary oxidant, see: Shu, L.; Shi, Y. J. Org. Chem. 2000, 65, 8807.
21. For alkene epoxidations mediated by stoichiometric amounts of 1,1,1-trifluoroacetone, see: (a) Denmark, S. E.; Forbes, D. C.; Hays, D. S.; DePue, J. S.; Wilde, R. G. J. Org. Chem. 1995, 60, 1391. (b) Yang, D.; Wong, M.-K.; Yip, Y.-C. J. Org. Chem. 1995, 60, 3887. (c) Denmark, S. E.; Wu, Z.; Crudden, C. M.; Matsuhashi, H. J. Org. Chem. 1997, 62, 8288.
22. Other common epoxidants also furnished (−)-33, albeit with slightly lower efficiency and diastereoselectivity. For example, exposure of a suspension of (−)-32 and sodium bicarbonate in dichloromethane to 2.6 molar equivalents of meta-chloroperbenzoic provided (−)-33 in 65% yield and (−)-34 in 10% yield.
23. Matsumori, N.; Kaneno, D.; Murata, M.; Nakamura, H.; Tachibana, K. J. Org. Chem. 1999, 64,866.
24. The stereochemical configuration at C8a was determined by nuclear Overhauser effect analysis of a derivative. See the Supporting Information for details.
25. Scheidt, K. A.; Chen, H.; Follows, B. C.; Chemler, S. R.; Coffey, D. S.; Roush, W. R. J. Org. Chem. 1998, 63, 6436.
26. The addition of water was necessary to suppress intramolecular trapping of the C8a-iminium with the sulfamide.
27. In a closely related system, calculations suggest that the C8a-CN provides 3.8 kcal/mol in favour of the observed stereochemical outcome. See the Supporting Information in reference 3b.
28. For in situ monitoring of the rearrangement by 1H NMR in CD30D, see the Supporting Information.
29. Deoxygenation suppresses the formation of minor side products derived from oxidation at the N8-methyl. For example, when (−)-44 was treated with TASF in non-degassed N,N-dimethylformamide, 5% (+)-6 and 1% (−)-5 were isolated in addition to 86% of (−)-4.
30. (a) Numata, A.; Takahashi, C.; Ito, Y.; Takada, T.; Kawai, K.; Usami, Y.; Matsumura, E.; Imachi, M.; Ito, T.; Hasegawa, T. Tetrahedron Lett. 1993, 34, 2355. (b) Jadulco, R.; Edrada, R. A.; Ebel, R.; Berg, A.; Schaumann, K.; Wray, V.; Steube, P.; Proksch, P. J Nat. Prod. 2004, 67, 78. (c) Hayashi, H.; Matsumoto, H.; Akiyama, K. Biosci., Biotechnol., Biochem. 2004, 68, 753. (d) Andersen, B.; Smedsgaard, J.; Frisvad, J. C. J. Agric. Food Chem. 2004, 52, 2421. (e) Dalsgaard, P. W.; Blunt, J. W.; Munro, M. G. H.; Frisvad, J. C.; Christophersen, C. J. Nat. Prod. 2005, 68, 258. (f) Fan, Y.-Q.; Li, P.-H.; Chao, Y.-X.; Chen, H.; Du, N.; He, Q.-X.; Liu, K.-C. Mar. Drugs. 2015, 13, 6489. For the structurally related perophoramidine, see: Verbitski, S. M.; Mayne, C. L.; Davis, R. A.; Concepcion, G. P.; Ireland, C. M. J. Org. Chem. 2002, 67, 7124.
31. For examples of chromium mediated oxidation of N-methyl amines to the corresponding formamides, see: (a) Cave, A.; Kan-Fan, C.; Potier, P.; Le Men, J.; Janot, M.-M. Tetrahedron 1967, 23, 4691. (b) Corey, E. J.; Balanson, R. D. J. Am. Chem. Soc. 1974, 96, 6516. (b) He, B.; Song, H.; Du, Y.; Qin, Y. J. Org. Chem. 2009, 74, 298. (c) Wu, H.; Xue, F.; Xiao, X.; Qin, Y. J. Am. Chem. Soc. 2010, 132, 14052. (d) Reference 2f.
32. Chemoselective oxindole reduction of the sulfamide derived from fragment (−)-22 and sulfamate (+)-52 could not be readily achieved thus it was elected to enter the fragment assembly with aminonitrile (+)-38.
33. The filtrate was monitored by TLC (4% diethyl ether in pentane, KMnO4) to ensure complete recovery of the sulfonyl chloride.
34. Procedure adapted from Han, X.; Civiello, R. L.; Fang, H.; Wu, D.; Gao, Q.; Chaturvedula, P. V.; Macor, J. E.; Dubowchik, G. M. J. Org. Chem. 2008, 73, 8502.
35. (a) When the corresponding sulfonyl chloride was used, the yield of sulfonamide S2 was only 20%. (b) For a review of sulfur(VI) fluorides and their use in organic synthesis, see Dong, J.; Krasnova, L.; Finn, M. G.; Sharpless, K. B. Angew. Chem. Int. Ed 2014, 53, 9430.
36. Kandukuri, S. R.; Schiffner, J. A.; Oestreich, M. Angew. Chem. Int. Ed 2012, 51, 1265.
37. Xie, W.; Jiang, G.; Liu, H.; Hu, J.; Pan, X.; Zhang, H.; Wan, X.; Lai, Y.; Ma, D. Angew. Chem. Int. Ed 2013, 52, 12924.
38. Hoffmann, S.; Seayad, A. M.; List, B. Angew. Chem., Int. Ed 2005, 44, 7424.
39. Further elution with 60% ethyl acetate in hexanes enables recovery of the (S)-TRIP catalyst.
40. Roizen, J. L.; Zalatan, D. L.; Du Bois, J. Angew. Chem. Int. Ed 2013, 52, 11343.
41. Prepared from tert-butyl 2-(trimethylsilyl)ethyl sulfoxide, according to Schwan, A. L.; Brillon, D.; Dufault, R. Can. J. Chem. 1994, 72, 325.
42. During this time, the internal temperature of the reaction mixture did not exceed −75° C.
43. For relevant studies on the use of (−)-diacetone-D-glucose (DAG) as a chiral controller for the preparation of chiral sulfoxides and sulfinamides, see: (a) Llera, J. M.; Fernández, I.; Alcudia, F. Tetrahedron Lett. 1991, 32, 7299. (b) Fernández, I.; Khiar, N.; Llera, J. M.; Alcudia, F. J Org. Chem. 1992, 57, 6789. (c) Khiar, N.; Fernández, I.; Alcudia, F. Tetrahedron Lett. 1994, 35, 5719. (d) Fernández, I.; Valdivia, V.; Khiar, N. J. Org. Chem. 2008, 73, 745. (e) Chelouan, A.; Recio, R.; Alcudia, A.; Khiar, N.; Fernández, I. Eur. J. Org. Chem. 2014, 6935.
44. The absolute configuration of alkanesulfinate (−)-S4 at sulfur was inferred by comparison to literature precedent (see ref. 43).
45. For structural characterization, a sample of (−)-S4 was purified by flash column chromatography on silica gel (eluent: 30→40% diethyl ether in hexanes). ¹H NMR (500 MHz, CDCl₃, 20° C., 97:3 dr): δ 5.90 (d, J=3.6 Hz, 1H), 4.73 (d, J=2.8 Hz, 1H), 4.62 (d, J=3.6 Hz, 1H), 4.32-4.23 (m, 2H), 4.08 (dd, J=8.5, 6.0 Hz, 1H), 4.00 (dd, J=8.5, 5.1 Hz, 1H), 2.79-2.62 (m, 2H), 1.50 (s, 3H), 1.42 (s, 3H), 1.33 (s, 3H), 1.30 (s, 3H), 0.94-0.74 (m, 2H), 0.04 (s, 9H). δ ¹³C NMR (150.9 MHz, CDCl₃, 20° C., 97:3 dr): δ 112.5, 109.3, 105.1, 83.8, 80.5, 79.0, 72.5, 66.8, 53.0, 26.9, 26.8, 26.4, 25.3, 7.6, −1.8. FTIR (thin film) cm⁻¹: 2988 (s), 2897 (m), 1456 (w), 1374 (s), 1251 (s), 1216 (s), 1164 (s), 1135 (s), 1075 (s), 1023 (s), 953 (m). HRMS (DART) (m/z): calc'd for C₁₇H₃₃O₇SSi [M+H]: 409.1716, found: 409.1715. [α]_{D}²³: −56 (c=1.51, CH₂Cl₂).
46. The absolute configuration of sulfinamide (−)-26 was inferred by comparison to literature precedent (see ref. 43) and by preparation of a derivative with a known stereochemical configuration as described later in this document.
47. (−)-Diacetone-D-glucose (12.4 g, 92.6%) was also recovered as an amorphous white solid, which can be recycled without further purification.
48. Strem Chemicals Inc. cat#93-2209, containing 5-15% isopropanol, was dispensed assuming 85 w/w % purity.
49. Sin, N.; Venables, B. L.; Liu, X.; Huang, S.; Gao, Q.; Ng, A.; Dalterio, R.; Rajamani, R.; Meanwell, N. A. J. Heterocyclic Chem. 2009, 46, 432.
50. (a) Sigma-Aldrich, cat#452874 (granular, 10.40 mesh, 98%). (b) When powdered sodium borohydride (5 equiv, Sigma-Aldrich cat#452882) was used, the intermediate aldehyde was often also observed, necessitating the addition of further reducing agent.
51. The same optical rotation was observed when (−)-S5 was prepared from the (Ss)-tert-butanesulfinamide analogue (compound (+)-44 in Lathrop, S. P.; Pompeo, M.; Chang, W.-T. T.; Movassaghi, M. J. Am. Chem. Soc. 2016, 138, 7763), which confirms the absolute stereochemical configuration of (−)-(S)-2-(trimethylsilyl)ethanesulfinamide (26). A representative procedure is as follows: A solution of hydrogen chloride in 1,4-dioxane (4.0 M, 81.0 μL, 324 μmol, 1.99 equiv) was added dropwise via syringe to a solution of the (S)-tert-butane sulfinamide derivative (63.3 mg, 163 μmol, 1 equiv) in methanol (3.30 mL) at 23° C. After 3.2 h, a saturated aqueous sodium bicarbonate solution (15 mL) and deionized water (5 mL) were added and the mixture was extracted with dichloromethane (3×10 mL). The combined organic extracts were washed with a saturated sodium chloride solution (20 mL), were dried over anhydrous sodium sulfate, were filtered, and were concentrated under reduced pressure. The resulting residue was purified by flash column chromatography on silica gel (eluent: 40%→50% acetone in dichloromethane) to afford amino alcohol (−)-S5 (16.7 mg, 35.9%) as a colourless film. [α]_{D}²³=−10 (c=0.83, CH₂Cl₂).
52. Acquisition of NMR spectra in DMSO-d6 at 90° C. resulted in simplification of the spectra by convergence of the signals derived from various conformational isomers. However, gradual sample decomposition during extended acquisition time was observed.
53. All glassware used for the epoxidation reaction was carefully washed to remove trace metals, which may catalyze the decomposition of H₂O₂. Round-bottom flasks, Erlenmeyer flasks, and graduated cylinders used to prepare any component of the reaction mixture were washed successively with concentrated aqueous nitric acid, a saturated aqueous Na4EDTA solution, and acetone (three times each), rinsing with deionized water between each component.
54. Sigma-Aldrich, cat#367877, 99.995% trace metal basis.
55. Sigma-Aldrich, cat#431788, 99.995% trace metal basis.
56. Sigma-Aldrich, cat#216763, 30 wt % in H2O with inhibitor (ACS reagent grade).
57. Acquisition of NMR spectra in DMSO-d6 at 130° C. resulted in simplification of the spectra by convergence of the signals derived from various conformational isomers. However, gradual sample decomposition with heating during extended acquisition time was observed.
58. Atropisomerism causes significant signal broadening and not all 13C resonances were observed. All expected 13C signals were observed in the product of the next step of synthesis, aminonitrile sulfamide (+)-40.
59. The reagent was prepared using a procedure adapted from Lengyel, I.; Cesare, V.; Stephani, R. Synth. Commun. 1998, 28, 1891. t-Butyl hypochlorite (3.68 mL, 32.5 mmol, 1.30 equiv) was added dropwise via syringe to a stirred solution of N-methylbenzamide (3.38 g, 25.0 mmol, 1 equiv) in dichloromethane (50.0 mL) at 0° C. in the dark. After 10 min, the ice-bath was removed and the pale-yellow solution was allowed to stir at 23° C. in the dark. After 67 h, the solution was concentrated under reduced pressure. The resulting residue was purified by flash column chromatography on silica gel (eluent: 15→20% diethyl ether in pentane) to yield N-chloro-N-methylbenzamide (3.97 g, 93.5%) as a pale-yellow oil. Spectral data were consistent with those reported in Lathrop, S. P.; Pompeo, M.; Chang, W.-T. T.; Movassaghi, M. J. Am. Chem. Soc. 2016, 138, 7763.
60. As a result of the sensitivity of this intermediate, its slow conformational equilibrium at ambient temperature, and its instability at elevated temperatures, diazene 21 was used immediately in the next step.
61. (a) Literature value: [α]_{D}²²=−58 (c=0.14, CHCl₃), see Numata, A.; Takahashi, C.; Ito, Y.; Takada, T.; Kawai, K.; Usami, Y.; Matsumura, E.; Imachi, M.; Ito, T.; Hasegawa, T. Tetrahedron Lett. 1993, 34, 2355. (b) Literature value: [α]_{D}²⁰=−174 (c=1.34, CHCl₃), see Hayashi, H.; Matsumoto, H.; Akiyama, K. Biosci. Biotechnol. Biochem. 2004, 68, 753. (c) Literature value: [α]_{D}³⁰=−163.5 (c=0.14, CHCl₃), see Zuo, Z.; Ma, D. Angew. Chem. Int. Ed. 2011, 50, 12008.
62. Numata, A.; Takahashi, C.; Ito, Y.; Takada, T.; Kawai, K.; Usami, Y.; Matsumura, E.; Imachi, M.; Ito, T.; Hasegawa, T. Tetrahedron Lett. 1993, 34, 2355.
63. The reference points for the residual protium and carbon resonances of the NMR solvent were not listed.
64. Hayashi, H.; Matsumoto, H.; Akiyama, K. Biosci. Biotechnol. Biochem. 2004, 68, 753.
65. Resonance frequencies and reference points for the residual protium and carbon resonances of the NMR solvent were not listed.
66. Zuo, Z.; Ma, D. Angew. Chem. Int. Ed. 2011, 50, 12008.
67. Similar chemical shift discrepancies for the C2 and C1″ resonances were noted by Ma and Zuo in their total synthesis report (ref 66).
68. More than the expected 33 13C resonances were observed due to the presence of multiple atropisomers. All observed resonances are listed.
69. Literature value: [α]_{D}²⁰=−156 (c=0.11, CHCl₃), see Hayashi, H.; Matsumoto, H.; Akiyama, K. Biosci. Biotechnol. Biochem. 2004, 68, 753.
70. The revised assignment of C3a and C3a′ resonances is supported by key gHMBC correlations (¹H,¹³C) in ppm: (6.75-6.66, 52.00), (3.89, 52.00), (6.17, 52.54), and (3.46, 52.54).
71. The reagent was prepared as follows: oxalyl chloride (2.18 mL, 25 mmol, 1 equiv) was added dropwise to a solution of sorbic acid (5.61 g, 50 mmol, 2.00 equiv), triethylamine (6.97 mL, 50 mmol, 2.00 equiv), and N,N-dimethylformamide (19.0 μL, 250 μmol, 0.0100 equiv) in dichloromethane (250 mL) at 0° C., during which time gentle gas evolution was noted. After 20 min, the ice bath was removed and the solution was allowed to stir at 23° C. After 6 h, the mixture was diluted with a saturated aqueous ammonium chloride solution (200 mL) and deionized water (100 mL). The layers were separated and the aqueous layer was extracted with dichloromethane (2×100 mL). The combined organic extracts were washed with a saturated aqueous sodium chloride solution (250 mL), were dried over anhydrous sodium sulfate, were filtered, and were concentrated under reduced pressure. The resulting residue was purified by flash column chromatography on silica gel (eluent: 60→80% dichloromethane in pentane) to afford sorbic anhydride (4.41 g, 85.4%) as a pale-yellow oil, which solidified on standing to an off-white waxy solid. ¹H NMR (500 MHz, CDCl₃): 7.43-7.30 (m, 2H), 6.32-6.17 (m, 4H), 5.81 (d, J=15.2 Hz, 2H), 1.89 (d, J=5.1 Hz, 6H). ¹³C NMR (125.8 MHz, CDCl₃): 163.0, 148.9, 142.4, 129.8, 117.8, 19.0. Spectral data were in agreement with those previously reported in the literature: Honda, T.; Namiki, H.; Kudoh, M.; Nagase, H.; Mizutani, H. Heterocycles 2003, 59, 169.
72. (a) Literature value: [α]_{D}²²=+8.7 (c=0.23, CHCl₃), see Numata, A.; Takahashi, C.; Ito, Y.; Takada, T.; Kawai, K.; Usami, Y.; Matsumura, E.; Imachi, M.; Ito, T.; Hasegawa, T. Tetrahedron Lett. 1993, 34, 2355. (b) Literature value: [α]_{D}=−58 (c=0.10, MeOH), see Jadulco, R.; Edrada, R. A.; Ebel, R.; Berg, A.; Schaumann, K.; Wray, V.; Steube, K.; Proksch, P. J. Nat. Prod. 2004, 67, 78. (c) Literature value: [α]_{D}²⁰=−74.9 (c=1.50, CHCl₃), see Hayashi, H.; Matsumoto, H.; Akiyama, K. Biosci. Biotechnol. Biochem. 2004, 68, 753. (d) Literature value: [α]_{D}³⁰=−51.3 (c=0.30, CHCl₃), see Zuo, Z.; Ma, D. Angew. Chem. Int. Ed. 2011, 50, 12008.
73. Numata, A.; Takahashi, C.; Ito, Y.; Takada, T.; Kawai, K.; Usami, Y.; Matsumura, E.; Imachi, M.; Ito, T.; Hasegawa, T. Tetrahedron Lett. 1993, 34, 2355.
74. The reference points for the residual protium and carbon resonances of the NMR solvent were not listed.
75. Hayashi, H.; Matsumoto, H.; Akiyama, K. Biosci. Biotechnol. Biochem. 2004, 68, 753.
76. Resonance frequencies and reference points for the residual protium and carbon resonances of the NMR solvent were not listed.
77. Zuo, Z.; Ma, D. Angew. Chem. Int. Ed. 2011, 50, 12008.
78. The reported integrals are an approximation due to the presence of multiple conformers and significant atropisomerism.
79. More than the expected 37 ¹³C resonances were observed due to the presence of multiple atropisomers. All observed resonances are listed.
80. Literature value: [α]D=−30 (c=0.038, MeOH), see Jadulco, R.; Edrada, R. U.; Ebel, R.; Berg, A.; Schaumann, K.; Wray, V.; Steube, K.; Proksch, P. J. Nat. Prod. 2004, 67, 78.
81. Jadulco, R.; Edrada, R. A.; Ebel, R.; Berg, A.; Schaumann, K.; Wray, V.; Steube, K.; Proksch, P. J. Nat. Prod 2004, 67, 78.
82. No ¹³C-NMR spectroscopic data were tabulated for the natural sample of (−)-5 in the isolation report. Based on analysis of the ¹H-NMR, gCOSY, and HRMS data, the authors state: “It was therefore clear that the new derivative [communesin C] is the N-demethyl derivative of [communesin B].”
83. These resonances were reported to be concealed by the residual water signal.
84. Literature value: [α]_{D}²⁰=+150 (c=0.14, CHCl₃), see Hayashi, H.; Matsumoto, H.; Akiyama, K. Biosci. Biotechnol. Biochem. 2004, 68, 753.
85. Hayashi, H.; Matsumoto, H.; Akiyama, K. Biosci. Biotechnol. Biochem. 2004, 68, 753.
86. Resonance frequencies and reference points for the residual protium and carbon resonances of the NMR solvent were not listed.
87. Literature value: [α]_{D}²⁵=−157 (c=0.021, MeOH), see Dalsgaard, P. W.; Blunt, J. W.; Munro, M. H. G.; Frisvad, J. C.; Christophersen, C. J. Nat. Prod 2005, 68,258.
88. Dalsgaard, P. W.; Blunt, J. W.; Munro, M. H. G.; Frisvad, J. C.; Christophersen, C. J. Nat. Prod 2005, 68, 258.
89. Proton NMR spectra were referenced from the residual protium in the NMR solvent (CHCl₃: δ 7.25).
90. Carbon-13 NMR spectra are referenced from the carbon resonances of the deuterated solvent (CDCl₃: δ 77.01)
91. The revised assignment of C3a and C3a′ resonances is supported by key gHMBC correlations (¹H, ¹³C) in ppm: (6.06, 51.6), (3.45, 51.6), (6.73, 52.1), and (3.89, 52.1).
92. The revised assignment of C5′ and C7′ resonances is supported by a key 4-bond gHMBC correlation (¹H, ¹³C) in ppm: (4.69, 117.1).
93. Literature value: [α]_{D}²⁵=−167 (c=0.024, MeOH), see Dalsgaard, P. W.; Blunt, J. W.; Munro, M. H. G.; Frisvad, J. C.; Christophersen, C. J. Nat. Prod 2005, 68,258.
94. The revised assignment of C3a and C3a′ resonances is supported by key gHMBC correlations (¹H,¹³C) in ppm: (6.07, 51.7), (3.46, 51.7), (6.73, 52.2), and (3.88, 52.2).
95. The revised assignment of C5′ and C7′ resonances is supported by a key 4-bond gHMBC correlation (¹H,¹³C) in ppm: (4.69, 117.1).
96. Kitir, B.; Baldry, M.; Ingmer, H.; Olsen, C. A. Tetrahedron 2014, 70, 7721.
97. The relative stereochemical configuration of aldol adducts (+)-48 and (+)-49 were determined by polarimetric analysis of the corresponding carboxylic acids after hydrolysis, as described later in this document.
98. Literature value: [α]D=+27 (c=1.2, CHCl3), see Hsiao, C.-N.; Liu, L.; Miller, M. J. J. Org. Chem. 1987, 52, 2201.
99. Literature value: [α]_{D}²⁵=−27.3 (c=2.1, CHCl₃), see Evans, D. A.; Bartroli, J.; Shih, T. L. J. Am. Chem. Soc. 1981, 103, 2127.
100. The structure of (−)-communesin I (10) with revised C3″-configuration is depicted.
101. Literature value: [α]_{D}²⁰=−59 (c=0.1, MeOH), see Fan, Y.-Q.; Li, P.-H.; Chao, Y.-X.; Chen, H.; Du, N.; He, Q.-X.; Liu, K.-C. Mar. Drugs. 2015, 13, 6489.
102. Fan, Y.-Q.; Li, P.-H.; Chao, Y.-X.; Chen, H.; Du, N.; He, Q.-X.; Liu, K.-C. Mar. Drugs. 2015, 13, 6489.
103. Heller, S. T.; Schultz, E. E.; Sarpong, R. Angew. Chem. Int. Ed. 2012, 51, 8304.
104. The reported integrals are an approximation due to the presence of multiple conformers and significant atropisomerism.
105. Atropisomerism causes significant signal broadening and not all ¹³C resonances were observed. All expected ¹³C signals were observed in the product of the next step of the synthesis, heterodimer (+)-55.
106. As a result of the sensitivity of this intermediate, its slow conformational equilibrium at ambient temperature, and its instability at elevated temperatures, diazene 54 was used immediately in the next step.
107. While 56 and 57 can be separated via flash chromatography on silica gel, the mixture was used directly in the next step since 56 rapidly converts to 57 upon treatment with ethanolic lithium tert-butoxide at 23° C. in the subsequent step. See later in this document for in situ monitoring of the rearrangement of pure 56 to (+)-58 by ¹H NMR spectroscopy.

1. A compound of Formula (I): 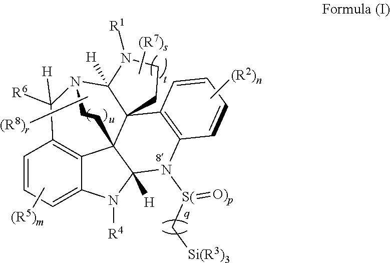
or a salt, tautomer, or stereoisomer thereof, wherein:
R¹ and R⁴ are each independently selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², substituted or unsubstituted aryl, substituted or unsubstituted heteroaryl, substituted or unsubstituted carbocyclyl, and substituted or unsubstituted heterocyclyl;
each instance of R² and R⁵ is independently selected from F, Cl, Br, I, —OH, —OR⁹, —OC(═O)R⁹, —S(═O)_{b}R¹², —NR⁹R¹⁰, substituted or unsubstituted C₂-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl;
each instance of R³ is independently selected from substituted or unsubstituted C₁-C₁₂ alkyl, substituted or unsubstituted aryl, substituted or unsubstituted heteroaryl, substituted or unsubstituted carbocyclyl, or substituted or unsubstituted heterocyclyl;
R⁶ is H, —OH, —OR⁹, —OC(═O)R⁹, —S(═O)_{b}R¹², —NR⁹R¹⁰, substituted or unsubstituted C₁-C₁₂ alkyl, substituted or unsubstituted C₁-C₁₂ heteroalkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, or substituted or unsubstituted heterocyclyl;
each instance of R⁷ and R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl, or wherein two R⁷ or two R⁸ groups taken together with the carbon atoms to which they are attached form a substituted or unsubstituted aryl, substituted or unsubstituted heteroaryl, substituted or unsubstituted carbocyclic, or substituted or unsubstituted heterocyclic ring;
each instance of R⁹ and R¹⁰ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R⁹ and R¹⁰ taken together with the carbon atoms to which they are attached form a substituted or unsubstituted heteroaryl or substituted or unsubstituted heterocyclic ring;
each instance of R¹² is independently substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)_{c}SiMe₃, or —(CH₂)_{c}R⁹;
m and t are each independently an integer from 0 to 3, inclusive;
n, r, and s are each independently an integer from 0 to 4, inclusive;
each instance of c is independently an integer from 0 to 6, inclusive;
each instance of b is independently 0, 1, or 2;
u is 0, 1, or 2;
p is an integer selected from 1 or 2; and
q is an integer from 1 to 6, inclusive.

2. (canceled)

3. The compound of claim 1, or a salt, tautomer, or stereoisomer thereof, wherein R⁶ is 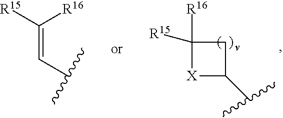
and wherein
X is O, NR⁹, or —S(═O)₂, or S;
v is an integer from 0 to 4, inclusive; and
each instance of R¹⁵ and R¹⁶ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R¹⁵ and R¹⁶ taken together with the carbon atoms to which they are attached form a substituted or unsubstituted carbocyclic, or substituted or unsubstituted heterocyclic ring.

4. (canceled)

5. The compound of claim 1, or a salt, tautomer, or stereoisomer thereof, wherein R⁴ is H, —C(═O)R⁹, substituted or unsubstituted C₁-C₁₂ alkyl, substituted or unsubstituted aryl, or substituted or unsubstituted heteroaryl.

6-9. (canceled)

10. The compound of claim 1, or a salt, tautomer, or stereoisomer thereof, wherein p is 2.

11-12. (canceled)

13. The compound of claim 1, or a salt, tautomer, or stereoisomer thereof, wherein R¹ is —C(═O)R⁹.

14-15. (canceled)

16. The compound of claim 1, or a salt, tautomer, or stereoisomer thereof, wherein u is 1.

17. The compound of claim 1, or a salt, tautomer, or stereoisomer thereof, wherein t is 1.

18-21. (canceled)

22. The compound of claim 1, wherein the compound is of the formula:

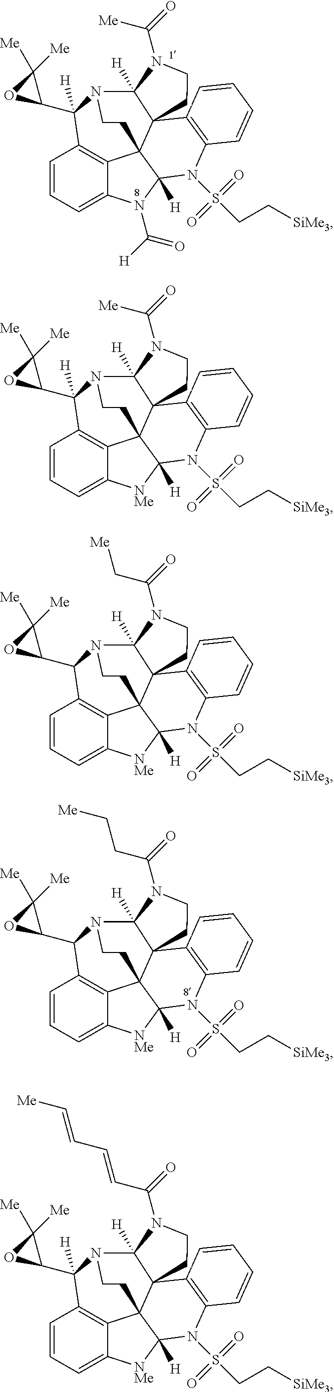

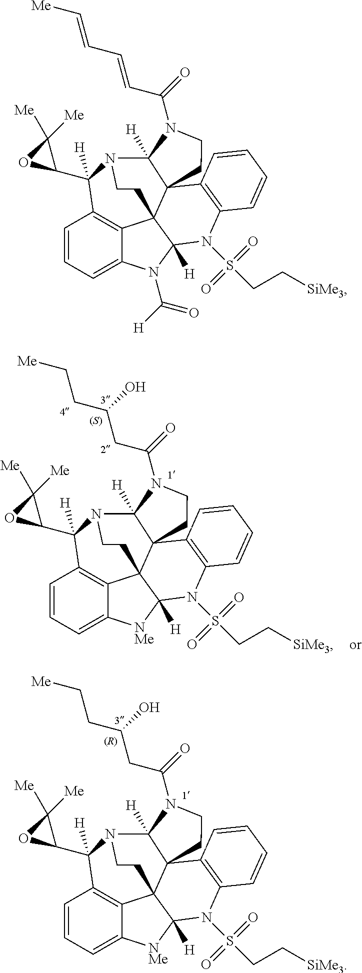

23. A composition comprising a compound of claim 1, or a salt, tautomer, or stereoisomer thereof, and an excipient.

24. (canceled)

25. A method of treating a disease, comprising administering an effective amount of a compound of claim 1, or a pharmaceutically acceptable salt, tautomer, or stereoisomer thereof, to a subject in need thereof.

26. A method of preventing a disease, comprising administering an effective amount of a compound of claim 1, or a pharmaceutically acceptable salt, tautomer, or stereoisomer thereof, to a subject in need thereof.

27-39. (canceled)

40. A method of treating an insect infestation comprising contacting the insect with an effective amount of a compound of claim 1, or a salt, tautomer, or stereoisomer thereof.

41. (canceled)

42. A compound of Formula (V): 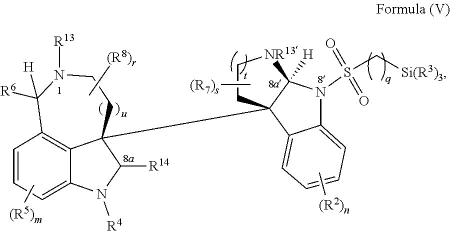
or a salt, tautomer, or stereoisomer thereof, wherein
R⁴ is selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², substituted or unsubstituted aryl, substituted or unsubstituted heteroaryl, substituted or unsubstituted carbocyclyl, and substituted or unsubstituted heterocyclyl;
each instance of R² and R⁵ are independently selected from F, Cl, Br, I, —OH, —OR⁹, —OC(═O)R⁹, —S(═O)_{b}R¹², —NR⁹R¹⁰, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted and heterocyclyl;
each instance of R³ is independently selected from substituted or unsubstituted C₁-C₁₂ alkyl, substituted or unsubstituted aryl, substituted or unsubstituted heteroaryl, substituted or unsubstituted carbocyclyl, and substituted or unsubstituted heterocyclyl;
R⁶ is 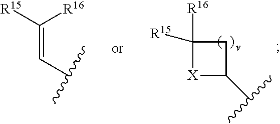
X is O, NR⁹, or —S(═O)_{b}R¹²;
each instance of R⁷ and R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl, or wherein two R⁷ or two R⁸ groups taken together with the carbon atoms to which they are attached form a substituted or unsubstituted aryl, substituted or unsubstituted heteroaryl, substituted or unsubstituted carbocyclic, or substituted or unsubstituted heterocyclic ring;
each instance of R⁹ and R¹⁰ are independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R⁹ and R¹⁰ taken together with the carbon atoms to which they are attached form a substituted or unsubstituted heteroaryl or substituted or unsubstituted heterocyclic ring;
each instance of R¹² is independently substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)_{c}SiMe₃, or —(CH₂)_{c}R⁹;
R¹³ and R^{13′} are each independently hydrogen, 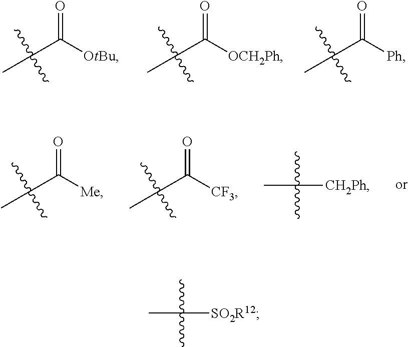
R¹⁴ is —CN, —OH, —OR⁹, —NR⁹R¹⁰, S(═O)_{b}R¹², or P(═O)(OR⁹)₂
each instance of R¹⁵ and R¹⁶ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R¹⁵ and R¹⁶ taken together with the carbon atoms to which they are attached form a substituted or unsubstituted carbocyclic ring, or substituted or unsubstituted heterocyclic ring;
m and t are each independently an integer from 0 to 3, inclusive;
n, r, and s are each independently an integer from 0 to 4, inclusive;
v is an integer from 0 to 4, inclusive;
each instance of c is independently an integer from 0 to 6, inclusive;
each instance of b is independently 0, 1, or 2;
u is 0, 1, or 2; and
q is an integer from 1 to 6, inclusive.

43. A method of making a compound of claim 1, or a salt, tautomer, or stereoisomer thereof, comprising forming a bond between the nitrogen atom at the position N1 and the carbon atom at the position C8a′, and a bond between the nitrogen atom at the position N8′ and the carbon atom at the position C8a in a compound of claim 42, or a salt, tautomer, or stereoisomer thereof.

44-49. (canceled)

50. A compound of Formula (III′): 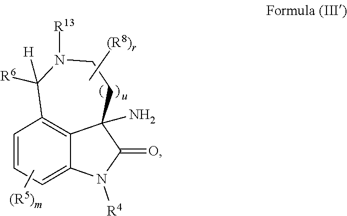
or a salt, tautomer, or stereoisomer thereof, wherein
R⁴ is selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², substituted or unsubstituted aryl, substituted or unsubstituted heteroaryl, substituted or unsubstituted carbocyclyl, and substituted or unsubstituted heterocyclyl;
each instance of R⁵ is independently selected from F, Cl, Br, I, —OH, —OR⁹, —OC(═O)R⁹, —S(═O)_{b}R¹², —NR⁹R¹⁰, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl;
R⁶ is 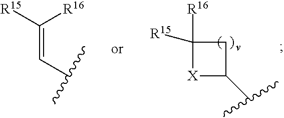
X is O, NR⁹, or —S(═O)_{b}R¹²;
each instance of R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl;
each instance of R⁹ and R¹⁰ is independently selected from H, C₁-C₁₂ alkyl, C₂-C₁₂ alkenyl, C₂-C₁₂ alkynyl, aryl, heteroaryl, carbocyclyl, and heterocyclyl, or wherein R⁹ and R¹⁰taken together with the carbon atoms to which they are attached form a substituted or unsubstituted heteroaryl or substituted or unsubstituted heterocyclic ring;
each instance of R¹² is independently substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)_{c}SiMe₃, or —(CH₂)_{c}R⁹;
R¹³ is 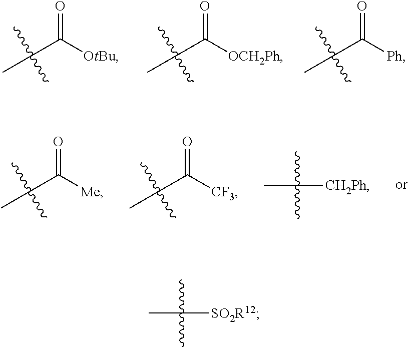
each instance of R¹⁵ and R¹⁶ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R¹⁵ and R¹⁶ taken together with the carbon atoms to which they are attached form a substituted or unsubstituted carbocyclic, or substituted or unsubstituted heterocyclic ring;
m is an integer from 0 to 3, inclusive;
v is an integer from 0 to 4, inclusive;
r is an integer from 0 to 4, inclusive;
each instance of c is independently an integer from 0 to 6, inclusive;
each instance of b is independently 0, 1, or 2; and
u is 0, 1, or 2.

51. A method of making a compound of claim 50, comprising desulfonylation of the sulfonamide at C3a in a compound of Formula (IX): 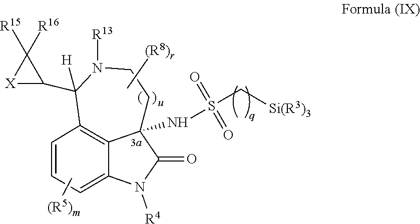
or a salt, tautomer, or stereoisomer thereof.

52-59. (canceled)

60. A compound of Formula (XIV): 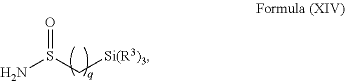
or a salt, tautomer, or stereoisomer thereof, wherein
each instance of R³ is independently substituted or unsubstituted C₁-C₁₂ alkyl, substituted or unsubstituted aryl, substituted or unsubstituted heteroaryl, substituted or unsubstituted carbocyclyl, and substituted or unsubstituted heterocyclyl; and
q is an integer from 1 to 6, inclusive.

61-62. (canceled)

63. A method of making a compound of claim 60, or a salt, tautomer, or stereoisomer thereof, comprising the steps of:
(1) reacting 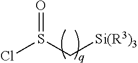
with a chiral controller in the presence of a base, wherein the chiral controller comprises a hydroxy moiety; and
(2) reacting with an H₂N⁻ source.

64-65. (canceled)

66. A method of making a compound of Formula (I′): 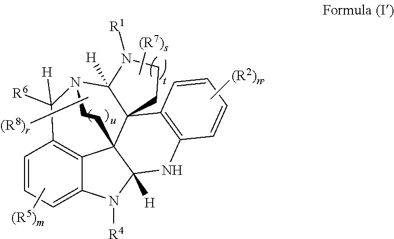
or a salt, tautomer, or stereoisomer thereof, wherein
R¹ and R⁴ are each independently selected from H, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², substituted or unsubstituted aryl, substituted or unsubstituted heteroaryl, substituted or unsubstituted carbocyclyl, and substituted or unsubstituted heterocyclyl;
each instance of R² and R⁵ is independently selected from F, Cl, Br, I, —OH, —OR⁹, —OC(═O)R⁹, —S(═O)_{b}R¹², —NR⁹R¹⁰, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl;
R⁶ is H, —OH, —OR⁹, —OC(═O)R⁹, —S(═O)_{b}R¹², —NR⁹R¹⁰, substituted or unsubstituted C₁-C₁₂ alkyl, substituted or unsubstituted C₁-C₁₂ heteroalkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, or substituted or unsubstituted heterocyclyl;
each instance of R⁷ and R⁸ is independently selected from H, halogen, substituted or unsubstituted C₁-C₁₂ alkyl, substituted or unsubstituted C₂-C₁₂ alkenyl, substituted or unsubstituted C₂-C₁₂ alkynyl, —C(═O)R⁹, —C(═O)NR⁹R¹⁰, —S(═O)_{b}R¹², —OH, —OR⁹, —OC(═O)R⁹, —NR⁹R¹⁰, substituted or unsubstituted aryl, substituted or unsubstituted heteroaryl, substituted or unsubstituted carbocyclyl, and substituted or unsubstituted heterocyclyl, or wherein two R⁷ or two R⁸ groups taken together with the carbon atoms to which they are attached form a substituted or unsubstituted aryl, substituted or unsubstituted heteroaryl, substituted or unsubstituted carbocyclic, or substituted or unsubstituted heterocyclic ring;
each instance of R⁹ and R¹⁰ is independently selected from H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, and substituted or unsubstituted heterocyclyl, or wherein R⁹ and R¹⁰ taken together with the carbon atoms to which they are attached form a substituted or unsubstituted heteroaryl or substituted or unsubstituted heterocyclic ring;
each instance of R¹² is independently substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heteroaryl, substituted or unsubstituted carbocyclyl, substituted or unsubstituted heterocyclyl, —(CH₂)_{c}SiMe₃, or —(CH₂)_{c}R⁹;
m and t are each independently an integer from 0 to 3, inclusive;
n, r, and s are each independently an integer from 0 to 4, inclusive;
each instance of c is independently an integer from 0 to 6, inclusive;
each instance of b is independently 0, 1, or 2; and
u is 0, 1, or 2;
comprising desulfonylation of position N8′ in a compound of claim 1, or a salt, tautomer, or stereoisomer thereof.

67-70. (canceled)

71. A compound of formula: 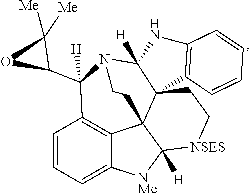
or a salt, tautomer, or stereoisomer thereof.
